SEQUENCE LISTING (2 sequences)

SEQ ID NO: 1 - protein, 645 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 226 aa, synthetic construct
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

(19) United States
(12) Patent Application Publication
(10) Pub. No.: US 2026/0132215 A1
(43) Pub. Date: May 14, 2026

(54) COMPOSITIONS AND METHODS RELATED TO TUMOR ACTIVATED ANTIBODIES TARGETING PSMA AND EFFECTOR CELL ANTIGENS

(71) Applicant: Janux Therapeutics, Inc., San Diego, CA (US)

(72) Inventors: David CAMPBELL, San Diego, CA (US); Byron ROBINSON, San Diego, CA (US); Zachariah Augustus MCIVER, San Diego, CA (US)

(21) Appl. No.: 19/444,725

(22) Filed: Jan. 9, 2026

Related U.S. Application Data
(63) Continuation of application No. PCT/US24/37777, filed on Jul. 12, 2024.
(60) Provisional application No. 63/513,625, filed on Jul. 14, 2023.
(60) Provisional application No. 63/557,413, filed on Feb. 23, 2024.

(51) Int. Cl. C07K 16/30; A61K 31/4164; A61K 31/454; A61K 31/502; A61K 31/5025; A61K 31/55; A61K 31/58; A61K 39/00; A61K 45/06; A61K 51/02; A61K 51/08; A61P 35/00; C07K 16/18; C07K 16/28

(52) U.S. Cl. CPC C07K 16/3069; A61K 31/4164; A61K 31/454; A61K 31/502; A61K 31/5025; A61K 31/55; A61K 31/58; A61K 45/06; A61K 51/025; A61K 51/088; A61P 35/00; C07K 16/18; C07K 16/2809; C07K 16/2818; A61K 2039/505; A61K 2039/545; C07K 2317/24; C07K 2317/31; C07K 2317/55; C07K 2317/569; C07K 2317/622; C07K 2317/73; C07K 2317/92; C07K 2317/94

(57) ABSTRACT

Described herein are compositions and formulations comprising a recombinant polypeptide. Related methods and uses of these compositions and formulations are also described herein.

### CROSS REFERENCE(S)

The present application is a continuation of International Patent Application No. PCT/US24/37777, filed Jul. 12, 2024, which claims the benefit of U.S. Provisional Application No. 63/513,625 filed on Jul. 14, 2023, and U.S. Provisional Application No. 63/557,413 filed on Feb. 23, 2024, each of which are incorporated herein by reference in their entirety.

### SEQUENCE LISTING

The instant application contains a Sequence Listing which has been submitted electronically in XML format and is hereby incorporated by reference in its entirety. Said XML copy, created on Jun. 26, 2024 is named 52426-756_301SL.xml and is 3,671 bytes in size.

### SUMMARY

Disclosed herein, in one aspect, is a method for treating cancer comprising administering to a subject in need thereof a first dose and a target dose of an isolated recombinant polypeptide complex, wherein the first dose is at least about 100 μg and the target dose is higher than the first dose, and wherein the isolated recombinant polypeptide complex comprises a first chain with an amino acid sequence having at least 80% sequence identity to SEQ ID NO: 1 and a second chain with an amino acid sequence having at least 80% sequence identity to SEQ ID NO: 2. In some embodiments, the first dose is about 100 μg to about 1 mg. In some embodiments, the first dose is at least about 150 μg. In some embodiments, the first dose is at least about 200 μg. In some embodiments, the first dose is at least about 250 μg. In some embodiments, the first dose is at least about 300 μg. In some embodiments, the first dose is at least about 400 μg. In some embodiments, the first dose is at least about 500 μg. In some embodiments, the first dose is at least about 600 μg. In some embodiments, the first dose is at least about 700 μg. In some embodiments, the first dose is at least about 800 μg. In some embodiments, the first dose is at least about 900 μg. In some embodiments, the first dose is about 100 μg. In some embodiments, the first dose is about 150 μg. In some embodiments, the first dose is about 200 μg. In some embodiments, the first dose is about 250 μg. In some embodiments, the first dose is about 300 μg. In some embodiments, the first dose is about 400 μg. In some embodiments, the first dose is about 500 μg. In some embodiments, the first dose is about 600 μg. In some embodiments, the first dose is about 700 μg. In some embodiments, the first dose is about 800 μg. In some embodiments, the first dose is about 900 μg. In some embodiments, the first dose is about 1 mg. In some embodiments, the target dose is about 450 μg to about 10 mg. In some embodiments, the target dose is at least about 450 μg. In some embodiments, the target dose is at least about 750 μg. In some embodiments, the target dose is at least about 1 mg. In some embodiments, the target dose is at least about 1.5 mg. In some embodiments, the target dose is at least about 2 mg. In some embodiments, the target dose is at least about 2.5 mg. In some embodiments, the target dose is at least about 3 mg. In some embodiments, the target dose is at least about 4 mg. In some embodiments, the target dose is at least about 5 mg. In some embodiments, the target dose is at least about 6 mg. In some embodiments, the target dose is at least about 7 mg. In some embodiments, the target dose is at least about 8 mg. In some embodiments, the target dose is at least about 9 mg. In some embodiments, the target dose is about 450 μg. In some embodiments, the target dose is about 750 μg. In some embodiments, the target dose is about 1 mg. In some embodiments, the target dose is about 1.5 mg. In some embodiments, the target dose is about 2 mg. In some embodiments, the target dose is about 2.5 mg. In some embodiments, the target dose is about 3 mg. In some embodiments, the target dose is about 4 mg. In some embodiments, the target dose is about 5 mg. In some embodiments, the target dose is about 6 mg. In some embodiments, the target dose is about 7 mg. In some embodiments, the target dose is about 8 mg. In some embodiments, the target dose is about 9 mg. In some embodiments, the target dose is about 10 mg.

In some embodiments, the method further comprises administering to the subject a second dose of the isolated recombinant polypeptide complex, wherein the second dose is higher than the first dose and lower than the target dose, and wherein the second dose is administered between the first dose and the target dose. In some embodiments, the second dose is about 300 μg to about 5 mg. In some embodiments, the second dose is at least about 300 μg. In some embodiments, the second dose is at least about 400 μg. In some embodiments, the second dose is at least about 500 μg. In some embodiments, the second dose is at least about 600 μg. In some embodiments, the second dose is at least about 700 μg. In some embodiments, the second dose is at least about 800 μg. In some embodiments, the second dose is at least about 900 μg. In some embodiments, the second dose is at least about 1 mg. In some embodiments, the second dose is at least about 2 mg. In some embodiments, the second dose is at least about 3 mg. In some embodiments, the second dose is at least about 4 mg. In some embodiments, the second dose is about 300 μg. In some embodiments, the second dose is about 400 μg. In some embodiments, the second dose is about 500 μg. In some embodiments, the second dose is about 600 μg. In some embodiments, the second dose is about 700 μg. In some embodiments, the second dose is about 800 μg. In some embodiments, the second dose is about 900 μg. In some embodiments, the second dose is about 1 mg. In some embodiments, the second dose is about 1.2 mg. In some embodiments, the second dose is about 1.5 mg. In some embodiments, the second dose is about 2 mg. In some embodiments, the second dose is about 2.5 mg. In some embodiments, the second dose is about 3 mg. In some embodiments, the second dose is about 3.5 mg. In some embodiments, the second dose is about 4 mg. In some embodiments, the second dose is about 4.5 mg. In some embodiments, the second dose is about 5 mg.

In some embodiments, the first dose, the second dose, and the target dose are about 100 μg, 300 μg, and 450 μg, respectively. In some embodiments, the first dose, the second dose, and the target dose are about 100 μg, 300 μg, and 1 mg, respectively. In some embodiments, the first dose, the second dose, and the target dose are about 200 μg, 600 μg, and 2 mg, respectively. In some embodiments, the first dose, the second dose, and the target dose are about 300 μg, 1 mg, and 3 mg, respectively. In some embodiments, the first dose, the second dose, and the target dose are about 400 μg, 1.2 mg, and 4 mg, respectively. In some embodiments, the first dose, the second dose, and the target dose are about 500 μg, 1.5 mg, and 5 mg, respectively. In some embodiments, the first dose, the second dose, and the target dose are about 600 μg, 2 mg, and 6 mg, respectively. In some embodiments, the first dose, the second dose, and the target dose are about 700 μg, 2.5 mg, and 7 mg, respectively. In some embodiments, the first dose, the second dose, and the target dose are about 800 μg, 3 mg, and 8 mg, respectively. In some embodiments, the first dose, the second dose, and the target dose are about 900 μg, 4 mg, and 9 mg, respectively. In some embodiments, the first dose, the second dose, and the target dose are about 1 mg, 5 mg, and 10 mg, respectively. In some embodiments, the first dose, the second dose, or the target dose is administered weekly. In some embodiments, the first dose, the second dose, or the target dose is administered once every two weeks. In some embodiments, the first dose, the second dose, or the target dose is administered once every three weeks. In some embodiments, the first dose, the second dose, and the target dose are administered weekly. In some embodiments, the first dose and the second dose are administered weekly, and wherein the target dose is administered once every two weeks. In some embodiments, the first dose and the second dose are administered weekly, and wherein the target dose is administered once every three weeks. In some embodiments, the method comprises a treatment cycle that starts on day 1. In some embodiments, the treatment cycle is 21 days or 28 days. In some embodiments, the first dose, the second dose, and the target dose are administered on day 1, one of day 7, 8 or 9, and one of day 14, 15, or 16, respectively, of the treatment cycle. In some embodiments, the first dose, the second dose, and the target dose are administered on day 1, day 4, and day 8, respectively, of the treatment cycle. In some embodiments, the target dose is administered weekly after the one of day 14, 15, or 16, of the treatment cycle. In some embodiments, the target dose is administered once every two weeks after the one of day 14, 15, or 16, of the treatment cycle. In some embodiments, the target dose is administered once every three weeks after the one of day 14, 15, or 16, of the treatment cycle. In some embodiments, the target dose is administered weekly after day 8 of the treatment cycle. In some embodiments, the target dose is administered once every two weeks after day 8 of the treatment cycle. In some embodiments, the target dose is administered once every three weeks after day 8 of the treatment cycle. In some embodiments, the target dose is administered at least once, at least twice, at least 3 times, at least 4 times, at least 5 times, at least 6 times, at least 7 times, at least 8 times, at least 9 times, at least 10 times, at least 15 times, at least 20 times, at least 25 times, at least 30 times, at least 35 times, at least 40 times, at least 45 times, at least 50 times, or more than 50 times. In some embodiments, the method further comprises at least 2 treatment cycles, at least 3 treatment cycles, at least 4 treatment cycles, at least 5 treatment cycles, at least 6 treatment cycles, at least 7 treatment cycles, at least 8 treatment cycles, at least 9 treatment cycles, at least 10 treatment cycles, or more than 10 treatment cycles. In some embodiments, the first dose, the second dose, and the target dose are administered on day 1, the one of day 7, 8 or 9, and the one of day 14, 15, or 16, respectively, of the treatment cycle, and wherein the target dose is administered weekly after day 14, 15, or 16 of the treatment cycle. In some embodiments, the first dose, the second dose, and the target dose are administered on day 1, the one of day 7, 8 or 9, and the one of day 14, 15, or 16, respectively, of the treatment cycle, and wherein the target dose is administered once every two weeks after day 14, 15, or 16 of the treatment cycle. In some embodiments, the first dose, the second dose, and the target dose are administered on day 1, the one of day 7, 8 or 9, and the one of day 14, 15, or 16, respectively, of the treatment cycle, and wherein the target dose is administered once every three weeks after day 14, 15, or 16 of the treatment cycle. In some embodiments, the first dose, the second dose, and the target dose are administered on day 1, day 4, and day 8, respectively, of the treatment cycle, and wherein the target dose is administered weekly after day 8 of the treatment cycle. In some embodiments, the first dose, the second dose, and the target dose are administered on day 1, day 4, and day 8, respectively, of the treatment cycle, and wherein the target dose is administered once every two weeks after day 8 of the treatment cycle. In some embodiments, the first dose, the second dose, and the target dose are administered on day 1, day 4, and day 8, respectively, of the treatment cycle, and wherein the target dose is administered once every three weeks after day 8 of the treatment cycle.

In some embodiments, the cancer comprises a cell that expresses or overexpresses prostate specific membrane antigen (PSMA). In some embodiments, the cancer comprises cells that are dependent on endothelial cells of neovasculature that express or overexpress PSMA. In some embodiments, the cancer comprises prostate cancer, lung cancer, breast cancer, colorectal cancer, or renal cell carcinoma. In some embodiments, the cancer comprises prostate cancer. In some embodiments, the cancer comprises metastatic castration resistant prostate cancer (mCRPC). In some embodiments, the progression of mCRPC is documented according to guidelines of the Prostate Cancer Clinical Trials Working Group 3 (PCWG3), Response Evaluation Criteria in Solid Tumors 1.1 (RECIST 1.1), or both. In some embodiments, the cancer comprises non-small cell lung cancer (NSCLC), or small cell lung cancer (SCLC). In some embodiments, the subject is a male of at least 18 years old. In some embodiments, the subject is a male of about 46 to about 85 years old. In some embodiments, the subject is a male of about 69 years old. In some embodiments, the subject is treated with a prior cancer therapy before the administering. In some embodiments, the prior cancer therapy comprises 2 to 6 cancer therapies. In some embodiments, the prior cancer therapy comprises about 4 cancer therapies. In some embodiments, the prior cancer therapy comprises a taxane, a radioligand therapy, an anti-androgen therapy, an androgen receptor pathway inhibitor therapy, a poly-adenosine diphosphate ribose polymerase (PARP) inhibitor, or a combination thereof. In some embodiments, the anti-androgen therapy comprises a radioligand therapy that targets PSMA. In some embodiments, the prior cancer therapy comprises a taxane and an anti-androgen therapy. In some embodiments, the cancer is mCRPC, and wherein the mCRPC progresses after the prior cancer therapy. In some embodiments, the prior cancer therapy comprises no taxane. In some embodiments, the prior cancer therapy comprises lutetium Lu 177 vipivotide tetraxetan. In some embodiments, the prior cancer therapy comprises olaparib, rucaparib, niraparib, talazoparib, or a combination thereof. In some embodiments, the subject has adequate organ function. In some embodiments, the subject has resolving acute effects of any prior therapy to baseline severity of Common Terminology Criteria for Adverse Events (CTCAE) grade of no more than 1. In some embodiments, the subject has adenocarcinoma of the prostate that is confirmed histologically or cytologically. In some embodiments, the subject is positive for a baseline prostate-specific membrane antigen positron emission tomography (PSMA-PET). In some embodiments, the subject has a baseline PSA level of about 1 ng/ml to about 10000 ng/ml. In some embodiments, the subject has a baseline PSA level of about 158 ng/ml. In some embodiments, the subject is or is not evaluable according to guidelines of response evaluation criteria in solid tumors 1.1 (RECIST 1.1) or PCWG3. In some embodiments, the subject has or does not have a bone metastasis, a lymph node metastasis, or a visceral metastasis. In some embodiments, the visceral metastasis comprises alive metastasis, a lung metastasis, an adrenal metastasis, an abdominal metastasis, or a retroperitoneal metastasis, or a combination thereof. In some embodiments, the cancer does not respond to a treatment with a radioligand therapy that targets PSMA before the administering. In some embodiments, the cancer responds to a treatment with a radioligand therapy that targets PSMA before the administering. In some embodiments, the treatment comprises lutetium Lu 177 vipivotide tetraxetan. In some embodiments, the subject does not have a prior solid organ transplant procedure before the administering. In some embodiments, the subject is not treated with a chimeric antigen receptor T (CAR-T) cell therapy that targets PSMA or a T cell engager therapy that targets PSMA, before the administering. In some embodiments, the subject does not have clinically significant cardiovascular disease. In some embodiments, the subject does not have an active and clinically significant infection. In some embodiments, the infection comprises a bacterial infection, a viral infection, a fungal infection, a mycobacterial infection, or a combination thereof. In some embodiments, the subject has a Homologous Recombination Repair (HRR) gene mutation, a BReast Cancer (BRCA) gene mutation, a BRCA1 gene mutation, a BRCA2 gene mutation, a homeobox B13 (HOXB13) gene mutation, an ataxia telangiectasia mutated (ATM) gene mutation, or a combination thereof.

In some embodiments, the administering comprises administering intravenously. In some embodiments, the subject exhibits a cytokine release syndrome (CRS) no more than grade 2. In some embodiments, the subject exhibits no treatment related adverse event (TRAE) that is related to a CRS. In some embodiments, the administering comprises administering to the subject a second treatment. In some embodiments, the second treatment comprises lutetium Lu 177 vipivotide tetraxetan, enzalutamide, abiraterone acetate (ABI), chemotherapy, radium Ra 223 dichloride, a poly-adenosine diphosphate ribose polymerase inhibitor (PARPI), pembrolizumab, or a combination thereof. In some embodiments, the second treatment comprises enzalutamide. In some embodiments, the administering results in a synergistic effect on the cancer compared with administering the isolated recombinant polypeptide complex alone or administering enzalutamide alone. In some embodiments, the synergistic effect comprises a therapeutic effect that is better than a therapeutic effect resulting from administering the isolated recombinant polypeptide complex alone or a therapeutic effect resulting from administering enzalutamide alone. In some embodiments, the cancer is refractory, non-responsive, or resistant, to treatment with enzalutamide alone before the administering. In some embodiments, the PARPI comprises olaparib, rucaparib, niraparib, talazoparib, or a combination thereof. In some embodiments, the administering results in a therapeutic effect in prostate specific antigen (PSA) response, best RECIST response, best response by PCWG3 criteria, best PET response, radiographic progression free survival (rPFS), overall response rate, overall survival, bone pain, tumor size, or a combination thereof, after the administering. In some embodiments, the subject exhibits a reduction of at least about 30% in PSA level after the administering. In some embodiments, the subject exhibits a reduction of at least about 50% in PSA level after the administering. In some embodiments, the subject exhibits a reduction of at least about 70% in PSA level after the administering. In some embodiments, the subject exhibits a reduction of at least about 90% in PSA level after the administering. In some embodiments, the subject exhibits a reduction of about 30% to about 100% in PSA level after the administering. In some embodiments, the subject exhibits a higher reduction in PSA level when the first dose increases. In some embodiments, the subject exhibits a higher reduction in PSA level when the second dose increases. In some embodiments, the subject exhibits a higher reduction in PSA level when the target dose increases. In some embodiments, the subject exhibits a higher reduction in PSA level when the first dose is at least about 200 μg compared with when the first dose is at least about 100 μg. In some embodiments, the subject exhibit a reduction of about 30% to about 100% in PSA level at about 2 weeks after the administering of at least 200 μg of the first dose. In some embodiments, the reduction in PSA level continues throughout the treatment with the method. In some embodiments, the subject exhibits a better RECIST response when the first dose increases. In some embodiments, the subject exhibits a better RECIST response when the second dose increases. In some embodiments, the subject exhibits a better RECIST response when the target dose increases. In some embodiments, the subject exhibits a better RECIST response when the first dose is at least about 200 μg compared with when the first dose is at least about 100 μg. In some embodiments, a reduction in PSA level correlates with an occurrence of a CRS. In some embodiments, the subject exhibits a reduction of about 0% to about 100% in tumor size after the administering. In some embodiments, the subject exhibits a reduction of about 5% to about 75% in tumor size after the administering. In some embodiments, the subject exhibits a reduction in bone skeletal lesion measured by PCWG3 criteria after the administering. In some embodiments, the change in tumor size is measured by RECIST 1.1. In some embodiments, the change in PSMA level is measured by standardized uptake value (SUV) by PSMA-PET. In some embodiments, the subject exhibits a RECIST partial response when the first dose is at least about 100 μg, at least about 200 μg, at least about 300 μg, at least about 400 μg, or at least about 500 μg. In some embodiments, the subject exhibits a RECIST partial response when the target dose is at least about 100 μg to at least about 10 mg. In some embodiments, the subject exhibits a reduction of 0%-100% in SUV measured by PSMA-PET. In some embodiments, the subject exhibits a reduction of about 72% in SUV measured by PSMA-PET. In some embodiments, the subject exhibits about 80% in best PSA reduction. In some embodiments, the subject exhibits a reduction in pain related to cancer. In some embodiments, the subject exhibits a reduction in bone pain. In some embodiments, the subject exhibits a best PET response. In some embodiments, the subject exhibits a best RECIST response or a best response measured by PCWG3. In some embodiments, the method further comprises treating the subject with a therapy for CRS, diarrhea, chills, alanine transaminase (ALT) increase, anaemia, aspartate aminotransferase (AS) increase, fatigue, decreased appetite, nausea, headache, blood bilirubin increase, hypoalbuminaemia, hypocalcaemia, hypophosphataemia, leukopenia, white blood cell count decrease, myalgia, platelet count decrease, thrombocytopenia, pyrexia, vomiting, blood alkaline phosphatase increase, dysgeusia, hypomagnesaemia, lipase increase, stomatitis, or a combination thereof. In some embodiments, the isolated recombinant polypeptide complex is cleaved by a cancer specific protease to generate an active T cell engager and a cleavage fragment after the administering. In some embodiments, the administering provides a plasma concentration of the isolated recombinant polypeptide complex that is below preclinical activity threshold. In some embodiments, the administering provides a plasma concentration of the active T cell engager that is below preclinical activity threshold.

In some embodiments, the administering of at least one dose of about 100 μg of the isolated recombinant polypeptide complex provides a maximum plasma concentration (Cmax) of the isolated recombinant polypeptide complex in an amount up to about 34 ng/ml. In some embodiments, the administering of at least one dose of about 300 μg of the isolated recombinant polypeptide complex provides a Cmax of the isolated recombinant polypeptide complex in an amount up to about 175 ng/ml. In some embodiments, the administering of at least one dose of about 450 μg of the isolated recombinant polypeptide complex provides a Cmax of the isolated recombinant polypeptide complex in an amount of up to about 131 ng/ml. In some embodiments, the administering of at least one dose of about 1 mg of the isolated recombinant polypeptide complex provides a Cmax of the isolated recombinant polypeptide complex in an amount up to about 283 ng/ml. In some embodiments, the administering provides a Cmax of the isolated recombinant polypeptide complex in an amount that correlates with a dose being administered. In some embodiments, the Cmax of the isolated recombinant polypeptide complex increases when the dose being administered increases. In some embodiments, the administering provides a Cmax of the isolated recombinant polypeptide complex in an amount that correlates with the number of doses that the subject has received. In some embodiments, the Cmax of the isolated recombinant polypeptide complex increases when the number of doses that the subject has received increases. In some embodiments, the administering provides a Cmax of the cleavage fragment in an amount up to about 4.5 ng/ml, and wherein the first dose, the second dose and the target dose being administered are about 100 μg, about 300 μg, and about 450 μg, respectively. In some embodiments, the administering provides a Cmax of the cleavage fragment in an amount up to about 14 ng/ml, and wherein the first dose, the second dose and the target dose being administered are about 100 μg, about 300 μg, and about 1 mg, respectively. In some embodiments, the administering provides a Cmax of the cleavage fragment in an amount that correlates with the target dose being administered. In some embodiments, the Cmax of the cleavage fragment increases when the target dose being administered increases. In some embodiments, the administering provides a Cmax of the cleavage fragment in an amount that correlates with the number of doses that the subject has received. In some embodiments, the Cmax of the cleavage fragment increases when the number of doses that the subject has received increase.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present invention will be obtained by reference to the following detailed description that sets forth illustrative embodiments, in which the principles of the disclosure are utilized, and the accompanying drawings (also “Figure” and “FIG.” herein), of which:

FIG. 1 illustrates a non-limiting example of design, structure and mechanism of action of polypeptide complex 1 (PC-1) according to an embodiment of the present disclosure.

FIG. 2 illustrates a non-limiting example of an outline of Planned Dose Escalation, Backfill and Dose Expansion according to an embodiment of the present disclosure.

FIG. 3 illustrates a non-limiting example of Study Dosing Schema and Imaging Assessments according to an embodiment of the present disclosure.

FIG. 4 illustrates a non-limiting Example Step Dose Regimen Schedules according to an embodiment of the present disclosure.

FIG. 5 illustrates a non-limiting example of Prior Medians and 95% Credibility Intervals CRS DLTs according to an embodiment of the present disclosure.

FIG. 6 illustrates a non-limiting example of Prior Medians and 95% Credibility Intervals Non-CRS DLTs according to an embodiment of the present disclosure.

FIG. 7 illustrates a non-limiting example of Prior Medians and 95% Credibility Intervals for the Joint CRS and Non-CRS DLTs according to an embodiment of the present disclosure.

FIG. 8 illustrates a non-limiting example of phase 1 trial design in mCRPC according to an embodiment of the present disclosure.

FIG. 9 illustrates a non-limiting example of human pharmacokinetics of PC-1 components according to an embodiment of the present disclosure.

FIG. 10 illustrates a non-limiting example of time on treatment for all subjects according to an embodiment of the present disclosure.

FIG. 11 illustrates a non-limiting example of Best Overall percentage Change in PSA Values From Baseline for all subjects according to an embodiment of the present disclosure.

FIG. 12A illustrates a non-limiting example of PSA responses at step of 0.1 mg according to an embodiment of the present disclosure.

FIG. 12B illustrates a non-limiting example of PSA responses at step of 0.2 mg according to an embodiment of the present disclosure.

FIG. 13 illustrates a non-limiting example of significant tumor burden reductions demonstrated by PSMA-PET in a subject according to an embodiment of the present disclosure.

### DETAILED DESCRIPTION

In the context of the present application, the following terms have the meanings ascribed to them unless specified otherwise:

As used throughout the specification and claims, the terms “a”, “an” and “the” are generally used in the sense that they mean “at least one”, “at least a first”, “one or more” or “a plurality” of the referenced components or steps, except in instances wherein an upper limit is thereafter specifically stated. For example, a “cleavage sequence”, as used herein, means “at least a first cleavage sequence” but includes a plurality of cleavage sequences. The operable limits and parameters of combinations, as with the amounts of any single agent, will be known to those of ordinary skill in the art in light of the present application.

The terms “polypeptide”, “peptide”, and “protein” are used interchangeably herein to generally refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein in the context of the structure of a polypeptide, “N-terminus” (or “amino terminus”) and “C-terminus” (or “carboxyl terminus”) generally refer to the extreme amino and carboxyl ends of the polypeptide, respectively.

The term “therapeutically effective amount,” as used herein, generally means the amount of a polypeptide variant or a polypeptide composition that, when administered to a patient for treating a disease or other undesirable medical condition, is sufficient to have a beneficial effect with respect to that disease or condition. The therapeutically effective amount will vary depending on the polypeptide variant or the polypeptide composition, the disease or condition and its severity, and the age, weight, etc. of the patient to be treated. Determining the therapeutically effective amount of a given polypeptide variant or a given polypeptide composition is generally within the ordinary skill of the art and requires no more than routine experimentation.

As used herein, the terms “about” and “approximately” are used interchangeably. Any numerals used herein with or without about/approximately are meant to cover any normal fluctuations appreciated by one of ordinary skill in the relevant art. For example, the term “about” may refer to a range of values ±10%, ±5%, ±2%, or ±1% of a specified value. By way of an example. For example, the phrase “about 50%” may include from 45% to 55%, from 48% to 52%, or from 49% to 51%.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present application.

### Recombinant Polypeptide Compositions

Metastatic castration-resistant prostate cancer (mCRPC) remains an incurable disease. Bispecific T cell engagers (TCEs) targeting prostate-specific membrane antigen (PSMA) on prostate tumor cells and cluster of differentiation 3 (CD3) on T cells have clinical efficacy for the treatment of mCRPC. These TCEs have issues of cytokine release syndrome (CRS) and poor pharmacokinetic (PK) profile. There remains a need for new immune therapy. Disclosed herein is a PSMA-targeted tumor-activated T cell engager (TRACTr) featuring enhanced safety and pharmacokinetics profiles.

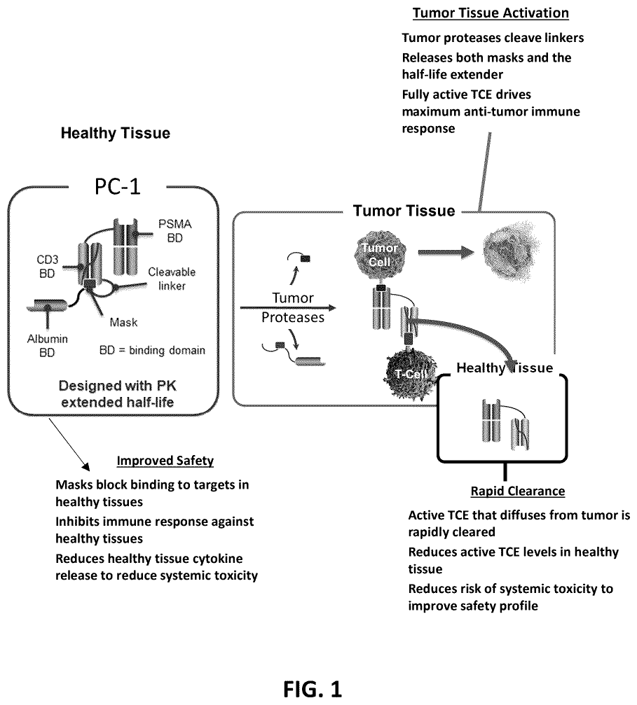

FIG. 1 illustrates design, structure and mechanism of action of polypeptide complex 1 (PC-1). PC-1 is a tumor-activated T cell engager with PSMA- and CD3-binding domains, an albumin-binding domain to extend circulating half-life, a peptide mask that inhibits CD3 engagement on T cells, and a tumor protease cleavable linker. Tumor-specific proteolysis of the cleavable linker in the tumor microenvironment (TME) separates the tandem mask and albumin-binding domain from PC-1. It enables TME restricted CD3 binding and subsequent T cell activation against PSMA expressing prostate cancer cells. Loss of the albumin-binding domain likely ensures that any activated PC-1 that migrates out of the tumor will be cleared rapidly and reduces its potential accumulation in healthy tissues that can contribute to safety risks.

Disclosed herein is a recombinant polypeptide. In some embodiments, the recombinant polypeptide comprises a tumor-activated T cell engager with PSMA- and CD3-binding domains, an albumin binding domain to extend circulating half-life, a peptide mask that inhibits CD3 engagement on T-cells, and a tumor protease cleavable linker. Tumor specific proteolysis of the cleavable linker in the tumor microenvironment can separate the tandem mask and albumin-binding domain from the recombinant polypeptide. The recombinant polypeptide can comprise chain 1 and chain 2 as described herein. In some embodiments, chain 1 comprises an amino acid sequence having at least 70%, 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1. In some embodiments, chain 2 comprises an amino acid sequence having at least 70%, 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 2.

In some embodiments, the recombinant polypeptide comprises an amino acid sequence having at least 80% sequence identity to SEQ ID NO: 1. In some embodiments, the recombinant polypeptide comprises an amino acid sequence having at least 85% sequence identity to SEQ ID NO: 1. In some embodiments, the recombinant polypeptide comprises an amino acid sequence having at least 90% sequence identity to SEQ ID NO: 1. In some embodiments, the recombinant polypeptide comprises an amino acid sequence having at least 91% sequence identity to SEQ ID NO: 1. In some embodiments, the recombinant polypeptide comprises an amino acid sequence having at least 92% sequence identity to SEQ ID NO: 1. In some embodiments, the recombinant polypeptide comprises an amino acid sequence having at least 93% sequence identity to SEQ ID NO: 1. In some embodiments, the recombinant polypeptide comprises an amino acid sequence having at least 94% sequence identity to SEQ ID NO: 1. In some embodiments, the recombinant polypeptide comprises an amino acid sequence having at least 95% sequence identity to SEQ ID NO: 1. In some embodiments, the recombinant polypeptide comprises an amino acid sequence having at least 96% sequence identity to SEQ ID NO: 1. In some embodiments, the recombinant polypeptide comprises an amino acid sequence having at least 97% sequence identity to SEQ ID NO: 1. In some embodiments, the recombinant polypeptide comprises an amino acid sequence having at least 98% sequence identity to SEQ ID NO: 1. In some embodiments, the recombinant polypeptide comprises an amino acid sequence having at least 99% sequence identity to SEQ ID NO: 1.

In some embodiments, the recombinant polypeptide comprises an amino acid sequence according to SEQ ID NO: 1.

In some embodiments, the recombinant polypeptide comprises an amino acid sequence having at least 80% sequence identity to SEQ ID NO: 2. In some embodiments, the recombinant polypeptide comprises an amino acid sequence having at least 85% sequence identity to SEQ ID NO: 2. In some embodiments, the recombinant polypeptide comprises an amino acid sequence having at least 90% sequence identity to SEQ ID NO: 2. In some embodiments, the recombinant polypeptide comprises an amino acid sequence having at least 91% sequence identity to SEQ ID NO: 2. In some embodiments, the recombinant polypeptide comprises an amino acid sequence having at least 92% sequence identity to SEQ ID NO: 2. In some embodiments, the recombinant polypeptide comprises an amino acid sequence having at least 93% sequence identity to SEQ ID NO: 2. In some embodiments, the recombinant polypeptide comprises an amino acid sequence having at least 94% sequence identity to SEQ ID NO: 2. In some embodiments, the recombinant polypeptide comprises an amino acid sequence having at least 95% sequence identity to SEQ ID NO: 2. In some embodiments, the recombinant polypeptide comprises an amino acid sequence having at least 9600 sequence identity to SEQ ID NO: 2. In some embodiments, the recombinant polypeptide comprises an amino acid sequence having at least 97% sequence identity to SEQ ID NO: 2. In some embodiments, the recombinant polypeptide comprises an amino acid sequence having at least 98% sequence identity to SEQ ID NO: 2. In some embodiments, the recombinant polypeptide comprises an amino acid sequence having at least 99% sequence identity to SEQ ID NO: 2.

In some embodiments, the recombinant polypeptide comprises an amino acid sequence according to SEQ ID NO: 2.

TABLE 1
Recombinant Polypeptide Amino Acid Sequences

| Construct | | SEQ |
|---|---|---|
| Descrip- | Amino Acid Sequence | ID |
| tion | (N to C) | NO: |
| Chain 1 | EVQLVESGGGLVQPGGSLRLSCAASGSTFYT | 1 |
| PC-1 | AVMGWVRQAPGKGLEWVAAIRWTALTTSYAD | |
| | SVKGRFTISRDGAKTTLYLQMNSLRPEDTAV | |
| | YYCAARGTLGLFTTADSYDYWGQGTLVTVSS | |
| | GGGGSGGGSGGVYCGPEFDESVGCMGGGGSG | |
| | GGLSGRSDAGSPLGLAGSGGGSEVQLVESGG | |
| | GLVQPGGSLKLSCAASGFTFNKYAMNWVRQA | |
| | PGKGLEWVARIRSKYNNYATYYADSVKDRFT | |
| | ISRDDSKNTAYLQMNNLKTEDTAVYYCVRHG | |
| | NFGNSYISYWAYWGQGTLVTVSSGGGGSGGG | |
| | GSGGGGSQTVVTQEPSLTVSPGGTVTLTCGS | |
| | STGAVTSGNYPNWVQQKPGQAPRGLIGGTKF | |
| | LAPGTPARFSGSLLGGKAALTLSGVQPEDEA | |
| | EYYCVLWYSNRWVFGGGTKLTVLGGGGSDIQ | |
| | MTQSPSSLSASVGDRVTITCRASQGISNYLA | |
| | WYQQKTGKVPKFLIYEASTLQSGVPSRFSGG | |
| | GSGTDFTLTISSLQPEDVATYYCQNYNSAPF | |
| | TFGPGTKVDIKRTVAAPSVFIFPPSDEQLKS | |
| | GTASVVCLLNNFYPREAKVQWKVDNALQSGN | |
| | SQESVTEQDSKDSTYSLSSTLTLSKADYEKH | |
| | KVYACEVTHQGLSSPVTKSFNRGEC | |
| Chain 2 | QVQLVESGGGVVQPGRSLRLSCAASGFAFSR | 2 |
| PC-1 | YGMHWVRQAPGKGLEWVAVIWYDGSNKYYAD | |
| | SVKGRFTISRDNSKNTQYLQMNSLRAEDTAV | |
| | YYCARGGDFLYYYYYGMDVWGQGTTVTVSSA | |
| | STKGPSVFPLAPSSKSTSGGTAALGCLVKDY | |
| | FPEPVTVSWNSGALTSGVHTFPAVLQSSGLY | |
| | SLSSVVTVPSSSLGTQTYICNVNHKPSNTKV | |
| | DKKVEPKSC | |

### Methods of Treatment

In some embodiments, are methods of treating cancer in a subject need in need thereof comprising administering to the subject an isolated recombinant polypeptide complex as described herein. In some embodiments, the cancer has cells that express PSMA. In some instances, the cancer is a solid tumor cancer. In some embodiments, the cancer is lung, breast (e.g. HER2+; ER/PR+; TNBC), cervical, ovarian, colorectal, pancreatic or gastric.

In some embodiments, are methods of treating prostate cancer in a subject in need thereof comprising administering to the subject an isolated recombinant polypeptide complex as described herein. In some embodiments, are methods of treating metastatic castrate-resistant prostate cancer (mCRPC) in a subject need in need thereof comprising administering to the subject an isolated recombinant polypeptide complex as described herein.

Provided herein, in some embodiments, are methods of treating metastatic castration-resistance prostate cancer (mCRPC) in a subject in need thereof comprising administering to the subject a recombinant polypeptide (such as any described herein) or a formulation (such as any described herein). In some embodiments, the subject has at least one symptom of mCRPC. In some embodiments, the subject has received a diagnosis of mCRPC.

For administration to a subject, the recombinant polypeptide as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions. In some embodiments, a pharmaceutical composition disclosed herein is administered intravenously to a subject in need thereof.

Dosages of the substances of the present disclosure can vary between wide limits, depending upon the disease or disorder to be treated, the age and condition of the individual to be treated, etc. and a physician will ultimately determine appropriate dosages to be used.

Disclosed herein, in an aspect, is a method for treating cancer. In some embodiments, the method comprises a step dosing regimen. In some embodiments, the step dosing regimen comprises providing to the subject two or more injections of the isolated recombinant polypeptide complex disclosed herein with increasing dosages. In some embodiments, the method comprises administering to a subject in need thereof a first dose and a target dose of an isolated recombinant polypeptide complex disclosed herein. In some embodiments, the target dose is higher than the first dose. In some embodiments, the target dose is at least about 100 microgram (μg). In some embodiments, the isolated recombinant polypeptide complex comprises a first chain and a second chain.

Disclosed herein, in an aspect, is a method for treating cancer. In some embodiments, the method comprising administering to a subject in need thereof a first dose and a target dose of the isolated recombinant polypeptide complex disclosed herein. In some embodiments, the first dose is at least about 100 μg and the target dose is higher than the first dose. In some embodiments, the isolated recombinant polypeptide complex comprises a first chain with an amino acid sequence having at least 80% sequence identity to SEQ ID NO: 1 and a second chain with an amino acid sequence having at least 80% sequence identity to SEQ ID NO: 2.

In some embodiments, the first chain comprises an amino acid sequence having at least 70% sequence identity to SEQ ID NO: 1. In some embodiments, the first chain comprises an amino acid sequence having at least 71% sequence identity to SEQ ID NO: 1. In some embodiments, the first chain comprises an amino acid sequence having at least 72% sequence identity to SEQ ID NO: 1. In some embodiments, the first chain comprises an amino acid sequence having at least 73% sequence identity to SEQ ID NO: 1. In some embodiments, the first chain comprises an amino acid sequence having at least 74% sequence identity to SEQ ID NO: 1. In some embodiments, the first chain comprises an amino acid sequence having at least 75% sequence identity to SEQ ID NO: 1. In some embodiments, the first chain comprises an amino acid sequence having at least 76% sequence identity to SEQ ID NO: 1. In some embodiments, the first chain comprises an amino acid sequence having at least 77% sequence identity to SEQ ID NO: 1. In some embodiments, the first chain comprises an amino acid sequence having at least 78% sequence identity to SEQ ID NO: 1. In some embodiments, the first chain comprises an amino acid sequence having at least 79% sequence identity to SEQ ID NO: 1. In some embodiments, the first chain comprises an amino acid sequence having at least 80% sequence identity to SEQ ID NO: 1. In some embodiments, the first chain comprises an amino acid sequence having at least 81% sequence identity to SEQ ID NO: 1. In some embodiments, the first chain comprises an amino acid sequence having at least 82% sequence identity to SEQ ID NO: 1. In some embodiments, the first chain comprises an amino acid sequence having at least 83% sequence identity to SEQ ID NO: 1. In some embodiments, the first chain comprises an amino acid sequence having at least 84% sequence identity to SEQ ID NO: 1. In some embodiments, the first chain comprises an amino acid sequence having at least 85% sequence identity to SEQ ID NO: 1. In some embodiments, the first chain comprises an amino acid sequence having at least 86% sequence identity to SEQ ID NO: 1. In some embodiments, the first chain comprises an amino acid sequence having at least 87% sequence identity to SEQ ID NO: 1. In some embodiments, the first chain comprises an amino acid sequence having at least 88% sequence identity to SEQ ID NO: 1. In some embodiments, the first chain comprises an amino acid sequence having at least 89% sequence identity to SEQ ID NO: 1. In some embodiments, the first chain comprises an amino acid sequence having at least 90% sequence identity to SEQ ID NO: 1. In some embodiments, the first chain comprises an amino acid sequence having at least 91% sequence identity to SEQ ID NO: 1. In some embodiments, the first chain comprises an amino acid sequence having at least 92% sequence identity to SEQ ID NO: 1. In some embodiments, the first chain comprises an amino acid sequence having at least 93% sequence identity to SEQ ID NO: 1. In some embodiments, the first chain comprises an amino acid sequence having at least 94% sequence identity to SEQ ID NO: 1. In some embodiments, the first chain comprises an amino acid sequence having at least 95% sequence identity to SEQ ID NO: 1. In some embodiments, the first chain comprises an amino acid sequence having at least 96% sequence identity to SEQ ID NO: 1. In some embodiments, the first chain comprises an amino acid sequence having at least 97% sequence identity to SEQ ID NO: 1. In some embodiments, the first chain comprises an amino acid sequence having at least 98% sequence identity to SEQ ID NO: 1. In some embodiments, the first chain comprises an amino acid sequence having at least 99% sequence identity to SEQ ID NO: 1. In some embodiments, the first chain comprises the amino acid sequence of SEQ ID NO: 1.

In some embodiments, the second chain comprises an amino acid sequence having at least 70% sequence identity to SEQ ID NO: 2. In some embodiments, the second chain comprises an amino acid sequence having at least 71% sequence identity to SEQ ID NO: 2. In some embodiments, the second chain comprises an amino acid sequence having at least 72% sequence identity to SEQ ID NO: 2. In some embodiments, the second chain comprises an amino acid sequence having at least 73% sequence identity to SEQ ID NO: 2. In some embodiments, the second chain comprises an amino acid sequence having at least 74% sequence identity to SEQ ID NO: 2. In some embodiments, the second chain comprises an amino acid sequence having at least 75% sequence identity to SEQ ID NO: 2. In some embodiments, the second chain comprises an amino acid sequence having at least 76% sequence identity to SEQ ID NO: 2. In some embodiments, the second chain comprises an amino acid sequence having at least 77% sequence identity to SEQ ID NO: 2. In some embodiments, the second chain comprises an amino acid sequence having at least 78% sequence identity to SEQ ID NO: 2. In some embodiments, the second chain comprises an amino acid sequence having at least 79% sequence identity to SEQ ID NO: 2. In some embodiments, the second chain comprises an amino acid sequence having at least 80% sequence identity to SEQ ID NO: 2. In some embodiments, the second chain comprises an amino acid sequence having at least 81% sequence identity to SEQ ID NO: 2. In some embodiments, the second chain comprises an amino acid sequence having at least 82% sequence identity to SEQ ID NO: 2. In some embodiments, the second chain comprises an amino acid sequence having at least 83% sequence identity to SEQ ID NO: 2. In some embodiments, the second chain comprises an amino acid sequence having at least 84% sequence identity to SEQ ID NO: 2. In some embodiments, the second chain comprises an amino acid sequence having at least 85% sequence identity to SEQ ID NO: 2. In some embodiments, the second chain comprises an amino acid sequence having at least 86% sequence identity to SEQ ID NO: 2. In some embodiments, the second chain comprises an amino acid sequence having at least 87% sequence identity to SEQ ID NO: 2. In some embodiments, the second chain comprises an amino acid sequence having at least 88% sequence identity to SEQ ID NO: 2. In some embodiments, the second chain comprises an amino acid sequence having at least 89% sequence identity to SEQ ID NO: 2. In some embodiments, the second chain comprises an amino acid sequence having at least 90% sequence identity to SEQ ID NO: 2. In some embodiments, the second chain comprises an amino acid sequence having at least 91% sequence identity to SEQ ID NO: 2. In some embodiments, the second chain comprises an amino acid sequence having at least 92% sequence identity to SEQ ID NO: 2. In some embodiments, the second chain comprises an amino acid sequence having at least 93% sequence identity to SEQ ID NO: 2. In some embodiments, the second chain comprises an amino acid sequence having at least 94% sequence identity to SEQ ID NO: 2. In some embodiments, the second chain comprises an amino acid sequence having at least 95% sequence identity to SEQ ID NO: 2. In some embodiments, the second chain comprises an amino acid sequence having at least 96% sequence identity to SEQ ID NO: 2. In some embodiments, the second chain comprises an amino acid sequence having at least 97% sequence identity to SEQ ID NO: 2. In some embodiments, the second chain comprises an amino acid sequence having at least 98% sequence identity to SEQ ID NO: 2. In some embodiments, the second chain comprises an amino acid sequence having at least 99% sequence identity to SEQ ID NO: 2. In some embodiments, the second chain comprises the amino acid sequence of SEQ ID NO: 2.

In some embodiments, the first chain comprises an amino acid sequence having at least 70% sequence identity to SEQ ID NO: 1 and the second chain comprises an amino acid sequence having at least 70% sequence identity to SEQ ID NO: 2. In some embodiments, the first chain comprises an amino acid sequence having at least 71% sequence identity to SEQ ID NO: 1 and the second chain comprises an amino acid sequence having at least 71% sequence identity to SEQ ID NO: 2. In some embodiments, the first chain comprises an amino acid sequence having at least 72% sequence identity to SEQ ID NO: 1 and the second chain comprises an amino acid sequence having at least 72% sequence identity to SEQ ID NO: 2. In some embodiments, the first chain comprises an amino acid sequence having at least 73% sequence identity to SEQ ID NO: 1 and the second chain comprises an amino acid sequence having at least 73% sequence identity to SEQ ID NO: 2. In some embodiments, the first chain comprises an amino acid sequence having at least 74% sequence identity to SEQ ID NO: 1 and the second chain comprises an amino acid sequence having at least 74% sequence identity to SEQ ID NO: 2. In some embodiments, the first chain comprises an amino acid sequence having at least 75% sequence identity to SEQ ID NO: 1 and the second chain comprises an amino acid sequence having at least 75% sequence identity to SEQ ID NO: 2. In some embodiments, the first chain comprises an amino acid sequence having at least 76% sequence identity to SEQ ID NO: 1 and the second chain comprises an amino acid sequence having at least 76% sequence identity to SEQ ID NO: 2. In some embodiments, the first chain comprises an amino acid sequence having at least 77% sequence identity to SEQ ID NO: 1 and the second chain comprises an amino acid sequence having at least 77% sequence identity to SEQ ID NO: 2. In some embodiments, the first chain comprises an amino acid sequence having at least 78% sequence identity to SEQ ID NO: 1 and the second chain comprises an amino acid sequence having at least 78% sequence identity to SEQ ID NO: 2. In some embodiments, the first chain comprises an amino acid sequence having at least 79% sequence identity to SEQ ID NO: 1 and the second chain comprises an amino acid sequence having at least 79% sequence identity to SEQ ID NO: 2. In some embodiments, the first chain comprises an amino acid sequence having at least 80% sequence identity to SEQ ID NO: 1 and the second chain comprises an amino acid sequence having at least 80% sequence identity to SEQ ID NO: 2. In some embodiments, the first chain comprises an amino acid sequence having at least 81% sequence identity to SEQ ID NO: 1 and the second chain comprises an amino acid sequence having at least 81% sequence identity to SEQ ID NO: 2. In some embodiments, the first chain comprises an amino acid sequence having at least 82% sequence identity to SEQ ID NO: 1 and the second chain comprises an amino acid sequence having at least 82% sequence identity to SEQ ID NO: 2. In some embodiments, the first chain comprises an amino acid sequence having at least 83% sequence identity to SEQ ID NO: 1 and the second chain comprises an amino acid sequence having at least 83% sequence identity to SEQ ID NO: 2. In some embodiments, the first chain comprises an amino acid sequence having at least 84% sequence identity to SEQ ID NO: 1 and the second chain comprises an amino acid sequence having at least 84% sequence identity to SEQ ID NO: 2. In some embodiments, the first chain comprises an amino acid sequence having at least 85% sequence identity to SEQ ID NO: 1 and the second chain comprises an amino acid sequence having at least 85% sequence identity to SEQ ID NO: 2. In some embodiments, the first chain comprises an amino acid sequence having at least 86% sequence identity to SEQ ID NO: 1 and the second chain comprises an amino acid sequence having at least 86% sequence identity to SEQ ID NO: 2. In some embodiments, the first chain comprises an amino acid sequence having at least 87% sequence identity to SEQ ID NO: 1 and the second chain comprises an amino acid sequence having at least 87% sequence identity to SEQ ID NO: 2. In some embodiments, the first chain comprises an amino acid sequence having at least 88% sequence identity to SEQ ID NO: 1 and the second chain comprises an amino acid sequence having at least 88% sequence identity to SEQ ID NO: 2. In some embodiments, the first chain comprises an amino acid sequence having at least 89% sequence identity to SEQ ID NO: 1 and the second chain comprises an amino acid sequence having at least 89% sequence identity to SEQ ID NO: 2. In some embodiments, the first chain comprises an amino acid sequence having at least 90% sequence identity to SEQ ID NO: 1 and the second chain comprises an amino acid sequence having at least 90% sequence identity to SEQ ID NO: 2. In some embodiments, the first chain comprises an amino acid sequence having at least 91% sequence identity to SEQ ID NO: 1 and the second chain comprises an amino acid sequence having at least 91% sequence identity to SEQ ID NO: 2. In some embodiments, the first chain comprises an amino acid sequence having at least 92% sequence identity to SEQ ID NO: 1 and the second chain comprises an amino acid sequence having at least 92% sequence identity to SEQ ID NO: 2. In some embodiments, the first chain comprises an amino acid sequence having at least 93% sequence identity to SEQ ID NO: 1 and the second chain comprises an amino acid sequence having at least 93% sequence identity to SEQ ID NO: 2. In some embodiments, the first chain comprises an amino acid sequence having at least 94% sequence identity to SEQ ID NO: 1 and the second chain comprises an amino acid sequence having at least 94% sequence identity to SEQ ID NO: 2. In some embodiments, the first chain comprises an amino acid sequence having at least 95% sequence identity to SEQ ID NO: 1 and the second chain comprises an amino acid sequence having at least 95% sequence identity to SEQ ID NO: 2. In some embodiments, the first chain comprises an amino acid sequence having at least 96% sequence identity to SEQ ID NO: 1 and the second chain comprises an amino acid sequence having at least 96% sequence identity to SEQ ID NO: 2. In some embodiments, the first chain comprises an amino acid sequence having at least 97% sequence identity to SEQ ID NO: 1 and the second chain comprises an amino acid sequence having at least 97% sequence identity to SEQ ID NO: 2. In some embodiments, the first chain comprises an amino acid sequence having at least 98% sequence identity to SEQ ID NO: 1 and the second chain comprises an amino acid sequence having at least 98% sequence identity to SEQ ID NO: 2. In some embodiments, the first chain comprises an amino acid sequence having at least 99% sequence identity to SEQ ID NO: 1 and the second chain comprises an amino acid sequence having at least 99% sequence identity to SEQ ID NO: 2. In some embodiments, the first chain comprises the amino acid sequence of SEQ ID NO: 1 and the second chain comprises the amino acid sequence of SEQ ID NO: 2.

In some embodiments, the first dose is about 100 μg to about 1 mg, e.g., about 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, or about 1000 μg, or any dose therebetween. In some embodiments, the first dose is about 100 μg. In some embodiments, the first dose is about 150 μg. In some embodiments, the first dose is about 200 μg. In some embodiments, the first dose is about 250 μg. In some embodiments, the first dose is about 300 μg. In some embodiments, the first dose is about 400 μg. In some embodiments, the first dose is about 500 μg. In some embodiments, the first dose is about 600 μg. In some embodiments, the first dose is about 700 μg. In some embodiments, the first dose is about 800 μg. In some embodiments, the first dose is about 900 μg. In some embodiments, the first dose is about 1 mg.

In some embodiments, the first dose is at least about 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, or at least about 1000 μg, or any dose therebetween. In some embodiments, the first dose is at least about 150 μg. In some embodiments, the first dose is at least about 200 μg. In some embodiments, the first dose is at least about 250 μg. In some embodiments, the first dose is at least about 300 μg. In some embodiments, the first dose is at least about 400 μg. In some embodiments, the first dose is at least about 500 μg. In some embodiments, the first dose is at least about 600 μg. In some embodiments, the first dose is at least about 700 μg. In some embodiments, the first dose is at least about 800 μg. In some embodiments, the first dose is at least about 900 μg.

In some embodiments, the target dose is at least about 450 μg, 455 μg, 460 μg, 465 μg, 470 μg, 475 μg, 480 μg, 485 μg, 490 μg, 495 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, 1000 μg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, or at least about 10 mg, or any dose therebetween. In some embodiments, the target dose is at least about 450 μg. In some embodiments, the target dose is at least about 750 μg. In some embodiments, the target dose is at least about 1 mg. In some embodiments, the target dose is at least about 1.5 mg. In some embodiments, the target dose is at least about 2 mg. In some embodiments, the target dose is at least about 2.5 mg. In some embodiments, the target dose is at least about 3 mg. In some embodiments, the target dose is at least about 4 mg. In some embodiments, the target dose is at least about 5 mg. In some embodiments, the target dose is at least about 6 mg. In some embodiments, the target dose is at least about 7 mg. In some embodiments, the target dose is at least about 8 mg. In some embodiments, the target dose is at least about 9 mg.

In some embodiments, the target dose is about 450 μg to about 10 mg, e.g., about 450 μg, 455 μg, 460 μg, 465 μg, 470 μg, 475 μg, 480 μg, 485 μg, 490 μg, 495 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, 1000 μg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, or about 10 mg, or any dose therebetween. In some embodiments, the target dose is about 450 μg. In some embodiments, the target dose is about 750 μg. In some embodiments, the target dose is about 1 mg. In some embodiments, the target dose is about 1.5 mg. In some embodiments, the target dose is about 2 mg. In some embodiments, the target dose is about 2.5 mg. In some embodiments, the target dose is about 3 mg. In some embodiments, the target dose is about 4 mg. In some embodiments, the target dose is about 5 mg. In some embodiments, the target dose is about 6 mg. In some embodiments, the target dose is about 7 mg. In some embodiments, the target dose is about 8 mg. In some embodiments, the target dose is about 9 mg. In some embodiments, the target dose is about 10 mg.

In some embodiments, the method further comprises administering to the subject a second dose of the isolated recombinant polypeptide complex, wherein the second dose is higher than the first dose and lower than the target dose, and wherein the second dose is administered between the first dose and the target dose. In some embodiments, the second dose is about 300 μg to about 5 mg, e.g., about 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, 1000 μg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, or about 5 mg, or any dose therebetween. In some embodiments, the second dose is about 300 μg. In some embodiments, the second dose is about 400 μg. In some embodiments, the second dose is about 500 μg. In some embodiments, the second dose is about 600 μg. In some embodiments, the second dose is about 700 μg. In some embodiments, the second dose is about 800 μg. In some embodiments, the second dose is about 900 μg. In some embodiments, the second dose is about 1 mg. In some embodiments, the second dose is about 1.2 mg. In some embodiments, the second dose is about 1.5 mg. In some embodiments, the second dose is about 2 mg. In some embodiments, the second dose is about 2.5 mg. In some embodiments, the second dose is about 3 mg. In some embodiments, the second dose is about 3.5 mg. In some embodiments, the second dose is about 4 mg. In some embodiments, the second dose is about 4.5 mg. In some embodiments, the second dose is about 5 mg. In some embodiments, the second dose is at least about 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, 1000 μg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, or at least about 5 mg, any dose therebetween. In some embodiments, the second dose is at least about 300 μg. In some embodiments, the second dose is at least about 400 μg. In some embodiments, the second dose is at least about 500 μg. In some embodiments, the second dose is at least about 600 μg. In some embodiments, the second dose is at least about 700 μg. In some embodiments, the second dose is at least about 800 μg. In some embodiments, the second dose is at least about 900 μg. In some embodiments, the second dose is at least about 1 mg. In some embodiments, the second dose is at least about 2 mg. In some embodiments, the second dose is at least about 3 mg. In some embodiments, the second dose is at least about 4 mg.

In some embodiments, the first dose, the second dose, and the target dose are about 100 μg, 300 μg, and 450 μg, respectively. In some embodiments, the first dose, the second dose, and the target dose are about 100 μg, 300 μg, and 1 mg, respectively. In some embodiments, the first dose, the second dose, and the target dose are about 200 μg, 600 μg, and 2 mg, respectively. In some embodiments, the first dose, the second dose, and the target dose are about 300 μg, 1 mg, and 3 mg, respectively. In some embodiments, the first dose, the second dose, and the target dose are about 400 μg, 1.2 mg, and 4 mg, respectively. In some embodiments, the first dose, the second dose, and the target dose are about 500 μg, 1.5 mg, and 5 mg, respectively. In some embodiments, the first dose, the second dose, and the target dose are about 600 μg, 2 mg, and 6 mg, respectively. In some embodiments, the first dose, the second dose, and the target dose are about 700 μg, 2.5 mg, and 7 mg, respectively. In some embodiments, the first dose, the second dose, and the target dose are about 800 μg, 3 mg, and 8 mg, respectively. In some embodiments, the first dose, the second dose, and the target dose are about 900 μg, 4 mg, and 9 mg, respectively. In some embodiments, the first dose, the second dose, and the target dose are about 1 mg, 5 mg, and 10 mg, respectively.

In some embodiments, the first dose, the second dose, or the target dose is administered weekly. In some embodiments, the first dose, the second dose, or the target dose is administered once every two weeks. In some embodiments, the first dose, the second dose, or the target dose is administered once every three weeks. In some embodiments, the first dose, the second dose, and the target dose are administered weekly. In some embodiments, the first dose and the second dose are administered weekly, and wherein the target dose is administered once every two weeks. In some embodiments, the first dose and the second dose are administered weekly, and wherein the target dose is administered once every three weeks. In some embodiments, the method comprises a treatment cycle that starts on day 1. In some embodiments, the treatment cycle is 21 days or 28 days. In some embodiments, the first dose, the second dose, and the target dose are administered on day 1, one of day 7, 8 or 9, and one of day 14, 15, or 16, respectively, of the treatment cycle. In some embodiments, the first dose, the second dose, and the target dose are administered on day 1, day 8, and day 15, respectively, of the treatment cycle. In some embodiments, the first dose, the second dose, and the target dose are administered on day 1, day 7, and day 14, respectively, of the treatment cycle. In some embodiments, the first dose, the second dose, and the target dose are administered on day 1, day 9, and day 16, respectively, of the treatment cycle. In some embodiments, the first dose, the second dose, and the target dose are administered on day 1, day 4, and day 8, respectively, of the treatment cycle. In some embodiments, the target dose is administered weekly after the one of day 14, 15, or 16, of the treatment cycle. In some embodiments, the target dose is administered weekly after day 14 of the treatment cycle. In some embodiments, the target dose is administered weekly after day 15 of the treatment cycle. In some embodiments, the target dose is administered weekly after day 16 of the treatment cycle. In some embodiments, the target dose is administered once every three weeks after the one of day 14, 15, or 16, of the treatment cycle. In some embodiments, the target dose is administered once every three weeks after day 14 of the treatment cycle. In some embodiments, the target dose is administered once every three weeks after day 15 of the treatment cycle. In some embodiments, the target dose is administered once every three weeks after day 16 of the treatment cycle. In some embodiments, the target dose is administered weekly after day 8 of the treatment cycle. In some embodiments, the target dose is administered once every two weeks after day 8 of the treatment cycle. In some embodiments, the target dose is administered once every three weeks after day 8 of the treatment cycle. In some embodiments, the target dose is administered once every two weeks after the one of day 14, 15, or 16, of the treatment cycle. In some embodiments, the target dose is administered once every two weeks after day 14 of the treatment cycle. In some embodiments, the target dose is administered once every two weeks after day 15 of the treatment cycle. In some embodiments, the target dose is administered once every two weeks after day 16 of the treatment cycle. In some embodiments, the target dose is administered at least once, at least twice, at least 3 times, at least 4 times, at least 5 times, at least 6 times, at least 7 times, at least 8 times, at least 9 times, at least 10 times, at least 15 times, at least 20 times, at least 25 times, at least 30 times, at least 35 times, at least 40 times, at least 45 times, at least 50 times, or more than 50 times. In some embodiments, the method further comprises at least 2 treatment cycles, at least 3 treatment cycles, at least 4 treatment cycles, at least 5 treatment cycles, at least 6 treatment cycles, at least 7 treatment cycles, at least 8 treatment cycles, at least 9 treatment cycles, at least 10 treatment cycles, or more than 10 treatment cycles.

In some embodiments, the first dose, the second dose, and the target dose are administered on day 1, the one of day 7, 8 or 9, and the one of day 14, 15, or 16, respectively, of the treatment cycle, and wherein the target dose is administered weekly after day 14, 15, or 16 of the treatment cycle. In some embodiments, the first dose, the second dose, and the target dose are administered on day 1, day 7, and day 14, respectively, of the treatment cycle, and wherein the target dose is administered weekly after day 14 of the treatment cycle. In some embodiments, the first dose, the second dose, and the target dose are administered on day 1, day 8, and day 15, respectively, of the treatment cycle, and wherein the target dose is administered weekly after day 15 of the treatment cycle. In some embodiments, the first dose, the second dose, and the target dose are administered on day 1, day 9, and day 16, respectively, of the treatment cycle, and wherein the target dose is administered weekly after day 16 of the treatment cycle. In some embodiments, the first dose, the second dose, and the target dose are administered on day 1, the one of day 7, 8 or 9, and the one of day 14, 15, or 16, respectively, of the treatment cycle, and wherein the target dose is administered once every two weeks after day 14, 15, or 16 of the treatment cycle. In some embodiments, the first dose, the second dose, and the target dose are administered on day 1, day 7, and day 14, respectively, of the treatment cycle, and wherein the target dose is administered once every two weeks after day 14 of the treatment cycle. In some embodiments, the first dose, the second dose, and the target dose are administered on day 1, day 8, and day 15, respectively, of the treatment cycle, and wherein the target dose is administered once every two weeks after day 15 of the treatment cycle. In some embodiments, the first dose, the second dose, and the target dose are administered on day 1, day 9, and day 16, respectively, of the treatment cycle, and wherein the target dose is administered once every two weeks after day 16 of the treatment cycle. In some embodiments, the first dose, the second dose, and the target dose are administered on day 1, the one of day 7, 8 or 9, and the one of day 14, 15, or 16, respectively, of the treatment cycle, and wherein the target dose is administered once every three weeks after day 14, 15, or 16 of the treatment cycle. In some embodiments, the first dose, the second dose, and the target dose are administered on day 1, day 7, and day 14, respectively, of the treatment cycle, and wherein the target dose is administered once every three weeks after day 14 of the treatment cycle. In some embodiments, the first dose, the second dose, and the target dose are administered on day 1, day 8, and day 15, respectively, of the treatment cycle, and wherein the target dose is administered once every three weeks after day 15 of the treatment cycle. In some embodiments, the first dose, the second dose, and the target dose are administered on day 1, day 9, and day 16, respectively, of the treatment cycle, and wherein the target dose is administered once every three weeks after day 16 of the treatment cycle. In some embodiments, the first dose, the second dose, and the target dose are administered on day 1, day 4, and day 8, respectively, of the treatment cycle, and wherein the target dose is administered weekly after day 8 of the treatment cycle. In some embodiments, the first dose, the second dose, and the target dose are administered on day 1, day 4, and day 8, respectively, of the treatment cycle, and wherein the target dose is administered once every two weeks after day 8 of the treatment cycle. In some embodiments, the first dose, the second dose, and the target dose are administered on day 1, day 4, and day 8, respectively, of the treatment cycle, and wherein the target dose is administered once every three weeks after day 8 of the treatment cycle.

In some embodiments, the cancer comprises a cell that expresses or overexpresses prostate specific membrane antigen (PSMA). In some embodiments, the cancer comprises cells that are dependent on endothelial cells of neovasculature that express or overexpress PSMA. In some embodiments, the cancer comprises prostate cancer, lung cancer, breast cancer, colorectal cancer, or renal cell carcinoma. In some embodiments, the cancer comprises prostate cancer. In some embodiments, the cancer comprises metastatic castration resistant prostate cancer (mCRPC). In some embodiments, the progression of mCRPC is documented according to guidelines of the Prostate Cancer Clinical Trials Working Group 3 (PCWG3), Response Evaluation Criteria in Solid Tumors 1.1 (RECIST 1.1), or both. In some embodiments, the cancer comprises non-small cell lung cancer (NSCLC), or small cell lung cancer (SCLC). In some embodiments, the subject is a male of at least 18 years old. In some embodiments, the subject is a male of about 46 to about 85 years old. In some embodiments, the subject is a male of about 69 years old. In some embodiments, the subject is treated with a prior cancer therapy before the administering. In some embodiments, the prior cancer therapy comprises 2 to 6 lines of cancer therapies. In some embodiments, the prior cancer therapy comprises 2 lines of cancer therapies. In some embodiments, the prior cancer therapy comprises 3 lines of cancer therapies. In some embodiments, the prior cancer therapy comprises 4 lines of cancer therapies. In some embodiments, the prior cancer therapy comprises 5 lines of cancer therapies. In some embodiments, the prior cancer therapy comprises 6 lines of cancer therapies. In some embodiments, the prior cancer therapy comprises a taxane, a radioligand therapy, an anti-androgen therapy, an androgen receptor pathway inhibitor therapy, a poly-adenosine diphosphate ribose polymerase (PARP) inhibitor, or a combination thereof. In some embodiments, the anti-androgen therapy comprises a radioligand therapy that targets PSMA. In some embodiments, the prior cancer therapy comprises a taxane and an anti-androgen therapy. In some embodiments, the cancer is mCRPC, and wherein the mCRPC progresses after the prior cancer therapy. In some embodiments, the prior cancer therapy comprises no taxane. In some embodiments, the prior cancer therapy comprises lutetium Lu 177 vipivotide tetraxetan. In some embodiments, the prior cancer therapy comprises olaparib, rucaparib, niraparib, talazoparib, or a combination thereof. In some embodiments, the subject has adequate organ function. In some embodiments, the subject has resolving acute effects of any prior therapy to baseline severity of Common Terminology Criteria for Adverse Events (CTCAE) grade of no more than 1. In some embodiments, the subject has adenocarcinoma of the prostate that is confirmed histologically or cytologically. In some embodiments, the subject is positive for a baseline prostate-specific membrane antigen positron emission tomography (PSMA-PET). In some embodiments, the subject has a Homologous Recombination Repair (HRR) gene mutation, a BReast Cancer (BRCA) gene mutation, a BRCA1 gene mutation, a BRCA2 gene mutation, a homeobox B13 (HOXB13) gene mutation, an ataxia telangiectasia mutated (ATM) gene mutation, or a combination thereof.

In some embodiments, the subject has a baseline PSA level of about 1 ng/ml to about 10000 ng/ml, e.g., about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, 20 ng/ml, 21 ng/ml, 22 ng/ml, 23 ng/ml, 24 ng/ml, 25 ng/ml, 26 ng/ml, 27 ng/ml, 28 ng/ml, 29 ng/ml, 30 ng/ml, 31 ng/ml, 32 ng/ml, 33 ng/ml, 34 ng/ml, 35 ng/ml, 36 ng/ml, 37 ng/ml, 38 ng/ml, 39 ng/ml, 40 ng/ml, 41 ng/ml, 42 ng/ml, 43 ng/ml, 44 ng/ml, 45 ng/ml, 46 ng/ml, 47 ng/ml, 48 ng/ml, 49 ng/ml, 50 ng/ml, 51 ng/ml, 52 ng/ml, 53 ng/ml, 54 ng/ml, 55 ng/ml, 56 ng/ml, 57 ng/ml, 58 ng/ml, 59 ng/ml, 60 ng/ml, 61 ng/ml, 62 ng/ml, 63 ng/ml, 64 ng/ml, 65 ng/ml, 66 ng/ml, 67 ng/ml, 68 ng/ml, 69 ng/ml, 70 ng/ml, 71 ng/ml, 72 ng/ml, 73 ng/ml, 74 ng/ml, 75 ng/ml, 76 ng/ml, 77 ng/ml, 78 ng/ml, 79 ng/ml, 80 ng/ml, 81 ng/ml, 82 ng/ml, 83 ng/ml, 84 ng/ml, 85 ng/ml, 86 ng/ml, 87 ng/ml, 88 ng/ml, 89 ng/ml, 90 ng/ml, 91 ng/ml, 92 ng/ml, 93 ng/ml, 94 ng/ml, 95 ng/ml, 96 ng/ml, 97 ng/ml, 98 ng/ml, 99 ng/ml, 100 ng/ml, 110 ng/ml, 120 ng/ml, 130 ng/ml, 140 ng/ml, 150 ng/ml, 160 ng/ml, 170 ng/ml, 180 ng/ml, 190 ng/ml, 200 ng/ml, 210 ng/ml, 220 ng/ml, 230 ng/ml, 240 ng/ml, 250 ng/ml, 260 ng/ml, 270 ng/ml, 280 ng/ml, 290 ng/ml, 300 ng/ml, 310 ng/ml, 320 ng/ml, 330 ng/ml, 340 ng/ml, 350 ng/ml, 360 ng/ml, 370 ng/ml, 380 ng/ml, 390 ng/ml, 400 ng/ml, 410 ng/ml, 420 ng/ml, 430 ng/ml, 440 ng/ml, 450 ng/ml, 460 ng/ml, 470 ng/ml, 480 ng/ml, 490 ng/ml, 500 ng/ml, 510 ng/ml, 520 ng/ml, 530 ng/ml, 540 ng/ml, 550 ng/ml, 560 ng/ml, 570 ng/ml, 580 ng/ml, 590 ng/ml, 600 ng/ml, 610 ng/ml, 620 ng/ml, 630 ng/ml, 640 ng/ml, 650 ng/ml, 660 ng/ml, 670 ng/ml, 680 ng/ml, 690 ng/ml, 700 ng/ml, 710 ng/ml, 720 ng/ml, 730 ng/ml, 740 ng/ml, 750 ng/ml, 760 ng/ml, 770 ng/ml, 780 ng/ml, 790 ng/ml, 800 ng/ml, 810 ng/ml, 820 ng/ml, 830 ng/ml, 840 ng/ml, 850 ng/ml, 860 ng/ml, 870 ng/ml, 880 ng/ml, 890 ng/ml, 900 ng/ml, 910 ng/ml, 920 ng/ml, 930 ng/ml, 940 ng/ml, 950 ng/ml, 960 ng/ml, 970 ng/ml, 980 ng/ml, 990 ng/ml, 1000 ng/ml, 1100 ng/ml, 1200 ng/ml, 1300 ng/ml, 1400 ng/ml, 1500 ng/ml, 1600 ng/ml, 1700 ng/ml, 1800 ng/ml, 1900 ng/ml, 2000 ng/ml, 2100 ng/ml, 2200 ng/ml, 2300 ng/ml, 2400 ng/ml, 2500 ng/ml, 2600 ng/ml, 2700 ng/ml, 2800 ng/ml, 2900 ng/ml, 3000 ng/ml, 3100 ng/ml, 3200 ng/ml, 3300 ng/ml, 3400 ng/ml, 3500 ng/ml, 3600 ng/ml, 3700 ng/ml, 3800 ng/ml, 3900 ng/ml, 4000 ng/ml, 4100 ng/ml, 4200 ng/ml, 4300 ng/ml, 4400 ng/ml, 4500 ng/ml, 4600 ng/ml, 4700 ng/ml, 4800 ng/ml, 4900 ng/ml, 5000 ng/ml, 5100 ng/ml, 5200 ng/ml, 5300 ng/ml, 5400 ng/ml, 5500 ng/ml, 5600 ng/ml, 5700 ng/ml, 5800 ng/ml, 5900 ng/ml, 6000 ng/ml, 6100 ng/ml, 6200 ng/ml, 6300 ng/ml, 6400 ng/ml, 6500 ng/ml, 6600 ng/ml, 6700 ng/ml, 6800 ng/ml, 6900 ng/ml, 7000 ng/ml, 7100 ng/ml, 7200 ng/ml, 7300 ng/ml, 7400 ng/ml, 7500 ng/ml, 7600 ng/ml, 7700 ng/ml, 7800 ng/ml, 7900 ng/ml, 8000 ng/ml, 8100 ng/ml, 8200 ng/ml, 8300 ng/ml, 8400 ng/ml, 8500 ng/ml, 8600 ng/ml, 8700 ng/ml, 8800 ng/ml, 8900 ng/ml, 9000 ng/ml, 9100 ng/ml, 9200 ng/ml, 9300 ng/ml, 9400 ng/ml, 9500 ng/ml, 9600 ng/ml, 9700 ng/ml, 9800 ng/ml, 9900 ng/ml, or about 10000 ng/ml, any amount therebetween. In some embodiments, the subject has a baseline PSA level of about 158 ng/ml.

In some embodiments, the subject is or is not evaluable according to guidelines of response evaluation criteria in solid tumors 1.1 (RECIST 1.1) or PCWG3. In some embodiments, the subject has or does not have a bone metastasis, a lymph node metastasis, or a visceral metastasis. In some embodiments, the visceral metastasis comprises alive metastasis, a lung metastasis, an adrenal metastasis, an abdominal metastasis, or a retroperitoneal metastasis, or a combination thereof. In some embodiments, the cancer does not respond to a treatment with a radioligand therapy that targets PSMA before the administering. In some embodiments, the cancer responds to a treatment with a radioligand therapy that targets PSMA before the administering. In some embodiments, the treatment comprises lutetium Lu 177 vipivotide tetraxetan. In some embodiments, the subject does not have a prior solid organ transplant procedure before the administering. In some embodiments, the subject is not treated with a chimeric antigen receptor T (CAR-T) cell therapy that targets PSMA or a T cell engager therapy that targets PSMA, before the administering. In some embodiments, the subject does not have clinically significant cardiovascular disease. In some embodiments, the subject does not have an active and clinically significant infection. In some embodiments, the infection comprises a bacterial infection, a viral infection, a fungal infection, a mycobacterial infection, or a combination thereof. In some embodiments, the administering comprises administering intravenously. In some embodiments, the subject exhibits a cytokine release syndrome (CRS) no more than grade 2. In some embodiments, the subject exhibits no treatment related adverse event (TRAE) that is related to a CRS. In some embodiments, the administering comprises administering to the subject a second treatment. In some embodiments, the second treatment comprises lutetium Lu 177 vipivotide tetraxetan, enzalutamide, abiraterone acetate (ABI), chemotherapy, radium Ra 223 dichloride, a poly-adenosine diphosphate ribose polymerase inhibitor (PARPI), pembrolizumab, or a combination thereof. In some embodiments, the second treatment comprises enzalutamide. In some embodiments, the administering results in a synergistic effect on the cancer compared with administering the isolated recombinant polypeptide complex alone or administering enzalutamide alone. In some embodiments, the synergistic effect comprises a therapeutic effect that is better than a therapeutic effect resulting from administering the isolated recombinant polypeptide complex alone or a therapeutic effect resulting from administering enzalutamide alone. In some embodiments, the cancer is refractory, non-responsive, or resistant, to treatment with enzalutamide alone before the administering. In some embodiments, the PARPI comprises olaparib, rucaparib, niraparib, talazoparib, or a combination thereof. In some embodiments, the administering results in a therapeutic effect in prostate specific antigen (PSA) response, best RECIST response, best response by PCWG3 criteria, best PET response, radiographic progression free survival (rPFS), overall response rate, overall survival, bone pain, tumor size, or a combination thereof, after the administering. In some embodiments, the subject exhibits a reduction of at least about 30% in PSA level after the administering. In some embodiments, the subject exhibits a reduction of at least about 50% in PSA level after the administering. In some embodiments, the subject exhibits a reduction of at least about 70% in PSA level after the administering. In some embodiments, the subject exhibits a reduction of at least about 90% in PSA level after the administering. In some embodiments, the subject exhibits a reduction of more than 0% to about 100% in PSA level after the administering, e.g., about 1%, 2%, 3%, 4%, 5%, 6%, 7%, 8%, 9%, 10%, 11%, 12%, 13%, 14%, 15%, 16%, 17%, 18%, 190%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97% 98%, 99%, or about 100%, or any percentage therebetween. In some embodiments, the subject exhibits a reduction of about 30% to about 100% in PSA level after the administering. In some embodiments, the subject exhibits a higher reduction in PSA level when the first dose increases. In some embodiments, the subject exhibits a higher reduction in PSA level when the second dose increases. In some embodiments, the subject exhibits a higher reduction in PSA level when the target dose increases. In some embodiments, the subject exhibits a higher reduction in PSA level when the first dose is at least about 200 μg compared with when the first dose is at least about 100 μg. In some embodiments, the subject exhibit a reduction of about 30% to about 100% in PSA level at about 2 weeks after the administering of at least 200 μg of the first dose. In some embodiments, the reduction in PSA level continues throughout the treatment with the method. In some embodiments, the subject exhibits a better RECIST response when the first dose increases. In some embodiments, the subject exhibits a better RECIST response when the second dose increases. In some embodiments, the subject exhibits a better RECIST response when the target dose increases. In some embodiments, the subject exhibits a better RECIST response when the first dose is at least about 200 μg compared with when the first dose is at least about 100 μg. In some embodiments, a reduction in PSA level correlates with an occurrence of a CRS.

In some embodiments, the subject exhibits a reduction of about 0% to about 100% in tumor size after the administering. In some embodiments, the subject exhibits a reduction up to about 100% in tumor size after the administering, e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or any percentage therebetween. In some embodiments, the subject exhibits a reduction of about 5% to about 75% in tumor size after the administering.

In some embodiments, the subject exhibits a reduction in bone skeletal lesion measured by PCWG3 criteria after the administering. In some embodiments, the change in tumor size is measured by RECIST 1.1. In some embodiments, the change in PSMA level is measured by standardized uptake value (SUV) by PSMA-PET. In some embodiments, the subject exhibits a RECIST partial response when the first dose is at least about 100 μg, at least about 200 μg, at least about 300 μg, at least about 400 μg, or at least about 500 μg. In some embodiments, the subject exhibits a RECIST partial response when the target dose is at least about 100 μg to at least about 10 mg. In some embodiments, the subject exhibits a reduction of 0%-100% in SUV measured by PSMA-PET, e.g., about 1%, 2%, 3%, 4%, 5%, 6%, 7%, 80%, 90%, 100%, 11%, 12%, 13%, 14%, 15%, 16%, 17%, 18%, 19%, 20%, 21%, 22% 23%, 24%, 25%, 26%, 27%, 28%, 29%, 30%, 31%, 32%, 33%, 34%, 35%, 3600, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% 60%, 61%, 62%, 63%, 64%, 65%, 66%, 67%, 68%, 69%, 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or any percentage therebetween. In some embodiments, the subject exhibits a reduction of about 72% in SUV measured by PSMA-PET. In some embodiments, the subject exhibits about 80% in best PSA reduction. In some embodiments, the subject exhibits a reduction in pain related to cancer. In some embodiments, the subject exhibits a reduction in bone pain. In some embodiments, the subject exhibits a best PET response. In some embodiments, the subject exhibits a best RECIST response or a best response measured by PCWG3. In some embodiments, the method further comprises treating the subject with a therapy for CRS, diarrhea, chills, alanine transaminase (ALT) increase, anaemia, aspartate aminotransferase (AS) increase, fatigue, decreased appetite, nausea, headache, blood bilirubin increase, hypoalbuminaemia, hypocalcaemia, hypophosphataemia, leukopenia, white blood cell count decrease, myalgia, platelet count decrease, thrombocytopenia, pyrexia, vomiting, blood alkaline phosphatase increase, dysgeusia, hypomagnesaemia, lipase increase, stomatitis, or a combination thereof.

In some embodiments, the isolated recombinant polypeptide complex is cleaved by a cancer specific protease to generate an active T cell engager and a cleavage fragment after the administering. In some embodiments, the administering provides a plasma concentration of the isolated recombinant polypeptide complex that is below preclinical activity threshold. In some embodiments, the administering provides a plasma concentration of the active T cell engager that is below preclinical activity threshold. In some embodiments, the administering of at least one dose of about 100 μg of the isolated recombinant polypeptide complex provides a maximum plasma concentration (Cmax) of the isolated recombinant polypeptide complex in an amount up to about 34 ng/ml. In some embodiments, the administering of at least one dose of about 300 μg of the isolated recombinant polypeptide complex provides a Cmax of the isolated recombinant polypeptide complex in an amount up to about 175 ng/ml. In some embodiments, the administering of at least one dose of about 450 μg of the isolated recombinant polypeptide complex provides a Cmax of the isolated recombinant polypeptide complex in an amount of up to about 131 ng/ml. In some embodiments, the administering of at least one dose of about 1 mg of the isolated recombinant polypeptide complex provides a Cmax of the isolated recombinant polypeptide complex in an amount up to about 283 ng/ml. In some embodiments, the administering provides a Cmax of the isolated recombinant polypeptide complex in an amount that correlates with a dose being administered. In some embodiments, the Cmax of the isolated recombinant polypeptide complex increases when the dose being administered increases. In some embodiments, the administering provides a Cmax of the isolated recombinant polypeptide complex in an amount that correlates with the number of doses that the subject has received. In some embodiments, the Cmax of the isolated recombinant polypeptide complex increases when the number of doses that the subject has received increases. In some embodiments, the administering provides a Cmax of the cleavage fragment in an amount up to about 4.5 ng/ml, and wherein the first dose, the second dose and the target dose being administered are about 100 μg, about 300 μg, and about 450 μg, respectively. In some embodiments, the administering provides a Cmax of the cleavage fragment in an amount up to about 14 ng/ml, and wherein the first dose, the second dose and the target dose being administered are about 100 μg, about 300 μg, and about 1 mg, respectively. In some embodiments, the administering provides a Cmax of the cleavage fragment in an amount that correlates with the target dose being administered. In some embodiments, the Cmax of the cleavage fragment increases when the target dose being administered increases. In some embodiments, the administering provides a Cmax of the cleavage fragment in an amount that correlates with the number of doses that the subject has received. In some embodiments, the Cmax of the cleavage fragment increases when the number of doses that the subject has received increase.

In some embodiments, the method comprises administering to a subject in need thereof the first dose and the target dose of the isolated recombinant polypeptide complex, wherein the target dose is higher than the first dose, wherein the target dose is at least about 100 μg, wherein the isolated recombinant polypeptide complex comprises the first chain with an amino acid sequence having at least 80% sequence identity to SEQ ID NO: 1 and the second chain with an amino acid sequence having at least 80% sequence identity to SEQ ID NO: 2.

In some embodiments, the target dose is at least about 1 mg, 1.05 mg, 1.1 mg, 1.15 mg, 1.2 mg, 1.25 mg, 1.3 mg, 1.35 mg, 1.4 mg, 1.45 mg, 1.5 mg, 1.55 mg, 1.6 mg, 1.65 mg, 1.7 mg, 1.75 mg, 1.8 mg, 1.85 mg, 1.9 mg, 1.95 mg, 2 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, 2.95 mg, 3 mg, 3.05 mg, 3.1 mg, 3.15 mg, 3.2 mg, 3.25 mg, 3.3 mg, 3.35 mg, 3.4 mg, 3.45 mg, 3.5 mg, 3.55 mg, 3.6 mg, 3.65 mg, 3.7 mg, 3.75 mg, 3.8 mg, 3.85 mg, 3.9 mg, 3.95 mg, 4 mg, 4.05 mg, 4.1 mg, 4.15 mg, 4.2 mg, 4.25 mg, 4.3 mg, 4.35 mg, 4.4 mg, 4.45 mg, 4.5 mg, 4.55 mg, 4.6 mg, 4.65 mg, 4.7 mg, 4.75 mg, 4.8 mg, 4.85 mg, 4.9 mg, 4.95 mg, 5 mg, 5.05 mg, 5.1 mg, 5.15 mg, 5.2 mg, 5.25 mg, 5.3 mg, 5.35 mg, 5.4 mg, 5.45 mg, 5.5 mg, 5.55 mg, 5.6 mg, 5.65 mg, 5.7 mg, 5.75 mg, 5.8 mg, 5.85 mg, 5.9 mg, 5.95 mg, 6 mg, 6.05 mg, 6.1 mg, 6.15 mg, 6.2 mg, 6.25 mg, 6.3 mg, 6.35 mg, 6.4 mg, 6.45 mg, 6.5 mg, 6.55 mg, 6.6 mg, 6.65 mg, 6.7 mg, 6.75 mg, 6.8 mg, 6.85 mg, 6.9 mg, 6.95 mg, 7 mg, 7.05 mg, 7.1 mg, 7.15 mg, 7.2 mg, 7.25 mg, 7.3 mg, 7.35 mg, 7.4 mg, 7.45 mg, 7.5 mg, 7.55 mg, 7.6 mg, 7.65 mg, 7.7 mg, 7.75 mg, 7.8 mg, 7.85 mg, 7.9 mg, 7.95 mg, 8 mg, 8.05 mg, 8.1 mg, 8.15 mg, 8.2 mg, 8.25 mg, 8.3 mg, 8.35 mg, 8.4 mg, 8.45 mg, 8.5 mg, 8.55 mg, 8.6 mg, 8.65 mg, 8.7 mg, 8.75 mg, 8.8 mg, 8.85 mg, 8.9 mg, 8.95 mg, 9 mg, 9.05 mg, 9.1 mg, 9.15 mg, 9.2 mg, 9.25 mg, 9.3 mg, 9.35 mg, 9.4 mg, 9.45 mg, 9.5 mg, 9.55 mg, 9.6 mg, 9.65 mg, 9.7 mg, 9.75 mg, 9.8 mg, 9.85 mg, 9.9 mg, 9.95 mg, or at least about 10 mg. In some embodiments, the target dose is about 1 mg, 1.05 mg, 1.1 mg, 1.15 mg, 1.2 mg, 1.25 mg, 1.3 mg, 1.35 mg, 1.4 mg, 1.45 mg, 1.5 mg, 1.55 mg, 1.6 mg, 1.65 mg, 1.7 mg, 1.75 mg, 1.8 mg, 1.85 mg, 1.9 mg, 1.95 mg, 2 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, 2.95 mg, 3 mg, 3.05 mg, 3.1 mg, 3.15 mg, 3.2 mg, 3.25 mg, 3.3 mg, 3.35 mg, 3.4 mg, 3.45 mg, 3.5 mg, 3.55 mg, 3.6 mg, 3.65 mg, 3.7 mg, 3.75 mg, 3.8 mg, 3.85 mg, 3.9 mg, 3.95 mg, 4 mg, 4.05 mg, 4.1 mg, 4.15 mg, 4.2 mg, 4.25 mg, 4.3 mg, 4.35 mg, 4.4 mg, 4.45 mg, 4.5 mg, 4.55 mg, 4.6 mg, 4.65 mg, 4.7 mg, 4.75 mg, 4.8 mg, 4.85 mg, 4.9 mg, 4.95 mg, 5 mg, 5.05 mg, 5.1 mg, 5.15 mg, 5.2 mg, 5.25 mg, 5.3 mg, 5.35 mg, 5.4 mg, 5.45 mg, 5.5 mg, 5.55 mg, 5.6 mg, 5.65 mg, 5.7 mg, 5.75 mg, 5.8 mg, 5.85 mg, 5.9 mg, 5.95 mg, 6 mg, 6.05 mg, 6.1 mg, 6.15 mg, 6.2 mg, 6.25 mg, 6.3 mg, 6.35 mg, 6.4 mg, 6.45 mg, 6.5 mg, 6.55 mg, 6.6 mg, 6.65 mg, 6.7 mg, 6.75 mg, 6.8 mg, 6.85 mg, 6.9 mg, 6.95 mg, 7 mg, 7.05 mg, 7.1 mg, 7.15 mg, 7.2 mg, 7.25 mg, 7.3 mg, 7.35 mg, 7.4 mg, 7.45 mg, 7.5 mg, 7.55 mg, 7.6 mg, 7.65 mg, 7.7 mg, 7.75 mg, 7.8 mg, 7.85 mg, 7.9 mg, 7.95 mg, 8 mg, 8.05 mg, 8.1 mg, 8.15 mg, 8.2 mg, 8.25 mg, 8.3 mg, 8.35 mg, 8.4 mg, 8.45 mg, 8.5 mg, 8.55 mg, 8.6 mg, 8.65 mg, 8.7 mg, 8.75 mg, 8.8 mg, 8.85 mg, 8.9 mg, 8.95 mg, 9 mg, 9.05 mg, 9.1 mg, 9.15 mg, 9.2 mg, 9.25 mg, 9.3 mg, 9.35 mg, 9.4 mg, 9.45 mg, 9.5 mg, 9.55 mg, 9.6 mg, 9.65 mg, 9.7 mg, 9.75 mg, 9.8 mg, 9.85 mg, 9.9 mg, 9.95 mg, or about 10 mg.

In some embodiments, the first dose is about 100 μg. In some embodiments, the first dose is about 150 μg. In some embodiments, the first dose is about 200 μg. In some embodiments, the first dose is about 250 μg. In some embodiments, the first dose is about 300 μg. In some embodiments, the first dose is about 350 μg. In some embodiments, the first dose is about 400 μg. In some embodiments, the first dose is about 450 μg. In some embodiments, the first dose is about 500 μg. In some embodiments, the first dose is about 450 μg. In some embodiments, the first dose is about 550 μg. In some embodiments, the first dose is about 450 μg. In some embodiments, the first dose is about 600 μg. In some embodiments, the first dose is about 450 μg. In some embodiments, the first dose is about 650 μg. In some embodiments, the first dose is about 450 μg. In some embodiments, the first dose is about 700 μg. In some embodiments, the first dose is about 450 μg. In some embodiments, the first dose is about 750 μg. In some embodiments, the first dose is about 450 μg. In some embodiments, the first dose is about 800 μg.

In some embodiments, the second dose is about 150 μg. In some embodiments, the second dose is about 200 μg. In some embodiments, the second dose is about 250 μg. In some embodiments, the second dose is about 300 μg. In some embodiments, the second dose is about 350 μg. In some embodiments, the second dose is about 400 μg. In some embodiments, the second dose is about 450 μg. In some embodiments, the second dose is about 500 μg. In some embodiments, the second dose is about 550 μg. In some embodiments, the second dose is about 600 μg. In some embodiments, the second dose is about 650 μg. In some embodiments, the second dose is about 700 μg. In some embodiments, the second dose is about 750 μg. In some embodiments, the second dose is about 800 μg. In some embodiments, the second dose is about 900 μg. In some embodiments, the second dose is about 1 mg. In some embodiments, the second dose is about 1.1 mg. In some embodiments, the second dose is about 1.2 mg. In some embodiments, the second dose is about 1.3 mg. In some embodiments, the second dose is about 1.4 mg. In some embodiments, the second dose is about 1.5 mg. In some embodiments, the second dose is about 1.6 mg. In some embodiments, the second dose is about 1.7 mg. In some embodiments, the second dose is about 1.8 mg. In some embodiments, the second dose is about 1.9 mg. In some embodiments, the second dose is about 2 mg. In some embodiments, the second dose is about 2.4 mg. In some embodiments, the second dose is about 2.8 mg. In some embodiments, the second dose is about 3.2 mg. In some embodiments, the second dose is about 3.6 mg. In some embodiments, the second dose is about 4 mg. In some embodiments, the second dose is about 4.4 mg. In some embodiments, the second dose is about 4.8 mg. In some embodiments, the second dose is about 5 mg.

In some embodiments, the first dose, the second dose, and the target dose are about 100 μg, 200 μg, and 600 μg, respectively. In some embodiments, the first dose, the second dose, and the target dose are about 200 μg, 400 μg, and 1.2 mg, respectively. In some embodiments, the first dose, the second dose, and the target dose are about 300 μg, 800 μg, and 2.4 mg, respectively. In some embodiments, the first dose, the second dose, and the target dose are about 400 μg, 1.6 mg, and 4.8 mg, respectively. In some embodiments, the first dose, the second dose, and the target dose are about 500 μg, 2 mg, and 6 mg, respectively. In some embodiments, the first dose, the second dose, and the target dose are about 600 μg, 2.4 mg, and 7.2 mg, respectively. In some embodiments, the first dose, the second dose, and the target dose are about 700 μg, 2.8 mg, and 8.4 mg, respectively. In some embodiments, the first dose, the second dose, and the target dose are about 800 μg, 3.2 mg, and 9.6 mg, respectively. In some embodiments, the first dose, the second dose, and the target dose are about 900 μg, 3.6 mg, and 10.8 mg, respectively. In some embodiments, the first dose, the second dose, and the target dose are about 1 mg, 4 mg, and 12 mg, respectively.

In some embodiments, a dose disclosed herein is repeated more than two times. In some embodiments, the administering comprises administering the target dose at least two times. In some embodiments, the administering comprises administering the target dose at least three times. In some embodiments, the administering comprises administering the target dose at least four times. In some embodiments, the administering comprises administering the target dose at least five times. In some embodiments, the administering comprises administering the target dose at least six times. In some embodiments, the administering comprises administering the target dose at least seven times. In some embodiments, the administering comprises administering the target dose at least eight times. In some embodiments, the administering comprises administering the target dose at least nine times. In some embodiments, the administering comprises administering the target dose at least ten times. In some embodiments, the administering comprises administering the target dose more than ten times.

In some embodiments, the administering comprises administering the first dose at least two times. In some embodiments, the administering comprises administering the first dose at least three times. In some embodiments, the administering comprises administering the first dose at least four times. In some embodiments, the administering comprises administering the first dose at least five times. In some embodiments, the administering comprises administering the first dose at least six times. In some embodiments, the administering comprises administering the first dose at least seven times. In some embodiments, the administering comprises administering the first dose at least eight times. In some embodiments, the administering comprises administering the first dose at least nine times. In some embodiments, the administering comprises administering the first dose at least ten times. In some embodiments, the administering comprises administering the first dose more than ten times.

In some embodiments, the administering comprises administering the second dose at least two times. In some embodiments, the administering comprises administering the second dose at least three times. In some embodiments, the administering comprises administering the second dose at least four times. In some embodiments, the administering comprises administering the second dose at least five times. In some embodiments, the administering comprises administering the second dose at least six times. In some embodiments, the administering comprises administering the second dose at least seven times. In some embodiments, the administering comprises administering the second dose at least eight times. In some embodiments, the administering comprises administering the second dose at least nine times. In some embodiments, the administering comprises administering the second dose at least ten times. In some embodiments, the administering comprises administering the second dose more than ten times.

In some embodiments, the first dose and the target dose are administered within 3 days, 4 days, 5 days, 6 days, 7 days, 8 days, 9 days, 10 days, or any period of time therebetween. In some embodiments, two doses of the isolated recombinant polypeptide complex are administered within 3 days, 4 days, 5 days, 6 days, 7 days, 8 days, 9 days, 10 days, or any period of time therebetween.

In some embodiments, the first dose and the target dose are administered within three days. In some embodiments, the first dose and the target dose are administered within four days. In some embodiments, the first dose and the target dose are administered within five days. In some embodiments, the first dose and the target dose are administered within six days. In some embodiments, the first dose and the target dose are administered within one week. In some embodiments, the first dose and the target dose are administered within two weeks. In some embodiments, the first dose and the target dose are administered within three weeks. In some embodiments, the first dose and the target dose are administered within four weeks. In some embodiments, the first dose and the target dose are administered within five weeks. In some embodiments, the first dose and the target dose are administered within six weeks. In some embodiments, the first dose and the target dose are administered within seven weeks. In some embodiments, the first dose and the target dose are administered within eight weeks. In some embodiments, the first dose and the target dose are administered within nine weeks. In some embodiments, the first dose and the target dose are administered within ten weeks. In some embodiments, the first dose and the target dose are administered within one month. In some embodiments, the first dose and the target dose are administered within two months. In some embodiments, the first dose and the target dose are administered within three months. In some embodiments, the first dose and the target dose are administered within four months. In some embodiments, the first dose and the target dose are administered within five months. In some embodiments, the first dose and the target dose are administered within six months. In some embodiments, the first dose and the target dose are administered within seven months. In some embodiments, the first dose and the target dose are administered within eight months. In some embodiments, the first dose and the target dose are administered within nine months. In some embodiments, the first dose and the target dose are administered within ten months. In some embodiments, the first dose and the target dose are administered within eleven months. In some embodiments, the first dose and the target dose are administered within twelve months. In some embodiments, the first dose and the target dose are administered within one and half years. In some embodiments, the first dose and the target dose are administered within two years. In some embodiments, the first dose and the target dose are administered within three years. In some embodiments, the first dose and the target dose are administered within four years. In some embodiments, the first dose and the target dose are administered within five years.

In some embodiments, the treatment cycle comprises 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days. In some embodiments, the treatment cycle comprises 1 to 60 days. In some embodiments, the treatment cycle comprises 1 to 30 days. In some embodiments, the treatment cycle comprises 21 days. In some embodiments, the treatment cycle comprises 28 days.

In some embodiments, the subject exhibits a reduction in the expression of prostate specific antigen (PSA) after the administering. In some embodiments, the subject exhibits a reduction in the expression of prostate specific antigen (PSA) after administration of the first dose, the target dose, a dose between the first dose and the target dose, or a repeat thereof. In some embodiments, the subject exhibits a reduction in the expression of PSA compared with the PSA expression level measured before administration of the first dose of the isolated recombinant polypeptide complex. In some embodiments, a reduction in the expression of PSA is about 1%, 2%, 3%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, or any percentage therebetween.

In some embodiments, the isolated recombinant polypeptide complex being administered is in a pharmaceutical composition comprising a pharmaceutically acceptable excipient. In some embodiments, the pharmaceutically acceptable excipient comprises a buffer, a stabilizing agent, a tonicity agent, a surfactant, or combinations thereof. In some embodiments, the pharmaceutically acceptable excipient comprises a buffer. In some embodiments, the pharmaceutically acceptable excipient comprises a stabilizing agent. In some embodiments, the pharmaceutically acceptable excipient comprises a tonicity agent. In some embodiments, the pharmaceutically acceptable excipient comprises a surfactant. In some embodiments, the buffer comprises an amino acid or a derivative thereof. In some embodiments, the amino acid or the derivative thereof comprises L-histidine, L-histidine monohydrochloride monohydrate, or combinations thereof. In some embodiments, the amino acid or the derivative thereof comprises L-histidine. In some embodiments, the amino acid or the derivative thereof comprises L-histidine monohydrochloride monohydrate. In some embodiments, the stabilizing agent comprises sugar. In some embodiments, the sugar comprises sucrose. In some embodiments, the tonicity agent comprises sugar. In some embodiments, the sugar comprises sucrose. In some embodiments, the surfactant comprises a polysorbate. In some embodiments, the surfactant comprises polysorbate 20. In some embodiments, the surfactant comprises polysorbate 40. In some embodiments, the surfactant comprises polysorbate 60. In some embodiments, the surfactant comprises polysorbate 80.

In some embodiments, the total amount of L-histidine in the pharmaceutical composition is in the forms of both L-histidine and L-histidine monohydrochloride monohydrate. In some embodiments, the total amount of L-histidine in the pharmaceutical composition is about 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM, 15 mM, 15.1 mM, 15.2 mM, 15.3 mM, 15.4 mM, 15.5 mM, 15.6 mM, 15.7 mM, 15.8 mM, 15.9 mM, 16 mM, 16.1 mM, 16.2 mM, 16.3 mM, 16.4 mM, 16.5 mM, 16.6 mM, 16.7 mM, 16.8 mM, 16.9 mM, 17 mM, 17.1 mM, 17.2 mM, 17.3 mM, 17.4 mM, 17.5 mM, 17.6 mM, 17.7 mM, 17.8 mM, 17.9 mM, 18 mM, 18.1 mM, 18.2 mM, 18.3 mM, 18.4 mM, 18.5 mM, 18.6 mM, 18.7 mM, 18.8 mM, 18.9 mM, 19 mM, 19.1 mM, 19.2 mM, 19.3 mM, 19.4 mM, 19.5 mM, 19.6 mM, 19.7 mM, 19.8 mM, 19.9 mM, or 20 mM, or any concentration therebetween. In some embodiments, the total amount of L-histidine in the pharmaceutical composition is about 10 mM. In some embodiments, the total amount of L-histidine in the pharmaceutical composition is in the form L-histidine. In some embodiments, the total amount of L-histidine in the pharmaceutical composition is in the form of L-histidine monohydrochloride monohydrate. In some embodiments, the total amount of L-histidine in the pharmaceutical composition is in the forms of both L-histidine and L-histidine monohydrochloride monohydrate. In some embodiments, the total amount of L-histidine in the pharmaceutical composition is about 10 mM in the forms of both L-histidine and L-histidine monohydrochloride monohydrate. In some embodiments, the molar ratio of L-histidine to L-histidine monohydrochloride monohydrate is about 1:1, 1:2, 1:3, 1:4, 1:5, 1:6, 2:1, 2:3, 2:5, 3:1, 3:2, 3:4, 3:5, 4:1, 4:3, 4:5, 5:1, 5:2, 5:3, 5:4, 5:6, 6:1, or 6:5. In some embodiments, the molar ratio of L-histidine to L-histidine monohydrochloride monohydrate is about 3:2.

In some embodiments, the pharmaceutical composition comprises sucrose in an amount of about 1% (w/v), 1.5% (w/v), 2% (w/v), 2.5% (w/v), 3% (w/v), 3.5% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), 10% (w/v), 10.5% (w/v), 11% (w/v), 11.5% (w/v), 12% (w/v), 12.5% (w/v), 13% (w/v), 13.5% (w/v), 14% (w/v), 14.5% (w/v), 15% (w/v), 15.5% (w/v), 16% (w/v), 16.5% (w/v), 17% (w/v), 17.5% (w/v), 18% (w/v), 18.5% (w/v), 19% (w/v), 19.5% (w/v), or 20% (w/v), or any concentration therebetween. In some embodiments, the pharmaceutical composition comprises sucrose in an amount of about 8% (w/v).

In some embodiments, the pharmaceutical composition comprises polysorbate 20 in an amount of at least 0.001% (w/v), 0.0015% (w/v), 0.002% (w/v), 0.0025% (w/v), 0.003% (w/v), 0.0035% (w/v), 0.004% (w/v), 0.0045% (w/v), 0.005% (w/v), 0.0055% (w/v), 0.006% (w/v), 0.0065% (w/v), 0.007% (w/v), 0.0075% (w/v), 0.008% (w/v), 0.0085% (w/v), 0.009% (w/v), 0.0095% (w/v), 0.01% (w/v), 0.0105% (w/v), 0.011% (w/v), 0.0115% (w/v), 0.012% (w/v), 0.0125% (w/v), 0.013% (w/v), 0.0135% (w/v), 0.014% (w/v), 0.0145% (w/v), 0.015% (w/v), 0.0155% (w/v), 0.016% (w/v), 0.0165% (w/v), 0.017% (w/v), 0.0175% (w/v), 0.018% (w/v), 0.0185% (w/v), 0.019% (w/v), 0.0195% (w/v), 0.02% (w/v), 0.0205% (w/v), 0.021% (w/v), 0.0215% (w/v), 0.022% (w/v), 0.0225% (w/v), 0.023% (w/v), 0.0235% (w/v), 0.024% (w/v), 0.0245% (w/v), 0.025% (w/v), 0.0255% (w/v), 0.026% (w/v), 0.0265% (w/v), 0.027% (w/v), 0.0275% (w/v), 0.028% (w/v), 0.0285% (w/v), 0.029% (w/v), 0.0295% (w/v), 0.03% (w/v), 0.0305% (w/v), 0.031% (w/v), 0.0315% (w/v), 0.032% (w/v), 0.0325% (w/v), 0.033% (w/v), 0.0335% (w/v), 0.034% (w/v), 0.0345% (w/v), 0.035% (w/v), 0.0355% (w/v), 0.036% (w/v), 0.0365% (w/v), 0.037% (w/v), 0.0375% (w/v), 0.038% (w/v), 0.0385% (w/v), 0.039% (w/v), 0.0395% (w/v), 0.04% (w/v), 0.0405% (w/v), 0.041% (w/v), 0.0415% (w/v), 0.042% (w/v), 0.0425% (w/v), 0.043% (w/v), 0.0435% (w/v), 0.044% (w/v), 0.0445% (w/v), 0.045% (w/v), 0.0455% (w/v), 0.046% (w/v), 0.0465% (w/v), 0.047% (w/v), 0.0475% (w/v), 0.048% (w/v), 0.0485% (w/v), 0.049% (w/v), 0.0495% (w/v), 0.05% (w/v), 0.055% (w/v), 0.06% (w/v), 0.065% (w/v), 0.07% (w/v), 0.075% (w/v), 0.08% (w/v), 0.085% (w/v), 0.09% (w/v), 0.095% (w/v), or about 0.1% (w/v), or any concentration therebetween. In some embodiments, the pharmaceutical composition comprises polysorbate 20 in an amount of at least 0.01% (w/v). In some embodiments, the pharmaceutical composition comprises polysorbate 20 in an amount of about 0.01% (w/v).

In some embodiments, the pharmaceutical composition comprises about 6 mM L-histidine. In some embodiments, the pharmaceutical composition comprises about 4 mM L-histidine monohydrochloride monohydrate. In some embodiments, the pharmaceutical composition comprises about 8% (w/v) sucrose. In some embodiments, the pharmaceutical composition comprises about 0.01% (w/v) polysorbate 20. In some embodiments, the pharmaceutical composition comprises about 6 mM L-histidine, about 4 mM L-histidine monohydrochloride monohydrate, about 8% (w/v) sucrose, and about 0.01% (w/v) polysorbate 20.

In some embodiments, the pharmaceutical composition comprises the isolated recombinant polypeptide complex in an amount of about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, 3 mg/ml, 3.1 mg/ml, 3.2 mg/ml, 3.3 mg/ml, 3.4 mg/ml, 3.5 mg/ml, 3.6 mg/ml, 3.7 mg/ml, 3.8 mg/ml, 3.9 mg/ml, 4 mg/ml, 4.1 mg/ml, 4.2 mg/ml, 4.3 mg/ml, 4.4 mg/ml, 4.5 mg/ml, 4.6 mg/ml, 4.7 mg/ml, 4.8 mg/ml, 4.9 mg/ml, 5 mg/ml, 5.1 mg/ml, 5.2 mg/ml, 5.3 mg/ml, 5.4 mg/ml, 5.5 mg/ml, 5.6 mg/ml, 5.7 mg/ml, 5.8 mg/ml, 5.9 mg/ml, 6 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, 9 mg/ml, 9.1 mg/ml, 9.2 mg/ml, 9.3 mg/ml, 9.4 mg/ml, 9.5 mg/ml, 9.6 mg/ml, 9.7 mg/ml, 9.8 mg/ml, 9.9 mg/ml, 10 mg/ml, 10.1 mg/ml, 10.2 mg/ml, 10.3 mg/ml, 10.4 mg/ml, 10.5 mg/ml, 10.6 mg/ml, 10.7 mg/ml, 10.8 mg/ml, 10.9 mg/ml, 11 mg/ml, 11.1 mg/ml, 11.2 mg/ml, 11.3 mg/ml, 11.4 mg/ml, 11.5 mg/ml, 11.6 mg/ml, 11.7 mg/ml, 11.8 mg/ml, 11.9 mg/ml, 12 mg/ml, 12.1 mg/ml, 12.2 mg/ml, 12.3 mg/ml, 12.4 mg/ml, 12.5 mg/ml, 12.6 mg/ml, 12.7 mg/ml, 12.8 mg/ml, 12.9 mg/ml, 13 mg/ml, 13.1 mg/ml, 13.2 mg/ml, 13.3 mg/ml, 13.4 mg/ml, 13.5 mg/ml, 13.6 mg/ml, 13.7 mg/ml, 13.8 mg/ml, 13.9 mg/ml, 14 mg/ml, 14.1 mg/ml, 14.2 mg/ml, 14.3 mg/ml, 14.4 mg/ml, 14.5 mg/ml, 14.6 mg/ml, 14.7 mg/ml, 14.8 mg/ml, 14.9 mg/ml, 15 mg/ml, 15.1 mg/ml, 15.2 mg/ml, 15.3 mg/ml, 15.4 mg/ml, 15.5 mg/ml, 15.6 mg/ml, 15.7 mg/ml, 15.8 mg/ml, 15.9 mg/ml, 16 mg/ml, 16.1 mg/ml, 16.2 mg/ml, 16.3 mg/ml, 16.4 mg/ml, 16.5 mg/ml, 16.6 mg/ml, 16.7 mg/ml, 16.8 mg/ml, 16.9 mg/ml, 17 mg/ml, 17.1 mg/ml, 17.2 mg/ml, 17.3 mg/ml, 17.4 mg/ml, 17.5 mg/ml, 17.6 mg/ml, 17.7 mg/ml, 17.8 mg/ml, 17.9 mg/ml, 18 mg/ml, 18.1 mg/ml, 18.2 mg/ml, 18.3 mg/ml, 18.4 mg/ml, 18.5 mg/ml, 18.6 mg/ml, 18.7 mg/ml, 18.8 mg/ml, 18.9 mg/ml, 19 mg/ml, 19.1 mg/ml, 19.2 mg/ml, 19.3 mg/ml, 19.4 mg/ml, 19.5 mg/ml, 19.6 mg/ml, 19.7 mg/ml, 19.8 mg/ml, 19.9 mg/ml, or 20 mg/ml, or any concentration therebetween. In some embodiments, the pharmaceutical composition comprises about 2 mg/ml of the isolated recombinant polypeptide complex.

In some embodiments, the pharmaceutical composition comprises a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0, or any value therebetween. In some embodiments, the pharmaceutical composition comprises a pH between about 5 and about 7. In some embodiments, the pharmaceutical composition comprises a pH of about 6.3.

### Disulfide Bond(s)

In some embodiments, the recombinant polypeptide comprises at least one disulfide bond formed by a pair of cysteine residues. In some embodiments, the recombinant polypeptide comprises at least one disulfide bond formed by a pair of cysteine residues in the first chain. In some embodiments, the recombinant polypeptide comprises at least one disulfide bond formed by a pair of cysteine residues in the second chain. In some embodiments, the recombinant polypeptide comprises at least one disulfide bond formed by a pair of cysteine residues in the first chain and the second chain. In some embodiments, the recombinant polypeptide comprises at least two, at least three, or at least four disulfide bonds formed by pairs of cysteine residues comprising the pair of cysteine residues. In some embodiments, the recombinant polypeptide comprises at least two disulfide bonds formed by pairs of cysteine residues comprising the pair of cysteine residues. In some embodiments, the recombinant polypeptide comprises at least three disulfide bonds formed by pairs of cysteine residues comprising the pair of cysteine residues. In some embodiments, the recombinant polypeptide comprises at least four disulfide bonds formed by pairs of cysteine residues comprising the pair of cysteine residues. In some embodiments, the recombinant polypeptide comprises at least five disulfide bonds formed by pairs of cysteine residues comprising the pair of cysteine residues. In some embodiments, the recombinant polypeptide comprises at least six disulfide bonds formed by pairs of cysteine residues comprising the pair of cysteine residues. In some embodiments, the recombinant polypeptide comprises at least seven disulfide bonds formed by pairs of cysteine residues comprising the pair of cysteine residues. In some embodiments, the recombinant polypeptide comprises at least eight disulfide bonds formed by pairs of cysteine residues comprising the pair of cysteine residues.

In some embodiments, a pair of cysteine residues that form a disulfide bond corresponds to amino acid positions: (i) Cysteine 22 and Cysteine 96 of SEQ ID NO: 1, or (ii) Cysteine 138 and Cysteine 148 of SEQ ID NO: 1, or (iii) Cysteine 199 and Cysteine 275 of SEQ ID NO: 1, or (iv) Cysteine 339 and Cysteine 407 of SEQ ID NO: 1, or (v) Cysteine 454 and Cysteine 519 of SEQ ID NO: 1, or(vi) Cysteine 565 and Cysteine 625 of SEQ ID NO: 1, or (vii) Cysteine 645 of SEQ ID NO: 1 and Cysteine 226 of SEQ ID NO: 2, or (viii) Cysteine 22 and Cysteine 96 of SEQ ID NO: 2, or (ix) Cysteine 150 and Cysteine 206 of SEQ ID NO: 2.

In some embodiments of the recombinant peptide, at least one disulfide bond formed by a pair of cysteine residues is formed by cysteine residues selected from and corresponding to positions Cysteine 22, Cysteine 96, Cysteine 138, Cysteine 148, Cysteine 199, Cysteine 275, Cysteine 339, Cysteine 407, Cysteine 454, Cysteine 519, Cysteine 565, or Cysteine 625 of SEQ ID NO: 1. In some embodiments of the recombinant polypeptide, at least one disulfide bond formed by a pair of cysteine residues is formed by cysteine residues selected from and corresponding to positions Cysteine 645 of SEQ ID NO: 1 and Cysteine 226 of SEQ ID NO: 2. In some embodiments of the recombinant peptide, at least one disulfide bond formed by a pair of cysteine residues is formed by cysteine residues selected from and corresponding to positions Cysteine 22, Cysteine 96, Cysteine 150, Cysteine 206 of SEQ ID NO: 2. The presence of disulfide bond(s) may be determined by mass spectrometry (MS).

In some embodiments, the recombinant polypeptide comprises at least one cysteine residue that is a free sulfhydryl. The presence of free sulfhydryl(s) may be determined by mass spectrometry (MS).

In some embodiments, the subject is diagnosed with cancer. In some embodiments, the cancer comprises prostate cancer. In some embodiments, the cancer comprises metastatic castration-resistant prostate cancer (mCRPC).

In some embodiments, the isolated recombinant polypeptide complex is cleaved by a tumor specific protease to generate an enzymatic product of the isolated recombinant polypeptide complex after the administering. In some embodiments, the tumor specific protease comprises two or more proteases. In some embodiments, the isolated recombinant polypeptide complex is cleaved by a first protease of the two or more proteases to generate a first metabolic product of the isolated recombinant polypeptide complex. In some embodiments, the isolated recombinant polypeptide complex is cleaved by a second protease of the two or more proteases to generate a second metabolic product of the isolated recombinant polypeptide complex. In some embodiments, the first protease comprises a serine protease. In some embodiments, the second protease comprises a matrix metalloprotease. In some embodiments, the serine protease comprises recombinant human matriptase (MTSP1). In some embodiments, the matrix metalloprotease comprises recombinant human matrix metalloprotease 9 (MMP9).

### Embodiments

Embodiment 1 comprises a method for treating cancer comprising administering to a subject in need thereof a first dose and a target dose of an isolated recombinant polypeptide complex, wherein the first dose is at least about 100 μg and the target dose is higher than the first dose, and wherein the isolated recombinant polypeptide complex comprises a first chain with an amino acid sequence having at least 80% sequence identity to SEQ ID NO: 1 and a second chain with an amino acid sequence having at least 80% sequence identity to SEQ ID NO: 2.
Embodiment 2 comprises the method of embodiment 1, wherein the first dose is about 100 μg to about 1 mg.
Embodiment 3 comprises the method of embodiment 1 or 2, wherein the first dose is at least about 150 μg.
Embodiment 4 comprises the method of embodiment 1 or 2, wherein the first dose is at least about 200 μg.
Embodiment 5 comprises the method of embodiment 1 or 2, wherein the first dose is at least about 250 μg.
Embodiment 6 comprises the method of embodiment 1 or 2, wherein the first dose is at least about 300 μg.
Embodiment 7 comprises the method of embodiment 1 or 2, wherein the first dose is at least about 400 μg.
Embodiment 8 comprises the method of embodiment 1 or 2, wherein the first dose is at least about 500 μg.
Embodiment 9 comprises the method of embodiment 1 or 2, wherein the first dose is at least about 600 μg.
Embodiment 10 comprises the method of embodiment 1 or 2, wherein the first dose is at least about 700 μg.
Embodiment 11 comprises the method of embodiment 1 or 2, wherein the first dose is at least about 800 μg.
Embodiment 12 comprises the method of embodiment 1 or 2, wherein the first dose is at least about 900 μg.
Embodiment 13 comprises the method of embodiment 1 or 2, wherein the first dose is about 100 μg.
Embodiment 14 comprises the method of embodiment 1 or 2, wherein the first dose is about 150 μg.
Embodiment 15 comprises the method of embodiment 1 or 2, wherein the first dose is about 200 μg.
Embodiment 16 comprises the method of embodiment 1 or 2, wherein the first dose is about 250 μg.
Embodiment 17 comprises the method of embodiment 1 or 2, wherein the first dose is about 300 μg.
Embodiment 18 comprises the method of embodiment 1 or 2, wherein the first dose is about 400 μg.
Embodiment 19 comprises the method of embodiment 1 or 2, wherein the first dose is about 500 μg.
Embodiment 20 comprises the method of embodiment 1 or 2, wherein the first dose is about 600 μg.
Embodiment 21 comprises the method of embodiment 1 or 2, wherein the first dose is about 700 μg.
Embodiment 22 comprises the method of embodiment 1 or 2, wherein the first dose is about 800 μg.
Embodiment 23 comprises the method of embodiment 1 or 2, wherein the first dose is about 900 μg.
Embodiment 24 comprises the method of embodiment 1 or 2, wherein the first dose is about 1 mg.
Embodiment 25 comprises the method of embodiment 1, wherein the target dose is about 450 μg to about 10 mg.
Embodiment 26 comprises the method of embodiment 1 or embodiment 25, wherein the target dose is at least about 450 μg.
Embodiment 27 comprises the method of embodiment 1 or embodiment 25, wherein the target dose is at least about 750 μg.
Embodiment 28 comprises the method of embodiment 1 or embodiment 25, wherein the target dose is at least about 1 mg.
Embodiment 29 comprises the method of embodiment 1 or embodiment 25, wherein the target dose is at least about 1.5 mg.
Embodiment 30 comprises the method of embodiment 1 or embodiment 25, wherein the target dose is at least about 2 mg.
Embodiment 31 comprises the method of embodiment 1 or embodiment 25, wherein the target dose is at least about 2.5 mg.
Embodiment 32 comprises the method of embodiment 1 or embodiment 25, wherein the target dose is at least about 3 mg.
Embodiment 33 comprises the method of embodiment 1 or embodiment 25, wherein the target dose is at least about 4 mg.
Embodiment 34 comprises the method of embodiment 1 or embodiment 25, wherein the target dose is at least about 5 mg.
Embodiment 35 comprises the method of embodiment 1 or embodiment 25, wherein the target dose is at least about 6 mg.
Embodiment 36 comprises the method of embodiment 1 or embodiment 25, wherein the target dose is at least about 7 mg.
Embodiment 37 comprises the method of embodiment 1 or embodiment 25, wherein the target dose is at least about 8 mg.
Embodiment 38 comprises the method of embodiment 1 or embodiment 25, wherein the target dose is at least about 9 mg.
Embodiment 39 comprises the method of embodiment 1 or embodiment 25, wherein the target dose is about 450 μg.
Embodiment 40 comprises the method of embodiment 1 or embodiment 25, wherein the target dose is about 750 μg.
Embodiment 41 comprises the method of embodiment 1 or embodiment 25, wherein the target dose is about 1 mg.
Embodiment 42 comprises the method of embodiment 1 or embodiment 25, wherein the target dose is about 1.5 mg.
Embodiment 43 comprises the method of embodiment 1 or embodiment 25, wherein the target dose is about 2 mg.
Embodiment 44 comprises the method of embodiment 1 or embodiment 25, wherein the target dose is about 2.5 mg.
Embodiment 45 comprises the method of embodiment 1 or embodiment 25, wherein the target dose is about 3 mg.
Embodiment 46 comprises the method of embodiment 1 or embodiment 25, wherein the target dose is about 4 mg.
Embodiment 47 comprises the method of embodiment 1 or embodiment 25, wherein the target dose is about 5 mg.
Embodiment 48 comprises the method of embodiment 1 or embodiment 25, wherein the target dose is about 6 mg.
Embodiment 49 comprises the method of embodiment 1 or embodiment 25, wherein the target dose is about 7 mg.
Embodiment 50 comprises the method of embodiment 1 or embodiment 25, wherein the target dose is about 8 mg.
Embodiment 51 comprises the method of embodiment 1 or embodiment 25, wherein the target dose is about 9 mg.
Embodiment 52 comprises the method of embodiment 1 or embodiment 25, wherein the target dose is about 10 mg.
Embodiment 53 comprises the method of any one of embodiments 1-52, further comprising administering to the subject a second dose of the isolated recombinant polypeptide complex, wherein the second dose is higher than the first dose and lower than the target dose, and wherein the second dose is administered between the first dose and the target dose.
Embodiment 54 comprises the method of embodiment 53, wherein the second dose is about 300 μg to about 5 mg.
Embodiment 55 comprises the method of embodiment 53 or 54, wherein the second dose is at least about 300 μg.
Embodiment 56 comprises the method of embodiment 53 or 54, wherein the second dose is at least about 400 μg.
Embodiment 57 comprises the method of embodiment 53 or 54, wherein the second dose is at least about 500 μg.
Embodiment 58 comprises the method of embodiment 53 or 54, wherein the second dose is at least about 600 μg.
Embodiment 59 comprises the method of embodiment 53 or 54, wherein the second dose is at least about 700 μg.
Embodiment 60 comprises the method of embodiment 53 or 54, wherein the second dose is at least about 800 μg.
Embodiment 61 comprises the method of embodiment 53 or 54, wherein the second dose is at least about 900 μg.
Embodiment 62 comprises the method of embodiment 53 or 54, wherein the second dose is at least about 1 mg.
Embodiment 63 comprises the method of embodiment 53 or 54, wherein the second dose is at least about 2 mg.
Embodiment 64 comprises the method of embodiment 53 or 54, wherein the second dose is at least about 3 mg.
Embodiment 65 comprises the method of embodiment 53 or 54, wherein the second dose is at least about 4 mg.
Embodiment 66 comprises the method of embodiment 53 or 54, wherein the second dose is about 300 μg.
Embodiment 67 comprises the method of embodiment 53 or 54, wherein the second dose is about 400 μg.
Embodiment 68 comprises the method of embodiment 53 or 54, wherein the second dose is about 500 μg.
Embodiment 69 comprises the method of embodiment 53 or 54, wherein the second dose is about 600 μg.
Embodiment 70 comprises the method of embodiment 53 or 54, wherein the second dose is about 700 μg.
Embodiment 71 comprises the method of embodiment 53 or 54, wherein the second dose is about 800 μg.
Embodiment 72 comprises the method of embodiment 53 or 54, wherein the second dose is about 900 μg.
Embodiment 73 comprises the method of embodiment 53 or 54, wherein the second dose is about 1 mg.
Embodiment 74 comprises the method of embodiment 53 or 54, wherein the second dose is about 1.2 mg.
Embodiment 75 comprises the method of embodiment 53 or 54, wherein the second dose is about 1.5 mg.
Embodiment 76 comprises the method of embodiment 53 or 54, wherein the second dose is about 2 mg.
Embodiment 77 comprises the method of embodiment 53 or 54, wherein the second dose is about 2.5 mg.
Embodiment 78 comprises the method of embodiment 53 or 54, wherein the second dose is about 3 mg.
Embodiment 79 comprises the method of embodiment 53 or 54, wherein the second dose is about 3.5 mg.
Embodiment 80 comprises the method of embodiment 53 or 54, wherein, the second dose is about 4 mg.
Embodiment 81 comprises the method of embodiment 53 or 54, wherein the second dose is about 4.5 mg.
Embodiment 82 comprises the method of embodiment 53 or 54, wherein the second dose is about 5 mg.
Embodiment 83 comprises the method of embodiment 53 or 54, wherein the first dose, the second dose, and the target dose are about 100 μg, 300 μg, and 450 μg, respectively.
Embodiment 84 comprises the method of embodiment 53 or 54, wherein the first dose, the second dose, and the target dose are about 100 μg, 300 μg, and 1 mg, respectively.
Embodiment 85 comprises the method of embodiment 53 or 54, wherein the first dose, the second dose, and the target dose are about 200 μg, 600 μg, and 2 mg, respectively.
Embodiment 86 comprises the method of embodiment 53 or 54, wherein the first dose, the second dose, and the target dose are about 300 μg, 1 mg, and 3 mg, respectively.
Embodiment 87 comprises the method of embodiment 53 or 54, wherein the first dose, the second dose, and the target dose are about 400 μg, 1.2 mg, and 4 mg, respectively.
Embodiment 88 comprises the method of embodiment 53 or 54, wherein the first dose, the second dose, and the target dose are about 500 μg, 1.5 mg, and 5 mg, respectively.
Embodiment 89 comprises the method of embodiment 53 or 54, wherein the first dose, the second dose, and the target dose are about 600 μg, 2 mg, and 6 mg, respectively.
Embodiment 90 comprises the method of embodiment 53 or 54, wherein the first dose, the second dose, and the target dose are about 700 μg, 2.5 mg, and 7 mg, respectively.
Embodiment 91 comprises the method of embodiment 53 or 54, wherein the first dose, the second dose, and the target dose are about 800 μg, 3 mg, and 8 mg, respectively.
Embodiment 92 comprises the method of embodiment 53 or 54, wherein the first dose, the second dose, and the target dose are about 900 μg, 4 mg, and 9 mg, respectively.
Embodiment 93 comprises the method of embodiment 53 or 54, wherein the first dose, the second dose, and the target dose are about 1 mg, 5 mg, and 10 mg, respectively.
Embodiment 94 comprises the method of any one of embodiments 1-93, wherein the first dose, the second dose, or the target dose is administered weekly.
Embodiment 95 comprises the method of any one of embodiments 1-93, wherein the first dose, the second dose, or the target dose is administered once every two weeks.
Embodiment 96 comprises the method of any one of embodiments 1-93, wherein the first dose, the second dose, or the target dose is administered once every three weeks.
Embodiment 97 comprises the method of any one of embodiments 1-96, wherein the first dose, the second dose, and the target dose are administered weekly.
Embodiment 98 comprises the method of any one of embodiments 1-96, wherein the first dose and the second dose are administered weekly, and wherein the target dose is administered once every two weeks.
Embodiment 99 comprises the method of any one of embodiments 1-96, wherein the first dose and the second dose are administered weekly, and wherein the target dose is administered once every three weeks.
Embodiment 100 comprises the method of any one of embodiments 1-99, wherein the method comprises a treatment cycle that starts on day 1.
Embodiment 101 comprises the method of embodiment 100, wherein the treatment cycle is 21 days or 28 days.
Embodiment 102 comprises the method of embodiment 100 or 101, wherein the first dose, the second dose, and the target dose are administered on day 1, one of day 7, 8 or 9, and one of day 14, 15, or 16, respectively, of the treatment cycle.
Embodiment 103 comprises the method of embodiment 100 or 101, wherein the first dose, the second dose, and the target dose are administered on day 1, day 4, and day 8, respectively, of the treatment cycle.
Embodiment 104 comprises the method of embodiment 102, wherein the target dose is administered weekly after the one of day 14, 15, or 16, of the treatment cycle.
Embodiment 105 comprises the method of embodiment 102, wherein the target dose is administered once every two weeks after the one of day 14, 15, or 16, of the treatment cycle.
Embodiment 106 comprises the method of embodiment 102, wherein the target dose is administered once every three weeks after the one of day 14, 15, or 16, of the treatment cycle.
Embodiment 107 comprises the method of embodiment 103, wherein the target dose is administered weekly after day 8 of the treatment cycle.
Embodiment 108 comprises the method of embodiment 103, wherein the target dose is administered once every two weeks after day 8 of the treatment cycle.
Embodiment 109 comprises the method of embodiment 103, wherein the target dose is administered once every three weeks after day 8 of the treatment cycle.
Embodiment 110 comprises the method of any one of embodiments 1-109, wherein the target dose is administered at least once, at least twice, at least 3 times, at least 4 times, at least 5 times, at least 6 times, at least 7 times, at least 8 times, at least 9 times, at least 10 times, at least 15 times, at least 20 times, at least 25 times, at least 30 times, at least 35 times, at least 40 times, at least 45 times, at least 50 times, or more than 50 times.
Embodiment 111 comprises the method of any one of embodiments 100-110, further comprising at least 2 treatment cycles, at least 3 treatment cycles, at least 4 treatment cycles, at least 5 treatment cycles, at least 6 treatment cycles, at least 7 treatment cycles, at least 8 treatment cycles, at least 9 treatment cycles, at least 10 treatment cycles, or more than 10 treatment cycles.
Embodiment 112 comprises the method of any one of embodiments 100-111, wherein the first dose, the second dose, and the target dose are administered on day 1, the one of day 7, 8 or 9, and the one of day 14, 15, or 16, respectively, of the treatment cycle, and wherein the target dose is administered weekly after day 14, 15, or 16 of the treatment cycle.
Embodiment 113 comprises the method of any one of embodiments 100-111, wherein the first dose, the second dose, and the target dose are administered on day 1, the one of day 7, 8 or 9, and the one of day 14, 15, or 16, respectively, of the treatment cycle, and wherein the target dose is administered once every two weeks after day 14, 15, or 16 of the treatment cycle.
Embodiment 114 comprises the method of any one of embodiments 100-111, wherein the first dose, the second dose, and the target dose are administered on day 1, the one of day 7, 8 or 9, and the one of day 14, 15, or 16, respectively, of the treatment cycle, and wherein the target dose is administered once every three weeks after day 14, 15, or 16 of the treatment cycle.
Embodiment 115 comprises the method of any one of embodiments 100-111, wherein the first dose, the second dose, and the target dose are administered on day 1, day 4, and day 8, respectively, of the treatment cycle, and wherein the target dose is administered weekly after day 8 of the treatment cycle.
Embodiment 116 comprises the method of any one of embodiments 100-111, wherein the first dose, the second dose, and the target dose are administered on day 1, day 4, and day 8, respectively, of the treatment cycle, and wherein the target dose is administered once every two weeks after day 8 of the treatment cycle.
Embodiment 117 comprises the method of any one of embodiments 100-111, wherein the first dose, the second dose, and the target dose are administered on day 1, day 4, and day 8, respectively, of the treatment cycle, and wherein the target dose is administered once every three weeks after day 8 of the treatment cycle.
Embodiment 118 comprises the method of any one of embodiments 1-117, wherein the cancer comprises a cell that expresses or overexpresses prostate specific membrane antigen (PSMA).
Embodiment 119 comprises the method of any one of embodiments 1-118, wherein the cancer comprises cells that are dependent on endothelial cells of neovasculature that express or overexpress PSMA.
Embodiment 120 comprises the method of any one of embodiments 1-119, wherein the cancer comprises prostate cancer, lung cancer, breast cancer, colorectal cancer, or renal cell carcinoma.
Embodiment 121 comprises the method of any one of embodiments 1-120, wherein the cancer comprises prostate cancer.
Embodiment 122 comprises the method of any one of embodiments 1-121, wherein the cancer comprises metastatic castration resistant prostate cancer (mCRPC).
Embodiment 123 comprises the method of embodiment 122, wherein the progression of mCRPC is documented according to guidelines of the Prostate Cancer Clinical Trials Working Group 3 (PCWG3), Response Evaluation Criteria in Solid Tumors 1.1 (RECIST 1.1), or both.
Embodiment 124 comprises the method of any one of embodiments 1-123, wherein the cancer comprises non-small cell lung cancer (NSCLC), or small cell lung cancer (SCLC).
Embodiment 125 comprises the method of any one of embodiments 1-124, wherein the subject is a male of at least 18 years old.
Embodiment 126 comprises the method of any one of embodiments 1-124, wherein the subject is a male of about 46 to about 85 years old.
Embodiment 127 comprises the method of any one of embodiments 1-124, wherein the subject is a male of about 69 years old.
Embodiment 128 comprises the method of any one of embodiments 1-127, wherein the subject is treated with a prior cancer therapy before the administering.
Embodiment 129 comprises the method of embodiment 128, wherein the prior cancer therapy comprises 2 to 6 cancer therapies.
Embodiment 130 comprises the method of embodiment 128, wherein the prior cancer therapy comprises about 4 cancer therapies.
Embodiment 131 comprises the method of any one of embodiments 128-130, wherein the prior cancer therapy comprises a taxane, a radioligand therapy, an anti-androgen therapy, an androgen receptor pathway inhibitor therapy, a poly-adenosine diphosphate ribose polymerase (PARP) inhibitor, or a combination thereof.
Embodiment 132 comprises the method of embodiment 131, wherein the anti-androgen therapy comprises a radioligand therapy that targets PSMA.
Embodiment 133 comprises the method of any one of embodiments 128-132, wherein the prior cancer therapy comprises a taxane and an anti-androgen therapy.
Embodiment 134 comprises the method of embodiment 133, wherein the cancer is mCRPC, and wherein the mCRPC progresses after the prior cancer therapy.
Embodiment 135 comprises the method of embodiment 128, wherein the prior cancer therapy comprises no taxane.
Embodiment 136 comprises the method of any one of embodiments 1-135, wherein the subject has adequate organ function.
Embodiment 137 comprises the method of any one of embodiments 1-136, wherein the subject has resolving acute effects of any prior therapy to baseline severity of Common Terminology Criteria for Adverse Events (CTCAE) grade of no more than 1.
Embodiment 138 comprises the method of any one of embodiments 1-137, wherein the subject has adenocarcinoma of the prostate that is confirmed histologically or cytologically.
Embodiment 139 comprises the method of any one of embodiments 1-138, wherein the subject is positive for a baseline prostate-specific membrane antigen positron emission tomography (PSMA-PET).
Embodiment 140 comprises the method of any one of embodiments 1-139, wherein the subject has a baseline PSA level of about 1 ng/ml to about 10000 ng/ml.
Embodiment 141 comprises the method of any one of embodiments 1-140, wherein the subject has a baseline PSA level of about 158 ng/ml.
Embodiment 142 comprises the method of any one of embodiments 1-141, wherein the subject is or is not evaluable according to guidelines of response evaluation criteria in solid tumors 1.1 (RECIST 1.1) or PCWG3.
Embodiment 143 comprises the method of any one of embodiments 1-142, wherein the subject has or does not have a bone metastasis, a lymph node metastasis, or a visceral metastasis.
Embodiment 144 comprises the method of embodiment 143, wherein the visceral metastasis comprises a live metastasis, a lung metastasis, an adrenal metastasis, an abdominal metastasis, or a retroperitoneal metastasis, or a combination thereof.
Embodiment 145 comprises the method of any one of embodiments 1-144, wherein the cancer does not respond to a treatment with a radioligand therapy that targets PSMA before the administering.
Embodiment 146 comprises the method of any one of embodiments 1-144, wherein the cancer responds to a treatment with a radioligand therapy that targets PSMA before the administering.
Embodiment 147 comprises the method of embodiment 145 or 146, wherein the treatment comprises lutetium Lu 177 vipivotide tetraxetan.
Embodiment 148 comprises the method of any one of embodiments 1-147, wherein the subject does not have a prior solid organ transplant procedure before the administering.
Embodiment 149 comprises the method of any one of embodiments 1-148, wherein the subject is not treated with a chimeric antigen receptor T (CAR-T) cell therapy that targets PSMA or a T cell engager therapy that targets PSMA, before the administering.
Embodiment 150 comprises the method of any one of embodiments 1-149, wherein the subject does not have clinically significant cardiovascular disease.
Embodiment 151 comprises the method of any one of embodiments 1-150, wherein the subject does not have an active and clinically significant infection.
Embodiment 152 comprises the method of embodiment 151, wherein the infection comprises a bacterial infection, a viral infection, a fungal infection, a mycobacterial infection, or a combination thereof.
Embodiment 153 comprises the method of any one of embodiments 1-152, wherein the administering comprises administering intravenously.
Embodiment 154 comprises the method of any one of embodiments 1-153, wherein the subject exhibits a cytokine release syndrome (CRS) no more than grade 2.
Embodiment 155 comprises the method of any one of embodiments 1-154, wherein the subject exhibits no treatment related adverse event (TRAE) that is related to a CRS.
Embodiment 156 comprises the method of any one of embodiments 1-155, wherein the administering comprises administering to the subject a second treatment.
Embodiment 157 comprises the method of embodiment 156, wherein the second treatment comprises lutetium Lu 177 vipivotide tetraxetan, enzalutamide, abiraterone acetate (ABI), chemotherapy, radium Ra 223 dichloride, a poly-adenosine diphosphate ribose polymerase inhibitor (PARPI), pembrolizumab, or a combination thereof.
Embodiment 158 comprises the method of embodiment 157, wherein the second treatment comprises enzalutamide.
Embodiment 159 comprises the method of embodiment 158, wherein the administering results in a synergistic effect on the cancer compared with administering the isolated recombinant polypeptide complex alone or administering enzalutamide alone.
Embodiment 160 comprises the method of embodiment 159, wherein the synergistic effect comprises a therapeutic effect that is better than a therapeutic effect resulting from administering the isolated recombinant polypeptide complex alone or a therapeutic effect resulting from administering enzalutamide alone.
Embodiment 161 comprises the method of embodiment 159 or 160, wherein the cancer is refractory, non-responsive, or resistant, to treatment with enzalutamide alone before the administering.
Embodiment 162 comprises the method of embodiment 157, wherein the PARPI comprises olaparib, rucaparib, niraparib, talazoparib, or a combination thereof.
Embodiment 163 comprises the method of any one of embodiments 1-162, wherein the administering results in a therapeutic effect in prostate specific antigen (PSA) response, best RECIST response, best response by PCWG3 criteria, best PET response, radiographic progression free survival (rPFS), overall response rate, overall survival, bone pain, tumor size, or a combination thereof, after the administering.
Embodiment 164 comprises the method of embodiment 163, wherein the subject exhibits a reduction of at least about 30% in PSA level after the administering.
Embodiment 165 comprises the method of embodiment 163, wherein the subject exhibits a reduction of at least about 50% in PSA level after the administering.
Embodiment 166 comprises the method of embodiment 163, wherein the subject exhibits a reduction of at least about 70% in PSA level after the administering.
Embodiment 167 comprises the method of embodiment 163, wherein the subject exhibits a reduction of at least about 90% in PSA level after the administering.
Embodiment 168 comprises the method of embodiment 163, wherein the subject exhibits a reduction of about 30% to about 100% in PSA level after the administering.
Embodiment 169 comprises the method of any one of embodiments 163-168, wherein the subject exhibits a higher reduction in PSA level when the first dose increases.
Embodiment 170 comprises the method of any one of embodiments 163-169, wherein the subject exhibits a higher reduction in PSA level when the second dose increases.
Embodiment 171 comprises the method of any one of embodiments 163-170, wherein the subject exhibits a higher reduction in PSA level when the target dose increases.
Embodiment 172 comprises the method of any one of embodiments 163-171, wherein the subject exhibits a higher reduction in PSA level when the first dose is at least about 200 μg compared with when the first dose is at least about 100 μg.
Embodiment 173 comprises the method of any one of embodiments 163-172, wherein the subject exhibits a reduction of about 30% to about 100% in PSA level at about 2 weeks after the administering of at least 200 μg of the first dose.
Embodiment 174 comprises the method of embodiment 173, wherein the reduction in PSA level continues throughout the treatment with the method.
Embodiment 175 comprises the method of any one of embodiments 163-174, wherein the subject exhibits a better RECIST response when the first dose increases.
Embodiment 176 comprises the method of any one of embodiments 163-175, wherein the subject exhibits a better RECIST response when the second dose increases.
Embodiment 177 comprises the method of any one of embodiments 163-176, wherein the subject exhibits a better RECIST response when the target dose increases.
Embodiment 178 comprises the method of any one of embodiments 163-177, wherein the subject exhibits a better RECIST response when the first dose is at least about 200 μg compared with when the first dose is at least about 100 μg.
Embodiment 179 comprises the method of any one of embodiments 163-178, wherein a reduction in PSA level correlates with an occurrence of a CRS.
Embodiment 180 comprises the method of any one of embodiments 163-179, wherein the subject exhibits a reduction of about 0% to about 100% in tumor size after the administering.
Embodiment 181 comprises the method of any one of embodiments 163-180, wherein the subject exhibits a reduction of about 5% to about 75% in tumor size after the administering.
Embodiment 182 comprises the method of any one of embodiments 163-181, wherein the subject exhibits a reduction in bone skeletal lesion measured by PCWG3 criteria after the administering.
Embodiment 183 comprises the method of any one of embodiments 163-182, wherein the change in tumor size is measured by RECIST 1.1.
Embodiment 184 comprises the method of any one of embodiments 163-183, wherein the change in PSMA level is measured by standardized uptake value (SUV) by PSMA-PET.
Embodiment 185 comprises the method of any one of embodiments 163-184, wherein the subject exhibits a RECIST partial response when the first dose is at least about 100 μg, at least about 200 μg, at least about 300 μg, at least about 400 μg, or at least about 500 μg.
Embodiment 186 comprises the method of any one of embodiments 163-185, wherein the subject exhibits a RECIST partial response when the target dose is at least about 100 μg to at least about 10 mg.
Embodiment 187 comprises the method of any one of embodiments 163-186, wherein the subject exhibits a reduction of 0%-100% in SUV measured by PSMA-PET.
Embodiment 188 comprises the method of any one of embodiments 163-187, wherein the subject exhibits a reduction of about 72% in SUV measured by PSMA-PET.
Embodiment 189 comprises the method of any one of embodiments 163-188, wherein the subject exhibits about 80% in best PSA reduction.
Embodiment 190 comprises the method of any one of embodiments 163-189, wherein the subject exhibits a reduction in pain related to cancer.
Embodiment 191 comprises the method of embodiment 190, wherein the subject exhibits a reduction in bone pain.
Embodiment 192 comprises the method of any one of embodiments 163-191, wherein the subject exhibits a best PET response.
Embodiment 193 comprises the method of any one of embodiments 163-192, wherein the subject exhibits a best RECIST response or a best response measured by PCWG3.
Embodiment 194 comprises the method of any one of embodiments 1-193, wherein the method further comprises treating the subject with a therapy for CRS, diarrhea, chills, alanine transaminase (ALT) increase, anaemia, aspartate aminotransferase (AS) increase, fatigue, decreased appetite, nausea, headache, blood bilirubin increase, hypoalbuminaemia, hypocalcaemia, hypophosphataemia, leukopenia, white blood cell count decrease, myalgia, platelet count decrease, thrombocytopenia, pyrexia, vomiting, blood alkaline phosphatase increase, dysgeusia, hypomagnesaemia, lipase increase, stomatitis, or a combination thereof.
Embodiment 195 comprises the method of any one of embodiments 1-194, wherein the isolated recombinant polypeptide complex is cleaved by a cancer specific protease to generate an active T cell engager and a cleavage fragment after the administering.
Embodiment 196 comprises the method of any one of embodiments 1-195, wherein the administering provides a plasma concentration of the isolated recombinant polypeptide complex that is below preclinical activity threshold.
Embodiment 197 comprises the method of embodiment 195 or 196, wherein the administering provides a plasma concentration of the active T cell engager that is below preclinical activity threshold.
Embodiment 198 comprises the method of any one of embodiments 1-197, wherein the administering of at least one dose of about 100 μg of the isolated recombinant polypeptide complex provides a maximum plasma concentration (Cmax) of the isolated recombinant polypeptide complex in an amount up to about 34 ng/ml.
Embodiment 199 comprises the method of any one of embodiments 1-198, wherein the administering of at least one dose of about 300 μg of the isolated recombinant polypeptide complex provides a Cmax of the isolated recombinant polypeptide complex in an amount up to about 175 ng/ml.
Embodiment 200 comprises the method of any one of embodiments 1-199, wherein the administering of at least one dose of about 450 μg of the isolated recombinant polypeptide complex provides a Cmax of the isolated recombinant polypeptide complex in an amount of up to about 131 ng/ml.
Embodiment 201 comprises the method of any one of embodiments 1-200, wherein the administering of at least one dose of about 1 mg of the isolated recombinant polypeptide complex provides a Cmax of the isolated recombinant polypeptide complex in an amount up to about 283 ng/ml.
Embodiment 202 comprises the method of any one of embodiments 1-201, wherein the administering provides a Cmax of the isolated recombinant polypeptide complex in an amount that correlates with a dose being administered.
Embodiment 203 comprises the method of embodiment 202, wherein the Cmax of the isolated recombinant polypeptide complex increases when the dose being administered increases.
Embodiment 204 comprises the method of any one of embodiments 1-203, wherein the administering provides a Cmax of the isolated recombinant polypeptide complex in an amount that correlates with the number of doses that the subject has received.
Embodiment 205 comprises the method of embodiment 204, wherein the Cmax of the isolated recombinant polypeptide complex increases when the number of doses that the subject has received increases.
Embodiment 206 comprises the method of any one of embodiments 195-205, wherein the administering provides a Cmax of the cleavage fragment in an amount up to about 4.5 ng/ml, and wherein the first dose, the second dose and the target dose being administered are about 100 μg, about 300 μg, and about 450 μg, respectively.
Embodiment 207 comprises the method of any one of embodiments 195-206, wherein the administering provides a Cmax of the cleavage fragment in an amount up to about 14 ng/ml, and wherein the first dose, the second dose and the target dose being administered are about 100 μg, about 300 μg, and about 1 mg, respectively.
Embodiment 208 comprises the method of any one of embodiments 196-207, wherein the administering provides a Cmax of the cleavage fragment in an amount that correlates with the target dose being administered.
Embodiment 209 comprises the method of embodiment 208, wherein the Cmax of the cleavage fragment increases when the target dose being administered increases.
Embodiment 210 comprises the method of any one of embodiments 195-209, wherein the administering provides a Cmax of the cleavage fragment in an amount that correlates with the number of doses that the subject has received.
Embodiment 211 comprises the method of embodiment 210, wherein the Cmax of the cleavage fragment increases when the number of doses that the subject has received increase.
Embodiment 212 comprises the method of embodiment 131, wherein the prior cancer therapy comprises lutetium Lu 177 vipivotide tetraxetan.
Embodiment 213 comprises the method of embodiment 131, wherein the prior cancer therapy comprises olaparib, rucaparib, niraparib, talazoparib, or a combination thereof.
Embodiment 214 comprises the method of any one of embodiments 1-213, wherein the subject has a Homologous Recombination Repair (HRR) gene mutation, a Breast Cancer (BRCA) gene mutation, a BRCA1 gene mutation, a BRCA2 gene mutation, a homeobox B13 (HOXB13) gene mutation, an ataxia telangiectasia mutated (ATM) gene mutation, or a combination thereof.
Embodiment 215 comprises method for treating cancer comprising administering to a subject in need thereof a first dose and a target dose of an isolated recombinant polypeptide complex, wherein the target dose is higher than the first dose, wherein the target dose is at least about 100 μg, wherein the isolated recombinant polypeptide complex comprises a first chain with an amino acid sequence having at least 80% sequence identity to SEQ ID NO: 1 and a second chain with an amino acid sequence having at least 80% sequence identity to SEQ ID NO: 2.
Embodiment 216 comprises the method of embodiment 215, wherein the target dose is about 100 μg to about 10 mg.
Embodiment 217 comprises the method of embodiment 215 or 216, wherein the target dose is at least about 150 μg.
Embodiment 218 comprises the method of embodiment 215 or 216, wherein the target dose is at least about 300 μg.
Embodiment 219 comprises the method of embodiment 215 or 216, wherein the target dose is at least about 600 μg.
Embodiment 220 comprises the method of embodiment 215 or 216, wherein the target dose is at least about 1.2 mg.
Embodiment 221 comprises the method of embodiment 215 or 216, wherein the target dose is at least about 2.4 mg.
Embodiment 222 comprises the method of embodiment 215 or 216, wherein the target dose is at least about 4.8 mg.
Embodiment 223 comprises the method of any one of embodiments 215-222, further comprising administering, between the first dose and the target dose, a second dose of the isolated recombinant polypeptide complex, wherein the second dose is equal to or higher than the first dose, and wherein the target dose is higher than the second dose.
Embodiment 224 comprises the method of any one of embodiments 215-223, wherein the first or second dose is about 5 μg to about 10 mg.
Embodiment 225 comprises the method of any one of embodiments 215-223, wherein the first dose is about 10 μg to about 500 μg.
Embodiment 226 comprises the method of any one of embodiments 215-223, wherein the second dose is about 25 μg to about 2 mg.
Embodiment 227 comprises the method of embodiment 224, wherein the first or second dose is at least about 25 μg, 50 μg, 100 μg, 200 μg, 300 μg, 400 μg, 500 μg, 600 μg, 700 μg, 800 μg, 900 μg, 1 mg, 1.2 mg, or 1.6 mg, 2.0 mg, 2.4 mg, 2.8 mg, 3.2 mg, 3.6 mg, 4.0 mg, 4.4 mg, 4.8 mg, 5.2 mg, 5.6 mg, 6 mg, 8 mg, 9 mg, or at least about 9.9 mg.
Embodiment 228 comprises the method of any one of embodiments 215-227, wherein the first dose, the second dose, and the target dose are about 15 μg, 30 μg, and 100 μg, respectively.
Embodiment 229 comprises the method of any one of embodiments 215-227, wherein the first dose, the second dose, and the target dose are about 25 μg, 50 μg, and 150 μg, respectively.
Embodiment 230 comprises the method of any one of embodiments 215-227, wherein the first dose, the second dose, and the target dose are about 50 μg, 100 μg, and 300 μg, respectively.
Embodiment 231 comprises the method of any one of embodiments 215-227, wherein the first dose, the second dose, and the target dose are about 100 μg, 200 μg, and 600 μg, respectively.
Embodiment 232 comprises the method of any one of embodiments 215-227, wherein the first dose, the second dose, and the target dose are about 200 μg, 400 μg, and 1.2 mg, respectively.
Embodiment 233 comprises the method of any one of embodiments 215-227, wherein the first dose, the second dose, and the target dose are about 300 μg, 800 μg, and 2.4 mg, respectively.
Embodiment 234 comprises the method of any one of embodiments 215-227, wherein the first dose, the second dose, and the target dose are about 400 μg, 1.6 mg, and 4.8 mg, respectively.
Embodiment 235 comprises the method of any one of embodiments 223-234, further comprising administering, between the first dose and the target dose, a third dose of the isolated recombinant polypeptide complex, wherein the third dose is equal to or higher than the first or second dose, and wherein the target dose is higher than the third dose.
Embodiment 236 comprises the method of embodiment 235, wherein the third dose is equal to the first or second dose.
Embodiment 237 comprises the method of embodiment 235, wherein the third dose is higher than the first or second dose.
Embodiment 238 comprises the method of any one of embodiments 235-237, further comprising administering, between the first dose and the target dose, a fourth dose of the isolated recombinant polypeptide complex, wherein the fourth dose is equal to or higher than the first, second, or third dose, and wherein the target dose is higher than the fourth dose.
Embodiment 239 comprises the method of embodiment 238, wherein the fourth dose is equal to the first, second, or third dose.
Embodiment 240 comprises the method of embodiment 238, wherein the fourth dose is higher than the first, second, or third dose.
Embodiment 241 comprises the method of any one of embodiments 238-240, further comprising administering, between the first dose and the target dose, a fifth dose of the isolated recombinant polypeptide complex, wherein the fifth dose is equal to or higher than the first, second, third, or fourth dose, and wherein the target dose is higher than the fifth dose.
Embodiment 242 comprises the method of embodiment 241, wherein the fifth dose is equal to the first, second, third, or fourth dose.
Embodiment 243 comprises the method of embodiment 241, wherein the fifth dose is higher than the first, second, third, or fourth dose.
Embodiment 244 comprises the method of any one of embodiments 241-243, wherein the third, fourth, or fifth dose is about 5 μg to about 10 mg.
Embodiment 245 comprises the method of embodiment 244, wherein the third, fourth, or fifth dose is at least about 25 μg, 50 μg, 100 μg, 200 μg, 300 μg, 400 μg, 500 μg, 600 μg, 700 μg, 800 μg, 900 μg, 1 mg, 1.2 mg, or 1.6 mg, 2.0 mg, 2.4 mg, 2.8 mg, 3.2 mg, 3.6 mg, 4.0 mg, 4.4 mg, 4.8 mg, 5.2 mg, 5.6 mg, 6 mg, 8 mg, 9 mg, or at least about 9.9 mg.
Embodiment 246 comprises the method of any one of embodiments 223-245, further comprising administering, between the first dose and the target dose, at least one dose of the isolated recombinant polypeptide complex, wherein each of the at least one dose is equal to or higher than the first or second dose, and wherein the target dose is higher than each of the at least one dose.
Embodiment 247 comprises the method of embodiment 246, wherein the at least one dose includes at least two doses.
Embodiment 248 comprises the method of embodiment 246, wherein the at least one dose includes at least three doses.
Embodiment 249 comprises the method of embodiment 246, wherein the at least one dose includes at least four doses.
Embodiment 250 comprises the method of embodiment 246, wherein the at least one dose includes at least five doses.
Embodiment 251 comprises the method of embodiment 246, wherein the at least one dose includes at least six doses.
Embodiment 252 comprises the method of embodiment 246, wherein the at least one dose includes at least seven doses.
Embodiment 253 comprises the method of embodiment 246, wherein the at least one dose includes at least eight doses.
Embodiment 254 comprises the method of embodiment 246, wherein the at least one dose includes at least nine doses.
Embodiment 255 comprises the method of embodiment 246, wherein the at least one dose includes at least ten doses.
Embodiment 256 comprises the method of embodiment 246, wherein the at least one dose includes more than ten doses.
Embodiment 257 comprises the method of any one of embodiments 246-256, wherein each of the at least one dose is about 5 μg to about 10 mg.
Embodiment 258 comprises the method of embodiment 257, wherein the each of the at least one dose is at least about 25 μg, 50 μg, 100 μg, 200 μg, 300 μg, 400 μg, 500 μg, 600 μg, 700 μg, 800 μg, 900 μg, 1 mg, 1.2 mg, or 1.6 mg, 2.0 mg, 2.4 mg, 2.8 mg, 3.2 mg, 3.6 mg, 4.0 mg, 4.4 mg, 4.8 mg, 5.2 mg, 5.6 mg, 6 mg, 8 mg, 9 mg, or at least about 9.9 mg.
Embodiment 259 comprises the method of any one of embodiments 215-258, wherein the administering comprises administering the target dose at least two times.
Embodiment 260 comprises the method of any one of embodiments 215-258, wherein the administering comprises administering the target dose at least three times.
Embodiment 261 comprises the method of any one of embodiments 215-258, wherein the administering comprises administering the target dose at least four times.
Embodiment 262 comprises the method of any one of embodiments 215-258, wherein the administering comprises administering the target dose at least five times.
Embodiment 263 comprises the method of any one of embodiments 215-258, wherein the administering comprises administering the target dose at least six times.
Embodiment 264 comprises the method of any one of embodiments 215-258, wherein the administering comprises administering the target dose at least seven times.
Embodiment 265 comprises the method of any one of embodiments 215-258, wherein the administering comprises administering the target dose at least eight times.
Embodiment 266 comprises the method of any one of embodiments 215-258, wherein the administering comprises administering the target dose at least nine times.
Embodiment 267 comprises the method of any one of embodiments 215-258, wherein the administering comprises administering the target dose at least ten times.
Embodiment 268 comprises the method of any one of embodiments 215-267, wherein the administering comprises administering the target dose more than ten times.
Embodiment 269 comprises the method of any one of embodiments 215-267, wherein the first dose and the target dose are administered within two days.
Embodiment 270 comprises the method of any one of embodiments 215-267, wherein the first dose and the target dose are administered within three days.
Embodiment 271 comprises the method of any one of embodiments 215-267, wherein the first dose and the target dose are administered within four days.
Embodiment 272 comprises the method of any one of embodiments 215-267, wherein the first dose and the target dose are administered within five days.
Embodiment 273 comprises the method of any one of embodiments 215-267, wherein the first dose and the target dose are administered within six days.
Embodiment 274 comprises the method of any one of embodiments 215-267, wherein the first dose and the target dose are administered within one week.
Embodiment 275 comprises the method of any one of embodiments 215-267, wherein the first dose and the target dose are administered within two weeks.
Embodiment 276 comprises the method of any one of embodiments 215-267, wherein the first dose and the target dose are administered within three weeks.
Embodiment 277 comprises the method of any one of embodiments 215-267, wherein the first dose and the target dose are administered within four weeks.
Embodiment 278 comprises the method of any one of embodiments 215-267, wherein the first dose and the target dose are administered within five weeks.
Embodiment 279 comprises the method of any one of embodiments 215-267, wherein the first dose and the target dose are administered within six weeks.
Embodiment 280 comprises the method of any one of embodiments 215-267, wherein the first dose and the target dose are administered within seven weeks.
Embodiment 281 comprises the method of any one of embodiments 215-267, wherein the first dose and the target dose are administered within eight weeks.
Embodiment 282 comprises the method of any one of embodiments 215-267, wherein the first dose and the target dose are administered within nine weeks.
Embodiment 283 comprises the method of any one of embodiments 215-267, wherein the first dose and the target dose are administered within ten weeks.
Embodiment 284 comprises the method of any one of embodiments 215-267, wherein the first dose and the target dose are administered within one month.
Embodiment 285 comprises the method of any one of embodiments 215-267, wherein the first dose and the target dose are administered within two months.
Embodiment 286 comprises the method of any one of embodiments 215-267, wherein the first dose and the target dose are administered within three months.
Embodiment 287 comprises the method of any one of embodiments 215-267, wherein the first dose and the target dose are administered within four months.
Embodiment 288 comprises the method of any one of embodiments 215-267, wherein the first dose and the target dose are administered within five months.
Embodiment 289 comprises the method of any one of embodiments 215-267, wherein the first dose and the target dose are administered within six months.
Embodiment 290 comprises the method of any one of embodiments 215-267, wherein the first dose and the target dose are administered within seven months.
Embodiment 291 comprises the method of any one of embodiments 215-267, wherein the first dose and the target dose are administered within eight months.
Embodiment 292 comprises the method of any one of embodiments 215-267, wherein the first dose and the target dose are administered within nine months.
Embodiment 293 comprises the method of any one of embodiments 215-267, wherein the first dose and the target dose are administered within ten months.
Embodiment 294 comprises the method of any one of embodiments 215-267, wherein the first dose and the target dose are administered within eleven months.
Embodiment 295 comprises the method of any one of embodiments 215-267, wherein the first dose and the target dose are administered within twelve months.
Embodiment 296 comprises the method of any one of embodiments 215-267, wherein the first dose and the target dose are administered within one and half years.
Embodiment 297 comprises the method of any one of embodiments 215-267, wherein the first dose and the target dose are administered within two years.
Embodiment 298 comprises the method of any one of embodiments 215-267, wherein the first dose and the target dose are administered within three years.
Embodiment 299 comprises the method of any one of embodiments 215-267, wherein the first dose and the target dose are administered within four years.
Embodiment 300 comprises the method of any one of embodiments 215-267, wherein the first dose and the target dose are administered within five years.
Embodiment 301 comprises the method of any one of embodiments 215-300, wherein two doses of the isolated recombinant polypeptide complex are administered separately in two consecutive days.
Embodiment 302 comprises the method of any one of embodiments 215-300, wherein administration of two doses of the isolated recombinant polypeptide complex is at least one day apart.
Embodiment 303 comprises the method of any one of embodiments 215-300, wherein administration of two doses of the isolated recombinant polypeptide complex is at least two days apart.
Embodiment 304 comprises the method of any one of embodiments 215-300, wherein administration of two doses of the isolated recombinant polypeptide complex is at least three days apart.
Embodiment 305 comprises the method of any one of embodiments 215-300, wherein administration of two doses of the isolated recombinant polypeptide complex is at least four days apart.
Embodiment 306 comprises the method of any one of embodiments 215-300, wherein administration of two doses of the isolated recombinant polypeptide complex is at least five days apart.
Embodiment 307 comprises the method of any one of embodiments 215-300, wherein administration of two doses of the isolated recombinant polypeptide complex is at least six days apart.
Embodiment 308 comprises the method of any one of embodiments 215-300, wherein administration of two doses of the isolated recombinant polypeptide complex is at least seven days apart.
Embodiment 309 comprises the method of any one of embodiments 215-300, wherein administration of two doses of the isolated recombinant polypeptide complex is at least eight days apart.
Embodiment 310 comprises the method of any one of embodiments 215-300, wherein administration of two doses of the isolated recombinant polypeptide complex is at least nine days apart.
Embodiment 311 comprises the method of any one of embodiments 215-300, wherein administration of two doses of the isolated recombinant polypeptide complex is at least ten days apart.
Embodiment 312 comprises the method of any one of embodiments 215-300, wherein administration of two doses of the isolated recombinant polypeptide complex is at least eleven days apart.
Embodiment 313 comprises the method of any one of embodiments 215-300, wherein administration of two doses of the isolated recombinant polypeptide complex is at least twelve days apart.
Embodiment 314 comprises the method of any one of embodiments 215-300, wherein administration of two doses of the isolated recombinant polypeptide complex is at least thirteen days apart.
Embodiment 315 comprises the method of any one of embodiments 215-300, wherein administration of two doses of the isolated recombinant polypeptide complex is at least fourteen days apart.
Embodiment 316 comprises the method of any one of embodiments 215-300, wherein administration of two doses of the isolated recombinant polypeptide complex is at least three weeks apart.
Embodiment 317 comprises the method of any one of embodiments 215-300, wherein administration of two doses of the isolated recombinant polypeptide complex is at least four weeks apart.
Embodiment 318 comprises the method of any one of embodiments 215-300, wherein administration of two doses of the isolated recombinant polypeptide complex is at least five weeks apart.
Embodiment 319 comprises the method of any one of embodiments 215-300, wherein administration of two doses of the isolated recombinant polypeptide complex is at least six weeks apart.
Embodiment 320 comprises the method of any one of embodiments 215-300, wherein administration of two doses of the isolated recombinant polypeptide complex is at least seven weeks apart.
Embodiment 321 comprises the method of any one of embodiments 215-300, wherein administration of two doses of the isolated recombinant polypeptide complex is at least eight weeks apart.
Embodiment 322 comprises the method of any one of embodiments 215-300, wherein administration of two doses of the isolated recombinant polypeptide complex is at least three months apart.
Embodiment 323 comprises the method of any one of embodiments 215-300, wherein administration of two doses of the isolated recombinant polypeptide complex is at least four months apart.
Embodiment 324 comprises the method of any one of embodiments 215-300, wherein administration of two doses of the isolated recombinant polypeptide complex is at least five months apart.
Embodiment 325 comprises the method of any one of embodiments 215-300, wherein administration of two doses of the isolated recombinant polypeptide complex is at least six months apart.
Embodiment 326 comprises the method of any one of embodiments 215-300, wherein administration of two doses of the isolated recombinant polypeptide complex is at least one year apart.
Embodiment 327 comprises the method of any one of embodiments 215-300, wherein administration of two doses of the isolated recombinant polypeptide complex is at least two years apart.
Embodiment 328 comprises the method of any one of embodiments 215-300, wherein administration of two doses of the isolated recombinant polypeptide complex is at least three years apart.
Embodiment 329 comprises the method of any one of embodiments 215-300, wherein administration of two doses of the isolated recombinant polypeptide complex is at least four years apart.
Embodiment 330 comprises the method of any one of embodiments 215-300, wherein administration of two doses of the isolated recombinant polypeptide complex is at least five years apart.
Embodiment 331 comprises the method of any one of embodiments 215-330, comprising a treatment cycle, wherein the treatment cycle comprises not administering a dose of the isolated recombinant polypeptide complex.
Embodiment 332 comprises the method of any one of embodiments 215-330, comprising a treatment cycle, wherein the treatment cycle comprises administering at least one dose of the isolated recombinant polypeptide complex.
Embodiment 333 comprises the method of any one of embodiments 215-330, comprising a treatment cycle, wherein the treatment cycle comprises administering at least two doses of the isolated recombinant polypeptide complex.
Embodiment 334 comprises the method of any one of embodiments 215-330, comprising a treatment cycle, wherein the treatment cycle comprises administering at least three doses of the isolated recombinant polypeptide complex.
Embodiment 335 comprises the method of any one of embodiments 215-330, comprising a treatment cycle, wherein the treatment cycle comprises administering at least four doses of the isolated recombinant polypeptide complex.
Embodiment 336 comprises the method of any one of embodiments 215-330, comprising a treatment cycle, wherein the treatment cycle comprises administering at least five doses of the isolated recombinant polypeptide complex.
Embodiment 337 comprises the method of any one of embodiments 215-330, comprising a treatment cycle, wherein the treatment cycle comprises administering at least six doses of the isolated recombinant polypeptide complex.
Embodiment 338 comprises the method of any one of embodiments 215-330, comprising a treatment cycle, wherein the treatment cycle comprises administering more than six doses of the isolated recombinant polypeptide complex.
Embodiment 339 comprises the method of any one of embodiments 331-338, comprising at least two treatment cycles.
Embodiment 340 comprises the method of any one of embodiments 331-338, comprising at least three treatment cycles.
Embodiment 341 comprises the method of any one of embodiments 331-338, comprising at least four treatment cycles.
Embodiment 342 comprises the method of any one of embodiments 331-338, comprising at least five treatment cycles.
Embodiment 343 comprises the method of any one of embodiments 331-338, comprising at least six treatment cycles.
Embodiment 344 comprises the method of any one of embodiments 331-338, comprising at least seven treatment cycles.
Embodiment 345 comprises the method of any one of embodiments 331-338, comprising at least eight treatment cycles.
Embodiment 346 comprises the method of any one of embodiments 331-338, comprising at least nine treatment cycles.
Embodiment 347 comprises the method of any one of embodiments 331-338, comprising at least ten treatment cycles.
Embodiment 348 comprises the method of any one of embodiments 331-338, comprising more than ten treatment cycles.
Embodiment 349 comprises the method of any one of embodiments 339-348, comprising at least two treatment cycles, wherein two of the at least two treatment cycles comprise not administering the isolated recombinant polypeptide complex.
Embodiment 350 comprises the method of any one of embodiments 331-349, wherein the treatment cycle comprises one to sixty days.
Embodiment 351 comprises the method of any one of embodiments 331-349, wherein the treatment cycle comprises one to thirty days.
Embodiment 352 comprises the method of any one of embodiments 331-349, wherein the treatment cycle comprises twenty one days.
Embodiment 353 comprises the method of any one of embodiments 331-349, wherein the treatment cycle comprises twenty eight days.
Embodiment 354 comprises the method of any one of embodiments 215-353, wherein the administering comprises administering through intravenous infusion or subcutaneous injection.
Embodiment 355 comprises the method of any one of embodiments 215-354, wherein the cancer comprises prostate cancer.
Embodiment 356 comprises the method of any one of embodiments 215-355, wherein the cancer comprises metastatic castration resistant prostate cancer (mCRPC).
Embodiment 357 comprises the method of any one of embodiments 215-356, further comprising treating the subject with a therapy for an infusion-related reaction before or after the administering.
Embodiment 358 comprises the method of embodiment 357, wherein the therapy for an infusion-related reaction comprises an antipyretic drug, an antihistamine drug, an antiemetic drug, or a corticosteroid.
Embodiment 359 comprises the method of embodiment 357, wherein the therapy for an infusion-related reaction comprises acetaminophen, paracetamol, or diphenhydramine.
Embodiment 360 comprises the method of any one of embodiments 215-359, further comprising treating the subject with a therapy for cytokine release syndrome (CRS) before or after administration of a dose of the isolated recombinant polypeptide complex.
Embodiment 361 comprises the comprises the method of any one of embodiments 215-360, further comprising treating the subject with a therapy for fever, tachycardia, hypotension, hypoxia, fatigue, nausea, headache, dyspnea, rigors, myalgia, arthralgia, anorexia, or a combination thereof, before or after administration of a dose of the isolated recombinant polypeptide complex.
Embodiment 362 comprises the method of embodiment 360 or 361, further comprising treating the subject with the therapy before or after administration of the first dose of the isolated recombinant polypeptide complex.
Embodiment 363 comprises the method of embodiment 360 or 361, further comprising treating the subject with the therapy before or after administration of the target dose of the isolated recombinant polypeptide complex.
Embodiment 364 comprises the method of any one of embodiments 360-363, wherein the therapy comprise an intravenous hydration procedure, an oxygen treatment, a corticosteroid, an immunosuppressant, a vasopressor, or an antiepileptic drug.
Embodiment 365 comprises the method of embodiment 364, wherein the oxygen treatment comprises mechanical ventilation.
Embodiment 366 comprises the method of embodiment 364, wherein the corticosteroid comprises dexamethasone.
Embodiment 367 comprises the method of embodiment 366, further comprising treating the subject with dexamethasone prior to or after administration of the first dose of the isolated recombinant polypeptide complex.
Embodiment 368 comprises the method of embodiment 366, further comprising treating the subject with dexamethasone prior to or after administration of the target dose of the isolated recombinant polypeptide complex.
Embodiment 369 comprises the method of embodiment 366, further comprising treating the subject with dexamethasone prior to or after administration of a dose of the isolated recombinant polypeptide complex, wherein the dose is between the first dose and the target dose.
Embodiment 370 comprises the method of embodiment 366, wherein about 8 mg dexamethasone is administered orally to the subject one day or 12-16 hours before administration of the first dose of the isolated recombinant polypeptide complex.
Embodiment 371 comprises the method of embodiment 366, wherein about 10 mg dexamethasone is administered orally to the subject at least 2-4 hours before administration of the target dose of the isolated recombinant polypeptide complex.
Embodiment 372 comprises the method of embodiment 366, wherein about 10 mg dexamethasone is administered intravenously to the subject at least 0.5-1.5 hours before administration of the target dose of the isolated recombinant polypeptide complex.
Embodiment 373 comprises the method of embodiment 366, wherein:
(a) about 8 mg dexamethasone is administered orally to the subject one day or 12-16 hours before administration of the first dose of the isolated recombinant polypeptide complex; and
(b) about 10 mg dexamethasone is administered orally to the subject at least 2-4 hours before administration of the target dose of the isolated recombinant polypeptide complex or about 10 mg dexamethasone is administered intravenously to the subject at least 0.5-1.5 hours before administration of the target dose of the isolated recombinant polypeptide complex.
Embodiment 374 comprises the method of embodiment 366, wherein a corticosteroid is administered to the subject after administration of the target dose of the isolated recombinant polypeptide complex.
Embodiment 375 comprises the method of any one of embodiments 360-373, wherein the therapy comprise an interleukin 6 (IL-6) receptor inhibitor.
Embodiment 376 comprises the method of any one of embodiments 360-373, wherein the therapy comprise an IL-6 inhibitor.
Embodiment 377 comprises the method of embodiment 375 or 376, wherein the therapy comprise tocilizumab, siltuximab, or sarilimab.
Embodiment 378 comprises the method of embodiment 375 or 376, wherein the therapy is administered to the subject before administration of the first dose of the isolated recombinant polypeptide complex.
Embodiment 379 comprises the method of embodiment 375 or 376, wherein the therapy is administered to the subject when the subject starts to exhibit a symptom of CRS.
Embodiment 380 comprises the method of any one of embodiments 375 or 376, wherein the therapy comprise a corticosteroid in combination with an IL-6 inhibitor or an IL-6 receptor inhibitor.
Embodiment 381 comprises the method of embodiment 378, further comprising (a) discontinuing an anti-hypertension therapy prior to administration of a dose of the isolated recombinant polypeptide complex; and optionally (b) resuming the anti-hypertension therapy prior to or after administration of another dose of the isolated recombinant polypeptide complex.
Embodiment 382 comprises the method of any one of embodiments 357-364, wherein the therapy comprises an anti-hypotension therapy administered to the subject after administration of the first dose, the target dose, or any dose between the first dose and target dose, of the isolated recombinant polypeptide complex.
Embodiment 383 comprises the method of embodiment 364, wherein the intravenous hydration procedure is performed prior to administration of a dose of the isolated recombinant polypeptide complex.
Embodiment 384 comprises the method of embodiment 359, wherein acetaminophen or paracetamol in combination with an anti-histamine drug is administered to the subject prior to administration of a dose of the isolated recombinant polypeptide complex.
Embodiment 385 comprises the method of any one of embodiments 215-384, wherein the first chain comprises at least 85% sequence identity to SEQ ID NO: 1.
Embodiment 386 comprises the method of any one of embodiments 215-384, wherein the first chain comprises at least 90% sequence identity to SEQ ID NO: 1.
Embodiment 387 comprises the method of any one of embodiments 215-384, wherein the first chain comprises at least 95% sequence identity to SEQ ID NO: 1.
Embodiment 388 comprises the method of any one of embodiments 215-384, wherein the first chain comprises at least 99% sequence identity to SEQ ID NO: 1.
Embodiment 389 comprises the method of any one of embodiments 215-384, wherein the first chain comprises the amino acid sequence according to SEQ ID NO: 1.
Embodiment 390 comprises the method of any one of embodiments 215-384, wherein the second chain comprises at least 85% sequence identity to SEQ ID NO: 2.
Embodiment 391 comprises the method of any one of embodiments 215-384, wherein the second chain comprises at least 90% sequence identity to SEQ ID NO: 2.
Embodiment 392 comprises the method of any one of embodiments 215-384, wherein the second chain comprises at least 95% sequence identity to SEQ ID NO: 2.
Embodiment 393 comprises the method of any one of embodiments 215-384, wherein the second chain comprises at least 99% sequence identity to SEQ ID NO: 2.
Embodiment 394 comprises the method of any one of embodiments 215-384, wherein the second chain comprises the amino acid sequence according to SEQ ID NO: 2.
Embodiment 395 comprises the method of any one of embodiments 215-394, wherein the subject exhibits a reduction in the expression of prostate specific antigen (PSA) after the administering.
Embodiment 396 comprises the method of embodiment 395, wherein the subject exhibits at least a 30% reduction in the expression of PSA compared with the PSA expression level measured before administration of the first dose of the isolated recombinant polypeptide complex.
Embodiment 397 comprises the method of embodiment 395, wherein the subject exhibits about 30% to about 70% reduction in the expression of PSA compared with the PSA expression level measured before administration of the first dose of the isolated recombinant polypeptide complex.
Embodiment 398 comprises the method of any one of embodiments 215-397, wherein the subject exhibits, after the administering, both a symptom of cytokine release syndrome (CRS) and a reduction in the expression of prostate specific antigen (PSA).
Embodiment 399 comprises the method of any one of embodiments 215-398, wherein the subject does not exhibit, a symptom of CRS after administration of the first dose of the isolated recombinant polypeptide complex.
Embodiment 400 comprises the method of any one of embodiments 215-399, wherein the subject exhibits a symptom of CRS after administration of the first dose of the isolated recombinant polypeptide complex.
Embodiment 401 comprises the method of any one of embodiments 215-400, wherein the subject does not exhibit a symptom of CRS after administration of a dose of the isolated recombinant polypeptide complex.
Embodiment 402 comprises the method of embodiment 401, wherein the dose is not the first dose.
Embodiment 403 comprises the method of embodiment 400, wherein the grade of CRS is lower than 3.
Embodiment 404 comprises the method of embodiment 400, wherein the grade of CRS is lower than 2.
Embodiment 405 comprises the method of any one of embodiments 215-404, wherein the subject is human.
Embodiment 406 comprises the method of any one of embodiments 215-405, wherein the isolated recombinant polypeptide complex being administered is in a pharmaceutical composition comprising a pharmaceutically acceptable excipient.
Embodiment 407 comprises the method of embodiment 406, wherein the pharmaceutically acceptable excipient comprises a buffer, a stabilizing agent, a tonicity agent, a surfactant, or combinations thereof.
Embodiment 408 comprises the method of embodiment 407, wherein the buffer comprises an amino acid or a derivative thereof.
Embodiment 409 comprises the method of embodiment 408, wherein the amino acid or the derivative thereof comprises L-histidine, L-histidine monohydrochloride monohydrate, or combinations thereof.
Embodiment 410 comprises the method of embodiment 407, wherein the stabilizing agent comprises sugar.
Embodiment 411 comprises the method of embodiment 410, wherein the sugar comprises sucrose.
Embodiment 412 comprises the method of embodiment 407, wherein the tonicity agent comprises sugar.
Embodiment 413 comprises the method of embodiment 412, wherein the sugar comprises sucrose.
Embodiment 414 comprises the method of embodiment 407, wherein the surfactant comprises polysorbate 20.
Embodiment 415 comprises the method of embodiment 409, wherein the total amount of L-histidine in the pharmaceutical composition is about 10 mM in the forms of both L-histidine and L-histidine monohydrochloride monohydrate.
Embodiment 416 comprises the method of embodiment 415, wherein the molar ratio of L-histidine to L-histidine monohydrochloride monohydrate is about 3:2.
Embodiment 417 comprises the method of any one of embodiments 406-416, wherein the pharmaceutical composition comprises about 8% (w/v) sucrose.
Embodiment 418 comprises the method of any one of embodiments 406-417, wherein the pharmaceutical composition comprises at least 0.01% (w/v) polysorbate 20.
Embodiment 419 comprises the method of any one of embodiments 406-418, wherein the pharmaceutical composition comprises about 6 mM L-histidine, about 4 mM L-histidine monohydrochloride monohydrate, about 8% (w/v) sucrose, and about 0.01% (w/v) polysorbate 20.
Embodiment 420 comprises the method of any one of embodiments 406-419, wherein the pharmaceutical composition comprises about 2 mg/ml of the isolated recombinant polypeptide complex.
Embodiment 421 comprises the method of any one of embodiments 406-420, wherein the pharmaceutical composition comprises a pH between about 5 and about 7.
Embodiment 422 comprises the method of any one of embodiments 407-421, wherein the pharmaceutical composition comprises a pH of about 6.3.
Embodiment 423 comprises the method of embodiment 395, wherein the subject exhibits a reduction of about 70% to 99% in the expression of PSA compared with the PSA expression level measured before administration of the first dose of the isolated recombinant polypeptide complex.
Embodiment 424 comprises the method of embodiment 395, wherein the subject exhibits a reduction of about 100% in the expression of PSA compared with the PSA expression level measured before administration of the first dose of the isolated recombinant polypeptide complex.
Embodiment 425 comprises the method of any one of embodiments 331-424, comprising at least two treatment cycles, wherein each of the at least two treatment cycles comprises not administering the isolated recombinant polypeptide complex.
Embodiment 426 comprises the method of any one of embodiments 331-425, comprising: (a) a first treatment cycle comprising administering a dose of the isolated recombinant polypeptide complex; and (b) a second treatment cycle comprising not administering the isolated recombinant polypeptide complex, wherein the first treatment cycle alternates with the second treatment cycle.
Embodiment 427 comprises the method of embodiment 426, comprising at least two of the first or second treatment cycle.
Embodiment 428 comprises the method of embodiment 426, comprising at least three of the first or second treatment cycle.
Embodiment 429 comprises the method of embodiment 426, comprising at least four of the first or second treatment cycle.
Embodiment 430 comprises the method of embodiment 426, comprising at least five of the first or second treatment cycle.
Embodiment 431 comprises the method of embodiment 426, comprising at least six of the first or second treatment cycle.
Embodiment 432 comprises the method of embodiment 426, comprising more than six of the first or second treatment cycle.
Embodiment 433 comprises the method of any one of embodiments 331-425, comprising: (a) a first series of a first treatment cycle, wherein the first treatment cycle comprises administering a dose of the isolated recombinant polypeptide complex; and (b) a second series of a second treatment cycle, wherein the second treatment cycle comprises not administering the isolated recombinant polypeptide complex, wherein the first or second series comprise consecutive treatment cycles, wherein the first series alternate with the second series.
Embodiment 434 comprises the method of embodiment 433, wherein the first or second series comprise at least two consecutive treatment cycles.
Embodiment 435 comprises the method of embodiment 433, wherein the first or second series comprise at least three consecutive treatment cycles.
Embodiment 436 comprises the method of embodiment 433, wherein the first or second series comprise at least four consecutive treatment cycles.
Embodiment 437 comprises the method of embodiment 433, wherein the first or second series comprise at least five consecutive treatment cycles.
Embodiment 438 comprises the method of embodiment 433, wherein the first or second series comprise at least six consecutive treatment cycles.
Embodiment 439 comprises the method of embodiment 433, wherein the first or second series comprise more than six consecutive treatment cycles.
Embodiment 440 comprises the method of any one of embodiments 331-425, comprising: (a) a first treatment cycle, wherein the first treatment cycle comprises administering a dose of the isolated recombinant polypeptide complex; and (b) a series of a second treatment cycle, wherein the second treatment cycle comprises not administering the isolated recombinant polypeptide complex, wherein the series comprise consecutive treatment cycles, wherein the first treatment cycle alternates with the second series.
Embodiment 441 comprises the method of embodiment 440, comprising at least two of the first treatment cycle.
Embodiment 442 comprises the method of embodiment 440, comprising at least three of the first treatment cycle.
Embodiment 443 comprises the method of embodiment 440, comprising at least four of the first treatment cycle.
Embodiment 444 comprises the method of embodiment 440, comprising at least five of the first treatment cycle.
Embodiment 445 comprises the method of embodiment 440, comprising at least six of the first treatment cycle.
Embodiment 446 comprises the method of embodiment 440, comprising more than six of the first treatment cycle.
Embodiment 447 comprises the method of embodiment 440, wherein the series comprise at least two consecutive treatment cycles.
Embodiment 448 comprises the method of embodiment 440, wherein the series comprise at least three consecutive treatment cycles.
Embodiment 449 comprises the method of embodiment 440, wherein the series comprise at least four consecutive treatment cycles.
Embodiment 450 comprises the method of embodiment 440, wherein the series comprise at least five consecutive treatment cycles.
Embodiment 451 comprises the method of embodiment 440, wherein the series comprise at least six consecutive treatment cycles.
Embodiment 452 comprises the method of embodiment 440, wherein the series comprise more than six consecutive treatment cycles.
Embodiment 453 comprises the method of any one of embodiments 331-425, comprising: (a) a series of a first treatment cycle, wherein the first treatment cycle comprises administering a dose of the isolated recombinant polypeptide complex; and (b) a second treatment cycle, wherein the second treatment cycle comprises not administering the isolated recombinant polypeptide complex, wherein the series comprise consecutive treatment cycles, wherein the second treatment cycle alternates with the series.
Embodiment 454 comprises the method of embodiment 453, comprising at least two of the second treatment cycle.
Embodiment 455 comprises the method of embodiment 453, comprising at least three of the second treatment cycle.
Embodiment 456 comprises the method of embodiment 453, comprising at least four of the second treatment cycle.
Embodiment 457 comprises the method of embodiment 453, comprising at least five of the second treatment cycle.
Embodiment 458 comprises the method of embodiment 453, comprising at least six of the second treatment cycle.
Embodiment 459 comprises the method of embodiment 453, comprising more than six of the second treatment cycle.
Embodiment 460 comprises the method of embodiment 453, wherein the series comprise at least two consecutive treatment cycles.
Embodiment 461 comprises the method of embodiment 453, wherein the series comprise at least three consecutive treatment cycles.
Embodiment 462 comprises the method of embodiment 453, wherein the series comprise at least four consecutive treatment cycles.
Embodiment 463 comprises the method of embodiment 453, wherein the series comprise at least five consecutive treatment cycles.
Embodiment 464 comprises the method of embodiment 453, wherein the series comprise at least six consecutive treatment cycles.
Embodiment 465 comprises the method of embodiment 452, wherein the series comprise more than six consecutive treatment cycles.
Embodiment 466 comprises the method of any one of embodiments 215-465, wherein the administering comprises administering through intravenous infusion.
Embodiment 467 comprises the method of any one of embodiments 215-465, wherein the administering comprises administering through subcutaneous injection.

### EXAMPLES

### Example 1: Clinical Effects of Polypeptide Complex 1 (PC-1)

This Example illustrates the clinical effects of a polypeptide complex 1(PC-1) disclosed herein.

PC-1 is a tumor activated T-cell engager (TRACTr) molecule designed to improve the therapeutic profile of prostate-specific membrane antigen (PSMA)-targeted T cell engagers (TCEs) in patients with metastatic castration-resistant prostate cancer (mCRPC).

### Pharmacology

Nonclinical pharmacology in vitro studies with PC-1 examined the following:
1. PC-1 binding to PSMA, cluster of differentiation 3 (CD3), and albumin antigens from mouse, rat, cynomolgus monkey, and human
2. PC-1 stability in serum of healthy human donors, mCRPC patients, and cynomolgus monkeys
3. The ability of PC-1 to induce cytokine production
4. The ability of PC-1 to induce T cell-mediated anti-tumor cytotoxic activity Results from the binding study concluded that PC-1 bound human and cynomolgus monkey PSMA, CD3, and albumin with low nanomolar affinity, while exhibiting minimal binding to mouse or rat antigens, and that CD3 binding affinity was cleavage dependent. The stability study showed that PC-1 demonstrated similar cleavage rates in healthy human serum (1%) relative to mCRPC patients' serum (2%) and that cleavage of PC-1 in cynomolgus monkey serum was 6% per day. The cytokine induction study showed that, while cleaved and non-masked versions of PC-1 exhibited dose-dependent ability to induce release of interferon γ(IFNγ), tumor necrosis factor (TNF), and interleukin (IL)-6 by immune cells, masking of PC-1 CD3-binding domain diminished its ability to induce cytokine production. This suggested that cytokine release required proteolytic PC-1 demasking. Cytokine production was not detected in the absence of target cells, suggesting that the activity of PC-1 depends on the presence of PSMA-expressing target cells. The presence of the mask also decreased the ability of PC-1 to induce T cell-mediated tumor cell killing. Additionally, the ability of PC-1 to induce T cell-mediated tumor cell killing was dose- and PSMA-dependent. The nonclinical pharmacology study examined the ability of PC-1 and PC-1-serine protease (SP) cleaved to induce cytokine release in human whole blood samples. The study examined cytokine release under 2 conditions, with soluble and wet-coated (plate-bound) test articles. No IL-2, IL-6, IL-10, TNF, and IFNγ release above the level of the untreated control (very low to no signal) was observed following stimulation with either soluble or wet-coated (plate-bound) PC-1-SP cleaved in any of the samples. No IL-2 or IFNγ cytokine release was observed in any samples following PC-1 stimulation. Following PC-1 stimulation in the plate-bound format, no IL-6, IL-10, or TNF cytokine release was observed; however, after treatment with soluble PC-1 at the highest concentrations tested (10 nM [917.4 ng/mL] and 100 nM [9174 ng/mL]), IL-6, IL-10, and TNF cytokine release was observed in a few samples. Parameters for nonclinical safety pharmacology included cardiovascular, CNS, and respiratory safety endpoints that were incorporated into a 4-week repeat-dose intravenous (IV) toxicity study in cynomolgus monkeys. There were no functional effects on the CNS or respiratory system based on clinical observations of the animals and detailed weekly examinations at doses of PC-1≤1.5 mg/kg/week. Additionally, there were no clearly defined effects from PC-1 administration on the cardiovascular systems of the animals on qualitative or quantitative electrocardiogram (ECG) parameters. Aside from a few episodes of non-test article-related arrhythmias, all ECGs were within normal limits.

### Phase 1 Clinical Study Design

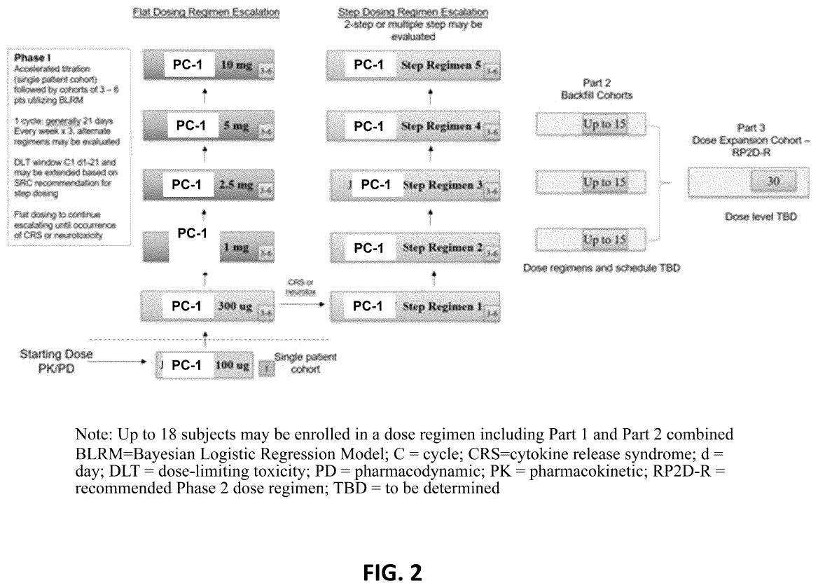

This study is a first-in-human (FIH), Phase 1, open-label, multicenter study to assess the safety, tolerability, PK, pharmacodynamics (PD), and preliminary efficacy of PC-1 administered as a single agent in adult subjects with mCRPC. As illustrated in FIG. 2, the study will be conducted in 3 parts: Dose Escalation (Part 1), Cohort Backfill Expansion (Part 2) with subjects enrolled across three dose levels, and Dose Expansion (Part 3) with subjects enrolled at the recommended Phase 2 dose. Dose Escalation (Part 1) will assess the safety, tolerability, PK, PD, and preliminary efficacy of PC-1 administered by IV infusion. Cohort Backfill Expansion (Part 2) will allow for further characterization of safety and activity of dose levels. Dose Expansion (Part 3) will determine additional safety, tolerability, PK, PD, and preliminary clinical activity data with PC-1 at a dose and schedule to be determined by the Safety Review Committee after reviewing all available safety, PK, PD, and preliminary efficacy data.

### Physical, Chemical, and Pharmaceutical Properties and Formulation

Physical and Biochemical Properties: PC-1 is a 92 kDa humanized tri-specific protein comprised of anti-PSMA antigen-binding fragment (Fab), anti-CD3 single-chain variable fragment (scFv), and anti-albumin single domain antibody (sdAb). The molecular formula for PC-1 is C₄₀₅₀H₆₂₀₃N₁₀₁O₁₂₈₅S₂₇. The calculated average molecular weight of PC-1 is 91,742 Da. PC-1 TRACTr consists of two protein chains connected by a single intermolecular disulfide bond between the light chain (LC) and heavy chain (HC) of the TRACTr, and 8 intramolecular disulfide bonds. The peptide mask contains a single internal disulfide bond. The LC of the CD3 scFv is fused to the N-terminal LC of the PSMA Fab via a short flexible amino acid linker. A tandem albumin-binding sdAb, peptide mask, and tumor protease cleavable amino acid linker are fused to the amino terminus of the CD3 scFv HC. The albumin-binding sdAb is attached to the mask via a short flexible linker (FIG. 1).

### Drug Product

The PC-1 solution for injection, specifically IV infusion (referred to as PC-1 DP), is supplied as a sterile aqueous solution formulated at a nominal concentration of 2 mg/mL in 10 mM histidine, 8% (w/v) sucrose, 0.01% (w/v) polysorbate 20 (Table 2). The PC-1 drug product is contained in a 2R, Type 1 borosilicate glass vial with a 13 mm nested cap consisting of a polypropylene snap-fit cap and an embedded Flurotec® laminated elastomeric stopper plug. The formulation of the PC-1 drug product is outlined in Table 2. The physical and chemical properties of PC-1 are summarized in Table 3.

TABLE 2
Formulation Composition of PC-1 Drug Product

| Component | Amount (g) | Function | Quality Standard |
|---|---|---|---|
| PC-1 | 16.0 | Active | CGMP |
| | | pharmaceutical | |
| | | ingredient | |
| L-histidine | 8.312 | Buffer | Ph Eur, USP, JP |
| L-histidine | 5.541 | Buffer | Ph Eur, BP, JP |
| monohydrochloride | | | |
| monohydrate | | | |
| Sucrose | 640 | Stabilizer and | USP, NF, Ph Eur, |
| | | tonicity agent | JP, Ch. P |
| Polysorbate 20 | 0.8 | Surfactant | USP/NF,Ph Eur, JP |
| Water for injection | Q.S. | Diluent | USP |

Abbreviations:
cGMP = Current good manufacturing practice;
BP = British Pharmacopoeia;
Ch. P = Chinese Pharmacopoeia;
JP = Japanese Pharmacopoeia;
Ph Eur = European Pharmacopoeia;
Q.S. = quantity sufficient;
USP/NF = US Pharmacopeia/National Formulary

TABLE 3
Physical and Chemical Properties of PC-1 Drug Product

| Appearance (color, clarity) | Colorless 1.0 NTU | |
|---|---|---|
| Molecular weight | 91.7 | kDa |
| Melting point Tm by DSC | 72.2° | C. |
| Osmolality | 270 | mOsmol/kg |
| Solution pH | 6.3 | |
| Isoelectric point | 9.52 | |
| Potency by ELISA binding assay after | 100% relative potency | |
| cleavage of CD3 mask (Relative binding | | |
| when compared to the Reference Standard) | | |

Abbreviations: CD3 = cluster of differentiation 3; DSC = differential scanning calorimetry; ELISA = enzyme-linked immunosorbent assay; NTU = nephelometric turbidity unit; Tm = melting temperature.

### Storage and Handling

The PC-1 DP vials are stored and shipped frozen (−20° C.).

### Synopsis of Phase 1 Trial Design

Study Design: This study is a FIH, Phase 1, open-label, multicenter study to assess the safety, tolerability, PK, PD, and preliminary efficacy of PC-1 administered as a single agent in adult subjects with mCRPC. The study will be conducted in 3 parts: Dose Escalation (Part 1) with approximately 20 to 30 subjects, Cohort Backfill Expansion (Part 2) with approximately 30 subjects enrolled across three dose levels, and Dose Expansion (Part 3) with approximately 30 subjects enrolled at the RP2D. Dose Escalation (Part 1) will assess the safety, tolerability, PK, PD, and preliminary efficacy of PC-1 administered by IV infusion. Dose escalation decisions will be made based on the recommendations of the Safety Review Committee, based on the review of safety data and available PK, PD, and preliminary efficacy data. Dose escalation may continue until the dose where at least 2 dose-limiting toxicities are observed (the MTD is exceeded). Cohort Backfill Expansion (Part 2) will allow for further characterization of safety and activity of dose levels. In Dose Expansion (Part 3), up to approximately 30 subjects will be enrolled to obtain additional safety, tolerability, PK, PD, and preliminary clinical activity data with PC-1 at a dose and schedule to be determined by the Safety Review Committee after reviewing all available safety, PK, PD, and preliminary efficacy data.

### Summary of Data and Guidance

Indications and Usage: PC-1 is in development for the treatment of mCRPC and is currently not approved for any indication.

Dosage and Administration: The FIH study is conducted with a starting dose followed by dose escalation. PC-1 will be administered IV different days of the 21-day or 28-day cycles. Subjects will be treated with multiple cycles.

Preparation Instructions: Parenteral drug products should be inspected visually for particulate matter and discoloration prior to administration, whenever solution and container permit. For more details, refer to the Pharmacy Manual (or study protocol).

Dosage Forms and Strengths: The PC-1 DP will be provided as a solution for injection for IV administration at a single strength presentation of 2 mg/mL.

Contraindications: The use of PC-1 is contraindicated in the following conditions: Known hypersensitivity to any drug component.

Warnings and Precautions: There has been no prior clinical experience with PC-1. PC-1 is an antibody protein construct. Like other molecules in this class, it is highly specific for its targets. Although antibody therapeutics are well-tolerated, they are ‘foreign’ proteins, and some patients may experience infusion-related reactions (IRRs) or develop an immune response against them. However, PC-1 was designed to have fewer risks downstream of CD3 activation relative to other T cell-engaging therapies. PC-1 is an experimental drug that should be administered only to patients within the context of a clinical study.

### Potential Adverse Reactions

Because PC-1 is a T cell redirecting antibody, CRS, neurotoxicity, and/or tumor lysis syndrome may occur, although possibly at a lower frequency/severity than with other T cell activating therapies, based on the PC-1 design. Published literature of experience with other monoclonal antibody therapies indicates that pyrexia, immunogenicity reactions (ie, formation of ADAs), and/or hypersensitivity reactions may be observed. These reactions can be both serious and systemic (eg, anaphylaxis) and may occur acutely or be delayed. For regulatory reporting purposes, all adverse events (AEs) will be assessed as being unexpected at this stage of the development program. Therefore, AEs that are serious and possibly related to PC-1 will be reported to the health authorities per applicable regulations.

### Infusion-Related Reactions

PC-1 is a recombinant protein based therapeutic, and administration of therapeutic proteins has been associated with infusion reactions with symptoms and signs including fever, rigors, rash, urticaria, dyspnea, hypotension, and/or nausea. To minimize the risk of infusion reactions, all subjects will be premedicated with acetaminophen/paracetamol and diphenhydramine, and for the first infusion, corticosteroids. Mild infusion reactions should be managed per institutional standards with antipyretics, antihistamines, and antiemetics, and for more severe reactions, with corticosteroids.

### Management of Infusion-Related Reactions

IRRs are defined by National Cancer Institute (NCI) Common Terminology Criteria for Adverse Events (CTCAE) v5.0 (under the category “Injury, poisoning, and procedural complications”) as “a disorder characterized by adverse reaction to the infusion of pharmacological or biological substances”. For the purposes of this study, the time frame for infusion reaction assessment is the 24-hour period beginning from the start of the infusion. Recommendations for the management of IRRs are provided below. For Grade 1 IRR: described as mild transient reaction; infusion interruption not indicated; intervention not indicated: ⋅ Remain at bedside and monitor the subject until recovery from symptoms. For Grade 2 IRR: described as infusion interruption indicated, but responds promptly to symptomatic treatment (e.g, antihistamines, nonsteroidal anti-inflammatory drugs, narcotics, IV fluids); and prophylactic medications indicated for ≤4 hours: ⋅ Stop the PC-1 infusion, begin an IV infusion of normal saline, and treat the subject with diphenhydramine 50 mg IV (or equivalent) and/or 500 to 750 mg oral acetaminophen. ⋅ Corticosteroid therapy may also be given at the discretion of the Investigator. Remain at bedside and monitor the subject until resolution of symptoms. ⋅ If the infusion is interrupted, wait until symptoms resolve, then restart the infusion at 50% of the original infusion rate. ⋅ If no further complications occur after 60 minutes, the rate may be increased to 100% of the original infusion rate. Monitor the subject closely. ⋅ If symptoms recur, stop the infusion and disconnect the subject from the infusion apparatus. ⋅ No further PC-1 will be administered at that visit. ⋅ The amount of PC-1 infused must be recorded on the case report form. ⋅ Subjects who experience a Grade 2 infusion reaction during the post-infusion observation period that does not resolve during that time should be observed until the AE resolves or stabilizes, with vital sign measurements every 4 hours and additional evaluations as medically indicated for the management of the AE.

For Grade 3 or Grade 4 IRR: Grade 3 is described as prolonged (e.g, not rapidly responsive to symptomatic medication and/or brief interruption of infusion) recurrence of symptoms following initial improvement; hospitalization indicated for other clinical sequelae (e.g, renal impairment, pulmonary infiltrates). Grade 4 is described as life-threatening consequences; urgent intervention indicated. ⋅ Immediately discontinue the infusion of PC-1. ⋅ Investigators should follow their institutional guidelines for the treatment of anaphylaxis or high-grade infusion reactions. ⋅ Consider treating the subject with an IV infusion of normal saline and administer corticosteroids, H1 and/or H2 blockers, bronchodilators, oxygen, or vasopressors as needed. ⋅ The subject should be monitored until the Investigator is comfortable that the symptoms will not recur. ⋅ All subjects with Grade≥3 IRRs will be observed until the AEs resolve or stabilize, with vital sign measurements and additional evaluations as medically indicated for the management of the AEs. ⋅ Subjects with Grade 3 IRR during Week 1 of Cycle 1 may also be hospitalized during or after the administration of the subsequent dose, with considerations for dose reduction

### Cytokine Release Syndrome

The identified risks of treatment with T cell engagers (TCE) are primarily related to cytokine release and CRS. PC-1 is designed to reduce the risk of CRS by requiring protease cleavage for activation, focusing molecular activity to the TME where proteases are overexpressed, dysregulated, and activated. This approach has been shown to markedly reduce systemic cytokine exposure in preclinical models. Data from other clinical development programs (of non-masked TCE) has shown that broad T cell activation can lead to cytokine release into the blood, which can cause acute effects of fever, hypotension, and hypoxia and may require care in the hospital. These side effects are short-lived and can be treated with IV fluids, tocilizumab, and supportive care, but occasionally have the potential to be severe. These vital signs and adverse effects will be carefully monitored for their appearance and followed closely in this clinical study. In the nonclinical toxicology studies described in Section 4, PC-1 can be dosed at 50- to 150-fold higher doses than the unmasked active form of TCE with equivalent safety and cytokine induction activity. This safety window observed in animal studies offers the promise that higher active doses may be achieved in patients and may offer an increased level of anti-tumor activity. Guidelines for treatment of CRS have been mainly driven by the CAR-T field, where CRS seen with anti-CD19 CAR-T cells can be severe and more prolonged (Neelapu, 2018). With bispecific antibodies, CRS is shorter duration, and generally less severe, but may still be dose-limiting, and is commonly mitigated by priming/step dosing approaches. Cytokine release is primarily a first dose, or first cycle phenomenon, and tolerance to cytokine induction occurs with subsequent infusions of TCEs. Preventive measures of cytokine release and CRS will include glucocorticoid premedication, IV pre-hydration, and holding of anti-hypertensive medication on day of infusion. Priming and step dosing will be initiated if CRS is seen during dose escalation.

CRS Management: Cytokine release can lead to fevers and tachycardia and can also become more severe and lead to fevers, tachycardia, hypotension and hypoxia, and needs to be followed closely. CRS may also be associated with fatigue, nausea, headache, dyspnea, rigors, myalgia/arthralgia, and anorexia. CRS will be graded using the American Society for Transplantation and Cellular Therapy (ASTCT) consensus grading scale (Table 4), adapted from (Lee et al., 2019). CRS will be managed with IV hydration and other supportive care measures, as necessary, and increased monitoring. More significant CRS may require oxygen, corticosteroids, and/or tocilizumab (anti-IL-6-receptor). Severe CRS may also require intensive care unit monitoring and management with vasopressors, antiepileptics, high-dose corticosteroids, and/or mechanical ventilation. Recommended guidelines for the treatment of CRS have been mainly driven by the CAR-T field, where CRS seen with anti-CD19 CAR-T cells can be severe and more prolonged (Lee et al., 2019). With immunoglobulin G format bispecific antibodies, CRS may occur and be dose-limiting.

TABLE 4
Grading Cytokine Release Syndrome (ASTCT Consensus Scale)

| Parameter | Grade 1 | Grade 2 | Grade 3 | Grade 4 |
|---|---|---|---|---|
| Fever | ≥38° C. | ≥38° C. | ≥38° C. | ≥38° C. |
| Hypotension | None | Responds | Requiring a | Requiring multiple |
| | | to IV | vasopressor | vasopressors (excluding |
| | | fluids | | vasopressin) |
| Hypoxia | None | Requiring | Requiring | Requiring positive |
| | | low-flow | high-flow nasal | pressure (eg, CPAP, |
| | | nasal | cannula, or mask | BiPAP, intubation and |
| | | cannula | | mechanical ventilation) |

Abbreviations: ASTCT = American Society for Transplantation and Cellular Therapy; BiPAP = bilevel positive airway pressure; CPAP = continuous positive airway pressure; IV = intravenous.

### For any Grade CRS:

Monitor clinical laboratory assessments.
Monitor C-reactive protein levels and serum PD cytokine laboratory assessments.
Draw extra safety laboratory samples and plasma for exploratory safety and PD biomarkers. For Grade 1 CRS: Many subjects may only manifest fever and tachycardia. Close monitoring is required to detect and react to early signs of progression.
Frequent monitoring of vital signs
   For Grade 2 CRS: Hypotension and/or Hypoxia Detected
Treat with aggressive IV hydration with normal saline
Supplemental oxygen as indicated
Consider cardiac telemetry and pulse oximetry
Consider corticosteroids (eg, dexamethasone 10 mg every 12 hours)
Consider tocilizumab or equivalent anti-IL-6 antibody (eg, siltuximab), or anti-IL-6-Receptor antibody (eg, sarilimab)

### For Grade 3 CRS: Hypotension not Responsive to IV Fluids

Management in monitored bed or intensive care unit
Corticosteroids as indicated dexamethasone 10 mg every 6 hours, or methylprednisolone 1 mg/kg IV twice per day
Tocilizumab (4 to 8 mg/kg IV over 1 hour, not to exceed 800 mg), or equivalent anti-IL-6 antibody (eg, siltuximab), or anti-IL-6-Receptor antibody (eg, sarilimab)
Vasopressor support
Oxygen therapy with mask if needed

Effective control of CRS, especially on the first dose of PC-1, enables the potential for a wide range of dose regimes (for example dose dense approaches). PC-1 may evaluate any or all of the following CRS risk mitigations:

Corticosteroid premedication consisting of dexamethasone (for example 8 mg) administered orally the day prior to dosing (12-16 hours prior).

Corticosteroid premedication consisting of dexamethasone (for example 10 mg) may be administered orally 3 hours (±1 hour) or IV 1 hour (±30 minutes) at a minimum, prior to administration of each infusion through receipt of the first target dose level.

Corticosteroid premedication consisting of dexamethasone (for example 8 mg) administered orally the day prior to dosing (12-16 hours prior) and an additional Corticosteroid premedication consisting of dexamethasone (for example 10 mg) may be administered orally 3 hours (±1 hour) or IV 1 hour (±30 minutes) at a minimum, prior to administration of each infusion through receipt of the first target dose level. Additional Corticosteroid may be administered post dosing as required.

Tocilizumab or equivalent anti-IL-6 antibody (eg, siltuximab), or anti-IL6 Receptor antibody (eg, sarilimab) may be considered early in the clinical course of CRS to help avoid CRS progression and severe clinical manifestations. Use of IL-6 antibodies or IL-6 receptor antibodies may be used prophylactically (before PC-1 administration) or at the onset of CRS.

Prophylactic Corticosteroid may also be used with Tocilizumab or equivalent.

To minimize the risk of CRS-associated hypotension, anti-hypertensive medicine may be held 48-72 hours prior to and after all infusions of PC-1 in Cycle 1 at the discretion of the Investigator and or treating cardiologist recommendation.

To minimize the risk of CRS-associated hypotension PC-1 may be administered to subjects pre-hydrated with normal saline (for example 500 cc). Hydration may be completed 10-30 minutes prior to administration of the Cycle 1 Day 1 dose. Pre-infusion hydration may be continued, at a minimum, prior to administration of each infusion through receipt of the first target dose level.

Premedication with acetaminophen (or paracetamol) and an anti-histamine regimen may be administered per standard institutional practice prior to administration of each dose of PC-1 as tolerated.

### Other Potential Toxicities

Tumor Lysis Syndrome; Subjects with a high disease burden may be at risk for developing tumor lysis syndrome (TLS) with PC-1 treatment. Prophylactic treatment/measures are strongly recommended for subjects considered to be at risk for TLS, per institutional or clinical standards. Subjects should be closely monitored for laboratory evidence of TLS (hyperuricemia, hyperkalemia, hyperphosphatemia, and hypocalcemia). In the case of evidence of TLS associated with PC-1, subjects will be admitted to the hospital, as clinically indicated. Standard management will include vigorous IV hydration, hypouricemic agents, and correction of acidosis, if present. Renal function, serum uric acid, calcium, phosphorus, and electrolytes should be closely monitored. Subjects with Grade 3 to 4 TLS during Week 1 or Cycle 1 may also be hospitalized for ≥24 hours after the end of the administration of the subsequent dose, with considerations for dose reduction as described in the study protocol.

Neurological Events: Administration of solid tumor-targeted bispecifics has shown less potential for severe neurologic toxicity (compared with CAR-T therapy or anti-CD19 BiTE format antibodies such as blinatumomab) in early-stage clinical studies. Although PC-1 at active doses has not shown a propensity for induction of cytokine levels that may be associated with neurologic toxicity in in vitro human cell cultures or in vivo cynomolgus monkey studies, the risk of neurotoxicity is unknown, and caution is warranted. Neurological events associated with cytokine release may include tremor, mental status changes, confusion, speech difficulties, and potentially seizures. Monitor subjects for neurological events and exclude other causes for neurological symptoms. Neurological events should be graded according to NCI CTCAE v5.0. Mild disorientation or expressive aphasia (trouble word-finding) may be the earliest and most specific signs. Provide supportive care as needed for any neurological events. Workup may include head magnetic resonance imaging and electroencephalogram and may require corticosteroids, or anti-seizure medications, if severe. The Medical Monitor should be contacted if there is any potentially treatment-related neurological toxicity.

### Infections

As seen with other TCE-based immunotherapies, serious infections, including fatal bacterial, fungal, and new or reactivated viral infections, may occur during and/or following the completion of PC-1-based therapy. New or reactivated viral infections may include cytomegalovirus, herpes simplex virus, parvovirus B19, varicella zoster virus, West Nile virus, hepatitis B virus (HBV), and hepatitis C virus (HCV). PC-1 should be discontinued if serious infections develop, and appropriate anti-infective therapy instituted. PC-1 is not recommended for use in subjects with severe, active infections.

Hepatitis B Reactivation: HBV reactivation can occur in patients treated with drugs classified as TCE antibodies. Cases have been reported in patients who are hepatitis B surface antigen (HBsAg)-negative but are hepatitis B core antibody (anti-HBc)-positive. HBV reactivation is defined as an abrupt increase in HBV replication manifesting as a rapid increase in serum HBV DNA levels or detection of HBsAg in a person who was previously HBsAg-negative and anti-HBc-positive. Reactivation of HBV replication is often followed by hepatitis (ie, increase in transaminase levels). In severe cases, increase in bilirubin levels, liver failure, and death can occur. It is recommended to monitor subjects with evidence of prior HBV infection (anti-HBc-positive) with HBV DNA testing monthly, and for clinical and laboratory signs of hepatitis during and for several months following PC-1 therapy.

### Immunization

The safety of immunization with live viral vaccines during or following PC-1 therapy has not been studied. Vaccination with live virus vaccines is not recommended for ≥2 weeks prior to the start of PC-1 treatment, during treatment, and until immune recovery following the last cycle of PC-1. Subjects are encouraged to get the COVID-19 vaccination/booster >2 weeks prior to starting the study. Vaccination efficacy on study is unknown; however, COVID-19 vaccination would be allowed in the study, after clearance of the first cycle, and with a 2-week dose hold.

### Other Potential Healthy Tissue Toxicities

While PSMA is most highly expressed in PC cells, and highly prostate restricted, it can be expressed at lower levels on some healthy tissues. By requiring tumor protease for activation of PC-1, there is another layer of protection for normal tissue T cell activation. Tissues would need to express PSMA, as well as express particular protease for local activation of PC-1 in non-tumor sites.

### Kidney, Gastrointestinal, and Liver

Immunohistochemistry studies have indicated that several non-prostatic tissues can express low levels of PSMA. These include kidney proximal tubule cells, and duodenal brush border cells, and rare cells colonic crypts (Silver et al., 1997; The Protein Atlas, 2022). PSMA-PET scanning, using a small molecule PSMA active site binder, has confirmed tissue accumulation in the small intestine, and liver. (Kidney accumulation cannot be resolved, as the reagent is excreted through the kidney and bladder). Renal or gastrointestinal toxicity has not been described as an issue with small molecule PSMA binders, such as 177-Lu-PSMA-617 (PLUVICTO) (Sartor et al., 2021), or with the PSMA-CD3 bispecific antibodies such as AMG160. However, transaminitis has been seen with AMG160, but has been mostly attributed to CRS (Tran et al., 2020).

Salivary and Lacrimal Glands: PSMA-PET scanning, using a small molecule PSMA active site binder, has demonstrated clear salivary and lacrimal gland uptake. It is not clear if there are other nonspecific or poorly understood mechanisms for this uptake (Morris et al., 2021; Sheehan et al., 2021). Small molecule PSMA binders, such as 177-Lu-PSMA617 (PLUVICTO), have shown a potential for a dry mouth and dry eye side effects (Sartor et al., 2021); these side effects are much less frequently seen with PSMA-CD3 bispecifics, but they can occur. (Tran et al., 2020).

Drug Interactions: The drug interaction profile of PC-1 is unknown, however, no clinically relevant drug-drug interactions would be expected with PC-1.

### Use in Specific Populations

### Teratogenic Effects

Whether PC-1 has teratogenic effects is unknown. Should a subject or female partner of a subject experience a pregnancy during the study, further treatment with PC-1 should be stopped, immediately.

### Nonteratogenic Effects

Whether PC-1 has nonteratogenic effects is unknown. Should a subject or female partner of a subject experience a pregnancy during the study, further treatment with PC-1 should be stopped, immediately.

### Geriatric Use

No studies in geriatric patients have been conducted. Since the average age at diagnosis of PC is 66 years (Cancer.org), elderly patients will be included in the initial clinical trials of PC-1. Elderly subjects should, however, be carefully monitored.

### Renal Impairment

The effect of renal impairment on PC-1 PK and/or function is not known. However, because PC-1 is a biologic compound, PK/functional alterations due to renal impairment are not anticipated (FDA 2020).

### Hepatic Impairment

The effect of hepatic impairment on PC-1 PK and/or function is not known.

### Overdosage

The effects of an overdose of PC-1 are not known. In the event of an overdose (ie, any dose greater than that defined in the protocol), supportive care will be provided.

### Description

PC-1 is a tri-specific TRACTr that incorporates the PSMA and CD3 binding domains, a tandem albumin-binding domain with peptide (mask) that inhibits PC-1 binding to CD3 on T cells, and a tumor protease cleavage sequence (FIG. 1). Intact PC-1 is designed to have reduced binding to CD3 on T cells (relative to unmasked versions) and minimize T cell activation and pharmacology in healthy tissue.

### Clinical Pharmacology

### Mechanism of Action

Target engagement by PC-1 requires proteolysis of its cleavable amino acid linker by proteases present in the TME. Once the amino acid cleavage sequence undergoes proteolysis by tumor proteases, the tandem mask and albumin-binding domains are released to enable CD3 binding. Simultaneous binding of bispecific components of cleaved PC-1 to CD3 and PSMA results in T cell activation in the tumor, leading to T cell-mediated killing of prostate tumor cells.

### Pharmacodynamics

Pharmacodynamics in humans have not been evaluated and will be assessed in clinical studies of PC-1.

### Pharmacokinetics

Pharmacokinetics in humans are assessed in clinical studies of PC-1.

How Supplied/Storage and Handling: PC-1 DP will be shipped frozen and should be stored at −20° C.

### Results

### Mechanism of Action

Target engagement by PC-1 requires proteolysis of its cleavable amino acid linker by proteases present in the TME. Once the amino acid cleavage sequence undergoes proteolysis by tumor proteases, the tandem mask and albumin-binding domains are released to enable CD3 binding. Simultaneous binding of bispecific components of cleaved PC-1 to CD3 and PSMA results in T cell activation in the tumor, leading to T cell-mediated killing of prostate tumor cells. (FIG. 1). The masking blocks binding to targets in healthy tissues and inhibits immune response against healthy tissues. It reduces healthy tissue cytokine release to reduce systemic toxicity, and improved safety. In the tumor environment, tumor proteases cleave linkers which releases both masks and the half-life extender. The fully active TCE drives maximum anti-tumor immune response. The active TCE diffuses from tumor and is rapidly cleared, reducing active TCE levels in healthy tissue and reduces risk of systemic toxicity to improve safety profile. (see FIG. 1).

### Effects in Humans

### Introduction

PC-1 is being evaluated in one ongoing clinical study. For complete patient eligibility criteria, please see the clinical study protocol disclosed herein.

TABLE 5
Overview of Ongoing Study with PC-1

| Protocol Status | Objective | Study Design | Treatment |
|---|---|---|---|
| Ongoing | To determine the | Phase 1, open-label, | PC-1 is |
| | tolerability, PK, PD | multicenter, safety, PK | administered as a |
| | preliminary efficacy of | and PD study of single- | weekly IV infusion |
| | PC-1 administered | agent PC-1 in adult | during 21-day cycles. |
| | as a single agent | subjects with | Flat dosing and |
| | | mCRPC. The study | step dosing |
| | | contains 3 parts, | regimens will be |
| | | (Part 1), Backfill | Alternate dosing |
| | | Expansion (Part 2) and | intervals (i.e. Q2W or |
| | | Dose Expansion (Part | Q3W) may be |
| | | 3) | considered for some |
| | | | subjects. |

IV = intravenous;
mCRPC = metastatic castration-resistant prostate cancer;
PD = pharmacodynamic;
PK = pharmacokinetic;
Q2W = every 2 weeks;
Q3W = every 3 weeks

### Pharmacokinetics and Metabolism in Humans

The PK of PC-1 is being investigated in the ongoing study. PK data analysis will be conducted at the completion of the study.

### Safety and Efficacy

The safety and efficacy of PC-1 has been evaluated in a Phase 1, open-label, multicenter study to assess the safety, tolerability, PK, PD and preliminary efficacy of PC-1 administered as a single agent in adult subjects with mCRPC.

Adverse events (AEs) are coded using the Medical Dictionary for Regulatory Activities (MedDRA) version 25. The severity of AEs is graded according to National Cancer Institute Common Terminology Criteria for Adverse Events version 5.0 (NCI CTCAE v5.0) except for cytokine release syndrome (CRS) where the American Society for Transplantation and Cellular Therapy (ASTCT) grading will be used (Lee 2019).

### Safety

PC-1 has been administered in one Phase 1 study to patients with mCRPC. PC-1 has been administered at flat doses of 100 μg and 300 μg as well as with a step dosing regimens, for example 50/100/300 μg. One subject discontinued study treatment from the 50/100/300 μg step dose regimen, due to a treatment related AE of acute kidney injury. After the data cut-off date, additional evaluations confirmed the event was considered related to progression of the subject's underlying disease and not PC-1. Of the 8 subjects dosed, 1 death, due to the disease under study, was reported after the 30-day follow up period.

### Eligibility Criteria

### Description

### Inclusion Criteria:

Male ≥18 years of age at the time of signing informed consent
Histologically or cytologically confirmed adenocarcinoma of the prostate
Having mCRPC that progressed after at least one novel anti-androgen therapy and at least one taxane containing regimen. Participants who have actively refused a taxane containing regimen or are medically unsuitable to receive taxane are eligible
Adequate organ function

### Exclusion Criteria:

Prior solid organ transplant
Prior treatment with PSMA-targeted CAR-T cell therapy
Clinically significant cardiovascular disease
Active clinically significant infection (bacterial, viral, fungal, mycobacteria or other)
Any medical condition or clinical laboratory abnormality likely to interfere with assessment of safety or efficacy of study treatment

### Design of Study

TABLE 6
Arms and Interventions

| Participant Group/Arm | Intervention/Treatment |
|---|---|
| Experimental: Dose Escalation | Biological: PC-1 |
| Weekly dosing during each 21-day cycle. | PC-1 is dosed via IV weekly |
| Dosage per cohort will increase to | in a 21-day cycle |
| determine the maximum tolerable dose. | |
| Experimental: Backfill Expansion | Biological: PC-1 |
| Weekly dosing during each 21-day cycle. | PC-1 is dosed via IV weekly |
| Subjects will be dosed at levels previously | in a 21-day cycle |
| declared tolerable. | |
| Experimental: Expansion | Biological: PC-1 |
| Weekly dosing during each 21-day cycle. | PC-1 is dosed via IV weekly |
| Subjects will be dosed at preliminary | in a 21-day cycle |
| recommended phase 2 dose (RP2D). | |

TABLE 7
Secondary Outcome Measures

| Outcome Measure | Measure Description | Time Frame |
|---|---|---|
| Area under the concentration | | Pre-dose and at multiple |
| time curve to infinity of PC- | | timepoints post-dose on |
| 1 (AUC_{0-inf}) | | Days 1, 2, 4, 8, 9, 15, 16, 18 |
| | | up to end of treatment (Up |
| | | to 3 years |
| Maximum observed | | Pre-dose and at multiple |
| concentration of PC-1 | | timepoints post-dose on |
| (Cmax) | | Days 1, 2, 4, 8, 9, 15, 16, 18 |
| | | up to end of treatment (Up |
| | | to 3 years) |
| Number of participants who | | Up to 3 years |
| develop anti-drug antibodies | | |
| against PC-1 | | |
| Duration of Response | Time from documentation of | Up to 3 years |
| | complete response or partial | |
| | response to disease | |
| | progression using RECIST | |
| | v1.1 and PCWG3 | |
| Prostate Specific Antigen | Best reduction in PSA level | Up to 3 years |
| (PSA) response | achieved | |
| Radiographic Progression | Time from treatment | Up to 3 years |
| Free Survival (rPFS) | initiation to radiographic | |
| | evidence of disease | |
| | progression using RECIST | |
| | v1.1 and PCWG3 | |
| Overall Response Rate | Proportion of participants | Up to 3 years |
| | who achieve a complete | |
| | response or partial response | |
| | using RECIST v1.1 and | |
| | PCWG3 | |
| Overall Survival | Time from treatment | Up to 3 years |
| | initiation until death from | |
| | any cause | |

### Example 2: Clinical Study Protocol of Polypeptide Complex 1 (PC-1)

This example illustrates a clinical study protocol of a phase 1, open-label, multicenter study of PC-1 disclosed herein in subjects with metastatic castration-resistant prostate cancer. (mCRPC)

### Protocol Synopsis

TABLE 8
Protocol Synopsis

| | |
|---|---|
| Title of Study: | A Phase 1, Open-Label, Multicenter Study of PC-1 in Subjects with |
| | Metastatic Castration-Resistant Prostate Cancer |
| Clinical Phase: | 1 |
| Population: | Subjects with metastatic castration-resistant prostate cancer (mCRPC) |
| Objectives: | Primary Objectives: |
| | To assess safety and tolerability in subjects with mCRPC |
| | To determine the maximum tolerated dose regimen (MTD-R) |
| | and/or maximally administered dose (MAD) |
| | To assess potential Phase 2 dose regimens and determine a |
| | recommended Phase 2 dose regimen (RP2D-R) |
| | Secondary Objectives: |
| | To evaluate the pharmacokinetic (PK) profile of PC-1 |
| | following intravenous (IV) administration |
| | To evaluate the immunogenicity of PC-1 |
| | To assess preliminary efficacy of PC-1 |
| | Exploratory Objectives: |
| | To evaluate the pharmacodynamic (PD) profile of PC-1 |
| | following IV administration |
| | To characterize the impact of PC-1 on systemic immune |
| | factors, including cytokines and immune cells |
| | To assess response by novel methods, prostate-specific |
| | membrane antigen (PSMA)-positron emission tomography (PET) |
| | evaluation, circulating tumor cells (CTC), and circulating tumor (ctDNA) |
| | quantitation |
| | To assess baseline and on-treatment tumor and immune |
| | biomarkers and evaluate for correlations with anti-tumor activity and |
| | safety |
| | To assess additional preliminary efficacy of PC-1 |
| Study Design: | This study is a first-in-human, Phase 1, open-label, multicenter study to |
| | assess the safety, tolerability, PK, PD, and preliminary efficacy of PC-1 |
| | administered as a single agent in adult subjects with mCRPC. |
| | This study will be conducted in 3 parts: Dose Escalation (Part 1) with |
| | approximately 20 to 30 subjects, Cohort Backfill (Part 2) with up to |
| | approximately 45 subjects, and Dose Expansion (Part 3) with up to |
| | approximately 30 subjects enrolled at the RP2D-R. An outline of the |
| | study is disclosed herein. |
| | PC-1 will be administered IV weekly on Days 1, 8, and 15 of 21-day |
| | cycles. Alternate dosing schedules [e.g. 4-day step dose schedule (Days |
| | 1, 4, 8 in the first week of Cycle 1), every 2 week (Q2W), every 3 week |
| | (Q3W), etc.] may also be evaluated. Cycle 1 may be longer than 21 days |
| | if a ≥3-step regimen or Q2W regimen is evaluated. Tumor assessments |
| | will include prostate-specific antigen (PSA) levels, computed |
| | tomography (CT), and bone scan imaging and will be performed at |
| | baseline, Week 9, Week 18 and then every 3 months. PSMA-PET will |
| | be at baseline, Week 9, Week 18 and after the determination of response |
| | by conventional imaging if response is first observed after Week 18. |
| | The dosing schema is disclosed herein. |
| | Dose Escalation (Part 1) |
| | Dose Escalation (Part 1) will use an accelerated titration design and |
| | start with a single-subject cohort at the 100 μg dose level. If no safety |
| | signals are detected during the dose-limiting toxicity (DLT) period, |
| | the study may progress to the next dose level. If a safety signal is |
| | observed, such as cytokine release syndrome (CRS), an additional 2 |
| | subjects will be enrolled at that dose level, and the study will convert |
| | to a standard 3 + 3 approach. This design is chosen to potentially |
| | reduce the number of subjects treated with doses likely to be below a |
| | minimally effective threshold. Dose escalation will utilize a standard |
| | 3 + 3 scheme starting at the 300 μg dose level. Beginning at Cohort 6, |
| | dose escalation will be guided by a Bayesian Logistic Regression |
| | Model (BLRM) with overdose control (EWOC). Step doses may be |
| | included to further mitigate CRS risk. |
| | Cohort management is under the supervision and direction of the Safety |
| | Review Committee (SRC). The SRC must clear a cohort prior to the |
| | initiation of new higher dose cohorts. |
| | All subjects in Dose Escalation (Part 1) will be required to be |
| | hospitalized as disclosed herein. |
| | The starting dose of 100 μg IV is based on the minimally anticipated |
| | biologic effect level (MABEL) in humans. Examples of PC-1 dose |
| | regimens that may be investigated are disclosed herein. |
| | For each dose escalation cohort, treatment with the first dose of PC-1 |
| | will be staggered such that the second subject in the cohort will |
| | initiate PC-1 treatment at least 3-7 days after the first subject |
| | depending on the regimen employed. Subsequent subjects (e.g., |
| | between the second and third subject) enrolled in each cohort will be |
| | staggered by at least 1 day. |
| | The SRC will review available safety, PK, PD, and preliminary efficacy |
| | data to provide recommendations for all dose escalation decisions. |
| | Cohort Backfill (Part 2) |
| | The study may allow for limited expansion of a prior cleared dose |
| | regimen (up to 15 subjects per cohort, 18 subjects per dose regimen in |
| | Parts 1 and 2 combined). Additionally, in Part 2, alternative dosing |
| | schedules [i.e., every 2 weeks (Q2W) or 3 weeks (Q3W)] may also be |
| | investigated. Cohort backfill will allow for further characterization of |
| | safety and activity of dose regimens prior to initiation of the formal dose |
| | expansion cohort (Part 3). |
| | The backfill cohorts may open concurrently with ongoing dose |
| | escalation. The dose regimen for the backfill cohorts will be selected |
| | based on SRC review of cumulative safety, PK, PD, and preliminary |
| | efficacy data. Available information from backfill cohorts will be |
| | used in the BLRM for the ongoing dose finding and will further |
| | support RP2D-R selection per review by the SRC. |
| | Hospitalization requirements in Cohort Backfill (Part 2) will be |
| | determined by the SRC in consultation with Investigators based on the |
| | safety observed in Dose Escalation (Part 1). |
| | Dose Expansion (Part 3) |
| | Up to approximately 30 subjects will be enrolled to obtain additional |
| | safety, tolerability, PK, PD, and preliminary clinical activity data with |
| | PC-1 at a dose and schedule to be determined by the SRC after |
| | reviewing all available safety, PK, PD, and preliminary efficacy data. At |
| | no time will the PC-1 dose regimen studied in Dose Expansion (Part 3) |
| | exceed the highest dose regimen that qualifies as an MTD-R in Dose |
| | Escalation (Part 1). |
| | Hospitalization requirements in Dose Expansion (Part 3) will be |
| | determined by the SRC in consultation with Investigators based on the |
| | safety observed in Dose Escalation and Backfill (Part 1 and Part 2). |
| Investigators and | Dose Escalation (Part 1): Approximately 8 to 10 sites |
| Study Sites: | Cohort Backfill (Part 2): Approximately 12 to 20 sites |
| | Dose Expansion (Part 3): Up to approximately 20 sites |
| Number of | Dose Escalation (Part 1): Approximately 20 to 30 subjects |
| Subjects: | Cohort Backfill (Part 2): Up to approximately 45 subjects |
| | Dose Expansion (Part 3): Up to approximately 30 subjects |
| | Total (Parts 1-3): Up to approximately 105 subjects |
| Inclusion Criteria: | See detailed protocol below |
| Exclusion | See detailed protocol below |
| Criteria: | |
| Duration of | Subject participation will include screening, treatment, and follow-up. |
| Individual Study | Screening will be conducted up to 28 days before the first dose of PC-1, |
| Participation: | during which the subject's eligibility and baseline characteristics will be |
| | determined. |
| | Subjects exhibiting acceptable safety and stable disease (SD) or better |
| | may continue to receive PC-1 until disease progression, unacceptable |
| | toxicity or no longer clinically benefiting (NLCB). |
| | Post-treatment, subjects will be followed for disease progression until |
| | the start of new anticancer therapy and survival until death, withdrawal |
| | of consent, or the end of the study, whichever occurs first. The expected |
| | median duration of the study treatment for individual subjects is |
| | approximately 18 to 36 weeks. |
| Duration of Study: | The planned duration of the study is approximately 4 years. |
| Test Product, | PC-1 is a recombinant trispecific biologic, which binds PSMA, CD3, |
| Dose, and Mode of | and albumin. PC-1 drug product is a sterile, clear, colorless to slightly |
| Administration: | yellow, preservative -free frozen liquid vial intended for IV infusion. |
| | PC-1 will be administered IV. Dosing independent of body weight or |
| | surface area will be used for PC-1. Premedication requirements are |
| | described herein. Premedication requirements may be modified per |
| | recommendation of the SRC. Changes to the premedication |
| | requirements for individual subjects must be approved by the Medical |
| | Monitor. |
| | PC-1 will be initially infused over 2 hours (±15 minutes). The infusion |
| | may be slowed or interrupted for subjects experiencing infusion- |
| | associated symptoms. Subjects in Dose Escalation (Part 1) will be |
| | hospitalized as described herein |
| | For visits that do not require hospitalization, subjects will be observed |
| | for at least 2 hours post administration for fever, chills, rigors, |
| | hypotension, nausea, or other signs and symptoms of infusion-related |
| | reactions. After Cycle 1, the infusion time and post observation period |
| | may be reduced at the discretion of the Investigator in consultation with |
| | the Medical Monitor. In the absence of infusion-related AEs, the |
| | infusion time may be successively reduced by up to 30 minutes to a |
| | minimum infusion time of 30 minutes. Similarly, the post administration |
| | observation period may be successively reduced by up to 30 minutes for |
| | a minimum observation period of 30 minutes. |
| Endpoints: | Primary Endpoints |
| | Incidence of AEs, SAEs, and DLTs according to National Cancer |
| | Institute |
| | Common Terminology Criteria for Adverse Events Version 5.0 (NCI |
| | CTCAE v5.0) |
| | Cytokine release syndrome (CRS) will be graded per American Society |
| | for |
| | Transplantation and Cellular Therapy (ASTCT) criteria (Lee et al 2019) |
| | Secondary Endpoints |
| | PK: area under the concentration-time curve from time 0 to time t |
| | (AUC0-t), area under the concentration-time curve extrapolated to |
| | infinity (AUC0-inf), clearance (CL), maximum concentration (Cmax), |
| | time of maximum concentration (Tmax), last measurable concentration |
| | (Clast), time of last measurable concentration (Tlast), terminal half-life |
| | (t½), and volume of distribution (Vd) of PC-1 |
| | Incidence and persistence of anti-drug antibodies (ADAs) against PC-1 |
| | and impact on the PK profile of PC-1 |
| | PSA response rate as determined by Investigators according to Prostate |
| | Cancer |
| | Clinical Trials Working Group 3 (PCWG3) |
| | Overall response rate (ORR), duration of response (DOR), and |
| | radiographic progression free survival (rPFS), as determined by |
| | Investigators according to |
| | Response Evaluation Criteria in Solid Tumors (RECIST) 1.1 and |
| | PCWG3 |
| | Overall survival (OS) |
| | Exploratory Endpoints |
| | Baseline and on-treatment cytokine and chemokine levels, immune cell |
| | characterization and activation profile |
| | Baseline PSMA expression level (PSMA-PET) and response assessment |
| | Baseline and on treatment CTC and ctDNA quantitation |
| | In the subset of subjects with biopsies, evaluation of tumor biology, |
| | tumor microenvironment characteristics, and T-cell infiltration |
| | PFS as determined by Investigators according to RECIST 1.1 |
| Statistical | This is an open-label clinical study and, in general, descriptive statistics |
| Considerations: | will be employed to analyze the data. More details will be provided in a |
| | Statistical Analysis Plan (SAP). |
| | Sample Size Considerations |
| | No formal hypothesis testing is planned. The number of subjects in each |
| | part of the study is based on safety and tolerability. |
| | Dose Escalation (Part 1) will enroll approximately 20 to 30 subjects. |
| | The study was initiated using a 3 + 3 escalation design. Beginning at |
| | Cohort 6, the study will begin utilizing BLRM. The exact number of |
| | subjects in Part 1 will depend on safety/tolerability, the |
| | recommendations for the SRC and hence the number of cohorts studied. |
| | Cohort Backfill (Part 2) will enroll up to approximately 45 subjects. Up |
| | to |
| | 15 subjects per cohort will be enrolled to gain further characterization of |
| | the safety, tolerability, PK, PD, and preliminary efficacy of defined dose |
| | regimens of PC-1. These data will also be used to select dose for Dose |
| | Expansion (Part 3). |
| | A sample size of up to approximately 30 subjects in Dose Expansion |
| | (Part 3) will further characterize and provide an estimation precision |
| | (as the maximum half-width of the 95% confidence interval [CI]) of |
| | approximately ±18% for any binary rate of outcome assessing safety, |
| | tolerability, exposure, immunogenicity, PD, or clinical activities of |
| | single agent PC-1. These levels of precision are deemed sufficient in |
| | the comprehensive review of the study data to guide future clinical |
| | development. |

TABLE 9
List of Abbreviations

| Abbreviation or Term | Definition/Explanation |
|---|---|
| ADA | anti-drug antibody |
| ADL | activities of daily living |
| ADT | androgen deprivation therapy |
| AE | adverse event |
| ALP | alkaline phosphatase |
| ALT | alanine aminotransferase |
| ANC | absolute neutrophil count |
| AST | aspartate aminotransferase |
| ASTCT | American Society for Transplantation and Cell Therapy |
| AUC_{0-inf} | area under the concentration-time curve extrapolated to infinity |
| AUC₀₋ₜ | area under the concentration-time curve from time 0 to time t |
| AV | atrioventricular |
| BD | binding domain |
| BiPAP | bilevel positive airway pressure |
| BLRM | Bayesian logistic regression model |
| BUN | blood urea nitrogen |
| CAR-T | chimeric antigen receptor T cell |
| CBC | complete blood count |
| CEA | carcinoembryonic antigen |
| CFR | Code of Federal Regulations |
| CI | confidence interval |
| CL | clearance |
| Cₗₐₛₜ | last measurable concentration |
| Cₘₐₓ | maximum concentration |
| CNS | central nervous system |
| CPAP | continuous positive airway pressure |
| CR | complete response |
| CRA | Clinical Research Associate |
| CRF | case report form |
| CRP | C-reactive protein |
| CRR | complete response rate |
| CRS | cytokine release syndrome |
| CSF | cerebrospinal fluid |
| CT | computed tomography |
| ctDNA | circulating tumor DNA |
| CTC | circulating tumor cell |
| CTCAE | Common Terminology Criteria for Adverse Events |
| D | Day |
| DAS | Dose-Limiting Toxicity Evaluable Analysis Set |
| DCR | disease control rate |
| DLT | dose-limiting toxicity |
| DOR | duration of response |
| DP | drug product |
| DVT | deep vein thrombosis |
| EC50 | half maximal effective concentration |
| ECG | electrocardiogram |
| ECHO | echocardiogram |
| ECOG | Eastern Cooperative Oncology Group |
| EDC | electronic data collection |
| EOT | End of Treatment |
| EWOC | escalation with overdose control |
| FAS | Full Analysis Set |
| FDA | Food and Drug Administration |
| FDG-PET | fluorodeoxyglucose-positron emission tomography |
| FIH | first-in-human |
| GCP | Good Clinical Practice |
| GLP | Good Laboratory Practice |
| HBsAg | hepatitis B surface antigen |
| HBV | hepatitis B virus |
| HBc | hepatitis B core |
| HCV | hepatitis C virus |
| HNSTD | highest non-severely toxic dose |
| HRQoL | health-related quality of life |
| IB | Investigator's Brochure |
| ICF | informed consent form |
| ICH | International Council for Harmonisation of Technical Requirements for |
| ID | identification |
| IEC | Independent Ethics Committee |
| IFN-γ | interferon-gamma |
| IgG | immunoglobulin G |
| IL | interleukin |
| IND | Investigational New Drug |
| INR | international normalized ratio |
| IRB | Institutional Review Board |
| IRR | infusion-related reaction |
| IV | intravenous(ly) |
| KM | Kaplan-Meier |
| LDH | lactate dehydrogenase |
| LVEF | left ventricular ejection fraction |
| MABEL | minimally anticipated biologic effect level |
| MAD | maximally administered dose |
| mCRPC | metastatic castration-resistant prostate cancer |
| MedDRA | Medical Dictionary for Drug Regulatory Activities |
| MRI | magnetic resonance imaging |
| MTD-R | maximum tolerated dose regimen |
| MUGA | multigated acquisition |
| NCI | National Cancer Institute |
| NE | not evaluable |
| NGS | next generation sequencing |
| 99mTc MDP | 99mTc methylene diphosphonate |
| NLCB | no longer clinically benefiting |
| NLR | neutrophil/lymphocyte ratio |
| NOAEL | no observed adverse effect level |
| ORR | objective response rate |
| OS | overall survival |
| PBMC | peripheral blood mononuclear cell |
| PC | prostate cancer |
| PCR | polymerase chain reaction |
| PCWG2 | Prostate Cancer Clinical Trials Working Group 2 |
| PCWG3 | Prostate Cancer Clinical Trials Working Group 3 |
| PD | pharmacodynamic(s) or progressive disease |
| PET | positron emission tomography |
| PFS | progression-free survival |
| PK | pharmacokinetic(s) |
| PKAS | Pharmacokinetic Analysis Set |
| PPAS | Per Protocol Analysis Set |
| PR | partial response |
| PRO | patient-reported outcome |
| PSA | prostate-specific antigen |
| PSADT | prostate-specific antigen doubling time |
| PSMA | prostate-specific membrane antigen |
| PT | prothrombin time |
| PTT | partial thromboplastin time |
| PVC | polyvinyl chloride |
| RECIST | Response Evaluation Criteria in Solid Tumors |
| RBC | red blood cell |
| RP2D-R | recommended Phase 2 dose regimen |
| SAE | serious adverse event |
| SAP | Statistical Analysis Plan |
| SD | stable disease |
| SGOT | serum glutamic-oxaloacetic transaminase |
| SGPT | serum glutamic-pyruvic transaminase |
| SOA | Schedule of Assessments |
| SOC | system organ class |
| SRC | Safety Review Committee |
| SUSAR | suspected unexpected serious adverse reaction |
| SUV | standard uptake value |
| t½ | terminal half-life |
| TAA | tumor-associated antigen |
| TBD | To be determined |
| TCE | T cell engager |
| TEAE | treatment-emergent adverse event |
| Tlast | time of last measurable concentration |
| TLS | tumor lysis syndrome |
| Tmax | time of maximum concentration |
| TMDD | target-mediated drug distribution |
| TME | tumor microenvironment |
| TNF-α | tumor necrosis factor-alpha |
| TRACTr | tumor activated T-cell engager |
| ULN | upper limit of normal |
| US | United States |
| Vd | volume of distribution |

### Background Information on PC-1

A TRACTr molecule (PC-1) was developed and designed to improve the therapeutic profile of PSMA-targeted TCFs in patients with mCRPC (FIG. 1). The TRACT consists of a core bispecific TCF that binds to PSMA (an antigen overexpressed on PC cells) and CD3 on T cells. The TCF construct is then made into a TRACTr using a peptide mask that binds to the CD3 binding domain of PC-1 and is connected by a tumor-related protease cleavable linker. The mask inhibits CD3 T-cell binding, limits activity outside the TME, and helps mitigate broad T-cell activation that contributes to CRS. In addition, PC-1 exhibits an extended half-life via incorporation of an albumin-binding domain also attached via the same protease cleavable linker on the CD3 mask (FIG. 1).

The conditional masking and half-life extension of PC-1 is tumor protease cleavage dependent. Published work describes the upregulation of many proteases in tumors relative to healthy tissue, including matrix metalloproteases and serine proteases. In addition, several protease activated biologics and imaging agents have been clinically validated across a broad spectrum of tumor types. By design, TRACTr molecules are highly sensitive to tumor selective proteases. Once the TRACTr reaches the TME, proteases cleave the specific substrates (1 serine protease substrate and 1 matrix metalloprotease substrate) within the cleavable linker, releasing the CD3 mask and albumin-binding domain. The result of protease cleavage is the conversion of the TRACTr to its active form, a TCE.

Notably, the active TCE (PC-1-TCE) has a very short serum half-life (<2 hours in monkey studies) meaning active TCE molecules that escape the TME should be cleared from the body before they can generate significant off-tumor toxicity (FIG. 1). Preclinical data described herein indicate that TRACTrs can drive potent anti-tumor responses while producing >100-fold less systemic interleukin-6 (IL-6) (a key marker/mediator of CRS) with 10-fold higher dose levels relative to non-masked TCEs. It is anticipated that the TRACTr approach will allow us to demonstrate differentiated safety and efficacy profiles in patients with PC. Accordingly, a first-in-human (FIH), Phase 1, multicenter, open-label study was carried out to determine the safety, PK, and recommended Phase 2 dose (RP2D) and to evaluate the preliminary anti-tumor activity of PC-1 administered as a single agent in adult subjects with mCRPC.

### Starting Dose Rationale

To determine an appropriate starting dose for FIH clinical studies, an approach based on the minimally anticipated biologic effect level (MABEL) was used (Saber et al 2017). The planned starting dose for this study is 100 μg, administered as an IV infusion every 7 days. The dosing regimen of every 7 days is planned as the terminal half-life (t½) could range between 1-5 days, due to expected target-mediated drug distribution (TMDD) in human with targeting PSMA (as observed with a competitor molecule HPN424). The in vitro assays are closed, static systems and are not expected to be representative of the open, dynamic in vivo system. Pharmacologically relevant in vivo models for assessment of PC-1 PK/pharmacodynamics (PD) and associated mouse efficacy model were not available, and therefore quantitative translation of in vitro/in vivo observations to in vivo target concentrations and doses in humans was not fully possible. The starting dose was calculated using an integrated data assessment approach and was based on the PK guided approach associated with translating to human dose from in vitro achieved half maximal effective concentration (EC50) in cytotoxicity (22Rv1 and LNCap cell lines) and cytokine induction assay with peripheral blood mononuclear cells (PBMCs) cocultured with 22Rv1 cells (IL-6, tumor necrosis factor [TNF] and interferon-gamma [IFNγ-]) assays. In addition, this dose was supported by the 4-week Good Laboratory Practice (GLP) toxicology study.

For these calculations, MABEL was defined as the EC50 of the most sensitive and relevant measure of pharmacological activity, T-cell cytotoxicity assays. PBMCs from multiple donors were cultured with PC cell lines (22Rv1 and LNCap) and found to have an average EC50 of ˜258 ng/mL and 57 ng/mL, respectively. The human volume of distribution (Vd) of 4640 mL was predicted based on the allometric scaling of cynomolgus monkey Vd of 116 mL (from PK/PD study) using 1 coefficient. The average 22Rv1 EC50 of 258 ng/mL is expected to correspond to approximately a maximum concentration (Cmax) from a 1.2 mg dose in an 80 kg human (˜1197 μg=4640 mL human predicted Vd×˜258 ng/mL). The average LNCap EC50 of 57 ng/mL is expected to correspond to approximately a Cmax from a 0.264 mg dose in an 80 kg human. In addition to using the most sensitive and relevant measure of pharmacological activity, T-cell cytotoxicity assays, cytokine release was assessed as well via in vitro cytokine induction assay. The starting dose based on cytokine induction (IL-6, IFN-γ and TNF) suggests a starting dose based on IL-6 EC50 of 324 ng/mL of 1.5 mg, based on IFN-γ EC50 of 609.9 ng/mL of 2.8 mg and based on TNF-α EC50 of 756.2 ng/mL of 3.5 mg.

In addition, the above approach starting dose is also supported as being more cautious than standard highest non-severely toxic dose (HNSTD)/no observed adverse effect level (NOAEL) approaches that can be used for protein therapeutics being used for treatment of late-stage cancer patients in accordance with International Council for Harmonisation of Technical Requirements for Pharmaceuticals for Human Use (ICH) S9. The safety profile of PC-1 was investigated in 4-week GLP toxicology studies performed in cynomolgus monkeys. Dose levels of 0, 0.1, 0.3, and 1.5 mg/kg, dosed weekly for 5 treatment doses were evaluated. The HNSTD/NOAEL was considered to be 1.5 mg/kg, the highest dose tested. The human equivalent dose based on the nearly identical binding affinity between human and cynomolgus monkey and the NOAEL/HNSTD in cynomolgus monkey is approximately 1.5 mg/kg. Based on the standard criteria used for determining the starting dose of an anticancer drug following ICH S9 guidance, the maximum permitted starting dose in cancer patients is ⅙^{th}×1.5 mg/kg, or 0.25 mg/kg or 20 mg administered weekly. However, this dose level is at the high end of the range anticipated for antitumor efficacy based on the in vitro cytotoxicity assays and studies as was considered too aggressive as a starting dose for PC-1.

The proposed starting dose of 100 ug is 12 times lower than the dose calculated based on cytotoxicity 22Rv1 assay, 2.6 times lower than the dose calculated based on cytotoxicity LNCap assay, in addition, it is 15-35 times lower than the dose calculated based on cytokine induction assay (IL-6, IFN-γ and TNF) and also approximately 200 times lower than the dose supported by GLP toxicology study. To be able to fully characterize the safety, PK, and PD, a starting dose of 100 ug is administered IV every 7 days.

### PC-1 Clinical Experience

Subjects were enrolled in this PSMA-007-001 study. Some subjects were treated with a flat dose of 100 ug and some subjects were treated with a flat dose of 300 ug. Based on the observance of CRS with flat doses of PC-1, step dosing was implemented, and some subjects were subsequently enrolled on study in a third cohort with planned step doses of 50 ug on Day 1, 100 ug on Day 8 and 300 ug on Day 15.

Across all dose levels no DLTs were observed. In the flat dose cohorts and the first step dose cohort, 8 subjects reported at least 1 treatment-emergent AE (TEAE). The most frequently reported (≥2 subjects) TEAEs were CRS (6 subjects [75%]) and fatigue (3 subjects [38%]). The most frequently reported treatment-related AEs (≥2 subjects) are CRS (6 subjects [75%]) and pyrexia (2 subjects [25%]).

Refer to the PC-1 IB for additional safety information.

### Potential Risks and Benefits

The identified risks of treatment with TCEs are primarily related to cytokine release and CRS. PC-1 is designed to reduce the risk of CRS by requiring protease processing for activation, focusing molecular activity to the TME where proteases are overexpressed, dysregulated, and activated. As described in the IB, this has been shown to markedly reduce systemic cytokine exposure in preclinical models.

Data from other clinical development programs (of non-masked TCE) has shown that broad systemic T-cell activation can lead to cytokine release into the blood, which can cause acute effects of fever, hypotension, and hypoxia and may require care in the hospital. These side effects are short-lived and can be treated with IV fluids, steroids, tocilizumab, and supportive care, but occasionally have the potential to be severe. These vital signs and adverse effects will be carefully monitored for their appearance and followed closely in this clinical study.

This study is intended to evaluate the safety, tolerability, PK, PD, and preliminary efficacy of PC-1. Potential benefits for qualifying subjects with mCRPC include the opportunity to receive an experimental treatment that may increase the probability of clinical response and extend the duration of response (DOR), survival, and/or symptomatic improvement.

### Objectives and Endpoints

TABLE 10
Objectives and Endpoints

| | |
|---|---|
| Objectives Endpoints | Objectives Endpoints |

Primary

| | |
|---|---|
| To assess safety and tolerability in | Incidence of AEs, SAEs, and DLTs |
| subjects with mCRPC | according to NCI CTCAE v5.0 |
| To determine the MTD-R or MAD | CRS will be graded per ASTCT criteria |
| To assess potential Phase 2 dose | (Lee et al 2019) |
| regimens and determine a RP2D-R | |

Secondary

| | |
|---|---|
| To evaluate the PK profile of PC-1 | PK: AUC0-t, AUC0-inf, CL, Cmax, |
| following IV administration | Tmax, Clast, Tlast, t½, and Vd of |
| To evaluate the immunogenicity of | PC-1 |
| PC-1 | Incidence and persistence of ADAs |
| To assess preliminary efficacy of PC-1 | against PC-1 and impact on the PK |
| | profile of PC-1 |
| | PSA response rate as determined by |
| | Investigators according to Prostate |
| | Cancer Working Group 3 (PCWG3) |
| | ORR, DOR, and rPFS, as determined |
| | by Investigators according to Response |
| | Evaluation Criteria in Solid Tumors |
| | (RECIST) 1.1 and PCWG3 |
| | OS |

Exploratory

| | |
|---|---|
| To evaluate the PD profile of PC-1 | Baseline and on-treatment cytokine and |
| following IV administration | chemokine levels, immune cell |
| To characterize the impact of PC-1 on | characterization |
| systemic immune factors, including | and activation profile |
| cytokines and immune cells | Baseline PSMA expression level (PSMA- |
| To assess response by novel methods, | PET) |
| PSMA-PET evaluation, CTC, and | and response assessment |
| ctDNA quantitation | Baseline and on treatment CTC and |
| To assess baseline and on-treatment | ctDNA |
| tumor and immune biomarkers, and | quantitation |
| evaluate for correlations with anti- | In the subset of subjects with biopsies, |
| tumor activity and safety | evaluation of tumor biology, TME |
| To assess additional preliminary | characteristics, and T-cell infiltration |
| efficacy of PC-1 | PFS as determined by Investigators |
| | according |
| | to RECIST 1.1 |

ADA = anti-drug antibody;
AE = adverse event;
ASTCT = American Society for Transplantation and Cellular Therapy;
AUC0_{-inf} = area under the concentration-time curve extrapolated to infinity;
AUC0₋ₜ = area under the concentration-time curve from time 0 to time t;
CL = clearance;
Clast = last measurable concentration;
Cmax = maximum concentration;
CRS = cytokine release syndrome;
CTC = circulating tumor cell;
ctDNA = circulating tumor DNA;
DLT = dose -limiting toxicity;
DOR = duration of response;
IV = intravenous;
MAD = maximally administered dose;
mCRPC = metastatic castration -resistant prostate cancer;
MTDR = maximum tolerated dose regimen;
NCI CTCAE v5.0 = National Cancer Institute Common Terminology Criteria for Adverse Events Version 5.0;
ORR = overall response rate;
OS = overall survival;
PCWG3 = Prostate Cancer Clinical Trials Working Group 3;
PD = pharmacodynamics;
PET = positron emission tomography;
PFS = progression-free survival;
PK = pharmacokinetics;
PSMA = prostate -specific membrane antigen;
PSA = prostate-specific antigen;
RECIST = Response Evaluation Criteria in Solid Tumors;
RP2D-R = recommended Phase 2 dose regimen;
rPFS = radiographic progression free survival;
SAE = serious adverse event;
Tlast = time of last measurable concentration;
Tmax = time of maximum concentration;
TME = tumor microenvironment;
t½ = terminal half -life;
Vd = volume of distribution

### Study Design

### Overall Study Design

This study is an FIH, Phase 1, open-label, multicenter study to assess the safety, tolerability, PK, PD, and preliminary efficacy of PC-1 administered as a single agent in adult subjects with mCRPC. An outline of the study is shown in FIG. 2.

The study will be conducted in 3 parts: Dose Escalation (Part 1) with approximately

20 to 30 subjects, Cohort Backfill (Part 2) with up to approximately 45 subjects, and Dose Expansion (Part 3) with up to approximately 30 subjects enrolled at the RP2D-R.

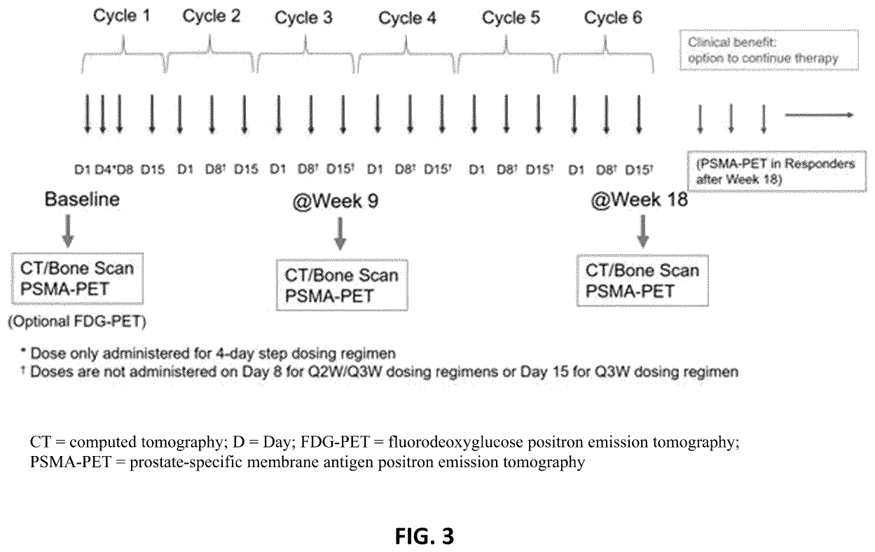

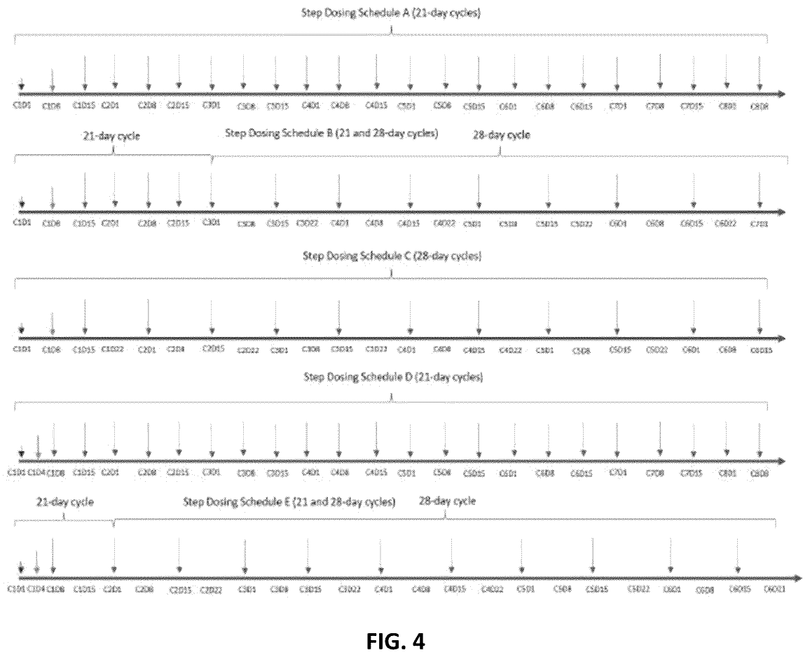

PC-1 will be administered IV weekly on Days 1, 8, and 15 of 21-day cycles. Alternate dose schedules [e.g. 4-day step dosing (Days 1, 4 and 8 in the first week of Cycle 1), every 2 weeks (Q2W), every 3 weeks (Q3W), etc.] may also be evaluated. Example step dosing schedules are shown in FIG. 4. Cycle 1 may be longer than 21 days if a ≥3-step regimen or Q2W regimen is evaluated. All subjects in Part 1 will require hospitalization to monitor for CRS as outlined in Section 6.3. Tumor assessments will include PSA levels, CT, and bone scan imaging at baseline, Week 9, Week 18 and then every 3 months. PSMA-PET will be obtained at baseline, Week 9, Week 18 and after the determination of response by conventional imaging if response is first observed after Week 18. Fluorodeoxyglucose-positron emission tomography (FDG-PET) for baseline assessment is optional. The dosing schema is depicted in FIG. 3.

Subjects exhibiting acceptable safety and stable disease (or are deemed clinically benefitting) may continue to receive PC-1 until disease progression or unacceptable toxicity.

Dose Escalation (Part 1) will assess the safety, tolerability, PK, PD, and preliminary efficacy of PC-1 administered by IV infusion. The starting dose of 100 μg is based on the anticipated MABEL in humans. Example PC-1 dose regimens are shown in Table 14 and Table 15. Part 1 of the study will use an accelerated titration design and start with a single-subject cohort at the 100 μg dose level. This design was chosen to potentially reduce the number of subjects treated with doses likely to be below a minimally effective threshold. Dose escalation will utilize a standard 3+3 scheme starting at the 300 μg dose level. Beginning at Cohort 6, dose escalation will be guided by the Bayesian Logistic Regression Model (BLRM) with overdose control (EWOC). Cohort management is under the supervision and direction of the Safety Review Committee (SRC). The SRC must clear a cohort prior to the initiation of new higher dose cohorts.

TABLE 11
Example PC-1 Flat Dose Escalation Regimens

| Dose Level¹ | Dose | | Incremental Dose Level Change | N |
|---|---|---|---|---|
| Dose Level −2 | 25 | μg² | — | (1+) |
| Dose Level −1 | 50 | μg | — | (1+) |
| Dose Level 1 | 100 | μg | — | (1+) |
| Dose Level 2 | 300 | μg | 3x | 3 + 3 |
| Dose Level 3 | 1 | mg | 3.3x | 3 − 6 |
| Dose Level 4 | 2.5 | mg | 2.5x | 3 − 6 |
| Dose Level 5 | 5 | mg | 2x | 3 − 6 |
| Dose Level 6 | 10 | mg | 2x | 3 − 6 |

¹Additional dose levels, or intermediate dose levels, may be explored if appropriate based on emerging safety, PK or PD data.
²Dose Level −2 if needed

If the highest dose regimen listed in Table 14 or Table 15 is well tolerated, and review of safety, PK, PD and biomarker data support for potential superior clinical benefit over the risk, additional dose regimen escalation cohorts may be projected after revising BLRM operating characteristics.

### Step Dose Escalation Approach

Bispecific T-cell engaging antibodies often have first dose effects, whereas toxicities on repeat dosing may be much diminished. This has resulted in most clinical TCE programs utilizing a step dosing approach. Some programs utilize a simple 2-step approach, while others may utilize a progressive multi-step approach. Individual subjects may present with heterogenous T-cell function, number, and states at baseline. Approaches that allow for lower initial doses followed by higher doses may enable the safest and most tolerable therapeutic profile for PC-1 administration while optimizing clinical benefit. Based on the above rationale, the SRC may also recommend additional dosing regimens to be explored in parallel, including a simple 2-step, multi-step or 4-day step regimen. The intent is to identify a well-tolerated first dose and also a well-tolerated subsequent and final dose. Alternative dosing cohorts will be designed with SRC oversight. For any alternative dosing regimen, the starting dose (first step) will not exceed the highest cleared first dose in a cohort demonstrated to not exceed the MTD. Examples of 2-step dosing schemes are specified in Table 5. An alternative multi-step dosing scheme may be evaluated to slow or enhance the dose escalation and may include 3 steps or higher. Exploration of a step dosing scheme, or related alternatives, will enable a safer dose escalation of PC-1 and identification of a more promising RP2D-R.

### Step Dose Initiation

The step dose strategy has been shown to lower the risk of CRS associated with the initiation of T cell engager therapy (Ball 2023). Dexamethasone or equivalent premedication will be used prior to each of the step doses as detailed in (Section 6.1.2.2).

Based on emerging safety data, the SRC may initiate a step dose approach to mitigate CRS. A new cohort will be initiated where a lower dose will be chosen as the initial dose (a previously cleared dose level) before a step up to the dosing level where CRS was seen. A 2-step, multiple step or 4-day step regimen may be evaluated during Part 1 where the step and target dose to be investigated will be determined by the SRC. Examples of step and target doses that may be investigated are listed in Table 5. Alternative and intermediate doses may also be considered based on emerging data.

TABLE 12
Example PC-1 2-Step Dose Escalation Regimens

| | | | |
|---|---|---|---|
| Dose Levels¹ | Step 1 | Step 2 | Target Dose |

| | | | | | | |
|---|---|---|---|---|---|---|
| Dose Level 1 | 50 | μg | 100 | μg | 300 | μg |
| Dose Level 2 | 100 | μg | 300 | μg | 450 | μg |
| Dose Level 3 | 100 | μg | 300 | μg | 1 | mg |
| Dose Level 4 | 200 | μg | 600 | μg | 2 | mg |
| Dose Level 5 | 300 | μg | 1.2 | mg | 4 | mg |

¹Additional dose regimens, or intermediate dose regimens, may be explored if appropriate based on emerging safety, PK or PD data.

Dose Escalation (Single-Subject, Cohort; 100 μg dose)

In the single-subject cohort, an initial subject will receive 100 μg IV on Cycle 1 Day 1 and be hospitalized for observation for at least 24-48 hours. If the subject tolerates the first dose without unacceptable toxicity, the subject may receive subsequent infusions at weekly intervals as an outpatient. After the subject is observed through the completion of the DLT window (Cycle 1, 21 days), data will be reviewed by the SRC to determine if the current dose level is tolerable and if dose escalation may proceed. Once Cohort 1 is cleared, subsequent subjects would then be cleared to receive the next highest dose (see Table 14 and Section 3.2.3). If a safety signal is observed at the initial dose, an additional 2 subjects will be enrolled, and the protocol will convert to a standard 3+3 design. If no safety signal is observed, the protocol will automatically convert to a standard 3+3 design and progress to Dose Level 2 (300 μg). If Grade>2 CRS is observed, a step dose approach may also be employed to mitigate CRS (see Section 3.2.1 for details).

The single-subject cohort will convert to the standard 3+3 design if one of the following occurs:
A DLT occurs following PC-1 administration.
   The single subject experiences Grade≥2 adverse event (AE) (regardless of attribution) during the DLT window.
   Review of safety data by the SRC identifies a safety concern (e.g., subclinical increases in safety laboratory assessments or inflammatory cytokines).
   Grade>2 CRS.

### Dose Escalation

The purpose of the Part 1 dose escalation is to identify the MTD-R of single agent PC-1. Toxicities after completion of the DLT observation period will be monitored as late-onset toxicities. The addition of step doses will be investigated to further mitigate CRS. The study was initiated using a standard 3+3 design. Beginning at Cohort 6, the study will switch from 3+3 to BLRM with EWOC. This switch is necessary as BLRM with EWOC is more suited to supporting the recommendations of the SRC when the space of possible subsequent doses is more complex; which is the case here due to the potential introduction of step dosing to mitigate CRS. A 3-parameter-step BLRM will be used to model the relationship between the dose of PC-1 and the overall probability of a DLT. The 3-step approach includes 2 simple 2-parameter BRLMs, one of which models the relationship between PC-1 dose and the probability of DLT due to CRS (p(CRS)), and the other of which models the relationship between PC-1 dose and the probability of DLT not due to CRS (p(non-CRS)). These probabilities are then combined to estimate the overall probability of DLT. Using DLT data at all tested dose levels and pre-specified prior distributions of model parameters, posterior probabilities of having a DLT falling into 3 dosing intervals (underdosing, targeted toxicity, overdosing) will be calculated for all dose levels/dose regimens. A dose may only be used for newly enrolled subjects in Part 1 if the risk of overdosing (i.e., probability of true DLT rate higher than 0.33 at that dose) is less than 25% (EWOC). The use of the EWOC principle limits the risk that potential next doses will exceed the MTD-R/MAD.

Dose escalation will proceed and stop according to the probabilistic decision rules set by the BLRM (Section 11.4.2).

### 3.2.3.1 Dose Escalation Rules

Dose escalation will be determined based on SRC review of all safety data according to the following rules:

### For All Cohorts

The dose increment for all cohorts will be no larger than half-log increment under BLRM with EWOC. The DLT observation period will be 21 days following the Cycle 1 Day 1 dose. For step dosing cohorts, an extension of the DLT assessment period will be implemented to maintain 7-day observation after the first target dose level administration if more than 2-step doses are investigated. Subjects who do not complete the DLT assessment period or are not considered DLT evaluable as defined in Section 3.2.4.1 may be replaced for the DLT evaluation.

### For Flat Dose Cohorts

If a subject experiences 1 DLT in a given dose cohort during the DLT period, dose escalation for the next cohort will not exceed more than 50% increment interval.
For any Grade 3 CRS not meeting DLT criteria, dose escalation for the next cohort will not exceed more than 100% increment interval.
If ≥2 subjects develop treatment-related Grade 2 AEs, dose escalation will not exceed more than 50% increment interval.
If a subject experiences two independent DLTs during the DLT period, limit the maximum dose increment to 30%.
For investigations of the flat dose schedule, treatment will be staggered such that the second subject will receive PC-1 treatment at least 3 days after the first subject receives the first dose of PC-1 to assess for any acute toxicities. Subsequent subjects (e.g., between the second and third subject) enrolled will be staggered by at least 1 day.

Planned dose levels to be evaluated with flat dosing are listed in Table 14.

### For Step Dose Cohorts

If a subject experiences Grade≥2 CRS during the DLT assessment period, dose escalation for the next cohort will not exceed more than 100% for the CRS-associated dose.
If >50% of subjects experience Grade≥2 CRS events during the DLT assessment period, dose escalation for the next cohort will not exceed more than 50% for the CRS-associated dose.
If a subject experiences 1 DLT in a given dose cohort during the DLT assessment period, dose escalation for the next cohort will not exceed more than 50% for all doses.
If a subject experiences two independent DLTs in a given dose cohort during the DLT assessment period, dose escalation for the next cohort will not exceed more than 30% for all doses.

For investigations of the weekly step dose schedule, treatment will be staggered such that the second subject will receive PC-1 treatment at least 3 days after the first subject receives the first dose of PC-1 to assess for any acute toxicities. For investigations of the 4-day step dose schedule, treatment in the first cohort will be staggered such that the second subject will receive PC-1 treatment at least 7 days after the first subject receives the first dose to assess for any acute toxicities. For subsequent cohorts, the first and second subjects treated will be staggered by at least 3 days. Subsequent subjects (e.g., between the second and third subject) enrolled in all cohorts will be staggered by at least 1 day.

Examples of potential step dosing regimens are outlined in Table 15.

### Maximum Tolerated Dose Regimen

The MTD-R will be defined as the highest dose of PC-1 in which the posterior probability of the true DLT rate in the target interval (0.16-0.33) of the MTD-R is above 0.50, the probability of overdose is <0.25, and 6 subjects have been treated at the confirmatory cohort. When step dosing is implemented, the study will aim to establish the MTD-R of a dosing regimen that includes step dose(s) and an independent MTD-R of a regimen that does not include step dosing in parallel.

### 3.2.3.3 Maximally Administered Dose

If the MTD-R is not defined during dose escalation, the highest dose administered in this study will be declared the maximally administered dose (MAD).

### 3.2.3.4 Dose Escalation Decisions

Dose escalation decisions will be made based on the recommendations of the SRC. For each cohort, dose escalation decisions will be made based on the review of safety data and available PK, PD, and preliminary efficacy data. On the basis of a review of real-time safety data and available PK, PD, and preliminary efficacy data, dose escalation may be halted or modified by the SRC as deemed appropriate.

### 3.2.4 Dose-Limiting Toxicities

Subjects in Dose Escalation (Part 1) will be assessed for DLTs. Assessment of DLTs for each dose regimen will be evaluated by the SRC prior to enrolling subjects in the next dose regimen. All AEs, including DLTs, will be reported according to Section 10 and graded according to National Cancer Institute Common Terminology Criteria for Adverse Events version 5.0 (NCI CTCAE v5.0), except for CRS where American Society for Transplantation and Cellular Therapy (ASTCT) grading will be used (Lee et al 2019). DLTs will be treated according to clinical practice and monitored through resolution.

For dose escalation purposes, the DLT assessment period is defined to be 21 days from the Cycle 1 Day 1 dose. For step dosing cohorts (Section 3.2.1), an extension of the DLT assessment period will be implemented to maintain 7-day observation after the first target dose level administration if more than 2-step doses are investigated.

### 3.2.4.1 Definition of Dose-Limiting Toxicity Evaluable Subjects

Determination of whether a subject is evaluable for DLT assessment will be as follows:
Subjects must have completed safety assessments during the DLT assessment period
Subjects who experience a DLT after receiving any dose of PC-1
For flat dosing, subjects without a DLT must receive at least 66% of intended dose of the study drug during Cycle 1
For step dosing, subjects without a DLT must have received all planned step doses and the target dose in Cycle 1.

### 3.2.4.2 Definition of Dose-Limiting Toxicity

A DLT will be defined as any of the following AEs occurring during the DLT assessment period during Dose Escalation (Part 1) (not including Cohort Backfill [Part 2] subjects). All study drug-related AEs regardless of attribution will be included unless they are clearly and directly related to underlying disease or clearly unrelated random or accidental events.

### Non-Hematologic DLT:

1. Grade 4 or 5 AE unless due to disease progression or non-study related causes
2. Grade≥3 infusion related reaction
3. Recurrent Grade 3 infusion related reaction despite adequate glucocorticoid pre-medication
4. Dose delay>2 weeks due to an AE
5. Grade≥3 non-hematologic toxicities
6. Grade 4 AST/ALT elevations without CRS, or Grade 3 AST/ALT elevations without CRS lasting longer than 72 hours
7. Liver function test abnormalities that meet the criteria for Hy's Law
   Concomitant elevations in aspartate aminotransferase (AST)/alanine aminotransferase (ALT) and bilirubin consistent with confirmed drug induced-liver injury meeting Hy's law criteria:
      AST/ALT>3× upper limit of normal (ULN); AND
      Total bilirubin>2×ULN; AND
      Alkaline phosphatase<2×ULN; AND
      No other reason for liver injury

### Hematologic DLT:

1. Grade 4 neutropenia (absolute neutrophil count [ANC]<0.5×10⁹ cells/L) lasting>3 days (growth factor support is allowed)
2. Febrile neutropenia (defined as ANC<1.0×10⁹ cells/L with a single temperature of >38.3° C. or 101° F., or a sustained temperature of ≥38° C. or 100.4° F. for >1 hour)
3. Grade 4 thrombocytopenia lasting>3 days
4. Grade≥3 thrombocytopenia with clinically significant bleeding

### Exceptions Include the Following:

Isolated Grade 3 laboratory abnormalities not associated with clinical sequelae and are corrected with appropriate management within 72 hours
Grade 3 amylase or lipase that is not associated with symptoms or clinical manifestations of pancreatitis
Grade 3 nausea/vomiting or diarrhea<72 hours with adequate anti-emetic and other supportive care
Grade 3 fatigue lasting<1 week
Grade 3 tumor pain that can be adequately managed with supportive care and/or lasts<1 week
Grade 3 bone pain due to T-cell expansion in marrow compartments lasting<1 week
Any grade lymphopenia not associated with clinical sequalae

### Dosing Duration

Subjects exhibiting acceptable safety and SD or better may continue to receive PC-1 until disease progression, unacceptable toxicity or no longer clinically benefiting (NLCB). Subjects who discontinue study treatment without disease progression will continue to be monitored, including regularly scheduled tumor assessments as outlined in Section 7.3, until discontinuation from the post-treatment follow-up.

### 3.2.4.3.1 Treatment Beyond Dose-Limiting Toxicity

For subjects who experience a DLT during the DLT assessment period (for first and only DLT), but in the assessment of the treating physician are not expected to have continued toxicity with additional administration of PC-1 (e.g., tumor lysis syndrome [TLS], CRS), continued study treatment may be considered (after recovery), provided they meet the criteria specified in Section 6.5 and with Medical Monitor approval. The dose may be escalated after confirmation of safety and tolerability of redosing. All other study treatment-related AEs from prior PC-1 administration must have decreased to Grade≤1 or the baseline grade by the start of the next PC-1 administration.

Exceptions on the basis of ongoing overall clinical benefit may be allowed after a careful assessment and discussion of the benefit-risk with the subject by the Investigator and with approval from the Medical Monitor.

### 3.2.5 Intra-Subject Dose Escalation

After a subject has completed 3 cycles with a specific dosing regimen, which may include escalating dose levels in step dosing, intra-subject dose escalation may be permitted for subjects in Dose Escalation (Part 1) who have been safely dosed. Step dosing and intra-subject dose escalation are independent. This is primarily intended for initial subjects who receive low doses of PC-1. This will offer them the chance for benefit with a higher already cleared dose. Subjects will be able to undergo intra-subject dose escalation of target dose only after completing at least 3 cycles at their originally assigned dose regimen (and their 9-week response and imaging assessments). Subjects must not have had a treatment-related Grade 3 AE considered at risk for recurrence upon escalation of dose per Investigator judgment. Prior incidence of CRS is allowed if subsequent infusions indicated tolerance. Subjects may then receive the next highest target dose, but only after that dose regimen cohort has been completed and cleared by the SRC. Following the first administration of the new target dose, the blood sampling collection will include vital signs, safety labs, PK and PD evaluations as outlined in the Schedule of Assessment for Cycle 1 Day 1 for step dosing. After the first infusion at the new target dose, the sampling requirements for the corresponding study visit for that subject should be followed. Decisions on intra-subject dose escalation should be made with the approval of the Medical Monitor in consultation with the Investigator.

### 3.3 Cohort Backfill (Part 2)

The study may allow for limited expansion of a prior cleared dose level cohort in Part 1 (up to 15 subjects per cohort, 18 subjects per dose level in Parts 1 and 2 combined). These backfill cohorts, will allow further characterization of safety and efficacy prior to initiation of the formal dose expansion cohort (Part 3).

Alternative dosing schedules (i.e., Q2W or Q3W) may also be investigated in the backfill cohorts. This will be performed to evaluate whether less frequent dosing reduces the T cell exhaustion associated with T cell engager administration, and whether this may impact efficacy. The SRC will decide the specific dosing schedules to investigate based on emerging data.

The backfill cohorts may open concurrently with ongoing dose escalation. The dose for the backfill cohorts will be selected based on SRC review of cumulative safety, PK, PD, and preliminary efficacy data. Data from backfill expansion cohorts will further support RP2D-R selection per review by the SRC.

### 3.4 Dose Expansion (Part 3)

Up to approximately 30 subjects will be enrolled to obtain additional safety, tolerability, PK, PD, and preliminary clinical activity data with PC-1 at a dose regimen to be determined by the SRC after reviewing all available safety, PK, PD, and preliminary efficacy data. At no time will the PC-1 dose regimen studied in Dose Expansion (Part 3) exceed the highest dose regimen that qualifies as an MTD-R in Dose Escalation (Part 1). The dose regimen for the expansion cohort will be selected based on SRC review of cumulative safety, PK, PD, and preliminary efficacy data. Data from the expansion cohort will further support RP2D-R selection per review by the SRC. A randomized cohort with two preliminary RP2D-Rs in Part 3 may be considered based on results from Part 2.

### 3.5 Dosing Interval

### The Terminal Half-Life of PC-1 in Cynomolgus Monkey at Relatively High Doses

(0.1-1.5 mg/kg) has been determined as 110 hours. However, the half-life and PK parameters of PC-1 in humans remains to be elucidated. Species differences in albumin half-life (longer in humans), and because the monkey models lack tumor, which express abundant target PSMA and TRACTr activating proteases, altered PK parameters may be seen in this study. In addition, the PD and immune effects are yet to be determined.

During the dose escalation phase of the study, evolving PK data may indicate a less frequent dosing interval is more appropriate during therapy. When the intact PC-1 enters the TME, cleavage by proteases and formation of the active TCE species is expected. If the amount of TCE formed in the TME is sufficient, it is expected to drive anti-tumor activity. However, if TCE escapes the TME and sufficiently persists in the blood compartment it is expected to drive pharmacology outside the TME. At lower dose levels of PC-1, the active TCE formed in the TME that then escapes into the blood compartment is likely to be below pharmacologically active concentrations. As the PC-1 dosages are increased, it is expected to increase exposures of the active TCE that can escape the TME and enter the blood compartment. If at the higher dose levels of PC-1 the exposure of the active TCE persists through the dosing interval at concentrations approaching those expected to be pharmacologically active, a less frequent dosing regimen may be investigated to avoid potential accumulation of the active TCE in the blood compartment. Additionally, longer intervals between infusions, may allow for T cells to rest prior to repeated activation and prevent T-cell exhaustion (Li et al, 2019, Philipp et al 2022), leading to better clinical responses. Subjects will be monitored for maintenance of clinical activity and T cell functionality.

Based on evolving PK, PD and safety data, the SRC may recommend evaluation of a less frequent dosing schedule (i.e., Q2W or Q3W). In addition, some subjects may experience difficulties visiting the site for weekly infusions and wish to discontinue treatment. After >18 weeks of treatment, a longer treatment interval (i.e., Q2W or Q3W) may be provided, unless such a subject is NLCB, after agreement between the Investigator and Medical Monitor.

### 3.6 Pseudoprogression

Experience with cancer immunotherapy and CD3-engaging therapies has demonstrated that responding tumors may initially increase in size due to the influx of immune cells, a phenomenon referred to as pseudoprogression (Ma 2019). Treatment with PC-1 may initially increase tumor size and metabolic activity by inducing the influx of T cells into the tumor. If the Investigator believes that a subject is deriving clinical benefit despite radiographic evidence of progressive disease (PD), that subject may continue study treatment until a confirmatory imaging scan can be obtained at a further 6 to 9 weeks (Scher 2016). Subjects will be consented to continuing treatment beyond initial diagnosis of radiologic disease progression. All decisions to continue treatment beyond progression must be discussed with the Medical Monitor, and an assessment of the benefit-risk of continuing with study treatment must be documented in the study records. Subjects continuing study treatment despite apparent radiographic progression will be strongly encouraged to undergo a repeat tumor biopsy to assess whether increases in the tumor volume are due to immune-cell infiltration or neoplastic proliferation, provided that such a biopsy can be performed safely. If true neoplastic progression is suspected based on the Investigator's judgement, clinical factors or biopsy findings, or if radiographic progression is confirmed at a subsequent tumor assessment, the subject will be ineligible to receive further study treatment.

### 3.7 Recommended Phase 2 Dose Regimen

All available PK/PD data (including changes in cytokines), safety and efficacy in the ongoing study will be used to set the appropriate RP2D-R. The RP2D-R for PC-1 as monotherapy is the dose regimen chosen for further investigation in Phase 2. The RP2D-R will be selected based on all available clinical data obtained from Part 1 through Part 3 of this study; all safety, efficacy, clinical PK and PD data will be aggregated and analyzed. The preliminary RP2D-R of PC-1 for Part 3 monotherapy expansion will be less than or equal to the MTD-R/MAD identified in Part 1. Pharmacologically optimized dose/doses may be included in Part 3 based on results from Part 2.

### 3.8 Study Oversight—Safety Review Committee

An SRC will oversee the conduct of the clinical study. The SRC will review cumulative safety data, as well as available PK, PD, and preliminary efficacy data, and make recommendations regarding dose escalation and overall study conduct on the basis of study data to ensure subject safety while receiving study treatment. These include recommendations to suspend subject enrollment in a given dose regimen or add subjects to further evaluate a given dose regimen in backfill cohorts. In addition, the SRC, based on the overall benefit-risk profile of PC-1 as a single agent during dose escalation, may make recommendations to change administration dose, schedule, or premedications required to ensure subject safety. The SRC will also review cumulative safety data to identify safety concerns that may emerge due to cumulative exposure beyond the DLT assessment period. The SRC will meet to evaluate clearance of all cohorts through dose escalation and at regular intervals through dose expansion.

### 3.9 Participating Sites

During Dose Escalation (Part 1), approximately 8 to 10 sites globally will be initially opened. During Cohort Backfill (Part 2), approximately 12 to 20 sites will be opened. During Dose Expansion (Part 3), up to approximately 20 sites will be opened. During the study, additional participating sites and countries may be added.

### 3.10 Number of Subjects

It is anticipated that approximately 105 subjects will be enrolled into this study as follows:
Dose Escalation (Part 1): Approximately 20 to 30 subjects
Cohort Backfill (Part 2): Up to approximately 45 subjects
Dose Expansion (Part 3): Up to approximately 30 subjects

### 3.11 Replacement of Subjects

In Dose Escalation (Part 1), subjects who do not complete the DLT assessment period or are not DLT evaluable per Section 3.2.4.1 may be replaced for the DLT evaluation.

In Cohort Backfill (Part 2) and Dose Expansion (Part 3), subjects who do not have at least 1 post-baseline radiographic tumor evaluation for reasons other than treatment discontinuation due to toxicity or death due to disease progression may be considered not evaluable for efficacy and may be replaced.

### 3.12 Estimated Study Duration and Study Completion

### 3.12.1 Study Duration

The planned duration of the study is approximately 4 years.

### 3.12.2 Study Duration for Individual Subjects

Subject participation will include screening, treatment, and follow-up. Screening will be conducted up to 28 days before the first dose of PC-1, during which the subject's eligibility and baseline characteristics will be determined. For subjects exhibiting acceptable safety and SD (or are deemed clinically benefitting), treatment with PC-1 may be continued until disease progression or unacceptable toxicity. Post-treatment, subjects will be followed for survival until death, withdrawal of consent, or the end of the study, whichever occurs first. Subjects who discontinue treatment prior to documented disease progression should be followed for disease progression until documented disease progression or initiation of a new anticancer treatment, whichever occurs first. The expected median duration of the study treatment for individual subjects is approximately 18 to 36 weeks.

### 3.12.3 Completion of the Study

The end of this study is defined as the date when the last subject's last visit occurs, or the date at which the last data point required for statistical analysis or protocol-defined safety monitoring is received from the last subject, whichever occurs later.

### 3.13 Suspension or Termination of the Study

The study can be suspended or terminated at any time by the Sponsor, the US FDA, ex-US regulatory authorities, at the recommendation of the SRC, or an Institutional Review Board (IRB)/Independent Ethics Committee (IEC). Circumstances that may warrant suspension or termination include, but are not limited to: Determination of unexpected, significant, or unacceptable risk to subjects. The study may resume after suspension once concerns about safety, protocol compliance, and data quality are addressed to the satisfaction of the Sponsor, IRB, and/or US FDA and other relevant health authorities.

### Subject Enrollment

All subjects must sign and date the IRB/IEC-approved ICF before initiating any study specific procedures or activities that are not part of a subject's routine care. After written informed consent has been obtained and all results from screening procedures are available, the study site will submit eligibility documentation to the Sponsor and obtain the Sponsor's approval prior to enrolling the subject. The Sponsor will provide the dose regimen assignment. See Section 7.1 and Section 8.1 for details.

### 4.1 Identification Number

Each subject who enters the screening period will receive a unique screening identification (ID) number. The screening ID number will remain the same for a subject in the event they re-screen as described in Section 7.1.2. Upon Sponsor approval, a separate subject ID number will be assigned through the electronic database. This number will be used to identify the subject throughout the study and must be used on all study documentation related to the subject. Furthermore, the subject ID number must remain constant throughout the entire clinical study; it must not be changed after enrollment.

### 4.2 Screening Log

Investigative sites will maintain a log of all screened subjects who were reviewed and evaluated for study participation. Information collected on the screening log should include limited information, such as the date of screening, the date the subject was enrolled, or the reason why the subject failed screening.

### Subject Eligibility

### 5.1 Inclusion Criteria

Each subject must meet the following criteria to be enrolled in this study:
1. Male subjects>18 years of age at the time of signing informed consent
2. Histologically or cytologically confirmed adenocarcinoma of the prostate
3. Serum testosterone<50 ng/dL (or <1.7 nmol/L) achieved by orchiectomy or ongoing luteinizing hormone-releasing hormone agonist/antagonist therapy within 28 days prior to the start of study drug
4. Documented progression defined in PCWG3 and/or RECIST 1.1 after treatment with at least 1 novel anti-androgen therapy (eg, abiraterone, enzalutamide, apalutamide, darolutamide, galeterone, orteronel, seviteronel, or equivalent) and have failed at least 1 taxane regimen, or who are medically unsuitable or have actively refused treatment with a taxane regimen
5. Progressive mCRPC or intolerance to the most recent therapy; Disease progression on the prior systemic regimen per PCWG3 criteria (Scher et al 2016) by one of the following:
   a. PSA progression as defined by PCWG3 criteria; PSA level>1 ng/ml that has increased on at least 2 successive occasions at least 1 week apart
   b. Appearance of >2 new lesions on bone scans, or
   c. Progressive visceral disease, or
   d. Progressive nodal disease; previously normal (<1.0 cm) lymph nodes must have grown by >5 mm in the short axis from baseline or nadir and be >1.0 cm in the short axis to be considered to have progressed
6. Collection of PSMA-PET imaging [⁶⁸Ga] or [¹⁸F]
7. For subjects previously treated with first-generation anti-androgens, discontinuation must have occurred>4 weeks (for flutamide or nilutamide) or >6 weeks (for bicalutamide) prior to the start of study drug, with no evidence of an anti-androgen withdrawal response (i.e., no decline in serum PSA)
8. For subjects previously treated with a second-generation anti-androgen (eg, enzalutamide or equivalent) or with abiraterone acetate, discontinuation must have occurred 14 days or 5 half-lives prior to the start of study drug
9. Eastern Cooperative Oncology Group (ECOG) performance status of 0 or 1
10. Adequate bone marrow function, including:
   a. ANC>1500/mm³ or >1.5×10⁹/L (ANC must be assessed at least 14 days from the last growth factor support)
   b. Hemoglobin>9 g/dL (hemoglobin must be assessed at least 7 days from the time of the prior transfusion)
   c. Platelets≥75,000/mm³ or ≥75×10⁹/L (platelet count must be assessed at least 7 days from the time of the prior transfusion)
11. Adequate renal function, including:
   a. Estimated creatinine clearance>45 mL/min as calculated using the Cockcroft Gault equation, or a method standard for the institution
12. Adequate liver function, including:
   a. Total serum bilirubin<1.5×ULN within normal limits unless the subject has documented Gilbert syndrome in which case the maximum total serum bilirubin should be <5 mg/dL
   b. AST and ALT<2.5×ULN (<5×ULN in case of liver metastasis)
   c. Serum albumin≥30 mg/mL
13. Adequate pulmonary function as evidenced by:
   a. Baseline resting oxygen saturation>92% on room air
14. Adequate cardiac function as evidenced by:
   a. Cardiac ejection fraction≥50%, by echocardiogram (ECHO) or multigated acquisition (MUGA)
   b. QTcF<480 ms
      Note: If the baseline uncorrected QT interval is >480 msec, this interval should be rate-corrected using the Fridericia method (QTcF) and the resulting QTcF should be used for decision making and reporting
15. Resolved acute effects of any prior therapy to baseline severity or CTCAE Grade<1 except for AEs not constituting a safety risk by Investigator judgement. Subjects receiving ongoing replacement hormone therapy for endocrine immune-related AEs without clinical symptoms will not be excluded.
16. Willing to use 2 highly effective methods of birth control (as defined in Section 5.2.1) during the treatment period, if the subject's partner is a female of childbearing potential. Female partners of childbearing potential should also use contraception
17. Willing to complete all scheduled visits and assessments
18. Able to read, understand and provide written informed consent

### Exclusion Criteria

Subjects who meet any of the following criteria will be excluded from this study:
1. Subjects whose tumors (either archival or fresh biopsy) exhibit significant neuroendocrine differentiation or small cell features by histopathology
2. Prior treatment with PSMA-targeted CAR-T cell therapy
3. Prior treatment with PSMA-CD3, PSMA-CD28, or other CD3-T cell engaging bispecific antibodies.
4. Prior solid organ transplant
5. Prior treatment with systemic immunotherapeutic agents, including anti-CTLA4, anti-PD-1, anti-PD-L1, or other checkpoint inhibiting therapeutic antibodies within 28 days prior to the start of study drug
6. Radiation therapy with expected marrow suppression within 28 days prior to the start of study drug. (Palliative radiation to a limited field is permitted up to 14 days prior to the start of study drug).
7. Treatment with any small molecule targeted therapy or chemotherapeutic agent (investigational or otherwise) within 5 half-lives (or 21 days, whichever is shorter) and has recovered from toxic effects prior to the start of study drug
8. Use of any herbal products that could decrease PSA levels (e.g., saw palmetto) within 28 days prior to the start of study drug. The subject must agree not to use such herbal products during study participation.
9. Subjects with a condition requiring systemic treatment with either corticosteroids (>10 mg daily prednisone or equivalents) or other immunosuppressive medications within 14 days prior to the start of study drug. Note: Inhaled or topical steroids are permitted.
10. Subjects with a history of CTCAE Grade≥3 immune-related AEs that were considered related to prior immune-oncology treatment
11. Active autoimmune disease requiring systemic therapy (exception[s]: subjects with vitiligo, resolved childhood atopic dermatitis, hypothyroidism, or hyperthyroidism that is clinically euthyroid at screening are allowed)
12. History of severe allergic or anaphylactic reactions to monoclonal antibodies (or recombinant antibody-related fusion proteins)
13. Known active clinically significant bacterial, viral (including COVID-19), fungal, mycobacterial, parasitic, or other infection (excluding fungal infections of nail beds) at study enrollment or any major episode of infection requiring treatment with IV antibiotics or hospitalization (relating to the completion of the course of antibiotics) within 28 days prior to the start of study drug. Subjects with elevated C-reactive protein (CRP) at screening should be discussed with the Medical Monitor and carefully evaluated to rule out active infection or autoimmune disease.
Positive serologic or polymerase chain reaction (PCR) test results for acute or chronic hepatitis B virus (HBV) infection
   a. Subjects whose HBV infection status cannot be determined by serologic test results must be negative for HBV by PCR to be eligible for study participation.
15. Acute or chronic hepatitis C virus (HCV) infection
   a. Subjects who are positive for HCV antibody must be negative for HCV by PCR to be eligible for study participation.
16. HIV infection
17. History of clinically significant cardiovascular disease such as:
   a. Significant congestive heart failure, such as New York Heart Association Class III or IV
   b. Unstable angina, myocardial infarction, cardiac angioplasty, or stenting within the last 6 months prior to screening
   c. Presence of an abnormal electrocardiogram (ECG) or history of rhythm disorder that is clinically significant in the Investigator's opinion, including uncontrolled atrial fibrillation/flutter or any unstable arrhythmias, second- or third-degree atrioventricular (AV) heart block (AV block treated with a pacemaker is allowed)
18. Uncontrolled hypertension, defined as systolic blood pressure >160 mmHg or diastolic blood pressure >100 mmHg which has been confirmed by 2 successive measurements despite optimal medical management
19. History of significant active pulmonary disease (e.g., obstructive pulmonary disease)
20. History of pulmonary embolism within 6 months, or symptomatic deep vein thrombosis (DVT) within 3 months of enrollment. Line-related/upper extremity DVT requires adequate treatment and clearing by ultrasound.
21. Subjects with a history of or active bleeding disorders who require chronic blood product support
22. Clinically active or chronic liver disease, including liver cirrhosis of Child-Pugh Class C
23. Subjects with new or progressive brain metastasis or leptomeningeal disease. Note: For previously treated brain metastases, subjects must have completed treatment for brain metastasis, and be neurologically stable off supraphysiologic doses of steroids, for at least 28 days prior to the start of study drug.
24. Current or past history of central nervous system (CNS) disease, such as stroke, epilepsy, CNS vasculitis, or neurodegenerative disease
   a. Subjects with a history of stroke who have not experienced a stroke or transient ischemic attack in the past 2 years and have no residual neurologic deficits as judged by the Investigator are allowed.
   b. Subjects with a history of epilepsy who have had no seizures in the past 2 years while not receiving any anti-epileptic medications are allowed in Dose Expansion (Part 3) only.
25. Subjects with untreated spinal cord compression. Subjects must be neurologically stable off steroids for at least 28 days prior to the start of study drug.
26. Second primary malignancy that has not been in remission for >3 years. Exceptions that do not require a 3-year remission: non-melanoma skin cancer, in situ carcinoma of any type, or non-muscle invasive urothelial carcinoma
27. Any medical condition or clinical laboratory abnormality likely to interfere with assessment of safety or efficacy of study treatment, or indicating the subject would unlikely have potential benefit, or complete the study
28. In the Investigator's judgment, the subject is unlikely to complete all protocol-required hospitalization, study visits, or procedures, including follow-up visits, or comply with the study requirements for participation
A superscan as seen in baseline bone scan

### Birth Control Methods

Subjects with female partners of childbearing potential must agree to use 2 forms of approved contraception (i.e., a highly effective method of hormonal/intrauterine contraception with a failure rate <10% per year and 1 additional barrier method) during and 2 months post last dose of study treatment according to the below list:

| Barrier Methods | Hormonal/Intrauterine Methods¹ |
|---|---|
| Male or female condom | Implants |
| with or without spermicide | Hormone shot or injection |
| Cap, diaphragm, or sponge each | Combined pill |
| with spermicide | Patch |
| | Copper T intrauterine device or |
| | levonorgestrel - releasing |

¹Highly effective (failure rate of <1% per year).

In addition, the following are approved forms of contraception:
Male sterilization with absence of sperm in the post vasectomy ejaculate
Female partner who meets the following criteria for non-childbearing potential:
   Have undergone a documented hysterectomy and/or bilateral oophorectomy
   Have medically confirmed ovarian failure; or
   Achieved postmenopausal status, defined as cessation of regular menses for at least 12 consecutive months with no alternative pathological or physiological cause

Subjects with a pregnant or breast-feeding partner should use barrier method contraception (condom plus spermicidal gel). All sexually active subjects must agree to prevent potential transfer of and exposure to drug through semen to their partners by using a condom consistently and correctly during and 2 months post last dose of study treatment.

### Study Treatment

### 6.1 PC-1

### 6.1.1 Formulation, Packaging, and Handling

The Investigational Medicinal Product, PC-1, will be supplied by the Sponsor. PC-1 is a recombinant trispecific biologic, which binds PSMA, CD3, and albumin. PC-1 is manufactured under current Good Manufacturing Practices. PC-1 drug product (DP) is a sterile, clear, colorless to slightly yellow, preservative-free frozen liquid vial intended for IV infusion. PC-1 is supplied in single-dose vials (2 mg/mL concentration), in a formulation of 10 mM Histidine, 8% (w/v) Sucrose, 0.01% (w/v) Polysorbate 20, at pH 6.3. For more detailed information on the formulation, packaging, and handling of PC-1, refer to the Pharmacy Manual and the PC-1 IB.

### 6.1.2 Dosage, Administration, and Compliance

Dosing independent of body weight or surface area will be used for PC-1. The dose of PC-1 for each subject will depend on their dose regimen cohort assignment as detailed in Section 3. Refer to the Pharmacy Manual for more detail regarding the formulation and administration of DP. Subjects must be able to receive PC-1 per the process outlined in the Pharmacy Manual to be considered eligible for inclusion. PC-1 will be administered in a setting with access to personnel and facilities equipped to respond to and manage medical emergencies. Hospitalization requirements for subjects receiving study treatment are described in Section 6.3. As seen with studies of prior T-cell engaging antibodies, it is anticipated that most IRR/CRS reactions will occur in the first cycle, and primarily with the first dose. Since this is an FIH study, careful observation is required. Contact the Medical Monitor if there is any infusion reaction on first infusion. The suggested management of IRRs is detailed in Section 6.4.2.1.

PC-1 will be initially infused over 2 hours (±15 minutes). First dose infusion times may be shortened, or prolonged, depending on ongoing safety and tolerability data during the study. This will be under the oversight of the SRC. After Cycle 1, in the absence of infusion-related AEs with the previous dose of PC-1, the infusion time of PC-1 for subsequent doses may be successively reduced by up to 30 minutes (±15 minutes) to a minimum infusion time of 30 minutes (±15 minutes) following discussion with the Medical Monitor. A 10% margin of error for infusion times is allowed. Following the first infusion not requiring inpatient observation, subjects should be observed for at least 2 hours for signs and symptoms of IRRs. In the absence of infusion-related AEs, the post-administration observation time may also be successively reduced by up to 30 minutes, to a minimum observation period of 30 minutes. The infusion may be slowed or interrupted for subjects experiencing infusion-associated symptoms. To minimize toxicities and upon recommendation of the SRC, alternative schedules such as splitting Cycle 1 Day 1 over different days, or using different infusion rates, may be evaluated as long as the total dose for a cycle does not change from the assigned dose level. Subjects who undergo intra-subject dose escalation, first infusion of any step dose, or who restart PC-1 after a delay (i.e., subjects who have not received PC-1 for >6 weeks) should have premedication as detailed in Section 6.1.2.1 and Section 6.1.2.2 and infusion of PC-1 over a minimum of 2 hours (±15 minutes) and must be hospitalized. Guidelines for PC-1 dosage and schedule modification and treatment interruption or discontinuation are provided in Section 6.5. Study treatment administration must be performed by qualified study site staff and documented in source documents and the appropriate case report form (CRF). Any overdose or incorrect administration of PC-1 should be reported to the Sponsor and IRB/IEC, in accordance with IRB/IEC policies. AEs associated with an overdose or incorrect administration of study treatment should be recorded on the AE CRF.

### 6.1.2.1 Pre-Medication and Observation Requirements (Flat Dosing)

To minimize the risk of CRS-associated hypotension, holding anti-hypertensive medicine should be considered for 48-72 hours prior to and after all infusions of PC-1 in Cycle 1. Holding of anti-hypertensive medications is per the discretion of the Investigator in consultation with the Medical Monitor and may include a consultation with the subject's treating cardiologist.

PC-1 will be administered to subjects pre-hydrated with 500 cc normal saline. Hydration should be completed 10-30 minutes prior to administration of the Cycle 1 Day 1 dose. Pre-infusion vital signs should be collected prior to starting the hydration and again after completion of the hydration before administration of Cycle 1 Day 1. Hydration with normal saline at approximately 75 cc/hour should be administered following collection of the Cycle 1 Day 1 end of infusion PK sample and should continue throughout the inpatient observation per discretion of the Investigator.

Corticosteroid premedication consisting of dexamethasone 8 mg should be administered orally the day prior to dosing (12-16 hours prior). Additional corticosteroid premedication consisting of dexamethasone 10 mg should be administered orally 3 hours (±1 hour) or IV 1 hour (±30 minutes) prior to the administration of the Cycle 1 Day 1 dose of PC-1. Corticosteroid premedication is also recommended for all subjects prior to the Cycle 1 Day 8

administration. Use of corticosteroid premedication prior to subsequent administrations should be discontinued or tapered per the Investigator's discretion. Premedication with acetaminophen (or paracetamol) and an anti-histamine regimen should be administered per standard institutional practice prior to administration of each dose of PC-1 as tolerated. If a subject has fever/IRR/CRS with any infusion, corticosteroid premedication should be used prior to subsequent infusion of PC-1. Premedication adjustments for individual subjects should be discussed with the Medical Monitor. Premedication requirements may be modified per recommendation of the SRC.

Hospitalization requirements following the Cycle 1 Day 1 administration are outlined in Section 6.3. At each subsequent PC-1 dose following Cycle 1 Day 1, subjects will be observed as outlined above in Section 6.1.2.

### 6.1.2.2 Pre-Medication and Observation Requirements (Step Dosing)

To minimize the risk of CRS-associated hypotension, holding anti-hypertensive medicine should be considered for 48-72 hours prior to and after all infusions of PC-1 in Cycle 1. Holding of anti-hypertensive medications is per the discretion of the Investigator in consultation with the Medical Monitor and may include a consultation with the subject's treating cardiologist.

PC-1 will be administered to subjects pre-hydrated with 500 cc normal saline. Hydration should be completed 10-30 minutes prior to administration of the Cycle 1 Day 1 dose. Pre-infusion vital signs should be collected prior to starting the hydration and again after completion of the hydration. Pre-infusion hydration should be continued, at a minimum, prior to administration of each infusion through receipt of the first target dose level. In addition, hydration with approximately 75 cc/hour normal saline should be administered following collection of the end of infusion PK sample and should continue throughout the inpatient observation following administration of each infusion through receipt of the first target dose level per discretion of the Investigator.

Corticosteroid premedication consisting of dexamethasone 8 mg should be administered orally the day prior to dosing (12-16 hours prior). Additional corticosteroid premedication consisting of dexamethasone 10 mg should be administered orally 3 hours (±1 hour) or IV 1 hour (±30 minutes) at a minimum, prior to administration of each infusion through receipt of the first target dose level. Use of corticosteroid premedication prior to subsequent administrations should be discontinued or tapered per the Investigator's discretion. Premedication with acetaminophen (or paracetamol) and an anti-histamine regimen should be administered per standard institutional practice prior to administration of each dose of PC-1 as tolerated.

If a subject has fever/IRR/CRS with any infusion, corticosteroid premedication should be used prior to subsequent infusion of PC-1. Premedication adjustments for individual subjects should be discussed with the Medical Monitor. Premedication requirements may be modified per recommendation of the SRC. Hospitalization requirements for Cyle 1 are outlined in Section 6.3. At each subsequent PC-1 dose following Cycle 1, subjects will be observed as outlined above in Section 6.1.2.

### 6.2 Study Treatment Administration

PC-1 will be initially administered IV weekly on Days 1, 8, and 15 of 21-day cycles. Alternate dose schedules (e.g. 4-step dosing (Days 1, 4 and 8 in the first week of Cycle 1), Q2W, Q3W, etc.) may also be evaluated. Cycle 1 may be longer than 21 days if a ≥3-step dose regimen or Q2W regimen is evaluated. Subjects exhibiting acceptable safety and SD or better (or are deemed clinically benefitting) may continue to receive PC-1 until disease progression or unacceptable toxicity.

Administration of the first dose of PC-1 will be performed in a clinical setting with immediate access to staff who are trained to monitor for and respond to medical emergencies. Neurology consultation services should be readily available to address any neurologic AEs that may arise as a result of PC-1 treatment and nephrology consultation with acute dialysis capabilities should be readily available to address any renal toxicity that might accompany TLS. Administration of PC-1 should occur on the day of the scheduled dose but may be given up to ±2 days from the scheduled date for scheduling reasons. PC-1 should be given ≥5 days apart for weekly dosing. For 4-day step dosing, each dose should be given ≥3 days apart in the first week. For Q2W dosing, PC-1 doses should be given ≥12 days apart. For Q3W dosing, PC-1 doses should be given ≥19 days apart. Attempts should be made to keep the subject's treatment cycle in the register (i.e., a missed dose will not delay initiation of the next cycle).

### 6.3 Hospitalization

Subjects enrolled in dose escalation cohorts will require close monitoring for potential CRS. This includes hospitalization for 24-48 hours to monitor for CRS. For flat dosing, hospitalization is required after completion of the Cycle 1 Day 1 PC-1 infusion. For step dose cohorts, hospitalization will also be required after the completion of the PC-1 infusion for all step doses through the first administration of the target dose in Cycle 1. During this time, subjects will be monitored closely clinically, and have safety laboratory assessments (complete blood count, chemistry panel, CRP). In addition, there will be multiple PK blood draws for drug levels, as well as serum and PBMCs for exploratory biomarkers.

Any modifications to this hospitalization and monitoring plan will be based on review of available safety data by the SRC, following consultation with Investigators. The following criteria are considered for the modifications:
Hospitalization requirements during subsequent doses will be determined on the basis of the clinical course during Cycle 1. Based on clinical observation of CRS with PC-1, hospitalization longer than 24-48 hours may be allowed per Investigator's discretion after discussion with Medical Monitor or may be considered for implementation at the study level by the SRC.
For subjects enrolled at a dose regimen previously cleared and deemed safe (e.g., subjects in the dose expansion cohorts), hospitalization may not be required.
For subjects on alternative dosing regimens, the hospitalization requirements and monitoring plan may be modified based on review of available safety data by the SRC, following consultation with Investigators.

Subjects with a Grade 3 to 4 IRR, TLS, or CRS may also be hospitalized for observation during or after the administration of the subsequent dose, with considerations for dose reduction as described in Section 6.5.3. Hospitalization requirements in Cohort Backfill (Part 2) and Dose Expansion (Part 3) will be determined by the SRC in consultation with Investigators based on the safety observed in Dose Escalation (Part 1).

### 6.4 Safety Monitoring and Management

This study represents the FIH study of PC-1 administration. As such, the actual risks are not known. The following information describing management of safety concerns is based on mechanism of action, nonclinical data, expected pharmacology, preclinical safety findings, and available data related to other tumor antigen-directed bispecific TCEs. Measures are to be taken to ensure the safety of subjects participating in this study, including the use of stringent inclusion and exclusion criteria, premedication, and close monitoring and management, as described below.

All subjects will be monitored closely for toxicity. Subjects will be assessed clinically for toxicity prior to each dose using the NCI CTCAE v5.0 grading scale unless otherwise stated. CRS severity will be graded according to the Cytokine Release Syndrome Grading System as described in Table 16. PC-1 should only be administered under the supervision of clinicians with experience of CRS management and in a facility with a readily available intensive care unit and has tocilizumab (or equivalent anti-IL-6 or anti-IL-6-receptor antibody) readily available at the site. All AEs will be recorded during the study and for up to 30 days after the last dose of study treatment or until the initiation of another systemic anticancer therapy, whichever occurs first.

### Evaluation and Management of Adverse Events

In addition to the standard AE workup, additional blood samples should be drawn to evaluate SAEs or Grade≥3 AEs (e.g., IRR, CRS, TLS). Depending on clinical context, clinical laboratory assessments for CRP, ferritin, and tryptase should be added, as well as plasma for exploratory safety and PD markers, and PK levels. Drawing samples for T-cell subsets and activation marker analysis, ADA, and viral reactivation is recommended. Specific anticipated or potential toxicities associated with administration of PC-1 as well as the measures taken intended to avoid or minimize such toxicities in this study are described in the following sections.

### 6.4.2 Infusion-Related Reactions

PC-1 is a recombinant protein based therapeutic, and administration of therapeutic proteins has been associated with infusion reactions with symptoms and signs including fever, rigors, rash, urticaria, dyspnea, hypotension, and/or nausea. To minimize the risk of infusion reactions, all subjects will be premedicated with acetaminophen or paracetamol and diphenhydramine, and corticosteroids per institutional guidelines. Infusion reactions should be managed per institutional standards and per the guidelines below with antipyretics, antihistamines, and antiemetics, and for more severe reactions, with corticosteroids.

### 6.4.2.1 Management of Infusion-Related Reactions

IRRs are defined by NCI CTCAE v5.0 (under the category “Injury, poisoning, and procedural complications”) as “a disorder characterized by adverse reaction to the infusion of pharmacological or biological substances”. For the purposes of this study, the time frame for infusion reaction assessment is the 24-hour period beginning from the start of the infusion. Recommendations for the management of IRRs are provided below. For Grade 1 IRR: described as mild transient reaction; infusion interruption not indicated; intervention not indicated:

Remain at bedside and monitor the subject until recovery from symptoms.

For Grade 2 IRR: described as infusion interruption indicated, but responds promptly to symptomatic treatment (e.g., antihistamines, nonsteroidal anti-inflammatory drugs, narcotics, IV fluids); and prophylactic medications indicated for ≤4 hours:
Stop the PC-1 infusion, begin an IV infusion of normal saline, and treat the subject with diphenhydramine 50 mg IV (or equivalent) and/or 500 to 750 mg oral acetaminophen.
Corticosteroid therapy may also be given at the discretion of the Investigator.
Remain at bedside and monitor the subject until resolution of symptoms.
If the infusion is interrupted, wait until symptoms resolve, then restart the infusion at 50% of the original infusion rate.
If no further complications occur after 60 minutes, the rate may be increased to 100% of the original infusion rate. Monitor the subject closely.
If symptoms recur, stop the infusion and disconnect the subject from the infusion apparatus. No further PC-1 will be administered at that visit.
The amount of PC-1 infused must be recorded on the CRF.
Subjects who experience a Grade 2 infusion reaction during the post-infusion observation period that does not resolve during that time should be observed until the AE resolves or stabilizes, with vital sign measurements every 4 hours and additional evaluations as medically indicated for the management of the AE.

For Grade 3 or Grade 4 IRR: Grade 3 is described as prolonged (e.g., not rapidly responsive to symptomatic medication and/or brief interruption of infusion) recurrence of symptoms following initial improvement; hospitalization indicated for other clinical sequelae (e.g., renal impairment, pulmonary infiltrates). Grade 4 is described as life-threatening consequences; urgent intervention indicated.
Immediately discontinue the infusion of PC-1.
Investigators should follow their institutional guidelines for the treatment of anaphylaxis or high-grade infusion reactions.
Consider treating the subject with an IV infusion of normal saline and administer corticosteroids, H1 and/or H2 blockers, bronchodilators, oxygen, or vasopressors as needed.
Consider adding to safety labs, plasma for exploratory cytokines and chemokines, CRP, tryptase, and lactate.
The subject should be monitored until the Investigator is comfortable that the symptoms will not recur.
All subjects with Grade≥3 IRRs will be observed until the AEs resolve or stabilize, with vital sign measurements and additional evaluations as medically indicated for the management of the AEs.
Subjects with Grade 3 IRR during Week 1 of Cycle 1 may also be hospitalized during or after the administration of the subsequent dose, with considerations for dose reduction as described in Section 6.5.3.

### 6.4.3 Cytokine Release Syndrome

The identified risks of treatment with TCEs are primarily related to cytokine release and CRS. PC-1 is designed to reduce the risk of CRS by requiring protease cleavage for activation, focusing molecular activity to the TME where proteases are overexpressed, dysregulated, and activated. This approach has been shown to markedly reduce systemic cytokine exposure in preclinical models. Data from other clinical development programs (of non-masked TCE) has shown that broad T-cell activation can lead to cytokine release into the blood, which can cause acute effects of fever, hypotension, and hypoxia and may require care in the hospital. These side effects are short-lived and can be treated with IV fluids, tocilizumab, and supportive care, but occasionally have the potential to be severe. These vital signs and adverse effects will be carefully monitored for their appearance and followed closely in this clinical study. In the nonclinical toxicology studies described in the IB, PC-1 can be dosed at 50- to 150-fold higher doses than the unmasked active form of TCE with equivalent safety and cytokine induction activity. This safety window observed in animal studies offers the promise that higher active doses may be achieved in patients and may offer an increased level of anti-tumor activity. Guidelines for treatment of CRS have been mainly driven by the CAR-T cell therapy field, where CRS seen with anti-CD19 CAR-T-cells can be severe and more prolonged (Neelapu 2018). With bispecific antibodies, CRS is shorter duration, and generally less severe, but may still be dose-limiting, and is commonly mitigated by step dosing approaches and CRS mitigation strategies. Cytokine release is primarily a first/step/or target dose phenomenon, and tolerance to cytokine induction occurs with subsequent infusions of T-cell engagers. CRS can infrequently occur at later infusions, especially if there is a treatment free interval of weeks/months (depending on the T cell engager). Preventive measures of CRS will include glucocorticoid premedication, IV hydration, and holding of anti-hypertensive medication. Step dosing will be initiated if CRS is seen during dose escalation. In addition, subjects should be encouraged to measure their blood pressure and temperature at home twice per day during Cycle 1 and call the research team to review any concerning results.

### CRS Management

Cytokine release can lead to fevers, tachycardia, hypotension and hypoxia, and needs to be followed closely. CRS may also be associated with fatigue, nausea, headache, dyspnea, rigors, myalgia/arthralgia, and anorexia. CRS will be graded using the ASTCT consensus grading scale (Table 16), adapted from Lee et al 2019. CRS will be managed with IV hydration and other supportive care measures, as necessary, and increased monitoring. More significant CRS may require oxygen, corticosteroids, and/or anti-IL-6 pathway inhibition. Severe CRS may also require intensive care unit monitoring and management with vasopressors, antiepileptics, high-dose corticosteroids, and/or mechanical ventilation. Tocilizumab or equivalent anti-IL-6 antibody (eg, siltuximab), or anti-IL6-receptor antibody (eg, sarilimab) should be considered early in the clinical course of CRS to help avoid CRS progression and severe clinical manifestations. Recommended guidelines for the treatment of CRS have been mainly driven by the CAR-T cell therapy field, where CRS seen with anti-CD19 CAR-T cells can be severe and more prolonged (Lee et al 2019). With immunoglobulin G (IgG) format bispecific antibodies, CRS may occur and be dose-limiting.

TABLE 13
Grading CRS (ASTCT Consensus Scale)

| Parameter | Grade 1 | Grade 2 | Grade 3 | Grade 4 |
|---|---|---|---|---|
| Fever | >+38° C. | >+38° C. | >+38° C. | >+38° C. |
| Hypotension | None | Responds to | Requiring a | Requiring multiple |
| | | IV fluids | vasopressor | vasopressors (excluding |
| | | | | vasopressin) |
| Hypoxia | None | Requiring | Requiring | Requiring positive pressure |
| | | low-flow | high-flow | (eg, CPAP, BiPAP, intubation |
| | | nasal cannula | nasal cannula, | and mechanical ventilation) |
| | | | or mask | |

ASTCT = American Society for Transplantation and Cellular Therapy;
BiPAP = bilevel positive airway pressure;
CPAP = continuous positive airway pressure;
CRS = cytokine release syndrome;
IV = intravenous

### For any Grade CRS:

Monitor clinical laboratory assessments.
Monitor CRP levels.
Draw extra safety laboratory samples including amylase, lipase, and lactate
Draw plasma for exploratory cytokines and chemokines before administering tocilizumab

For Grade 1 CRS: Many subjects may only manifest fever and tachycardia. Close monitoring is required to detect and react to early signs of progression.
Frequent monitoring of vital signs
Consider tocilizumab or equivalent anti-IL-6 antibody (eg, siltuximab), or anti-IL-6 receptor antibody (eg, sarilimab)

For Grade 2 CRS: Hypotension and/or hypoxia detected
Treat with aggressive IV hydration with normal saline.
Supplemental oxygen as indicated
Consider cardiac telemetry and pulse oximetry
Consider corticosteroids (eg, dexamethasone 10 mg every 12 hours)
Consider tocilizumab or equivalent anti-IL-6 antibody (eg, siltuximab), or anti-IL-6 receptor antibody (eg, sarilimab)

For Grade 3 CRS: Hypotension not responsive to IV fluids
Management in monitored bed or intensive care unit
Corticosteroids as indicated dexamethasone 10 mg every 6 hours, or methylprednisolone 1 mg/kg IV twice per day
Tocilizumab (4 to 8 mg/kg IV over 1 hour, not to exceed 800 mg), or equivalent anti-IL-6 antibody (eg, siltuximab), or anti-IL-6 receptor antibody (eg, sarilimab)
Vasopressor support
Oxygen therapy with mask if needed

For Grade 4 CRS: Life-threatening symptoms, requirements for ventilator support or continuous veno-venous hemodialysis
High-dose corticosteroids (methylprednisolone 1000 mg/day IV)
Consider multiple pressor agents
Mechanical ventilation and/or renal replacement therapy may be required.

### 6.4.4 Other Potential Toxicities

### 6.4.4.1 Tumor Lysis Syndrome

Subjects with a high disease burden may be at risk for developing TLS with PC-1 treatment. Prophylactic treatment/measures are strongly recommended for subjects considered to be at risk for TLS, per institutional or clinical standards. Subjects should be closely monitored for laboratory evidence of TLS (hyperuricemia, hyperkalemia, hyperphosphatemia, and hypocalcemia). In the case of evidence of TLS associated with PC-1, subjects will be admitted to the hospital, as clinically indicated. Standard management will include vigorous IV hydration, hypouricemic agents, and correction of acidosis, if present. Renal function, serum uric acid, calcium, phosphorus, and electrolytes should be closely monitored. Subjects with Grade 3 to 4 TLS during Week 1 or Cycle 1 Day 1 may also be hospitalized for ≥24 hours after the end of the administration of the subsequent dose, with considerations for dose reduction as described in Section 6.5.3.

### 6.4.4.2 Neurological Events

Neurological events associated with cytokine release may include tremor, mental status changes, confusion, speech difficulties, and potentially seizures. Monitor subjects for neurological events and exclude other causes for neurological symptoms. Neurological events should be graded according to NCI CTCAE v5.0. The Immune Effector Cell-Associated Encephalopathy (ICE) score should be utilized to evaluate the grade of any suspected immune effector cell-associated neurotoxicity syndrome (ICANS) (Lee 2019). Mild disorientation or expressive aphasia (trouble word-finding) may be the earliest and most specific signs. Provide supportive care as needed for any neurological events. Workup may include head magnetic resonance imaging (MRI) and electroencephalogram and may require corticosteroids, or anti-seizure medications, if severe. Contact the Medical Monitor if there is any potentially treatment-related neurological toxicity.

### 6.4.4.3 Infections

TCE-based immunotherapies have shown some complications with serious infections, particularly with anti-B cell TCE in lymphoma and myeloma. Bacterial, fungal, and new or reactivated viral infections, may occur during and/or following the completion of PC-1 based therapy. PC-1 should be held or discontinued for serious infections, and appropriate anti-infective therapy should be instituted. New or reactivated viral infections may include cytomegalovirus, herpes simplex virus, parvovirus B19, varicella zoster virus, West Nile virus, and HBV/HCV. PC-1 is not recommended for use in subjects with severe, active infections.

### 6.4.4.4 Hepatitis B Reactivation

HBV reactivation can occur in patients treated with drugs classified as TCE antibodies. Cases have been reported in patients who are hepatitis B surface antigen (HBsAg) negative but are hepatitis B core antibody (anti-HBc) positive. HBV reactivation is defined as an abrupt increase in HBV replication manifesting as a rapid increase in serum HBV DNA levels or detection of HBsAg in a person who was previously HBsAg negative and anti-HBc positive. Reactivation of HBV replication is often followed by hepatitis (i.e., increase in transaminase levels). In severe cases, increase in bilirubin levels, liver failure, and death can occur. It is recommended to monitor subjects with evidence of prior HBV infection (anti-HBc positive) with HBV DNA testing monthly, and for clinical and laboratory signs of hepatitis during and for several months following PC-1 therapy (Appendix C).

### 6.4.4.5 Immunization

The safety of immunization with live viral vaccines during or following PC-1 therapy has not been studied. Vaccination with live virus vaccines is not recommended for ≤2 weeks prior to the start of PC-1 treatment, during treatment, and until immune recovery following the last cycle of PC-1. Subjects are encouraged to get the COVID-19 vaccination/booster >2 weeks prior to PC-1 treatment initiation. Vaccination efficacy on study is unknown; however, COVID-19 vaccination would be allowed on study, after clearance of the first cycle, and with a 2-week dose hold post COVID-19 vaccination.

### Potential Healthy Tissue Toxicities

While PSMA is most highly expressed in prostate cancer cells, and highly prostate restricted, it can be expressed at lower levels on some healthy tissues. As a result, PSMA-targeted therapy may cause on-target, off-tumor toxicity in health tissues. As PC-1 requires tumor specific protease activity, toxicity should be limited at non-tumor sites.

### 6.4.5.1 Kidney, Gastrointestinal, and Liver

Immunohistochemistry studies have indicated that several non-prostatic tissues can express low levels of PSMA. These include kidney proximal tubule cells, and duodenal brush border cells, and rare cells colonic crypts. (Silver et al, 1997, The Protein Atlas, 2022). PSMA-PET scanning, using a small molecule PSMA active site binder, has confirmed tissue accumulation in the small intestine, and liver. (Kidney accumulation cannot be resolved as the reagent is excreted through the kidney and bladder). Renal or gastrointestinal toxicity has not been described as an issue with small molecule PSMA binders such as 177-Lu-PSMA-617 (PLUVICTO), (Sartor et al, 2021) or with the PSMA-CD3 bispecific antibodies such as AMG160. However, transaminitis has been seen with AMG160, but has been mostly attributed to CRS. (Tran et al, 2020). These major organ functions will be closely monitored during the study. Upon observation of clinical symptoms or clinically significant laboratory data, including urinalysis, additional blood sampling outside of the schedule outlined in the SOA is recommended to evaluate organ function prior to dosing.

### 6.4.5.2 Salivary and Lacrimal Glands

PSMA-PET scanning, using a small molecule PSMA active site binder, has demonstrated clear salivary and lacrimal gland uptake. It is not clear if there are other non-specific or poorly understood mechanisms for this uptake. (Morris et al, 2021, Sheehan et al, 2021). Small molecule PSMA binders such as 177-Lu-PSMA617 (PLUVICTO), have shown a potential for a dry mouth and dry eye side effects, (Sartor et al, 2021), but these side effects are much less frequently seen with PSMA-CD3 bispecifics, but they can occur.

### (Tran et al, 2020).

Dry mouth and dry eyes will be closely monitored during the study.

### 6.5 Dose and Schedule Modifications

Every effort should be made to administer study drug per the planned dose and schedule.

In the event of significant toxicity, dosing may be delayed and/or reduced as described below. Dose modification should be based on the highest toxicity observed in the event of multiple toxicities. The Investigator should instruct subjects to notify them at the first occurrence of any adverse symptom. Dose modification may occur in the following way for any toxicity:
Within a cycle: dosing interruption until adequate recovery and dose reduction, if required, during a given treatment cycle;
Between cycles: next cycle administration may be delayed due to persisting toxicity when a new cycle is due to begin;
In the next cycles: dose reduction may be required in a subsequent cycle based on toxicity experienced in previous cycle.

### 6.5.1 Dose Interruption

Subjects experiencing Grade 3 or 4 potentially related toxicity or intolerable Grade 2 toxicity despite supportive care should have their treatment interrupted.

Appropriate follow up assessments should be done until adequate recovery occurs as assessed by the investigator. Criteria required before treatment can resume are described in Section 6.5.3. Discontinue treatment for any AE, recurring AE, laboratory abnormality, or intercurrent illness which, in the Investigator's clinical judgement, presents a substantial risk to the subject with continued PC-1 treatment. Appropriate follow-up assessments should be performed until adequate recovery occurs, as assessed by the Investigator. Depending on when the AE resolves, treatment interruption may lead to a delay in the initiation of the subsequent cycle. If scheduled dosing coincides with a holiday that precludes dosing, dosing should commence on the nearest following date, with subsequent dosing continuing on a 7-day, 14-day or 21-day schedule as applicable. Subjects who require a treatment interruption due to a treatment-related adverse event of >2 weeks should discontinue PC-1 unless, in the opinion of the treating investigator and in consultation with the Medical Monitor, the subject is deriving benefit from study treatment. In any situation where a treatment-related adverse event results in an interruption of >4 weeks, PC-1 should be discontinued.

### 6.5.2 Dose Delay

Dosing following treatment interruption for treatment-related toxicity on or at the start of any new cycle may occur when all the following parameters have been met:
ANC≥1,000/mm³
Platelet count ≥50,000/mm³
Nonhematologic toxicities have returned to baseline or Grade≤1 severity (or, at the Investigator's discretion, Grade 2 if not considered a safety risk for the subject).
Study treatment may be delayed or dose reduced as appropriate for management of toxicity. Documented approval by the Medical Monitor and the Investigator of all decisions regarding dose and schedule modifications for treatment emergent events is required.
Treatment delays of >4 weeks not due to treatment-related AE (e.g., for an unrelated medical condition with expected recovery, vacation) must be approved by the Medical Monitor.

### 6.5.3 Dose Reduction

Following dosing interruption or cycle delay due to toxicity, the PC-1 dose may be reduced when treatment is resumed. No specific dose adjustments are recommended for Grade ½ treatment related toxicity. However, investigators should always manage subjects according to their medical judgment, based on the clinical circumstances. Dose reduction of PC-1 by 1, if needed, 2 or 3 dose levels may be allowed depending on the type and severity of toxicity observed (Table 14). Subjects should be discontinued from the study if dose reduction below 25 μg is required. Subjects requiring more than 2 dose reductions will be discontinued from the treatment and entered into the follow-up phase, unless otherwise agreed between the Investigator and the Sponsor. A subject who experiences a DLT will require Medical Monitor approval prior to restarting treatment with PC-1.

TABLE 14
Guidelines for PC-1 Dose Reduction

| Dose Reduction Level of Target | |
|---|---|
| Dose or Ongoing Treatment Dose | PC-1 |
| 1 | 50% reduction * |
| 2 | 75% reduction * |
| 3 | 87.5% reduction * |

* PC-1 dose reduction below 25 μg is not allowed.

Subjects who have been dose reduced due to a toxicity may potentially re-escalate dosing. Subjects may re-escalate dosing by 2-fold or less if the toxicity does not recur while on treatment at the lower dose level. The subject may then sequentially escalate if the toxicity does not recur, as assessed by the Investigator in consultation with and approval of the Medical Monitor.

Recommended dose modification/reduction is described in Table 15.

TABLE 15
Dose Modification/Reduction Guidelines for PC-1

| Event | Action |
|---|---|
| Grade 3 or 4 non-hematologic | Hold PC-1 until subject recovers to Grade ≤1 or |
| toxicity (except CRS) considered | baseline and reduce by 1 dose level. |
| related to PC-1 per investigator | If toxicity (Grade 3-4) reoccurs despite |
| judgement (including persistent | reduction, subject may be dose reduced again |
| nausea, vomiting, diarrhea despite | level (except for the first cohort) upon recovery to |
| optional medical therapy), | Grade ≤1 or baseline. |
| clinically manageable electrolyte | Prompt palliative and supportive measures |
| abnormalities. | by local standard of care (e.g., antiemetic). |
| | Subjects who experience Grade 4 non- |
| | hematologic toxicities despite intervention |
| | should be discontinued from study treatment, |
| | unless restarting treatment at reduced dose level |
| | Investigator judgement and after consultation with |
| | Medical Monitor (e.g., clear clinical benefit |
| | and no alternative treatment options). |
| Cytokine Release Syndrome | Grade 2: Hold PC-1 until resolved to Grade ≤1. |
| | Resume at the next planned dose. |
| | Grade 3: Hold PC-1 until resolved to Grade ≤1. |
| | Resume at the next planned dose or a |
| | modification of the next dose can be considered, |
| | including a repeat of the current dose level or a |
| | reduction by at least 1 dose level per Investigator |
| | discretion and in consultation with the Medical |
| | Monitor. |
| | If the reduced dose is tolerated with no |
| | signs/symptoms of Grade 3 or higher |
| | CRS, the subject may return to the prior |
| | dose. |
| | PC-1. |
| | Grade 4: Permanently discontinue PC-1. |
| Hematological toxicity considered | Hold PC-1 until recovery of ANC to ≥1.0 × 109 |
| related to PC-1 per Investigator | cells/L (ANC ≥1,000/mm3) and platelet |
| judgement | to ≥75 × 109 cells/L |
| Grade 4 neutropenia (absolute | (ANC ≥75,000/mm3) and resume |
| neutrophil count [ANC] <0.5 × 109 | at the same dose after discussion with the Medical |
| cells/L) lasting >3 days | Monitor. |
| (growth factor support is | If toxicity reoccurs (grades as described to the left) |
| allowed) or | reduce by 1 dose level. Subjects may either be held |
| Febrile neutropenia (defined as | until recovery and continuation at same dose or |
| ANC <1.0 × 109 cells/L with a | undergo further dose reduction by 1 more dose level |
| single temperature of >38.3° C. or | (except for the first cohort). |
| 101° F., or a sustained | |
| temperature of ≥38° C. or 100.4° F. | |
| for >1 hour) or | |
| Grade 4 thrombocytopenia | |
| lasting >3 days | |
| or Grade ?3 thrombocytopenia with | |
| Clinically significant bleeding | |
| No recovery of toxicities within | Discontinue treatment, unless restarting treatment at |
| 4 weeks of scheduled PC-1 | reduced dose level is in the subject's best interest per |
| infusion | Investigator judgement and after consultation with the |
| | Medical Monitor (e.g., clear clinical benefit and |
| | no alternative treatment options) |

### Concomitant Therapy

### 6.6.1 Concomitant Medications

All treatments that the Investigator considers necessary for a subject's welfare may be administered at the discretion of the Investigator in keeping with the standards of medical care. However, if a subject requires a medication that is prohibited per protocol, the Medical Monitor must be notified, and the subject may be required to discontinue from study treatment. All medications received within 28 days prior to the first dose and for 30 days after the last dose of PC-1 should be recorded on the CRF, including all prescription, over-the-counter, and IV medications and fluids. The reason for treatment, name of the drug, dosage, route, and start and stop dates of administration for each concomitant medication will also be reported on the CRF. All changes (e.g., changes in dose, discontinuation, etc) in concomitant treatments that occur during the study should also be reported. Subjects receiving bisphosphonates, denosumab, zoledronic acid or similar therapy prior to enrollment may be maintained on this therapy during the study. Bisphosphonates can be stopped or started at the discretion of the Investigator throughout the study. However, no changes in bisphosphonates should be made during the DLT assessment period. Subjects should be discouraged from use of illicit drugs, herbal remedies, self-prescribed drugs, tobacco products, or excessive alcohol at any time during the clinical study.

6.6.2 Concomitant Treatment to be Used with Caution

Inflammation and cytokines have been shown to suppress CYP450 enzymes, which may cause drug-drug interactions (de Jong et al, 2020). Cytokines transiently released by T-cell redirecting antibody and CRS may effect concomitant drug metabolism. Caution needs to be paid where subjects are receiving substrates of CYP enzymes with narrow therapeutic index window. Subjects should be monitored closely and have doses for the concomitant treatments adjusted as necessary. The website below lists drugs which fit these parameters for CYP3A4, the main enzyme effected by cytokine signaling.

CYP Weblink—Cytochrome P-450 CYP3A4 Substrates with a Narrow Therapeutic Index. https://go.drugbank.com/categories/DBCAT004028

### 6.6.3 Excluded Medications

Medications that may interfere with the mechanism of action of PC-1 or the ability to interpret study-specific endpoints are not allowed during the study. All questions regarding permitted and prohibited concomitant medications should be discussed with the Medical Monitor. Listed below are specific restrictions for concomitant therapy during the treatment period of the study (Note: There are no prohibited therapies during the post-treatment long-term follow-up period):
Anticancer systemic chemotherapy, immunotherapy, or biological therapy other than that specified in the protocol (PC-1)
Investigational/experimental agents other than PC-1
Radiation therapy
   Note: Radiation therapy to a symptomatic solitary lesion, to the brain, or for palliative purposes may be allowed after consultation with the Medical Monitor.
Systemic glucocorticoids at supraphysiologic doses (>10 mg/day prednisone or equivalents) for reasons other than for the management of toxicities with suspected immunologic etiology (e.g., CRS) are not permitted. Topical, intranasal, and inhaled steroids are permitted.
Use of any herbal products that could decrease PSA levels (e.g., saw palmetto) within 28 days of the first infusion. The subject must agree not to use such herbal products during study participation.

### Description of Study Periods

Research staff should refer to the SOA for an outline of the required procedures.

An overview of study assessments/procedures is outlined below. The timings of study visits and procedures are presented in the SOA. Refer to the CRF completion guidelines for data collection requirements.

### 7.1 Screening

The screening period begins on the date the subject signs the IRB/IEC-approved ICF and continues through confirmation of eligibility and enrollment into the study. Informed consent must be obtained before completion of any non-standard of care study-specific procedures. Subjects who have fully consented to participation in the study will undergo screening assessments within 28 days prior to administration of the first dose of PC-1 (unless otherwise stated). Procedures that are part of standard of care are not considered study-specific and, therefore, may be performed before obtaining consent and used to confirm eligibility, provided they occur within the time allowance outlined below and in the SOA. For those subjects who meet all of the eligibility criteria, an enrollment form will be completed and submitted to the Sponsor or designee confirming eligibility. If eligibility is confirmed, subjects will then be enrolled and assigned a treatment cohort. Refer to the SOA timing and details of study procedures to be completed during the screening period.

### 7.1.1 Screen Failures

Only subjects who meet all of the eligibility criteria listed in Section 5 will be enrolled into the study. If at any time before enrollment the subject fails to meet the eligibility criteria, the subject should be designated as a screen failure, and the reason(s) for failing screening should be recorded.

### 7.1.2 Rescreening

Subjects who are screen failures will be permitted to rescreen. Subjects will retain the same screening number assigned at the original screening. If screening procedures are repeated within 28 days prior to administration of the first dose of PC-1, it is only necessary to perform the procedure(s)/assessment(s) that did not meet the eligibility criteria; all other screening procedures/assessments do not need to be repeated. Subjects whose assessments are to be repeated more than 2 times should be discussed with the Medical Monitor.

Subjects being rescreened outside of the 28-day window must be reconsented.

### Study Treatment Period

For the study treatment period procedures, see the SOA and Section 8.

It is permissible to see the subject for an examination, ECG, and research laboratory assessments on the day prior to each infusion. Should a ≥3 weekly step dose schedule be evaluated, the Cycle 1 Day 8 visit assessments detailed in the SOA should be performed at the time each dose level is increased prior to the first target dose administration. Cycle 1 will be more than 21 days in duration with the Day 15 visit assessments to be performed at the first target dose administration.

### 7.3 End of Treatment

The EOT Visit must occur within 30 days (±7 days) of the decision to discontinue treatment and prior to initiation of any new anticancer therapy/regimen. Subjects who discontinue treatment prior to documented disease progression should continue to have imaging disease assessments as outlined in the SOA, until disease progression or initiation of a new anticancer treatment, whichever occurs first. All subjects discontinuing study treatment for any reason should undergo the EOT procedures as indicated in the SOA. All subjects should return to the clinic to have the EOT Visit assessments performed, irrespective of when treatment is discontinued.

### 7.4 Long-Term Follow-Up Period

Unless consent is withdrawn, all subjects will enter into the long-term follow-up period of the study after the EOT Visit to obtain survival data. Survival data may be obtained either by clinic visit or remote communication (e.g., telephone call or email). Sites must follow up with subjects approximately every 3 months (±14 days) to assess the following data:
Date of PD (if not documented on study)
Subsequent anticancer therapies
Date of subsequent anticancer therapies
Survival status

For subjects lost to follow-up or whose survival status is unknown, every effort will be made to determine the date such subjects were last known to be alive. Such efforts may include phone calls, certified mail, and the checking of public records.

7.5 Withdrawal from Study

Each subject has the right to withdraw from the study at any time. In addition, the Investigator has the right to remove subjects from the study for any reason including the occurrence of an AE or noncompliance with the protocol. The Sponsor or its designee must be notified if a subject is withdrawn from study treatment or the study. Samples collected prior to when a subject withdraws from the study may still be analyzed, unless the subject requests that the samples be destroyed.

### Study Procedures and Assessments

### 8.1 Informed Consent

Before a subject participates in the clinical study, the Investigator is responsible for obtaining written informed consent from the subject after adequately explaining the study design, anticipated benefits, and potential risks. The consent process and the subject's agreement or refusal to participate in the study must be documented in the subject's medical records. If the subject agrees to participate, the ICF must be signed and dated by the subject and the person who conducted the informed consent discussion, and also by an impartial witness (if required by IRB, IEC, or local requirements) before his participation in the study. The original signed ICF will be retained in accordance with institution policy and IRB/IEC requirements, and a copy of the ICF will be provided to the subject. All subjects who are enrolled into the study should be re-consented with any updated version of the IRB/IEC-approved ICF if the new version is relevant to their participation.

### 8.2 Demographic Data

Demographic data will be collected as per country and local regulations and guidelines. Where applicable, demographic data will include sex, age, race, ethnicity, and country of enrollment to study a possible association between these variables and subject safety and treatment effectiveness.

### 8.3 Medical and Treatment History

Relevant medical history before the start of AE reporting will be collected. Relevant medical history is defined as data on the subject's current medical condition that would typically be obtained to capture relevant underlying conditions. In addition to the medical history, all history related to the subject's PC treatment and response to treatment will be collected and must date back to the original diagnosis. All findings will be recorded in the CRFs.

### 8.4 Physical Examination, Vital Signs, and Eastern Cooperative Oncology Group Performance Status

A complete physical examination, including height and weight, should be performed at screening. Thereafter, limited physical examinations are acceptable and may also include routine examination of the following systems: cardiovascular, pulmonary, gastrointestinal, CNS, and skin (including fingers, toes, and ears). Vital signs, including blood pressure, heart rate, respiratory rate, body temperature and pulse oximetry while the subject is in a sitting or semi-recumbent position, will be monitored and recorded at screening and timepoints outlined in the SOA. On dosing days where pre-infusion hydration is required, pre-infusion vitals should be obtained prior to starting the pre-infusion hydration and again after completion of the hydration. If the infusion is administered over 30 minutes, vital signs should be collected prior to the infusion and at the end of the infusion. In addition to the timepoints outlined in the SOA, vital signs are monitored as clinically indicated and if there are any new or worsening clinically significant changes since the last examination; changes will be reported on the appropriate CRF page. ECOG performance status will be performed to quantify the subject's general well-being and ability to perform activities of daily life.

### 8.4.1 Cardiac Function

### 8.4.1.1 Electrocardiogram

A baseline 12-lead ECG will be performed during the screening period and timepoints outlined in the SOA using local institutional ECG equipment. Guidance for ECG collection includes the following:

When an ECG is scheduled at the same time as a blood collection, the ECG will be obtained prior to the blood collection. Food should not be consumed 1 hour before the ECG collections; only oral fluids are allowed within 1 hour before the ECG collections. If the ECG occurs near a meal, the ECG will take place prior to the meal. ECGs will be recorded after the subject has been in the supine or semi-recumbent position for at least 10 minutes. Subjects will be instructed to remain completely stationary (no talking, laughing, deep breathing, sleeping, or swallowing) for approximately 10 seconds during the ECG recording. While ECGs are being acquired, subjects and staff should turn off devices in the room (e.g., cellular telephones, fans, heaters, etc) that emit electrical interference.

### 8.4.1.2 Left Ventricular Ejection Fraction

Baseline left ventricular ejection fraction (LVEF) will be assessed during screening by ECHO or MUGA and must be reviewed prior to administration of PC-1. An ECHO or MUGA assessment that was performed after the subject's last chemotherapy treatment may also be used to confirm eligibility, provided that it occurred ≤28 days before signing the ICF. ECHO is the preferred method since it can detect wall motion abnormalities. However, in situations where an ECHO machine is not readily available or is technically limited, a MUGA scan may be obtained to evaluate LVEF. The same methodology should be used throughout the study and, to the greatest extent possible, at the same institution/facility.

### Neurological Assessments

### 8.5.1 Neurological Examinations

A neurological examination will be performed at screening, and the following abnormalities will be recorded: cranial nerves, motor system, reflexes, coordination, sensory system, and neuropsychological findings (e.g., speech, cognition, and emotion). Thereafter, symptom-directed neurological examinations are acceptable. A neurological assessment should be done before each PC-1 infusion if Grade≥2 CRS or any neurological symptom is observed on a prior infusion. Subjects will be specifically asked about changes in neurological status since the previous examination, as noted in the SOA. The ICE score should be utilized to evaluate the grade of any suspected ICANS.

### 8.5.2 Neurological Imaging

Subjects with neurological signs or symptoms will undergo a screening brain MRI/head MRI to rule out CNS metastasis during the screening period. If an MRI is contraindicated, then a head CT may be performed instead.

### 8.6 Disease Assessments

Subjects will be evaluated for disease response by the site Investigator at the timepoints indicated in the SOA. Disease assessments will be evaluated per RECIST 1.1 for soft tissue disease and PCWG3 for bone disease and PSA (Appendix D). Flow cytometric, molecular, or cytogenetic studies will not be used to determine response. Scans will be archived for possible evaluation by a blinded, centralized independent radiology review at a later time. The timing for this central review will be determined by the Sponsor.

### 8.6.1 Imaging

### 8.6.1.1 Imaging at Baseline

To confirm eligibility and/or to establish a baseline, CT and bone scans are required. PSMA-PET is also required at baseline. CT scans of the chest, abdomen, and pelvis, along with the appropriate imaging of all other sites of disease, are required at screening. Imaging performed following the subject's last line of therapy and prior to signing the ICF may be used for confirmation of eligibility if it is within 42 days prior to enrollment and no other anticancer treatment has been administered. If imaging is performed >42 days prior to enrollment, the scans must be repeated to establish a new baseline. Subjects who cannot have contrast CT scans due to contrast allergy or impaired renal function may be evaluated with non-contrast chest CT and MRI scans. The same radiographic assessment modality should be used for all response evaluations as was used at baseline. Imaging should use the same protocol for consistency across different timepoints (e.g., CT with the same contrast protocol for CT scans).

FDG-PET is optional for baseline evaluation. FDG-PET can be used for prognostic or predictive potential, and correlative assessment for response or safety.

### 8.6.1.2 Post-Treatment Response Assessment

The first planned post-treatment tumor assessment will occur at Week 9 (±5 days). The second tumor assessment will occur at Week 18 (±5 days). PSMA-PET will be obtained at Week 9, Week 18 and after Week 18 if the demonstration of first formal response (PSA30 or radiologic CR or PR) is demonstrated after Week 18. PSMA-PET should be scheduled to occur at less than 4 weeks from determination of response. Imaging will then continue at 3-month intervals (±7 days) through Month 12, and every 6 months (±7 days) thereafter, or until disease progression or the EOT Visit. Subjects with symptoms suggestive of disease progression should be evaluated for progression at the time symptoms occur even if this requires an unscheduled visit. EOT tumor assessment may be omitted if the subject has documented disease progression or if disease/response assessment was performed within the preceding 6 weeks.

### 8.6.2 Prostate-Specific Antigen Assessments (Serum)

Two serum samples for PSA will be drawn according to the SOA unless indicated that only a local sample is to be collected. When two samples are collected, one will be used to evaluate PSA at the site's local laboratory and the second sample will be sent to the central laboratory for analysis.

### 8.7 Laboratory Assessments

### 8.7.1 Safety Laboratory Assessment

Assessments listed in Table 16 will be performed at the local laboratory at the timepoints indicated in the SOA. The Investigator may choose to repeat any abnormal test once to rule out laboratory or sample collection error. Pre-dose samples may be collected up to 2 days before dosing.

TABLE 16
Local Safety Laboratory Assessments

| Serum | | | | Other Lab |
|---|---|---|---|---|
| Chemistry | Hematology | Coagulation | Urinalysis | Assessments |
| Sodium | CBC with | PT or INR | Urine specific | HBsAg³ |
| Potassium | differential² | PTT or aPTT | gravity | HbcAb³ |
| Chloride | WBC | D-Dimer | Urine pH | Anti-HCV³ |
| Bicarbonate total | Neutrophils | | Protein | Anti-HIV-1 and -²^{³} |
| BUN or urea¹ | Lymphocytes | | Glucose | SARS-CoV2^{3, 4} |
| Creatinine | Monocytes | | Bilirubin | CRP⁵ |
| Glucose | Basophils | | Ketones | Ferritin |
| Calcium total | Eosinophils | | Microscopic | Tryptase⁶ |
| Inorganic | RBC | | findings (e.g. | Amylase⁷ |
| phosphorus | Hemoglobin | | RBCs, WBC, | Lipase |
| Magnesium total | Hematocrit | | epithelial cells, | Lactate/lactic |
| Uric acid² | Platelets | | etc.) | acid |
| LDH | | | | EBV qPCR⁸ |
| Albumin | | | | CMV qPCR⁸ |
| Bilirubin total | | | | |
| (if total elevated - | | | | |
| add direct | | | | |
| Bilirubin) | | | | |
| ALP | | | | |
| ALT/SGPT | | | | |
| AST/SGOT | | | | |

ALP = alkaline phosphatase;
ALT = alanine aminotransferase;
aPTT = activated partial thromboplastin time;
AST = aspartate aminotransferase;
BUN = blood urea nitrogen;
CBC = complete blood count;
CRP = C-reactive protein;
IFN-γ; interferon-gamma;
IL = interleukin;
INR = international normalized ratio;
LDH = lactate dehydrogenase;
PT = prothrombin time;
PTT = partial thromboplastin time;
RBC = red blood cell;
SGOT = serum glutamic-oxaloacetic transaminase;
SGPT = serum glutamic-pyruvic transaminase;
TNF-α = tumor necrosis factor-alpha;
WBC = white blood cell
¹If BUN test cannot be analyzed by the local laboratory, urea should be analyzed.
²Per institutional guidelines.
³Screening only
⁴Antigen or PCR acceptable per institutional practice
⁵Subjects with elevated CRP during screening should be discussed with the Medical Monitor and carefully evaluated to rule out active infection or autoimmune disease.
⁶To be collected at baseline and during any suspected IRR or CRS event.
⁷If total amylase is elevated, evaluation of the isoenzyme analysis (S-type vs P-type) may be conducted.
⁸Titer levels for both EBV and CMV will be collected. If EBV or CMV DNA levels are detected (positive), collect anti-EBNA IgM, anti-EBNA IgG for EBV and anti-CMV IgM and anti-CMV IgG respectively.

### Research Laboratory Analyses

Biomarker analyses will be performed on plasma, serum, whole blood, PBMC, and tumor samples to evaluate PK, immunogenicity, and PD markers for PC-1. Prognostic markers related to the tumor immune environment may also be evaluated. Exploratory biomarker research may include, but will not be limited to, analysis of proteins, metabolites, or selected genetic aberrations associated with prostate cancer, or immune function, and inflammation. Research may involve circulating tumor DNA analysis and cell-free DNA quantitation. Circulating tumor cells may be quantified and analyzed. In addition, RNA transcriptomes of cancer cells and/or immune cells may be assessed.

In general, testing and analysis of the samples will follow the SOA. Additional samples (e.g., complete blood, urine, cerebrospinal fluid [CSF], tissue, etc) may be collected as needed for further safety testing. Multiple specialty laboratories may be employed for specific analyses. Detailed information regarding the collection, handling, and shipment of samples for PK, PD, immunogenicity, and biomarker assessment is provided in the Laboratory Manual. Clinical biospecimens (e.g., plasma, serum, PBMC, tumor tissue, CSF, or other bodily fluids) will be sent for centralized testing at locations selected by Sponsor. Certain plasma and serum samples will require local processing on site, prior to aliquoting, freezing, and shipping. CSF and other bodily fluids may be used as supporting information for any SAE/AE that were related to study drug or impact an event (these are not required but may be able to support evaluation of the outcome of the event). Allocation of samples to specific testing may be modified where sample material is limited. There may be circumstances where a decision is made to stop biomarker sample collection or not perform or discontinue analysis due to either practical or scientific reasons (e.g., low sample quality or data do not provide useful information). Alternatively, additional blood and tissue markers may be measured in case a strong scientific rationale develops (e.g., emerging preclinical and clinical data or published literature). The decision to terminate or add sample collection may be made by the Sponsor at any time. All samples, including those collected from subjects who do not enroll in the study, may be used for future research as well as development of tests/tools related to the treatment or disease under study. These samples, along with any derivatives, will be stored for up to 15 years. The exception is for remaining archival tumor blocks (Section 8.9.1.1), which will be returned to the site upon request or no later than after the clinical study report has been completed.

Each subject will have the right to have the sample material destroyed at any time by contacting the Investigator who, in turn, can contact the Sponsor. The Investigator should provide the Sponsor with the study and subject ID number so that the sample can be located and destroyed. For subjects who withdraw consent, any samples that were not requested to be returned or destroyed will remain with the Sponsor and may be used for research purposes. Complete details concerning these analyses will be provided in separate documents regarding bioanalytical analyses.

### Pharmacokinetic Assessments (Plasma)

PK assessment of PC-1 levels in plasma will be determined by an immunoassay specific for PC-1. Plasma samples will be obtained for PK analysis according to the SOA. Post infusion PK timepoints are calculated based on the end of infusion time. Additional PK samples may be collected in the event of significant toxicities, development of ADAs, or inconsistent drug exposure. The PK samples must be drawn from a location that is different from the site of study treatment administration. For example, if the study treatment is administered via a vein in the arm, the PK samples must be drawn from a vein in the contralateral arm. When done at the same visit as vital signs, PK samples should be drawn after obtaining vital signs.

### 8.8.2 Immunogenicity Assessments (Serum)

Immune responses to PC-1 will be evaluated throughout the study with an ADA assay to detect the presence of serum antibodies that bind any part of the PC-1 molecule. Serum for these assessments will be collected according to the SOA. The samples deemed ADA positive by the ADA assay may be further used to characterize the ADA response (e.g. neutralizing Ab, sub-domain characterization).

### 8.8.3 Exploratory Cytokines and Chemokines

Exploratory cytokines and chemokines will also be measured in plasma as markers of immune activation and inflammation. Biomarker assessments will be performed to evaluate innate and adaptive immune system characteristics that may be associated with potential PC-1-related infusion reactions, CRS, and/or anti-tumor activity. Potential correlations between cytokine/chemokine production and efficacy and/or toxicity will be assessed. Sub-clinical signals may be detected prior to any potential associated AEs at higher dose levels.

### 8.8.4 Exploratory T-Cell Immunophenotyping

Whole blood will be collected for PBMC isolation and flow cytometry according to the SOA to assess and quantitate peripheral blood immune cell subsets (T-cell, NK-cell, and monocyte), as well as including T-cell phenotyping and activation markers. On-study whole blood samples will assess changes in immune cell numbers, activation status, and memory phenotype.

### 8.8.5 Exploratory Response Assessments (Circulating Tumor Cells)

Blood will be collected for CTC quantitation and characterization according to the SOA.

### Exploratory Response Assessments (Circulating Tumor DNA)

Plasma will be collected for circulating tumor DNA (ctDNA) quantitation and analysis according to the SOA.

### 8.9 Tumor Biopsies

Pre-treatment, on-treatment, and post-treatment biopsy samples may be obtained to assess elimination of cancer cells, activation of anticancer immunity, or other biomarkers associated with the biologic mechanism of action of PC-1 or the malignant disease. Additional biopsies may also be performed if clinically indicated at the Investigator's discretion (e.g., EOT, treatment continuation beyond 24 weeks, suspected pseudoprogression, etc). Where possible, tissue from these biopsies should be provided to the Sponsor for exploratory analysis. All tissue must have an associated pathology report. Methods may include immunohistochemistry. Samples would be assessed for myeloid and T-cell markers and other immune activation markers.

Tumor tissue should be of good quality based on total and viable tumor content (≥20% for excisional biopsy and >50% for core biopsy). Samples must contain a minimum of 50 viable tumor cells that preserve cellular context and tissue architecture regardless of needle gauge or retrieval method. Biopsy/tissue handling procedures are detailed in the Laboratory Manual. Tumor samples should be stored according to institutional procedures until shipment.

### 8.9.1 Pre-Treatment Tumor Biopsies

### 8.9.1.1 Archival Tumor Tissue

Archival tumor tissue at baseline (obtained up to 2 years pre-study) is encouraged, with samples collected after the most recent therapy prior to PC-1 preferred. Exploratory analyses may include the following:
Tumor cell immunophenotyping (tumor markers, PSMA expression)
Tumor infiltrating T-cell analysis
Gene expression related to malignant biology, therapeutic response, and immunologic response
Tumor immune microenvironment characterization

Sample availability should be confirmed prior to enrollment. A representative formalin-fixed, paraffin-embedded tumor specimen in a paraffin block (preferred) or 15 to 20 slides containing unstained, freshly cut, serial sections must be submitted upon enrollment, along with an associated pathology report. Samples collected via resection, excision, or core-needle biopsy are acceptable. Cytological or fine-needle aspiration (defined as samples that do not preserve tissue architecture and yield cell suspension and/or smears) samples are not acceptable. Tumor tissue from bone metastases that have been decalcified is not acceptable.

### Optional Pre-Treatment Tumor Biopsy

In addition to archival tumor tissue, an optional pre-treatment biopsy can be performed from safely accessible tumor sites between the last dose of prior cancer therapy and the first dose of PC-1. Biopsy sampling would include fresh and formalin fixed specimens. This will allow for assessment of tumor cell profiling and TME immunophenotyping. Samples collected via resection, excision, or core-needle biopsy (minimum 18-gauge diameter) are acceptable. Cytological or fine-needle aspiration (defined as samples that do not preserve tissue architecture and yield cell suspension and/or smears) samples are not acceptable. Tumor tissue from bone metastases that have been decalcified is not acceptable. Fixed samples may be assayed for PSMA expression by immunohistochemistry or RNA transcript quantitation. These samples should have at least 3 cores aligned and embedded on a single block. Fresh biopsy samples may be assessed for PSMA expression levels if available at the Investigator's site; these may also be sent to a central laboratory for other downstream analysis. For fresh samples, at least 2 additional cores are recommended.

### 8.9.1.3 Diagnosis Confirmation and Molecular Subtyping

Pre-treatment biopsies may also allow for the further characterization of PC subtype. Tumor cells may be immunophenotyped, as well as examined for expression of key markers such as expression of prognostic markers (e.g., p53, BRCA, PTEN, etc). This may confirm or refine diagnosis. On a more exploratory basis, genotype mutations may be determined, gene expression profiled, and assessed for correlations with anti-tumor response, or safety.

### 8.9.2 Optional On-Treatment Tumor Biopsies

Investigators are encouraged to perform an optional on-treatment biopsy at any time during Cycle 2 prior to tumor assessment and submit a portion of the tumor tissue to the central laboratory for exploratory biomarker analysis. Biopsy sampling should include fresh and formalin-fixed specimens. In addition, optional biopsies could be considered in subjects with SD/PR at the time of response. This will allow for assessment of tumor cell and TME immunophenotyping. On-treatment biopsies may enable assessment of PD, tumor-cell depletion, as well as quantitation of PC-1 penetration into the TME. Importantly, on-study biopsies could show T-cell infiltrates, as well as other inflammatory cells, and could help determine mechanisms of action of PC-1 responses. On-treatment biopsy should be performed on the same lesion on which the screening biopsy was performed.

### Optional Post-Treatment (Progression) Tumor Biopsies

In the event of disease progression, Investigators are encouraged to collect an optional tumor biopsy and submit a portion of the tumor tissue to the central laboratory for exploratory biomarker analysis. Biopsy sampling would include fresh and formalin-fixed specimens. This will allow for assessment of tumor cell and TME immunophenotyping. The aim would be to assess mechanisms of resistance, such as PSMA antigen or epitope loss, or upregulation of checkpoint inhibitors or other immunosuppressive mechanisms.

### Safety Monitoring and Reporting

### 10.1 Adverse Events

In accordance with the ICH E2A guideline, and 21 Code of Federal Regulations (CFR) § 312.32, an AE is defined as any untoward medical occurrence in a clinical study subject administered a pharmaceutical product, which does not necessarily have a causal relationship with this treatment.

### This Includes the Following:

AEs not previously observed in the subject that emerge during the protocol-specified AE reporting period, including signs or symptoms associated with a subject's cancer that were not present prior to the AE reporting period (see Section 10.2)
Complications that occur as a result of protocol-mandated interventions (e.g., invasive procedures such as biopsies) prior to PC-1 treatment
Pre-existing medical conditions, judged by the Investigator to have worsened in severity or frequency or changed in character during the protocol-specified AE reporting period
   What should NOT be Reported as an AE:
Any abnormal laboratory findings or other abnormal safety assessments which are associated with the underlying disease, unless judged by the Investigator to be more severe than expected for the subject's condition
The disease/disorder being studied or expected progression, signs, or symptoms of the disease/disorder being studied, unless more severe than expected for the subject's condition
Medical or surgical procedure (e.g., endoscopy, appendectomy): the condition that leads to the procedure is the AE
Situations in which an untoward medical occurrence did not occur (social and/or convenience admission to a hospital)
Anticipated day-to-day fluctuations of pre-existing disease(s) or condition(s) present or detected at the start of the study that do not worsen

### Reporting of Adverse Events

The AE reporting period begins at signed informed consent and continues until 30 days after the last dose of study treatment, or until initiation of subsequent anticancer therapy, whichever occurs first. After signing of informed consent but prior to the first dose of study treatment, only AEs and SAEs caused by a protocol-mandated study intervention will be collected (e.g., AEs related to invasive procedures such as biopsies). AEs will be assessed until resolution, return to baseline, or are stabilized, per the Investigator's assessment. After the first dose of study treatment, all AEs regardless of attribution will be collected. AEs will be reported by the subject (or, when appropriate, by a caregiver, surrogate, or the subject's legally authorized representative). The Investigator and any qualified designees are responsible for detecting, documenting, and recording events that meet the definition of an AE or SAE and remain responsible for following up on AEs that are serious, considered related to the study intervention or study procedures, or that caused the subject to discontinue PC-1. Investigators should report all AE details that can be gathered immediately on the AE eCRF. The primary mechanism for reporting an SAE to the Sponsor or designee will be a paper SAE report form. The study site will enter the event onto the SAE report form within 24 hours of the Investigator's awareness of the event (see Section 10.7).

### 10.3 Grading and Intensity of Adverse Events

AEs, with the exception of CRS, will be graded using NCI CTCAE v5.0. CRS will be graded according to the grading scale in Table 17. The reported verbatim term should be the most descriptive medical diagnosis and does not have to be found in CTCAE.

The AE grading (severity) scale NCI CTCAE v5.0 will be used for AE reporting as shown in Table 20. Regardless of severity, some events may also meet regulatory serious criteria (see Section 10.6).

TABLE 17
AE Grade (Severity) Scale

| Grad | Severity | Alternate Description¹ |
|---|---|---|
| 1 | Mild; asymptomatic or mild symptoms; | Transient or mild discomfort (<48 hours); |
| | clinical or diagnostic observations only; | no interference with the subject's daily |
| | intervention not indicated. (apply event- | activities; |
| | specific NCI CTCAE v5.0 grading | no medical intervention/therapy required |
| | criteria) | |
| 2 | Moderate; minimal, local or noninvasive | Mild to moderate interference with the |
| | intervention indicated; limiting | subject's daily activities; no or minimal |
| | age -appropriate instrumental ADL². | medical intervention/therapy required |
| | (apply event-specific NCI | |
| | CTCAE v5.0 grading | |
| | criteria) | |
| 3 | Severe or medically significant but not | Considerable interference with the |
| | immediately life-threatening; | subject's |
| | hospitalization | daily activities; medical |
| | or prolongation of hospitalization | intervention/therapy |
| | indicated; | required; hospitalization possible |
| | disabling; limiting self care ADL³. (apply | |
| | event-specific NCI CTCAE v5.0 grading | |
| | criteria) | |
| 4 | Life-threatening consequences; urgent | Extreme limitation in activity; significant |
| | intervention indicated. (apply event- | medical intervention/therapy required, |
| | specific | hospitalization probable |
| | NCI CTCAE v5.0 grading criteria) | |
| 5 | Death related to AE | |

ADL = activities of daily living;
AE = adverse event;
CTCAE = Common Terminology Criteria for Adverse Events;
NCI = National Cancer Institute
¹Use the alternative descriptions for Grade 1, 2, 3, and 4 events when the observed or reported AE does not appear in the NCI CTCAE v5.0 listing.
²Instrumental ADL refer to preparing meals, shopping for groceries or clothes, using the telephone, managing money, etc.
³Self care ADL refer to bathing, dressing and undressing, feeding self, using the toilet, taking medications, and not bedridden.
Source: NCI CTCAE v5.0

### Relationship to Study Treatment

The Investigators are required to assess whether there is a reasonable possibility that the study treatment caused or contributed to an AE. The Investigators should use their knowledge of the subject, the circumstances surrounding the event, and an evaluation of any potential alternative causes to determine whether or not an AE is considered to be related to the study treatment by indicating “yes” or “no” accordingly. Some considerations are presented in Table 18.

TABLE 18
Relatedness

| | |
|---|---|
| Related | There is a reasonable possibility and/or evidence to suggest a causal |
| | relationship between study treatment and the AE and no other more likely |
| | alternative cause (concomitant drugs, therapies, disease complications, |
| Not | There is no reasonable possibility and/or evidence to suggest a causal |
| Related | relationship between study treatment and the AE and another more likely |
| | alternative cause (concomitant drugs or therapies, disease complications, |

AE = adverse event

### Recording Adverse Events

All AEs/SAEs must be recorded in the subject's medical records and on the CRF in accordance with the reporting criteria for different time periods as defined in Section 10.2. Each AE is to be evaluated for the following:
AE diagnosis or syndrome (if not known, signs or symptoms)
Dates of onset and resolution
Severity (including grade changes as per the CRF completion guidelines; see Section 10.3)
Causal relationship with PC-1 or protocol-mandated procedures (see Section 10.4)
Seriousness (see Section 10.6)
Action taken
Outcome
   10.5.1 Recording a Diagnosis versus Signs and Symptoms

Whenever possible, a unifying diagnosis should be reported as opposed to a listing of individual symptoms. However, symptoms should be grouped into a diagnosis only if each sign or symptom is a medically confirmed component of that diagnosis as evidenced by current standard medical textbooks. If any aspect of a sign or symptom does not fit into a classic pattern of the diagnosis, the individual symptom should be reported as a separate AE.

### 10.5.2 Pre-Existing Medical Conditions

A pre-existing medical condition is one that is present at the Screening Visit for this study. Such conditions should be recorded as General Medical History and Baseline Conditions.

A pre-existing medical condition should be recorded as an AE only if the frequency, severity, or character of the condition worsens during the study. Clinical Laboratory Abnormalities and Other Abnormal Assessments

Any laboratory abnormality (e.g., clinical chemistry or hematology) or other abnormal assessment findings (e.g., ECG or vital signs) that meets any of the following criteria should be recorded as an AE or SAE:
Associated with clinical signs and/or symptoms
Requires medical or surgical intervention
May require a change in current therapy, and leads to study treatment discontinuation, delay, or interruption
Otherwise clinically significant as determined by the Investigator

Whenever possible, the clinical diagnosis, rather than the laboratory result, should be reported by the Investigator (e.g., anemia versus low hematocrit).

Clinically significant abnormal laboratory values occurring during the study will be followed until repeat tests return to normal, stabilize, or are no longer clinically significant.

Laboratory abnormalities that are not deemed clinically significant by the Investigator should not be reported as AEs.

### 10.6 Serious Adverse Event

An SAE is defined as an event that, at any dose, meets any of the criteria in Table 19.

TABLE 19
Definitions of SAEs

| Criteria | Description |
|---|---|
| Fatal | The AE resulted in death. |
| Life-threatening | The AE placed the subject at immediate risk of death. (This |
| | classification does not apply to an AE that hypothetically might have |
| | caused death if it had been more severe.) |
| Hospitalization/ | The AE resulted in hospitalization or prolongation of |
| prolongation of | hospitalization (Section 10.6.3). Hospitalization/prolongation of |
| hospitalization | hospitalization for the purpose of post infusion observation, as |
| | required per protocol, is not considered an SAE. |
| Disability/ | The AE resulted in a disability, significant incapacity, or |
| incapacity | substantial disruption of the subject's ability to conduct normal |
| Congenital | The AE was an adverse outcome in a child or fetus of a subject |
| anomaly/birth | exposed to the study treatment regimen before conception or during |
| defect | [symbol] |
| Medically | The AE was a medically important event that did not meet any of the |
| important | above criteria but may have jeopardized the subject and may have |
| | required medical or surgical intervention to prevent 1 of the |
| | outcomes listed above (examples include allergic bronchospasm that |
| | required treatment in an emergency room, seizures that do not result |
| | in hospitalization, or blood dyscrasias). |

AE = adverse event;
SAE = serious adverse event [symbol]
indicates data missing or illegible when filed

The terms “severe” and “serious” are not synonymous. Severity refers to the intensity of an AE (as in mild, moderate, or severe pain); the event itself may be of relatively minor medical significance (such as severe headache). “Serious” is a regulatory definition and is based on subject or event outcome or action criteria usually associated with events that pose a threat to a subject's life or vital functions. Seriousness (not severity) serves as the guide for defining regulatory reporting obligations.

Severity and seriousness should be independently assessed when recording AEs and SAEs on the CRF.

Special considerations for AE reporting, including events that should always be reported as SAEs in this study, are presented in Table 20.

TABLE 20
Special Considerations for SAE Reporting

| Criteria | Description |
|---|---|
| Other events that | New/secondary malignancies |
| should always be | New onset or exacerbation of a pre-existing neurologic disorder |
| reported in the | New onset of rheumatologic or other autoimmune |
| manner as an SAE | disorder New onset hematologic disorder (e.g., |
| (refer to Section | Rare and unexpected disorders with and unknown etiology (e.g., |
| 10.7) through 30 | llain--Barre, Stevens-Johnson's syndrome) |
| the last dose of study | |
| drug | |

FDA = Food and Drug Administration;
SAE = serious adverse event;
US = United States

### 10.6.1 Recording Serious Adverse Events

The following should be considered when recording SAEs:

For hospitalizations or surgical or diagnostic procedures, the illness leading to the surgical or diagnostic procedure should be recorded as the SAE, not the procedure itself. The procedure should be captured in the narrative as part of the action taken in response to the illness.

### 10.6.2 Deaths

All deaths that occur during the protocol-specified AE reporting period (see Section 10.2), regardless of attribution, will be recorded as SAEs. This includes death attributed to progression of disease. When recording a death on the SAE Report Form, the event or condition that caused or contributed to the fatal outcome should be recorded as the single medical concept. Death is an outcome of an event. The event that resulted in death (e.g., sepsis, stroke, myocardial infarction, etc) should be recorded on the SAE Report Form. If the death is attributed to progression of disease, record “disease progression” as the SAE term on the SAE Report Form. Deaths that are attributed to disease progression will be considered as an expected AE and will not be subject to expedited reporting. The term “unexplained death” should be captured if the cause of death is not known. However, every effort should be made to capture the established cause of death, which may become available later on (e.g., after autopsy).

### Hospitalization and Prolonged Hospitalization

Any AE that results in hospitalization or prolonged hospitalization should be documented and reported as an SAE as described in Section 10.7.

The following hospitalization scenarios are not considered to be SAEs:
Planned hospitalization/prolongation of hospitalization required by the protocol (e.g., for monitoring of the subject after the first dose of PC-1 or to perform an efficacy measurement for the study)
Hospitalization for palliative care or hospice care
Admissions for social or situational reasons (eg, no place to stay, lives too far away to come for hospital visits) in the absence of any clinical AE
Admissions for elective or pre-planned treatment or procedure for a pre-existing condition that is unrelated to the condition under study and has not worsened since providing informed consent
Admissions for routine treatment (eg, platelet transfusion) or monitoring of the condition under study not associated with any deterioration in condition
Admissions for routine procedures (eg, bone marrow aspiration) associated with the disease under study
Emergency outpatient treatment or observation that does not result in admission, unless fulfilling other seriousness criteria above

### 10.6.4 Serious Adverse Event Reporting Periods

From the time of informed consent until the first dose of PC-1, only SAEs considered related to study-specific procedures are required to be reported. All SAEs reported from the first dose of PC-1 must be reported as follows:

All SAEs from the first dose of PC-1 through 30 days following last dose of study drug, or until initiation of another systemic anticancer therapy, whichever occurs first. SAEs must be followed until they return to baseline, the event stabilizes or is no longer considered clinically significant by the Investigator, the subject dies or withdraws consent, or study closure. Certain non-serious treatment-emergent AEs, as requested by the Sponsor, may be followed until resolution, return to baseline, or study closure.

### 10.6.5 Other Reportable Information

Certain information, although not considered an SAE, must be recorded, reported, and followed up as indicated for an SAE. This includes pregnancy and overdose, as described in the following sections.

### Pregnancy

To ensure subject safety, each pregnancy occurring in the female partner of a male subject from the time of informed consent until 2 months after the last dose of study treatment must be reported to the Sponsor or designee within 24 hours of learning of its occurrence. Email transmission of the paper Pregnancy Report Form is the preferred method to transmit safety event information to IQVIA Biotech with facsimile as a back-up method, if necessary. Pregnancy follow-up should be recorded on the same form and should be submitted to the Sponsor or designee within 24 hours of awareness. If the pregnancy is discovered following the first dose of PC-1, the pregnancy should be followed to determine outcome. All pregnancies should be monitored for the full duration, and all perinatal and neonatal outcomes should be reported. Pregnancy in a study subject's partner is not considered an AE. However, any SAE experienced during pregnancy must be reported on the SAE Report Form and must be reported to the Sponsor or designee within 24 hours of the site becoming aware of the event, per Section 10.7. Abortion, whether accidental, therapeutic, or spontaneous, should be reported as an SAE. Congenital anomalies or birth defects, as defined by the “seriousness criteria” in Section 10.6, should be reported as SAEs.

### 10.6.5.2 Overdose

Any overdose or incorrect administration of PC-1 should be reported to the Sponsor and IRB/IEC in accordance with IRB/IEC policies. Consider obtaining clinical laboratory assessments (eg, complete blood count, chemistry panel, CRP, ferritin) but also PK levels, serum for safety cytokines, and plasma for exploratory laboratory assessments. AEs associated with an overdose or incorrect administration of study treatment should be recorded on the AE CRF.

### 10.7 Expedited Reporting Requirements for Serious Adverse Events

The primary mechanism for reporting an SAE to the Sponsor or designee will be the paper SAE report form. The study site will submit the paper SAE report form within 24 hours of the Investigator's awareness of the event. Further details regarding how to submit SAEs to the Sponsor or designee are provided in the Site Manual. Reports should also include supporting documentation (eg, hospital admission notes, test results, etc).

For all SAE reports, the following minimum criteria must be reported on the Safety Event Report Form:
Subject ID number
Date of event onset
Description of event
Study treatment
Relationship to study treatment

Relevant follow-up information is to be submitted via paper SAE form to the Sponsor or designee by the same method as outlined above as soon as it becomes available but no later than 24 hours from Investigator's awareness. The Sponsor or its designee will report SAEs and/or suspected unexpected serious adverse reactions (SUSARs) as required to regulatory authorities, Investigators/institutions, and central IRBs/IECs in compliance with reporting requirements according to local regulations and Good Clinical Practice (GCP). The Investigator is to notify the appropriate local IRB/IEC of SAEs occurring at the site and other AE reports received from the Sponsor or designee in accordance with local procedures and statutes.

10.8 Type and Duration of Follow-Up of Subjects after Adverse Events

All AEs (regardless of relationship to study intervention) and SAEs determined not to be study intervention related (i.e., not related and unlikely related) will be followed through the EOT visit and be noted as “continuing” if not resolved at this visit.

Any SAE that is determined to be possibly study drug related will be followed to resolution or stabilization, is determined to be irreversible by the Investigator, the subject is lost to follow up, or it has been determined that the study treatment is not the cause of the SAE.

Resolution of AEs and SAEs (with dates) should be documented on the AE CRF, the SAE Report Form, and in the subject's medical record to facilitate source data verification.

### Statistical Considerations

This is an open-label clinical study and, in general, descriptive statistics will be employed to analyze the data. Data will be tabulated and analyzed with respect to subject enrollment and disposition, demographic and baseline characteristics, prior and concomitant medications, efficacy, PK, PD, and safety measures. More details will be provided in a Statistical Analysis Plan (SAP) covering the dose escalation, backfill, and dose expansion parts of this study. The SAP will detail all data handling rules, including the management of missing values and the handling of data for withdrawn subjects. No discrepancies are expected between the SAP and the protocol. However, if there are discrepancies, the SAP will override the protocol and outline protocol deviation criteria. Any deviations from the planned analyses specified or populations defined within the SAP will be justified in writing and presented within the final clinical study report. Clinical database lock will occur after all study data are reconciled for all subjects. The SAP will be finalized and signed before database lock.

### 11.1 Sample Size Considerations

No formal hypothesis testing is planned. The number of subjects in each part of the study is based on safety and tolerability. Dose Escalation (Part 1) will utilize BLRM with EWOC beginning at Cohort 6. With this type of study design the exact number of subjects needed to complete dose escalation is unknown as it depends on the number of subjects enrolled in each cohort and the number of cohorts studied, both of which are contingent on the recommendations made by the SRC. Approximately 20 to 30 subjects are expected to be treated in Part 1 for the BLRM model to have reasonable operating characteristics related to its RP2D-R recommendation. Cohort Backfill (Part 2) will enroll up to approximately 45 subjects. The study may allow for limited expansion of a prior cleared dose level cohort (up to 15 subjects per cohort, 18 subjects per dose level in Parts 1 and 2 combined), based on SRC review to gain further characterization of the safety, tolerability, PK, PD, and preliminary efficacy of defined doses of PC-1. DLT observation data available at the time of each dose level review will be included in the BLRM model. These data will also be used to select the dose for Dose Expansion (Part 3). A sample size of approximately 30 subjects in Dose Expansion (Part 3) will further characterize and provide an estimation precision (as the maximum half-width of the 95% confidence interval [CI]) of approximately ±18% for any binary rate of outcome assessing safety, tolerability, exposure, immunogenicity, PD, or clinical activities of single agent PC-1. These levels of precision are deemed sufficient in the comprehensive review of the study data to guide future clinical development.

### 11.2 Analysis Sets

### 11.2.1 Full Analysis Set

The Full Analysis Set (FAS) includes all subjects who receive at least 1 dose of study treatment PC-1.

### 11.2.2 Dose-Limiting Toxicity Evaluable Analysis Set

The DLT Evaluable Analysis Set (DAS) includes all subjects in Dose Escalation (Part 1) considered DLT evaluable as defined in Section 3.2.4.1. This analysis set will be used for dose-finding purposes, to define the MTD-R, and to identify the RP2D-R.

### 11.2.3 Pharmacokinetic Analysis Set

The PK Analysis Set (PKAS) includes all subjects in the FAS who have at least 1 blood sample that provides evaluable PK data.

### 11.2.4 Per Protocol Analysis Set

The Per Protocol Analysis Set (PPAS) includes all enrolled subjects who do not have any major protocol deviations that might affect the efficacy outcomes.

### 11.3 Statistical Methods

All analyses will be descriptive and hypothesis-generating in nature. Descriptive statistics will be provided for all safety, PK, PD, and efficacy endpoints. All analyses will be conducted separately for subjects in Dose Escalation (Part 1), Cohort Backfill (Part 2), and Dose Expansion (Part 3) parts of the study. In addition, PK, PD, efficacy, and safety analyses may be conducted for pooled analysis treated at relevant doses. Details regarding the statistical analysis to be conducted, including the handling of missing data and subject withdrawal, will be provided in the SAP.

### 11.3.1 Missing Data/Discontinuation

Due to the dose escalation and exploratory Phase 1 design of the study, no imputation of missing values will be done for any analysis. Imputation will be done for missing partial dates of AEs and concomitant medications. Reasons for discontinuation of the study treatment and the study will be listed and summarized.

### Demographics, Medical History, Prior Medication and Other Baseline Characteristics

Demographic characteristics, prior anti-cancer therapies and surgeries, medical history, concomitant medication and other baseline data will be listed and summarized using descriptive statistics for numerical data and contingency tables for categorical data. Medical history and concomitant medications will be listed. Prior anti-cancer therapies will be coded by World Health Organization (WHO) Anatomical, Therapeutic and Chemical (ATC) terms and summarized.

### 11.3.3 Study Treatment

Exposure to PC-1 will be summarized with descriptive statistics for the total number of infusions received. The total amount of time on PC-1 (duration of dosing in weeks) will also be derived and summarized. The number of infusions with interruptions and dose modifications (regardless of reason) will be summarized by dose cohort and overall.

### 11.3.4 Concomitant Medication

Concomitant medications summarized by WHO ATC term and significant non-drug therapies after the start of study treatment will be listed in contingency tables.

### 11.4 MTD-R Determination

The MTD-R will be determined using the DAS Analysis Set.

Subjects who do not meet the criteria for being considered DLT evaluable as defined in Section 3.2.4.1 will be considered for the full analysis but may be replaced by an additional subject for DLT assessment.

### 11.4.1 Bayesian Adaptive Approach

The frequency of DLTs will be tabulated by dose for subjects in the dose escalation phase, and information about the DLTs will be listed by dose. An adaptive 3-step BLRM approach guided by the EWOC principle will be used in the dose escalation. The use of Bayesian adaptive models of the dose-response relationship has been advocated by the European Medicines Agency adopted guideline on small populations (EMA 2006) and by Rogatko et al 2007 and is one of the key elements of the FDA's Critical Path Initiative. Standardized dose regimens will be used such that one of the dose regimens (d*) equals 1, e.g., dose regimens are rescaled as d/d*. All information currently available about the dose-DLT relationship of PC-1 is summarized in a prior distribution. For this study, this includes non-clinical data about the starting dose of PC-1 as well as current information available from the ongoing dose escalation. The information available from the ongoing dose escalation includes the data outlined in Section 1.9 as well as data from 3 subjects enrolled on study in a fourth cohort with step doses of 100 μg on Day 1, 300 μg on Day 8 and 450 μg on Day 15 where no DLTs were observed. This prior distribution is then updated after each cohort of subjects with all of the DLT data available in the DAS from the current trial. Once updated, the distribution summarizes the probability that the true DLT rate for each dose regimen lies in the following categories:
0% to <16%: under-dosing;
≥16% to <33%: targeted toxicity; and
≥33% to 100%: excessive toxicity.

The EWOC principal mandates that any dose of PC-1 that has a ≥25% chance of being in the excessive toxicity category is not considered for the next dose group. A clinical synthesis of the toxicity information (including AEs that are not DLTs), recommendations from the BLRM as well as available PK and PD data will be used to determine the PC-1 dose for the next dose group. Dose escalation recommendations will be made by the SRC.

### 11.4.2 Statistical Modeling for MTD-R or RP2D-R Determination

The objective of the design is to determine the MTD-R as defined in Section 3.2.3.2. The modelling will be guided by a 3-step BRLM approach, which is implemented as follows:

CRS Model: the probability of a DLT due to CRS (p(CRS)) is modelled as a function of the dose under the time-dose regimen curve (‘area under dose’ or AUD), using a two-parameter BLRM

Non-CRS DLT: the probability of any other DLT (p(non-CRS)) will be modelled as a function of the cumulative dose across the dose regimen, using a separate two-parameter BLRM

Joint DLT model: the overall probability of a DLT given a particular dose regimen is calculated by combining the two probabilities detailed above.

The probabilities provided by the joint DLT model will then be used to inform the recommendations of the SRC. If required, the sub-probabilities of CRS DLT and non-CRS DLT will also be made available to the SRC.

### CRS Model

The CRS model is formulated as follows:

logit(p(CRS_{I}))=log(α1)+β1*log(AUD_{I}/AUD*)
where logit(p)=log(p/(1−p)). p(CRS_{I}) represents the probability of having a CRS DLT at dose regimen I, AUD*=23.6 mg is the AUD of the reference regimen, which corresponds to example dose regimen 5 (see Table 25). This allows for the interpretation of α as the odds of a DLT at dose regimen 5, and θ1=(log(ci1), log(β1)) with ci1, β1>0 is the parameter vector of the model. The area under the time-dose curve captures both the average rate of dose increase for a regimen, as well as the cumulative dose. This allows the CRS model to relate the rate at which a regimen exposes subjects to PC-1 to the probability of CRS. AUD_{I} is the dot product of the vector of doses of regimen I with the vector of follow-up lengths in days before administration of the next dose or (in the case of the final dose of a regimen) before the end of the DLT observation period.

### Non-CRS Model

The non-CRS model is formulated as follows:

logit(p(non-CRSI))=log(ci2)+β2*log(DcumI/Dcum*)

Where p(non-CRSI) is the probability of experiencing a DLT other than CRS at dose regimen I, DcumI is the cumulative dose of dose regimen I, and Dcum*=3.5 mg is the cumulative dose of the reference regimen (regimen 5, as in the CRS model, above). θ2=(log(ci2), log(β2)) with ci2, β2>0 is the parameter vector of the model. DcumI is given by the sum of the doses administered during the DLT observation period for regimen I.

### Joint DLT Model

The final probability of a DLT for dose regimen I is given by 1 minus the joint probability of no CRS DLT and no non-CRS DLT, i.e.,

$$p ( DLTI ) = 1 - p ( no CRSI , no non - CRSI ) .$$

Assuming that CRS and non-CRS DLTs are independent we can write,

$$p ( no CRSI , no non - CRSI ) = p ( no CRSI ) p ( no non - CRSI ) = [ 1 - p ( CRSI ) ] [ 1 - p ( non - CRSI ) ] .$$

Hence, the probability of DLT for dose regimen I is given by,

$$p ( DLTI ) = 1 - [ 1 - p ( CRSI ) ] [ 1 - p ( non - CRSI ) ]$$

### 11.4.3 Prior Derivation

The advent of the BLRM model at Cohort 6 meant that the data from prior cohorts (see Section 11.4.1) could be used to build the prior for the subsequent BLRM. Priors on the four parameters of the model, log(ci1), log(β1), log(ci2), log(β2) are assumed to be Gaussian. To kickstart the inference process, we first implemented a weakly informative prior which makes the basic assumption that we will reach some toxicity towards the upper end of the dose regimens suggested in Table 25. For non-CRS DLTs we assumed a true non-CRS DLT rate of 0.15 at Dose Regimen 1, and a true DLT rate of 0.24 at Dose Regimen 6. For CRS DLTs we assumed a true CRS DLT rate of 0.075 at Dose Regimen 1, and a true DLT rate of 0.12 at Dose Regimen 6. This reflects the assumption that any other DLT is more likely than CRS, but that some CRS is likely (the transition to BLRM having been motivated by observation of increased cytokine response in preceding cohorts).

Table 21 summarizes the parameters of the prior distribution based on these assumptions.

TABLE 21
Prior Distributions

| Model | Prior Component | Mean-Vector | SD | α-β Correlation |
|---|---|---|---|---|
| CRS | Minimally | −1.8402 | 2 | 0 |
| | informative | 0.572 | 1 | |
| Non-CRS | Minimally | −0.952 | 2 | 0 |
| DLT | informative | 0.716 | 1 | |

Following this data from preceding cohorts D was combined with this prior to infer the posterior distribution over α1, β1, α2, β2 using Bayes' rule:

p(α1,β1,α2,β2|D)=p(D|α1,β1,α2,β2)p(α1,β1,α2,β2)/p(D).

Table 22 summarizes the historical data D.

TABLE 22
Historical Data

| | | AUD | Cumulative | | #non- | |
|---|---|---|---|---|---|---|
| | N | Dose | Dose | #CRS | CRS | #Total |
| Dose Regimen | patients | (mg) | (mg) | DLTs | DLTs | DLTs |
| Flat 100 μg | 2 | 2.10 | 0.30 | 0 | 0 | 0 |
| Flat 100 μg, last | 1 | 1.40 | 0.20 | 0 | 0 | 0 |
| dose missed | | | | | | |
| Flat 300 μg | 1 | 6.30 | 0.90 | 0 | 0 | 0 |
| Step | 4 | 3.50 | 0.50 | 0 | 0 | 0 |
| 50/50/100/300 μg | | | | | | |
| Step | 3 | 5.95 | 0.85 | 0 | 0 | 0 |
| 100/300/450 μg | | | | | | |

Table 23 summarizes the parameters of this posterior, which will then be used as the prior for the BLRM used in the dose escalation from Cohort 6.

TABLE 23
Parameters Used in the BLRM Prior

| Model | Prior Component | Mean-Vector | SD | α-β Correlation |
|---|---|---|---|---|
| CRS | Historical | −3.251 | 1.526 | 0.258 |
| | | 0.809 | 1.042 | |
| Non-CRS | Historical | −2.450 | 1.585 | 0.397 |
| DLT | | 1.100 | 1.028 | |

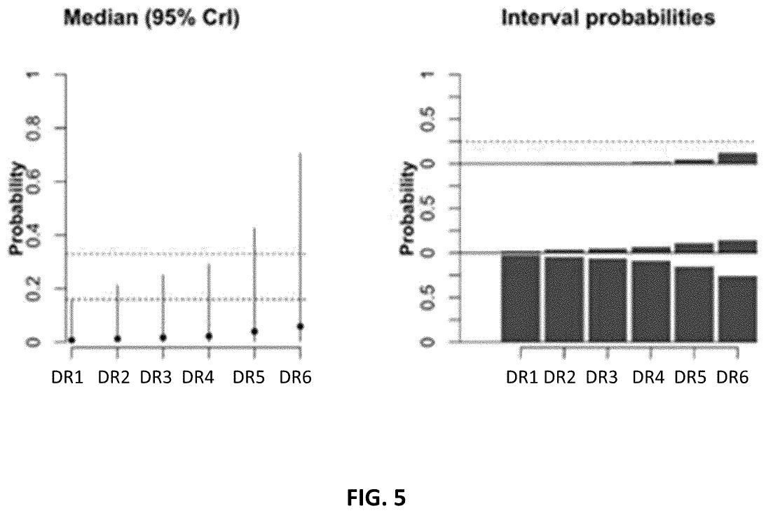

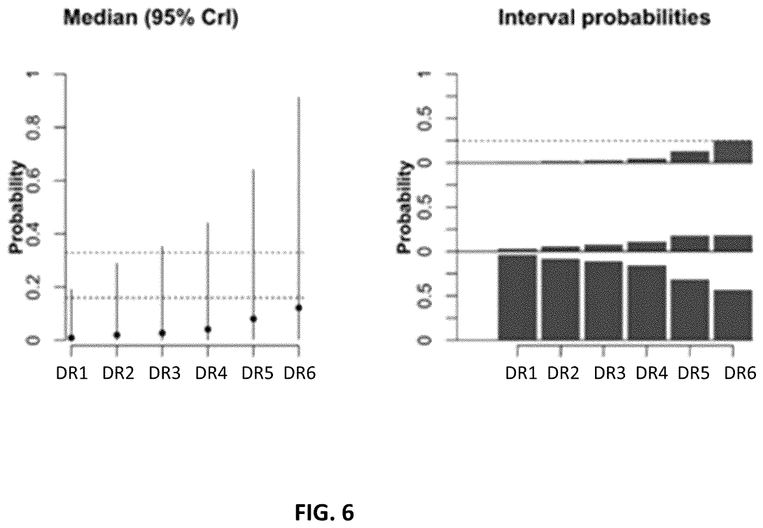

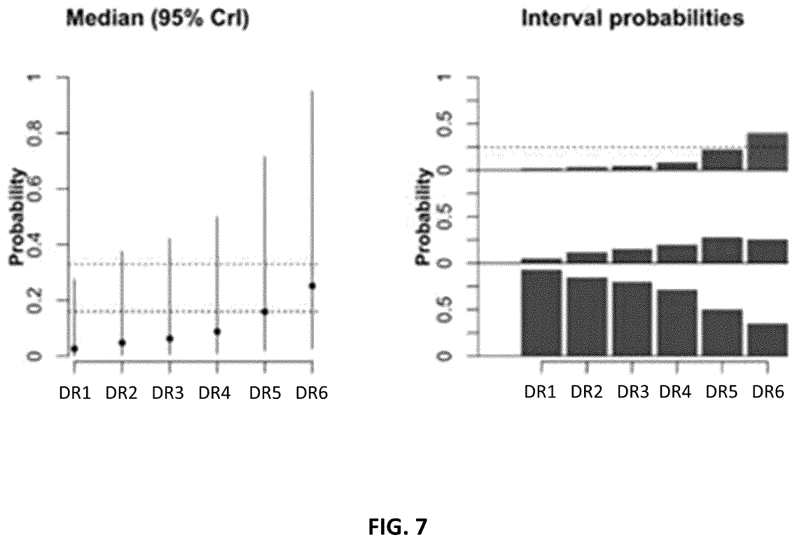

A summary of the prior probabilities of CRS, non-CRS DLTs and overall for different regimens, as well as the corresponding probability of under-targeted and overdosing, are shown in Table 27, Table 28 and Table 29. Graphically, the prior medians with accompanying 95% credible intervals are shown in FIG. 5, FIG. 6 and FIG. 7. Uncertainty about parameters is expressed by large prior standard deviations for all derived parameters in both the CRS and non-CRS components.

TABLE 24
Prior Probabilities of CRS for Each Dose Regimen

| Dose | Probability of true DLT rate in | | | | | | | |
|---|---|---|---|---|---|---|---|---|
| Regi- | [0- | [0.16- | [0.33- | | | Quantiles | | |
| men¹ | 0.16] | 0.33) | 1] | Mean | StD² | 2.5% | 50% | 97.5% |
| DR1 | 0.977 | 0.018 | 0.005 | 0.024 | 0.051 | 0.000 | 0.007 | 0.154 |
| DR2 | 0.954 | 0.036 | 0.01 | 0.036 | 0.065 | 0.000 | 0.012 | 0.212 |
| DR3 | 0.936 | 0.05 | 0.014 | 0.043 | 0.073 | 0.000 | 0.017 | 0.251 |
| DR4 | 0.913 | 0.068 | 0.019 | 0.053 | 0.083 | 0.001 | 0.022 | 0.291 |
| DR5 | 0.848 | 0.109 | 0.043 | 0.083 | 0.115 | 0.002 | 0.039 | 0.427 |
| DR6 | 0.739 | 0.141 | 0.119 | 0.133 | 0.181 | 0.002 | 0.058 | 0.705 |

¹The dose regimens are ordered from lowest to highest in terms of AUD
StD = standard deviation

TABLE 25
Prior Probabilities of Non-CRS DLT for Each Dose Regimen

| Dose | Probability of true DLT rate in | | | | | | | |
|---|---|---|---|---|---|---|---|---|
| Regi- | [0- | [0.16- | [0.33- | | | Quantiles | | |
| men¹ | 0.16] | 0.33) | 1] | Mean | StD² | 2.5% | 50% | 97.5% |
| DR1 | 0.962 | 0.03 | 0.008 | 0.030 | 0.059 | 0.000 | 0.009 | 0.191 |
| DR2 | 0.922 | 0.059 | 0.019 | 0.049 | 0.082 | 0.000 | 0.019 | 0.289 |
| DR3 | 0.893 | 0.078 | 0.029 | 0.062 | 0.094 | 0.000 | 0.027 | 0.353 |
| DR4 | 0.842 | 0.11 | 0.047 | 0.085 | 0.114 | 0.002 | 0.041 | 0.441 |
| DR5 | 0.688 | 0.181 | 0.131 | 0.147 | 0.171 | 0.003 | 0.080 | 0.641 |
| DR6 | 0.567 | 0.182 | 0.251 | 0.229 | 0.253 | 0.004 | 0.122 | 0.913 |

Note:
Doses printed in bold type meet the overdose criterion, P(overdose) < 0.25.
¹The dose regimens are ordered from lowest to highest in terms of AUD
²stD = standard deviation

TABLE 26
Prior Probabilities of Overall DLT for Each Dose Regimen

| Dose | Probability of true DLT rate in | | | | | | | |
|---|---|---|---|---|---|---|---|---|
| Regi- | [0- | [0.16- | [0.33- | | | Quantiles | | |
| men¹ | 0.16] | 0.33) | 1] | Mean | StD² | 2.5% | 50% | 97.5% |
| DR1 | 0.929 | 0.054 | 0.017 | 0.053 | 0.076 | 0.001 | 0.026 | 0.276 |
| DR2 | 0.849 | 0.118 | 0.033 | 0.083 | 0.100 | 0.003 | 0.047 | 0.376 |
| DR3 | 0.797 | 0.151 | 0.051 | 0.103 | 0.113 | 0.005 | 0.062 | 0.421 |
| DR4 | 0.713 | 0.2 | 0.087 | 0.133 | 0.133 | 0.009 | 0.088 | 0.500 |
| DR5 | 0.502 | 0.277 | 0.221 | 0.218 | 0.186 | 0.019 | 0.159 | 0.715 |
| DR6 | 0.345 | 0.257 | 0.399 | 0.332 | 0.264 | 0.026 | 0.252 | 0.951 |

¹The dose regimens are ordered from lowest to highest in terms of AUD
²StD = standard deviation

The MTD-R may be considered reached if both of the following criteria is fulfilled:

The posterior probability of the true DLT rate in the target interval (16%-33%) of the MTD-R is above 50% and the probability of overdosing is below 25%. At least 6 subjects have been treated at the MTD-R, including the confirmatory cohort.

The model was assessed using two different metrics:

Hypothetical data scenarios: for various potential data constellations as they could occur in the current study, the maximal next doses as allowed by the model and by the escalation limit are investigated. Data scenarios thus provide a way to assess the ‘on-study’ behavior of the model. Simulated operating characteristics: these illustrate for different assumed true dose-toxicity relationships, how often a correct dose would be declared as MTD-R by the model. They are a way to assess the ‘long-run’ behavior of the model. In summary, the model showed very good behavior as assessed by these metrics. More details can be found in Appendix E.

### 11.5 Safety Analysis

The statistical analysis of safety data will be conducted for subjects in the FAS and the DAS and include subjects with non-missing data for the particular safety endpoint being analyzed. Safety variables may include, but are not limited to: DLTs, treatment-emergent adverse events (TEAEs), vital signs, physical examination findings, and clinically significant laboratory test results. Data will be presented by dose cohort and in escalation and expansion cohorts. Some safety data may be summarized over all dose cohorts and across both escalation and expansion cohorts. Data may be graphed, summarized, or listed, depending on the amount of data to be reported.

### 11.5.1 Adverse Events

AE severity will be graded according to NCI CTCAE v5.0 with the exception of CRS events which will be graded per ASTCT criteria, and terms recorded on the CRFs will be classified by system organ class (SOC) and preferred term using the Medical Dictionary for Drug Regulatory Activities (MedDRA) version 25, with seventies classified using the CTCAE v5.0 criteria.

All collected AE data will be listed. Separately, all SAEs will also be listed. All TEAEs, regardless of attribution, will be summarized by cohort as follows:
All DLTs by SOC, preferred term, and maximum grade—DLT evaluable population
All TEAEs by SOC, preferred term, and maximum grade (regardless of attribution)
All Grade 3/4/5 TEAEs
All TEAE by preferred term and descending frequency
All study treatment-related TEAEs by SOC, preferred term, and maximum grade
All TEAEs leading to study drug or study discontinuations by SOC and preferred term
All serious TEAEs, including deaths
All study treatment-related serious TEAEs by SOC, preferred term, and maximum grade

A separate listing of on-study deaths (other than those related to PD) will be presented.

All AEs will be summarized according to the SOC and preferred term within the SOC. AEs will be tallied for overall frequency (number and percentage of subjects), worst reported severity, and relationship to the investigational study treatment for each preferred term per subject. SAEs will be similarly summarized. AEs that occurred during screening but before exposure to study treatment will be reported in the AE line listings and appropriately identified as non-TEAEs.

### Safety Laboratory Assessments

For select laboratory parameters, changes of laboratory values over time, grade shifts in laboratory value from baseline to worst on-study value and Grade≥3 laboratory toxicities will be summarized. Details will be provided in the SAP.

### 11.5.3 Exploratory Safety and Pharmacodynamic Assessments

Baseline and on-treatment biomarkers may be assessed and evaluated for correlations with safety for subjects with available biomarker data. Longitudinal exploratory multiplex cytokines/chemokines may be assessed on a batched basis. Blood samples may be analyzed by central laboratory to assess immune cell subsets and activation status. In addition, PBMC samples from multiple timepoints will be retained for exploratory analysis. Additional post hoc statistical analyses not specified in the protocol or SAP, such as alternative modeling approaches, may be conducted. All analyses are based on available data.

### 11.6 Pharmacokinetic Analysis

The PKAS will be used for summaries of PK concentration data and PK parameters.

For subjects with sufficient data, the following PK parameters for intact PC-1, the unmasked/active PC-1 (TCE), and cleaved fragments will be estimated from the individual concentration-time profiles using a non-compartmental analysis approach:
Area under the concentration-time curve from time 0 to time t (AUC₀₋ₜ)
Area under the concentration-time curve extrapolated to infinity (AUC_{0-inf})
Clearance (CL)
Maximum concentration (Cₘₐₓ)
Last measurable concentration (Cₗₐₛₜ)
Time of maximum concentration (Tₘₐₓ)
Time of last measurable concentration (Tₗₐₛₜ)
Terminal half-life (t_{1/2})
Volume of distribution (V_{d})

Parameters will be calculated using data following the relevant doses if sufficient data are available. Additional analyses may be conducted if deemed useful and appropriate. PK parameter summary statistics may include mean, standard deviation, geometric mean and standard deviation, median, 25th to 75th percentile, minimum, and maximum. PK parameter summary statistics will be presented by dose cohort. All concentrations below the limit of detection or quantitation, or missing data, will be labeled as such in the concentration data listings. Concentrations below the limit of detection will be treated as 0 in summary statistics.

### 11.7 Immunogenicity Analysis

A validated 3 tier immunogenicity assay will be used. Immunogenicity assessment will include prevalence (subjects with pre-existing antibodies that bind to PC-1), incidence and persistence of immunogenicity (subjects with treatment-induced antibodies that bind to PC-1). Data will be further analyzed to determine the proportion of subjects who make transient versus sustained antibody responses. In addition, the potential correlation between immunogenicity and other endpoints (PK, major safety, efficacy, and biomarker parameters) will be evaluated. Batch analyses will be conducted throughout the study. Confirmed immunogenicity positive samples will be banked to assess the neutralizing antibody presence when the neutralizing antibody assay is available.

### 11.8 Efficacy Analyses

The analysis of efficacy data will be conducted for subjects in the FAS and the PPAS. Response to treatment will be evaluated using RECIST 1.1 and PCWG3 (Appendix D). All subjects will have efficacy evaluated by assessing PSA responses and PSA progression per PCWG3. Summaries of subjects with PSA reductions of 30%, 50%, 75%, and 90% will be tabulated. For subjects with measurable soft-tissue tumor lesions, response to treatment and progression will be evaluated per RECIST 1.1. Bone disease will be evaluated per PCWG3. ORR, DOR, rPFS, and OS will be calculated. PFS may also be calculated.

Definitions for response and survival outcomes are provided in Table 27.

TABLE 27
Efficacy Endpoints for Individual Subjects and Summary Statistics

| Efficacy | Description | Summary |
|---|---|---|
| Endpoints | of Endpoints | Statistics |
| PSA30, 50, 75, 90 | The best reduction in PSA level achieved during | Proportion of subjects who |
| | study participation | achieved PSA reduction of |
| | | 30%, 50%, 75%, and 90% |
| ORR | CR or PR achieved during study participation | Proportion of subjects who |
| | according to RECIST 1.1 for soft-tissue disease and | achieved CR or PR |
| | without evidence of bone progression according to | according to RECIST 1.1 |
| | PCWG3 | for soft-tissue disease |
| | | without evidence of bone |
| | | progression according to |
| | | PCWG3 |
| DOR | Time from documentation of objective tumor | Probability of response at |
| | response (PR or CR) according to RECIST 1.1 for | selected timepoints, median |
| | soft-tissue disease and without evidence of bone | DOR, if estimable |
| | progression according to PCWG3 to date of first | |
| | documented evidence of radiographic disease | |
| | progression as defined in the PCWG3 or RECIST | |
| | 1.1 for soft-tissue disease. This endpoint only includes | |
| rPFS | Time from treatment initiation until documented | Probability of rPFS at |
| | disease progression or death. Radiographic | selected timepoints, median |
| | progressive disease is defined as progression of | rPFS, if it can be estimated |
| | soft tissue lesions measured by CT or MRI as defined | |
| | by | |
| PFS | Time from treatment initiation until documented | Probability of PFS at |
| | disease progression, clinical or radiographic, or | selected timepoints, median |
| | death, whichever occurs first | PFS, if it can be estimated |
| OS | Time from treatment initiation until death from | Probability of survival at |
| | any cause | selected timepoints, median |
| | | survival, if estimable |

CR = complete response;
DOR = duration of response;
ORR = objective response rate;
OS = overall survival;
PFS = progression-free survival;
PR = partial response;
PSA = prostate-specific antigen;
rPFS = radiographic progression-free survival;
SD = stable disease

The KM method will be used to provide estimates of the probability that subjects are free of the event of interest (like disease progression or death) at specific timepoints. Where observed data allow for estimation of the median DOR, rPFS, and OS, these will be reported.

### Secondary Efficacy Endpoints

A sensitivity analysis of the secondary efficacy endpoints will be conducted on the PPAS if >10% of subjects in the FAS are excluded from the PPAS.

### Overall Response Rate

The analysis of ORR will be conducted on the FAS. The point estimate of the ORR and the corresponding exact binomial 2-sided 95% CI will be generated.

### Duration of Response

The analysis of DOR will be conducted on the responders only.

The KM estimate and corresponding 2-sided 95% CI for the median and quartiles will be provided, if estimable. The probability of response at specific timepoints will be reported. The KM plot may also be provided.

Radiographic Progression-Free Survival and Overall Survival The analysis of rPFS and OS will be conducted on the FAS.

The KM estimates and corresponding 2-sided 95% CI for the median and quartiles will be provided, if estimable. The probabilities of rPFS and OS at specific timepoints will be reported. The KM plot may also be provided.

### 11.8.2 Exploratory Efficacy Endpoints

The KM estimates and corresponding 2-sided 95% CI for the median and quartiles may be provided for PFS, if estimable. The probability of PFS at specific timepoints may be reported. The KM plot may also be provided.

Baseline and on-treatment biomarkers may be assessed and evaluated for correlations with anti-tumor activity. Subjects will have pre-treatment PSMA-PET scans to determine baseline PSMA expression level and assess for correlation with response. In addition, on-treatment PSMA-PET scans will be evaluated for response and progression assessment. In the subset of subjects with pre-treatment and/or on treatment biopsies, assessment of tumor marker expression, TME characteristics, and T-cell infiltration may be evaluated for correlations with response.

### 11.9 Biomarker Analyses

The study sample size is not powered for definitive biomarker analyses; therefore, all biomarker analyses will be exploratory in nature. There may be circumstances where a decision is made to stop biomarker sample collection, or not perform or discontinue the analysis of blood due to either practical or sample quality reasons. In such circumstances, the number of samples may be insufficient to perform a complete data analysis, and as such, the available data will be listed and summarized. Additional analyses that may be performed after the completion of the clinical study report will be documented in separate reports.

### Response Criteria

Subjects will be assessed for radiographic and clinical progression based on RECIST 1.1 and PCWG3 as described here. Specifically for radiographic assessments, soft-tissue disease (nodal and visceral) will be evaluated by modified RECIST whereas non-measurable bone disease will be evaluated by PCWG3. In addition, clinical progression is defined by PSA values according to PCWG3. Requirements for disease evaluations are outlined below.

### 15.1 Modified RECIST Criteria for Evaluation of Soft-Tissue Disease From: Eisenhauer et al 2009

### Measurability of Tumor at Baseline

### Definitions

At baseline, tumor lesions/lymph nodes will be categorized measurable or non-measurable as follows.

### Measurable Tumor Lesions

Tumor lesions must be accurately measured in at least 1 dimension (longest diameter in the plane of measurement is to be recorded) with a minimum size of:
10 mm by CT scan (CT scan slice thickness no greater than 5 mm)
10 mm caliper measurement by clinical exam (lesions which cannot be accurately measured with calipers should be recorded as non-measurable)
20 mm by chest X-ray
Malignant lymph nodes: To be considered pathologically enlarged and measurable, a lymph node must be ≥15 mm in short axis when assessed by CT scan (CT scan slice thickness recommended to be no greater than 5 mm). At baseline and in follow-up, only the short axis will be measured and followed. See also section below on ‘Baseline documentation of target and non-target lesions’ for information on lymph node measurement.
Non-measurable tumor lesions
Non-measurable tumor lesions encompass small lesions (longest diameter <10 mm or pathological lymph nodes with ≥10 to <15 mm short axis) as well as truly non-measurable lesions. Lesions considered truly non-measurable include: leptomeningeal disease, ascites, pleural or pericardial effusion, inflammatory breast disease, lymphangitic involvement of skin or lung, peritoneal spread, abdominal masses/abdominal organomegaly identified by physical exam that is not measurable by reproducible imaging techniques.
Special considerations regarding lesion measurability
Bone lesions, cystic lesions, and lesions previously treated with local therapy require particular comment:
Bone lesions:
   Bone scan, PET scan or plain films are not considered adequate imaging techniques to measure bone lesions. However, these techniques can be used to confirm the presence or disappearance of bone lesions.
   Lytic bone lesions or mixed lytic-blastic lesions, with identifiable soft tissue components, that can be evaluated by cross sectional imaging techniques such as CT or MRI can be considered as measurable lesions if the soft tissue component meets the definition of measurability described above.
   Blastic bone lesions are non-measurable.
Cystic lesions:
   Lesions that meet the criteria for radiographically defined simple cysts should not be considered as malignant lesions (neither measurable nor non-measurable) since they are, by definition, simple cysts
   ‘Cystic lesions’ thought to represent cystic metastases can be considered as measurable lesions, if they meet the definition of measurability described above. However, if non-cystic lesions are present in the same subject, these are preferred for selection as target lesions
      Lesions with Prior Local Treatment:
Tumor lesions situated in a previously irradiated area, or in an area subjected to other loco-regional therapy, are usually not considered measurable unless there has been demonstrated progression in the lesion. Study protocols should detail the conditions under which such lesions would be considered measurable.

### Specifications by Methods of Measurements

### Measurement of Lesions

All measurements should be recorded in metric notation, using calipers if clinically assessed. All baseline evaluations should be performed as close as possible to the treatment start and never more than 4 weeks before the beginning of the treatment.

### Method of Assessment

The same method of assessment and the same technique should be used to characterize each identified and reported lesion at baseline and during follow-up. Imaging based evaluation should always be done rather than clinical examination unless the lesion(s) being followed cannot be imaged but are assessable by clinical exam.

Clinical lesions: Clinical lesions will only be considered measurable when they are superficial and ³10 mm diameter as assessed using calipers (eg, skin nodules). For the case of skin lesions, documentation by color photography including a ruler to estimate the size of the lesion is suggested. As noted above, when lesions can be evaluated by both clinical exam and imaging, imaging evaluation should be undertaken since it is more objective and may also be reviewed at the end of the study.

Chest X-ray: Chest CT is preferred over chest X-ray, particularly when progression is an important endpoint, since CT is more sensitive than X-ray, particularly in identifying new lesions. However, lesions on chest X-ray may be considered measurable if they are clearly defined and surrounded by aerated lung. Still, non-contrast CT is preferred over chest X-ray.

CT, MRI: CT is the best currently available and reproducible method to measure lesions selected for response assessment. This guideline has defined measurability of lesions on CT scan based on the assumption that CT slice thickness is 5 mm or less. When CT scans have slice thickness greater than 5 mm, the minimum size for a measurable lesion should be twice the slice thickness. MRI is also acceptable in certain situations (eg, for body scans).

If prior to enrollment it is known that a subject is not able to undergo CT scans with IV contrast due to allergy or renal insufficiency, the decision as to whether a non-contrast CT or MRI (with or without IV contrast) will be used to evaluate the subject at baseline and follow-up, should be guided by the tumor type under investigation and the anatomic location of the disease. For subjects who develop contraindications to contrast after baseline contrast CT is done, the decision as to whether non-contrast CT or MRI (enhanced or non-enhanced) will be performed, should also be based on the tumor type, anatomic location of the disease and should be optimized to allow for comparison to the prior studies if possible. Each case should be discussed with the radiologist to determine if substitution of these other approaches is possible and, if not, the subject should be considered not evaluable from that point forward.

Ultrasound: Ultrasound is not useful in assessment of lesion size and should not be used as a method of measurement. Ultrasound examinations cannot be reproduced in their entirety for independent review at a later date and, because they are operator dependent, it cannot be guaranteed that the same technique and measurements will be taken from 1 assessment to the next. If new lesions are identified by ultrasound in the course of the study, confirmation by CT or MRI is advised. If there is concern about radiation exposure at CT, MRI may be used instead of CT in selected instances.

Endoscopy, laparoscopy: The utilization of these techniques for objective tumor evaluation is not advised. However, they can be useful to confirm complete pathological response when biopsies are obtained or to determine relapse in trials where recurrence following complete response or surgical resection is an endpoint.

Cytology, histology: These techniques can be used to differentiate between PR and CR in rare cases if required by protocol (for example, residual lesions in tumor types such as germ cell tumors, where known residual benign tumors can remain). When effusions are known to be a potential adverse effect of treatment (eg, with certain taxane compounds or angiogenesis inhibitors), the cytological confirmation of the neoplastic origin of any effusion that appears or worsens during treatment can be considered if the measurable tumor has met criteria for response or stable disease in order to differentiate between response (or stable disease) and PD.

### Soft-Tissue Tumor Response Evaluation

### Assessment of Overall Tumor Burden and Measurable Disease

To assess objective response or future progression, it is necessary to estimate the overall tumor burden at baseline and use this as a comparator for subsequent measurements. Only subjects with measurable disease at baseline should be included in protocols where objective tumor response is the primary endpoint. Measurable disease is defined by the presence of at least 1 measurable lesion (as detailed above). In studies where the primary endpoint is tumor progression (either time to progression or proportion with progression at a fixed date), the protocol must specify if entry is restricted to those with measurable disease or whether subjects having non-measurable disease only are also eligible.

### Baseline Documentation of ‘Target’ and ‘Non-Target’ Lesions

When more than 1 measurable lesion is present at baseline all lesions up to a maximum of 5 lesions total (and a maximum of 2 lesions per organ) representative of all involved organs should be identified as target lesions and will be recorded and measured at baseline.

This means in instances where subjects have only 1 or 2 organ sites involved a maximum of 2 (1 site) and 4 lesions (2 sites), respectively, will be recorded. Other lesions in that organ will be recorded as non-measurable lesions (even if size is greater than 10 mm by CT scan).

Target lesions should be selected on the basis of their size (lesions with the longest diameter), be representative of all involved organs, but in addition should be those that lend themselves to reproducible repeated measurements. It may be the case that, on occasion, the largest lesion does not lend itself to reproducible measurement in which circumstance the next largest lesion which can be measured reproducibly should be selected.

Lymph nodes merit special mention since they are normal anatomical structures which may be visible by imaging even if not involved by tumor. Pathological nodes which are defined as measurable and may be identified as target lesions must meet the criterion of a short axis of ³15 mm by CT scan. Only the short axis of these nodes will contribute to the baseline sum. The short axis of the node is the diameter normally used by radiologists to judge if a node is involved by solid tumor. Nodal size is normally reported as 2 dimensions in the plane in which the image is obtained (for CT scan this is almost always the axial plane; for MRI the plane of acquisition may be axial, sagittal, or coronal). The smaller of these measures is the short axis. For example, an abdominal node which is reported as being 20 mm×30 mm has a short axis of 20 mm and qualifies as a malignant, measurable node. In this example, 20 mm should be recorded as the node measurement. All other pathological nodes (those with short axis ³10 mm but <15 mm) should be considered non-target lesions. Nodes that have a short axis <10 mm are considered non-pathological and should not be recorded or followed.

A sum of the diameters (longest for non-nodal lesions, short axis for nodal lesions) for all target lesions will be calculated and reported as the baseline sum diameters. If lymph nodes are to be included in the sum, then as noted above, only the short axis is added into the sum. The baseline sum diameters will be used as reference to further characterize any objective tumor regression in the measurable dimension of the disease.

All other lesions (or sites of disease) including pathological lymph nodes should be identified as non-target lesions and should also be recorded at baseline. Measurements are not required, and these lesions should be followed as ‘present’, ‘absent’, or in rare cases ‘unequivocal progression.’ In addition, it is possible to record multiple non-target lesions involving the same organ as a single item on the case report form (eg, ‘multiple enlarged pelvic lymph nodes’ or ‘multiple liver metastases’).

### Response Criteria

This section provides the definitions of the criteria used to determine objective tumor response for target lesions.

### Evaluation of Target Lesions

Complete Response (CR): Disappearance of all target lesions. Any pathological lymph nodes (whether target or non-target) must have reduction in short axis to <10 mm.

Partial Response (PR): At least a 30% decrease in the sum of diameters of target lesions, taking as reference the baseline sum diameters.

Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of 1 or more new soft-tissue lesions is also considered progression).

Stable Disease (SD): Neither sufficient shrinkage to qualify for PR nor sufficient increase to qualify for PD, taking as reference the smallest sum diameters while on study.

Inevaluable (NE): PD has not been documented, and one of the following conditions has been met:
1 or more target lesion has not been assessed
Assessment methods used were inconsistent with those used at baseline
1 or more target lesions cannot be measured accurately (eg, poorly visible unless due to being too small to measure)
1 or more target lesions were excised or irradiated and have not reappeared or increased

### Special Notes on the Assessment of Target Lesions

Lymph nodes: Lymph nodes identified as target lesions should always have the actual short axis measurement recorded (measured in the same anatomical plane as the baseline examination), even if the nodes regress to below 10 mm on study. This means that when lymph nodes are included as target lesions, the ‘sum’ of lesions may not be zero even if complete response criteria are met, since a normal lymph node is defined as having a short axis of <10 mm. Case report forms or other data collection methods may therefore be designed to have target nodal lesions recorded in a separate section where, in order to qualify for CR, each node must achieve a short axis <10 mm. For PR, SD, and PD, the actual short axis measurement of the nodes is to be included in the sum of target lesions.

Target lesions that become ‘too small to measure’: while on study, all lesions (nodal and non-nodal) recorded at baseline should have their actual measurements recorded at each subsequent evaluation, even when very small (eg, 2 mm). However, sometimes lesions or lymph nodes which are recorded as target lesions at baseline become so faint on CT scan that the radiologist may not feel comfortable assigning an exact measure and may report them as being ‘too small to measure’. When this occurs, it is important that a value be recorded on the case report form:

If it is the opinion of the radiologist that the lesion has likely disappeared, the measurement should be recorded as 0 mm. If the lesion is believed to be present and is faintly seen but too small to measure, a default value of 5 mm should be assigned and BML (below measurable limit) should be ticked (Note: It is less likely that this rule will be used for lymph nodes since they usually have a definable size when normal and are frequently surrounded by fat such as in the retroperitoneum; however, if a lymph node is believed to be present and is faintly seen but too small to measure, a default value of 5 mm should be assigned in this circumstance as well and BML should also be ticked).

This default value is derived from the 5 mm CT slice thickness (but should not be changed with varying CT slice thickness). The measurement of these lesions is potentially non-reproducible, therefore providing this default value will prevent false responses or progressions based on measurement error. To reiterate, however, if the radiologist is able to provide an actual measure, that should be recorded, even if it is below 5 mm and in that case BML should not be ticked. (BML is equivalent to a less than sign <.) Lesions that split or coalesce on treatment: when non-nodal lesions ‘fragment’, the longest diameters of the fragmented portions should be added together to calculate the target lesion sum. Similarly, as lesions coalesce, a plane between them may be maintained that would aid in obtaining maximal diameter measurements of each individual lesion. If the lesions have truly coalesced such that they are no longer separable, the vector of the longest diameter in this instance should be the maximal longest diameter for the ‘coalesced lesion’.

### Evaluation of Non-Target Lesions

This section provides the definitions of the criteria used to determine the tumor response for the group of non-target lesions. While some non-target lesions may actually be measurable, they need not be measured and instead should be assessed only qualitatively at the timepoints specified in the protocol. Complete Response (CR): Disappearance of all non-target lesions. All lymph nodes must be non-pathological in size (<10 mm short axis).

Non-CR/Non-PD: Persistence of 1 or more non-target lesion(s).

Progressive Disease (PD): Unequivocal progression of existing non-target lesions.
(Note: the appearance of 1 or more new soft-tissue lesions is also considered progression).

Inevaluable (NE): PD has not been determined and 1 or more non-target sites were not assessed, or assessment methods were inconsistent with those used at baseline.

### Special Notes on Assessment of Progression of Non-Target Disease

The concept of progression of non-target disease requires additional explanation as follows: When the subject also has measurable disease: in this setting, to achieve ‘unequivocal progression’ on the basis of the non-target disease, there must be an overall level of substantial worsening in non-target disease in a magnitude that, even in presence of SD or PR in target disease, the overall tumor burden has increased sufficiently to merit discontinuation of therapy. A modest ‘increase’ in the size of 1 or more non-target lesions is usually not sufficient to qualify for unequivocal progression status. The designation of overall progression solely on the basis of change in non-target disease in the face of SD or PR of target disease will therefore be extremely rare.

When the subject has only non-measurable disease: this circumstance arises in some phase III trials when it is not a criterion of study entry to have measurable disease. The same general concepts apply here as noted above, however, in this instance there is no measurable disease assessment to factor into the interpretation of an increase in non-measurable disease burden. Because worsening in non-target disease cannot be easily quantified (by definition: if all lesions are truly non-measurable) a useful test that can be applied when assessing subjects for unequivocal progression is to consider if the increase in overall disease burden based on the change in non-measurable disease is comparable in magnitude to the increase that would be required to declare PD for measurable disease: i.e., an increase in tumor burden representing an additional 73% increase in ‘volume’ (which is equivalent to a 20% increase diameter in a measurable lesion). Examples include an increase in a pleural effusion from ‘trace’ to ‘large’, an increase in lymphangitic disease from localized to widespread, or may be described in protocols as ‘sufficient to require a change in therapy’. If ‘unequivocal progression’ is seen, the subject should be considered to have had overall PD at that point. While it would be ideal to have objective criteria to apply to non-measurable disease, the very nature of that disease makes it impossible to do so; therefore, the increase must be substantial.

### New Lesions

The appearance of new malignant soft-tissue lesions denotes disease progression; therefore, some comments on detection of new lesions are important. There are no specific criteria for the identification of new radiographic lesions; however, the finding of a new lesion should be unequivocal: i.e., not attributable to differences in scanning technique, change in imaging modality or findings thought to represent something other than tumor. This is particularly important when the subject's baseline lesions show partial or complete response. For example, necrosis of a liver lesion may be reported on a CT scan report as a ‘new’ cystic lesion, which it is not.

A lesion identified on a follow-up study in an anatomical location that was not scanned at baseline is considered a new lesion and will indicate disease progression. An example of this is the subject who has visceral disease at baseline and while on study has a brain CT or MRI ordered which reveals metastases. The subject's brain metastases are considered to be evidence of PD even if he/she did not have brain imaging at baseline.

If a new soft-tissue lesion is equivocal, for example because of its small size, continued therapy and follow-up evaluation will clarify if it represents truly new disease. If repeat scans confirm there is definitely a new lesion, then progression should be declared using the date of the initial scan.

### (18)F-Fluorodeoxyglucose Positron Emission Tomography (FDG-PET)

While FDG-PET response assessments need additional study, it is sometimes reasonable to incorporate the use of FDG-PET scanning to complement CT scanning in assessment of progression (particularly possible ‘new’ disease). New lesions on the basis of FDG-PET imaging can be identified according to the following algorithm:

Negative FDG-PET at baseline, with a positive FDG-PET at follow-up is a sign of PD based on a new lesion.

No FDG-PET at baseline and a positive FDG-PET at follow-up:
If the positive FDG-PET at follow-up corresponds to a new site of disease confirmed by CT, this is PD.
If the positive FDG-PET at follow-up is not confirmed as a new site of disease on CT, additional follow-up CT scans are needed to determine if there is truly progression occurring at that site (if so, the date of PD will be the date of the initial abnormal FDG-PET scan).
If the positive FDG-PET at follow-up corresponds to a pre-existing site of disease on CT that is not progressing on the basis of the anatomic images, this is not PD.

### Evaluation of Best Overall Response

The best overall response is the best response recorded from the start of the study treatment until the end of treatment taking into account any requirement for confirmation. For this study, responses should be confirmed at least 4 weeks after the initial response is documented. Post-treatment assessments can be considered in determination of best overall response. Protocols must specify how any new therapy introduced before progression will affect best response designation. The subject's best overall response assignment will depend on the findings of both target and non-target soft-tissue disease and will also take into consideration the appearance of new soft-tissue lesions.

### Timepoint Response

It is assumed that at each protocol specified timepoint, a response assessment occurs. Table 33 provides a summary of the overall soft-tissue response status calculation at each timepoint for subjects who have measurable disease at baseline.

When subjects have non-measurable (therefore non-target) soft-tissue disease only, Table 34 is to be used.

Missing Assessments and not-Evaluable Designation

When no imaging/measurement is done at all at a particular timepoint, the subject is not evaluable at that timepoint. If only a subset of lesion measurements are made at an assessment, usually the case is also considered not evaluable at that timepoint, unless a convincing argument can be made that the contribution of the individual missing lesion(s) would not change the assigned timepoint response. This would be most likely to happen in the case of PD. For example, if a subject had a baseline sum of 50 mm with 3 measured lesions and at follow-up only 2 lesions were assessed, but those gave a sum of 80 mm, the subject will have achieved PD status, regardless of the contribution of the missing lesion. If 1 or more target lesions were not assessed either because the scan was not done or could not be assessed because of poor image quality or obstructed view, the Response for Target Lesions should be “Unable to Assess” since the subject is not evaluable. Similarly, if 1 or more non-target lesions are indicated as ‘not assessed’, the response for non-target lesions should be “Unable to Assess” (except where there is clear progression). Overall response would be “Unable to Assess” if either the target response or the non-target response is “Unable to Assess” (except where this is clear evidence of progression) as this equates with the case being not evaluable at that timepoint.

Best overall response: all timepoints

The best overall response will be determined by statistical programming once all the data for the subject is known.

TABLE 28
Timepoint Response: Subjects with Soft-
Tissue Target (±Non-Target) Disease

| | | New | |
|---|---|---|---|
| Target | Non-Target | Soft-Tissue | Overall |
| Lesions | Lesions | Lesions | Response |
| CR | CR | No | CR |
| CR | Non-CR/Non-PD | No | PR |
| CR | Not evaluated | No | PR |
| PR | Non-PD or not | No | PR |
| | all evaluated | | |
| SD | Non-PD or not | No | SD |
| | all evaluated | | |
| Not all evaluated | Non-PD | No | NE |
| PD | Any | Yes or No | PD |
| Any | PD | Yes or No | PD |
| Any | Any | Yes | PD |

CR = complete response;
NE = inevaluable;
PD = progressive disease;
PR = partial response;
SD = stable disease

TABLE 29
Timepoint Response: Subjects with
Soft-Tissue Non-Target Disease Only

| Non-Target Lesions | New Soft-Tissue Lesions | Overall Response |
|---|---|---|
| CR | No | CR |
| Non-CR/Non-PD | No | Non-CR/Non-PD¹ |
| Not all evaluated | No | NE |
| Unequivocal PD | Yes or No | PD |
| Any | Yes | PD |

CR = complete response;
NE = inevaluable;
PD = progressive disease;
PR = partial response;
SD = stable disease
¹‘Non-CR/Non-PD’ is preferred over ‘stable disease’ for non-target disease since SD is increasingly used as endpoint for assessment of efficacy in some trials so to assign this category when no lesions can be measured is not advised.

TABLE 30
Overall Response Rate When Confirmation
of CR and PR Required

| Overall | Overall | |
|---|---|---|
| Response | Response | |
| First | Subsequent | Overall |
| Timepoint | Timepoint | Response |
| CR | CR | CR |
| CR | PR | SD, PD or PR¹ |
| CR | SD | SD provided minimum criteria for SD |
| | | duration met, otherwise, PD |
| CR | PD | SD provided minimum criteria for SD |
| | | duration met, otherwise, PD |
| CR | NE | SD provided minimum criteria for SD |
| | | duration met, otherwise, NE |
| PR | CR | PR |
| PR | PR | PR |
| PR | SD | SD |
| PR | PD | SD provided minimum criteria for SD |
| | | duration met, otherwise, PD |
| PR | NE | SD provided minimum criteria for SD |
| | | duration met, otherwise, NE |
| NE | NE | NE |

CR = complete response;
NE = inevaluable;
PD = progressive disease;
PR = partial response;
SD = stable disease
¹If a CR is truly met at first timepoint, then any disease seen at a subsequent timepoint, even disease meeting PR criteria relative to baseline, makes the disease PD at that point (since disease must have reappeared after CR). Best response would depend on whether minimum duration for SD was met. However, sometimes ‘CR’ may be claimed when subsequent scans suggest small lesions were likely still present and in fact the subject had PR, not CR at the first timepoint. Under these circumstances, the original CR should be changed to PR and the best response is PR.

### Special Notes on Response Assessment

When nodal disease is included in the sum of target lesions and the nodes decrease to ‘normal’ size (<10 mm), they may still have a measurement reported on scans. This measurement should be recorded even though the nodes are normal in order not to overstate progression should it be based on increase in size of the nodes. As noted earlier, this means that subjects with CR may not have a total sum of ‘zero’ on the CRF. In trials where confirmation of response is required, repeated ‘NE’ timepoint assessments may complicate best response determination. The analysis plan for the trial must address how missing data/assessments will be addressed in determination of response and progression. For example, in most trials it is reasonable to consider a subject with timepoint responses of PR-NE-PR as a confirmed response. Subjects with a global deterioration of health status requiring discontinuation of treatment without objective evidence of disease progression at that time should be reported as ‘symptomatic deterioration’. Every effort should be made to document objective progression even after discontinuation of treatment. Symptomatic deterioration is not a descriptor of an objective response: it is a reason for stopping study therapy. The objective response status of such subjects is to be determined by evaluation of target and non-target disease as shown in Table 31, Table 32, and Table 33.

Conditions that define ‘early progression, early death and non-evaluability’ are study specific and should be clearly described in each protocol (depending on treatment duration, treatment periodicity). In some circumstances it may be difficult to distinguish residual disease from normal tissue. When the evaluation of complete response depends upon this determination, it is recommended that the residual lesion be investigated (fine needle aspirate/biopsy) before assigning a status of complete response. FDG-PET may be used to upgrade a response to a CR in a manner similar to a biopsy in cases where a residual radiographic abnormality is thought to represent fibrosis or scarring. The use of FDG-PET in this circumstance should be prospectively described in the protocol and supported by disease specific medical literature for the indication. However, it must be acknowledged that both approaches may lead to false positive CR due to limitations of FDG-PET and biopsy resolution/sensitivity. For equivocal findings of progression (eg, very small and uncertain new lesions; cystic changes or necrosis in existing lesions), treatment may continue until the next scheduled assessment. If at the next scheduled assessment, progression is confirmed, the date of progression should be the earlier date when progression was suspected.

### 15.2 PCWG3 Criteria for Evaluation of Bone Disease and PSA Progressive Disease on Bone Scan

Disease progression by bone scan will be defined as ≥2 new bone lesions at the first post-treatment scan, with ≥2 additional lesions on the next confirmatory scan performed ≥6 weeks later. For scans after the first post-treatment scan, ≥2 new lesions relative to the first post-treatment scan must be confirmed on a subsequent scan. If ≥2 additional new lesions are seen on the confirmatory scan, the date of progression is the date of the first scan when the first new lesions were documented.

Ambiguous results should be confirmed by other imaging modalities (e.g., CT or MRI). Clinical Progression by PSA

Favorable effects on PSA may be delayed for ≥12 weeks. Therefore, early rises in PSA prior to 12 weeks should not be considered progression. The first ≥25% increase from baseline and ≥2 ng/mL above the nadir (confirmed by a second value 3 or more weeks later) will be defined as clinical disease progression. Subjects who progress by raising PSA only should continue to be followed for radiographic progression.

### Results

FIG. 2 illustrates an outline of Planned Dose Escalation, Backfill and Dose Expansion.

FIG. 3 illustrates Study Dosing Schema and Imaging Assessments.

FIG. 4 illustrates Example Step Dose Regimen Schedules.

FIG. 5 illustrates Prior Medians and 95% Credibility Intervals CRS DLTs.

FIG. 6 illustrates Prior Medians and 95% Credibility Intervals Non-CRS DLTs.

FIG. 7 illustrates Prior Medians and 95% Credibility Intervals for the Joint CRS and Non-CRS DLTs.

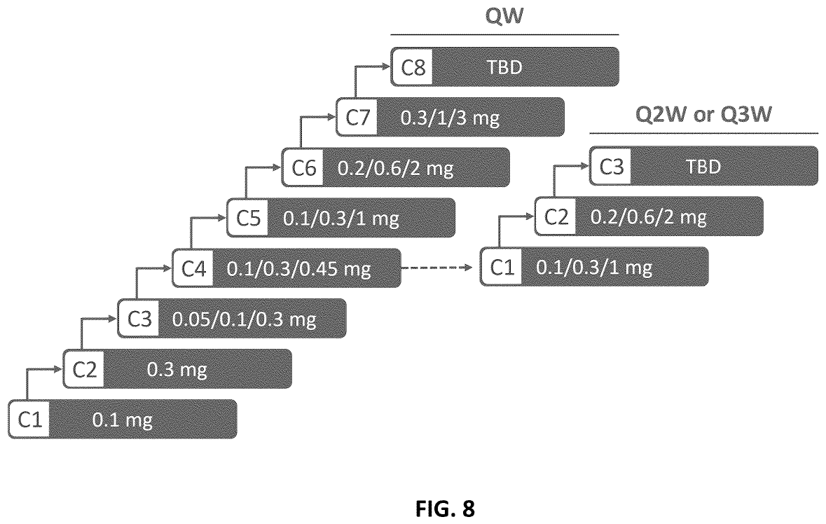

FIG. 8 illustrates phase 1 trial design in mCRPC.

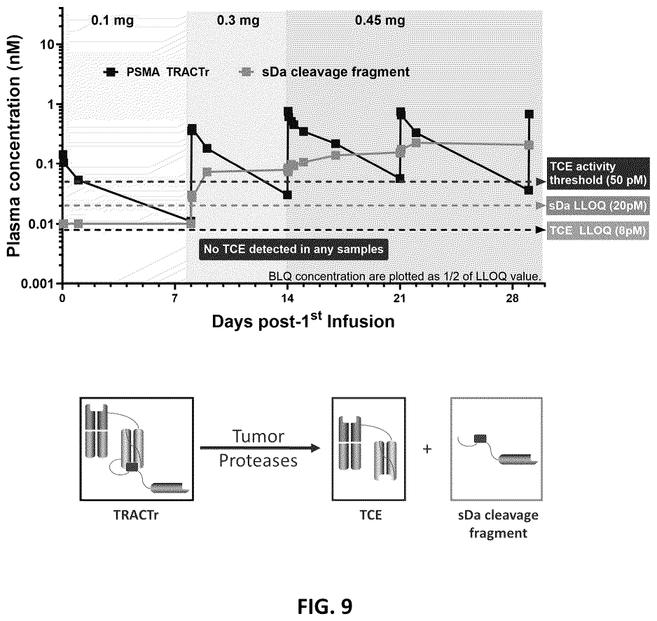

FIG. 9 illustrates Human Exposure of TRACTr Components. FIG. 9 shows TRACTr activation without TCE accumulation, that TRACTr plasma levels are below preclinical activity threshold, that the existence of sDa cleavage fragment indicates TRACTr activation is occurring, and that TCE plasma levels are below preclinical activity threshold. FIG. 9 indicates that PD effects are not from systemic TCE exposures and clinical PK data is consistent with tumor mediated TRACTr activation.

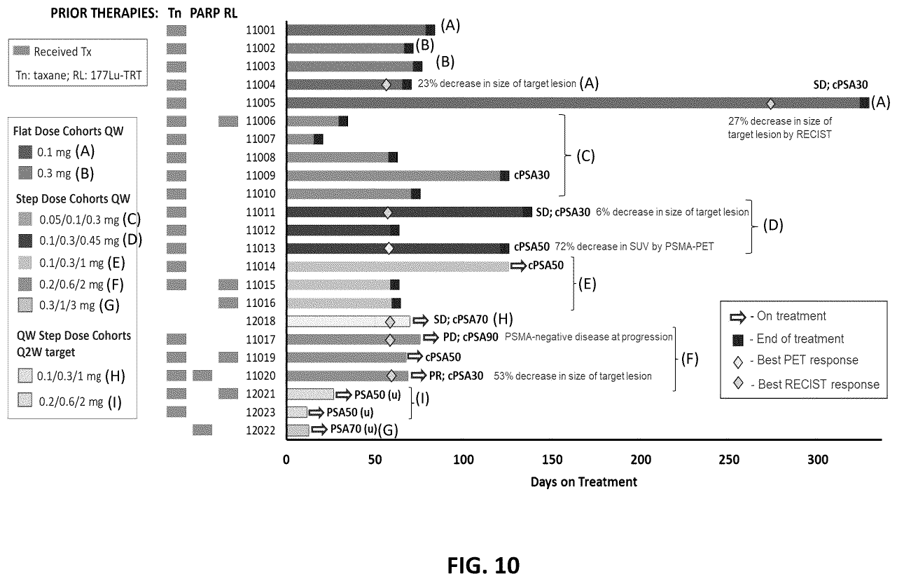

FIG. 10 illustrates Time on treatment for all subjects.

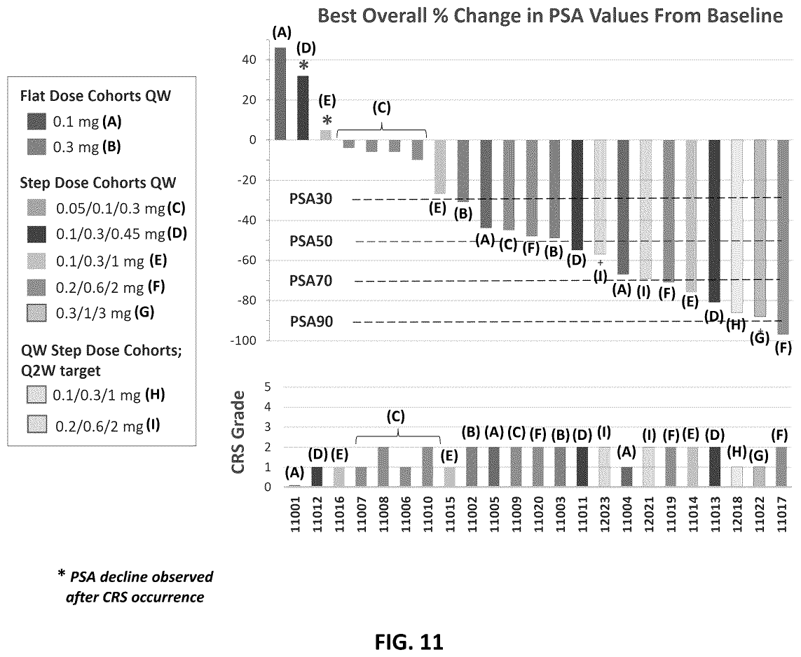

FIG. 11 illustrates Best Overall percentage Change in PSA Values From Baseline for all subjects. FIG. 11 shows PSA responses deepening with increased doses while maintaining low grade CRS. In FIG. 11, CRS was observed only in subjects with PSA reductions. FIG. 11 shows evidence of antitumor activity since PSA declines were observed in the majority of subjects. FIG. 11 shows increasing depth of PSA response as doses are increased: 57% of subjects have at least 50% reduction in PSA level (PSA50) for first step dose of 0.1 mg and 83% PSA50 for first step dose ≥0.2 mg. FIG. 11 shows encouraging CRS profile: Transient, grade ½ occurring in cycle 1; CRS only observed in subjects with PSA declines. FIG. 11 indicates PSA reduction combined with low-grade CRS profile consistent with tumor specific activation.

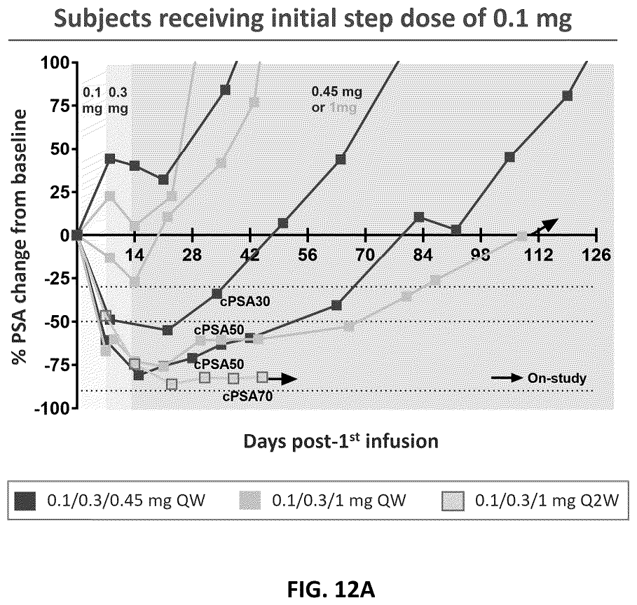

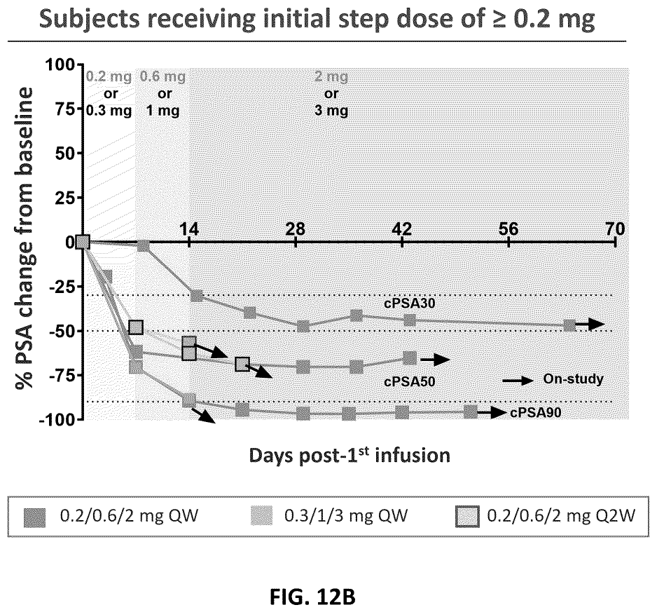

FIG. 12A illustrates PSA responses at step of 0.1 mg. FIG. 12B illustrates PSA responses at step of 0.2 mg. FIGS. 12A and 12B shows improved PSA responses observed at higher doses of PC-1 and that cohorts having 1st step dose of at least 0.2 mg achieve deeper and more durable PSA responses while maintaining low-grade CRS.

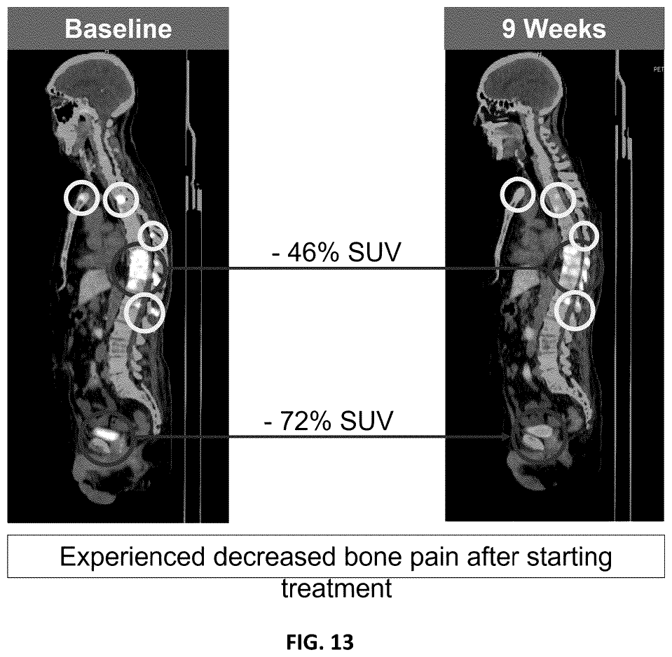

FIG. 13 illustrates significant tumor burden reductions demonstrated by PSMA-PET in a subject. The Subject in FIG. 13 was from the cohort 4, 0.1/0.3/0.45 mg. The patient is 75-year-old, and had Gleason score 9/stage IVB. The patient was heavily treated with 6 prior lines of therapy. After the PC-1 treatment, the patient has a best PSA decline of −81% and achieved a confirmed PSA50 response. During treatment, the patient had Grade 2 CRS with low grade TRAEs (G½). The patient experienced decreased bone pain after starting treatment.

The above results indicate that PC-1 had favorable safety profile and there was no CRS of more than Grade 2 observed. Deepening PSA and RECIST responses with increased dose levels were observed. For efficacy, Subjects with first step dose of at least 0.2 mg (n=6) had the following PSA response: 100% with PSA30; 83% with PSA50; 17% with PSA90. Subjects with first dose of at least 0.1 mg (n=18) had the following PSA response: 78% with PSA30; 56% with PSA50; 6% with PSA90. PC-2 showed continued deepening of PSA reductions with increased dose levels while maintaining low-grade CRS and TRAE profiles. For safety, No CRS of more than Grade 2 for any cohort was observed, PSA declined consistently observed after CRS, there was non-CRS related TRAEs, majority of TRAEs were low grade (G½) occurring predominantly in cycle 1, low incidence of Grade 3 TRAEs was observed, and no Grade 4 or 5 observed. PC-1 showed a high response rate. Majority of subjects experienced PSA declines. There were greater percentage of subjects achieving ≥50% reductions as dose level is increased. There were no Grade 3 CRS—only G½ CRS easily managed. Subjects with PSA reduction exhibited CRS

### Patient Profile

TABLE 31
Patient Characteristics

| Characteristic | All subjects, n = 23 |
|---|---|
| Median age, years (range) | 69 (46-75) |
| Race | |
| White, n(%) | 17 (74) |
| Asian, n(%) | 1 (4) |
| Black, n(%) | 4 (17) |
| Number of prior lines of | 4 (2-6) |
| therapy, median (range) | |
| Prior taxane, n (%) | 20/23 (87) |
| Baseline PSMA-PET positivity, n (%) | 23/23 (100) |
| Prior PSMA-targeting | 5/23 (23) |
| radioligand therapy, n(%) | |
| Baseline PSA, ng/mL, median (range) | 158.5 (1.3-1991.6) |
| RECIST evaluable, n (%) | 13/19 (68) |
| Bone metastases, n (%) | 16/19 (84) |
| Lymph node metastases, n (%) | 13/19 (68) |
| Visceral metastases, n (%) | 8/19 (42) |
| Liver | 3/19 (16) |
| Lung | 3/19 (16) |
| Adrenal | 1/19 (5) |

Treatment related adverse events were observed in more than 2 subjects and majority low-grade AEs occurred in cycle 1 of treatment, as shown in Table 32.

TABLE 32
Treatment-Related Adverse Effects

| | All Subjects (n = 23) | | | |
|---|---|---|---|---|
| Preferred Term | Grade 1 | Grade 2 | Grade ≥3 | All Grades |
| Cytokine release syndrome | 8 (35) | 13 (57) | 0 | 21 (91) |
| Diarrhoea | 6 (26) | 2 (9) | 0 | 8 (35) |
| Chills | 4 (17) | 2 (9) | 0 | 6 (26) |
| ALT increased | 3 (13) | 1 (4) | 1 (4) | 5 (22) |
| Anaemia | 1 (4) | 2 (9) | 2 (9) | 5 (22) |
| AST increased | 4 (17) | 1 (4) | 0 | 5 (22) |
| Fatigue | 2 (9) | 2 (9) | 0 | 4 (17) |
| Decreased appetite | 4 (17) | 0 | 0 | 4 (17) |
| Nausea | 3 (13) | 1 (4) | 0 | 4 (17) |
| Headache | 3 (13) | 0 | 0 | 3 (13) |
| Blood bilirubin increased | 2 (9) | 1 (4) | 0 | 3 (13) |
| Hypoalbuminaemia | 2 (9) | 1 (4) | 0 | 3 (13) |
| Hypocalcaemia | 3 (13) | 0 | 0 | 3 (13) |
| Hypophosphataemia | 1 (4) | 2 (9) | 0 | 3 (13) |
| Leukopenia/white blood | 3 (13) | 0 | 0 | 3 (13) |
| cell count decreased | | | | |
| Myalgia | 1 (4) | 2 (9) | 0 | 3 (13) |
| Platelet count decreased/ | 2 (9) | 1 (4) | 0 | 3 (13) |
| thrombocytopenia | | | | |
| Pyrexia | 2 (9) | 1 (4) | 0 | 3 (13) |
| Vomiting | 0 | 2 (9) | 0 | 3 (13) |
| Blood alkaline | 2 (9) | 0 | 0 | 2 (9) |
| phosphatase increased | | | | |
| Dysgeusia | 2 (9) | 0 | 0 | 2 (9) |
| Hypomagnesaemia | 2 (9) | 0 | 0 | 2 (9) |
| Lipase increased | 0 | 1 (4) | 1 (4) | 2 (9) |
| Stomatitis | 2 (9) | 0 | 0 | 2 (9) |

### Summary

The summary of efficacy and safety for PC-1 is shown in Table 33.

TABLE 33
Efficacy and Safety of PC-1

| Characteristic | PC-1 (n = 18) | PC-1 (n = 6) |
|---|---|---|
| Selected cohorts | 1st dose ≥ 0.1 mg | 1st step dose ≥ 0.2 mg |
| ≥30% PSA | 78% | 100% |
| ≥50% PSA | 56% | 83% |
| ≥90% PSA | 6% | 17% |
| ≥G3 CRS | 0% | 0% |
| ≥G3 TRAEs | 28% | 17% |

Table 34 illustrates PC-1 exposure for each dose level.

TABLE 34
PC-1 Exposure for each dose level

| | Max Cmax | Mean Cmax | | Max Cmax | Mean Cmax | |
|---|---|---|---|---|---|---|
| Dose (Pt ID included) | (ng/mL) | (ng/mL) | ±SD | (nM) | (nM) | ±SD |
| 0.05 mg Dose Overall | 14.72 | 10.76 | 2.86 | 0.16 | 0.12 | 0.03 |
| (Pt 11006, 11007, 11008, 11009, 11010) | | | | | | |
| Pts Received ≤2 Infusion at this level | 14.72 | 10.76 | 2.86 | 0.16 | 0.12 | 0.03 |
| Pts Received >2 Infusions at this level | NA | NA | NA | NA | NA | NA |
| 0.1 mg Dose Overall | 33.71 | 20.48 | 7.31 | 0.37 | 0.22 | 0.08 |
| (Pt 11001, 11004, 11005, 11006, 11007, 11008, 11009, | | | | | | |
| 11010, 11012, 11013, 11014, 11015, 11016) | | | | | | |
| Pts Received ≤2 Infusion at this level | 33.71 | 18.06 | 6.40 | 0.37 | 0.20 | 0.07 |
| Pts Received >2 Infusions at this level | 32.98 | 28.55 | 3.13 | 0.36 | 0.31 | 0.03 |
| 0.3 mg Dose Overall | 174.73 | 70.86 | 37.34 | 1.91 | 0.77 | 0.41 |
| (Pt 11003, 11006, 11008, 11009, 11010, 11012, 11013, | | | | | | |
| 11014, 11015, 11016) | | | | | | |
| Pts Received ≤2 Infusion at this level | 61.86 | 48.48 | 10.54 | 0.67 | 0.53 | 0.11 |
| Pts Received >2 Infusions at this level | 174.73 | 93.24 | 40.93 | 1.91 | 1.02 | 0.45 |
| 0.45 mg Dose Overall | 130.75 | 100.01 | 30.75 | 1.43 | 1.09 | 0.34 |
| (Pt 11012, 11013) | | | | | | |
| Pts Received ≤2 Infusion at this level | NA | NA | NA | NA | NA | NA |
| Pts Received >2 Infusions at this level | 130.75 | 100.01 | 30.75 | 1.43 | 1.09 | 0.34 |
| 1 mg Dose Overall | 282.98 | 214.31 | 52.11 | 3.08 | 2.33 | 0.57 |
| (Pt 11014, 11015, 11016) | | | | | | |
| Pts Received ≤2 Infusion at this level | NA | NA | NA | NA | NA | NA |
| Pts Received >2 Infusions at this level | 282.98 | 214.31 | 52.11 | 3.08 | 2.33 | 0.57 |

Table 35 illustrates PC-1 exposure for each cohort.

TABLE 35
PC-1 Exposure for each cohort

| | Cohort Max | Cohort Mean | | Cohort Max | Cohort Mean | |
|---|---|---|---|---|---|---|
| Cohorts (Pt ID) | Cmax (ng/mL) | Cmax (ng/mL) | ±SD | Cmax (nM) | Cmax (nM) | ±SD |
| Cohort 1 (0.1 mg flat) | 32.98 | 28.55 | 3.13 | 0.36 | 0.31 | 0.03 |
| Pt 11001, 11004, 11005 | | | | | | |
| Cohort 2 (0.3 mg flat) | 67.99 | 67.99 | 0.00 | 0.74 | 0.74 | 0.00 |
| Pt 11003 | | | | | | |
| Cohort 3 (0.05/0.1/0.3 mg step-up) | 174.73 | 99.56 | 43.53 | 1.91 | 1.09 | 0.47 |
| Pt 11006, 11008, 11009, 11010 | | | | | | |
| Cohort 4 (0.1/0.3/0.45 mg step-up) | 130.75 | 100.01 | 30.75 | 1.43 | 1.09 | 0.34 |
| Pt 11012, 11013 | | | | | | |
| Cohort 5 (0.1/0.3/1 mg step-up) | 282.98 | 214.31 | 52.11 | 3.08 | 2.33 | 0.57 |
| Pt 11014, 11015, 11016 | | | | | | |

Table 36 illustrates exposure of a metabolic product of PC-1, sDa, for each cohort.

TABLE 36
sDa 1 Exposure for each cohort

| | Cohort Max | Cohort Mean | | Cohort Max | Cohort Mean | |
|---|---|---|---|---|---|---|
| Cohorts (Pt ID) | Cmax (ng/mL) | Cmax (ng/mL) | ±SD | Cmax (nM) | Cmax (nM) | ±SD |
| Cohort 1 (0.1 mg flat) | 0.98 | 0.87 | 0.08 | 0.06 | 0.05 | 0.00 |
| Pt 11001, 11004, 11005 | | | | | | |
| Cohort 2 (0.3 mg flat) | 2.83 | 2.83 | 0.00 | 0.17 | 0.17 | 0.00 |
| Pt 11003 | | | | | | |
| Cohort 3 (0.05/0.1/0.3 mg step-up) | 2.60 | 2.09 | 0.33 | 0.15 | 0.12 | 0.02 |
| Pt 11006, 11008, 11009, 11010 | | | | | | |
| Cohort 4 (0.1/0.3/0.45 mg step-up) | 4.42 | 4.09 | 0.33 | 0.26 | 0.24 | 0.02 |
| Pt 11012, 11013 | | | | | | |
| Cohort 5 (0.1/0.3/1 mg step-up) | 13.68 | 8.56 | 4.07 | 0.80 | 0.50 | 0.24 |
| Pt 11014, 11015, 11016 | | | | | | |

### PC-1 PK Assessment:

Cmax of TRACTr PK exposure (ng/mL or nM) was evaluated for individual subjects receiving similar PC-1 dose
Max Cmax corresponding to each PC-1 dose level calculated
Mean Cmax (±SD) corresponding to each PC-1 dose level calculated. This was further narrowed down to:
Mean Cmax (±SD) for pts receiving ≤2 infusions at the given dose level
Mean Cmax (±SD) for pts receiving >2 infusions at the given dose level
Max and Mean sDa Cmax was therefore calculated for each cohort (cohort 1 to 5)

### sDa PK Assessment

Max and Mean sDa Cmax was therefore calculated for each cohort (cohort 1 to 5)

PSMA is a clinically validated (Pluvicto®) and highly expressed target that is expressed in >80%† of mCRPC patients. PC-1 showed single-agent efficacy and safety data in heavily pre-treated, late-stage mCRPC patients. The data support single-agent development in 3 L+ and 2 L+ settings. There was non-overlapping toxicity provides opportunity to treat Pluvicto®® responders and non-responders, to switch maintenance therapy to PC-1 following Pluvicto®® provides opportunity to deepen and prolong responses. There was additional opportunity for PSMA directed therapy for Pluvicto® non-responders, opportunity in 2 L and 3 L settings. PC-1 can be combined with another therapy, including enzalutamide. Enzalutamide upregulates PSMA, the target for PC-1 treatment. Combination with enzalutamide may provide synergy to overcome enzalutamide resistance mechanisms and presents opportunity in 1 L and 2 L settings. PC-1 may be combined with lutetium Lu 177 vipivotide tetraxetan, enzalutamide, abiraterone acetate (ABI), chemotherapy, radium Ra 223 dichloride, a poly-adenosine diphosphate ribose polymerase inhibitor (PARPI), pembrolizumab, or a combination thereof. PC-1 can be novel therapies for 2 L and 3 L+ patients and non-chemo therapies for 1 L patients.

### REFERENCES

Ball K, Dovedi S J, Vajjah P, Phipps A. Strategies for clinical dose optimization of T cell-engaging therapies in oncology. MAbs. 2023 January-December; 15(1):2181016.
Beer T M, Armstrong A J, Rathkopf D E, Loriot Y, Sternberg C N, Higano C S, et al. Enzalutamide in metastatic prostate cancer before chemotherapy. N Engl J Med. 2014; 371(5):424-433.
De Bono J S, Fong L, Beer T M, Gao X, Geynisman D M, Burris H A, et al. Results of an ½ going phase ½ a dose escalation study of HPN424, a tri-specific half-life extended PSMA-targeting T-cell engager, in patients with metastatic castration-resistant prostate cancer (mCRPC). J Clin Oncol. 2021; 39(suppl_15):5013.
De Jong L M, Jiskoot W, Swen J J, Manson M L. Distinct Effects of Inflammation on Cytochrome P450 Regulation and Drug Metabolism: Lessons from Experimental Models and a Potential Role for Pharmacogenetics. Genes (Basel). 2020 Dec. 16; 11(12):1509.
Eisenberger M, Hardy-Bessard A C, Kim C S, Geczi L, Ford D, Mourey L, et al. Phase III study comparing a reduced dose of cabazitaxel (20 mg/m2) and the currently approved dose (25 mg/m2) in postdocetaxel patients with metastatic castration-resistant prostate cancer-PROSELICA. J Clin Oncol. 2017; 35(28):3198-3206.
Eisenhauer E A, Therasse P, Bogaerts J, Schwartz L H, Sargent D, Ford R, et al. New
response evaluation criteria in solid tumours: revised RECIST guideline (version 1.1). Eur J Cancer. 2009; 45(2):228-247.
FDA News Release. FDA approves first PSMA-targeted PET imaging drug for men with prostate cancer. 1 Dec. 2020. US Food and Drug Administration. https://www.fda.gov/news-events/press-announcements/fda-approves-first-psma-targeted-pet-imaging-drug-men-prostate-cancer
FDA News Release. FDA approves second PSMA-targeted PET imaging drug for men with prostate cancer. 27 May 2021. US Food and Drug Administration. https://www.fda.gov/drugs/news-events-human-drugs/fda-approves-second-psma-targeted-pet-imaging-drug-men-prostate-cancer
Fendler W P, Calais J, Eiber M, Flavell R R, Mishoe A, Feng F Y, et al. Assessment of 68Ga-PSMA-11 PET accuracy in localizing recurrent prostate cancer: a prospective single-arm clinical trial. JA MA On col. 2019; 5(6):856-863.
Friedrich M, Raum T, Lutterbuese R, Voelkel M, Deegen P, Rau D, et al. Regression of human prostate cancer xenografts in mice by AMG 212/BAY2010112, a novel PSMA/CD3-Bispecific BiTE antibody cross-reactive with non-human primate antigens. Mol Cancer Ther. 2012; 11(12):2664-2673.
Guideline on clinical trials in small populations. Committee for Medicinal Products for Human Use, European Medicines Agency (EMA), 2006.
Hummel H-D, Kufer P, Grüllich C, Seggewiss-Bernhardt R, Deschler-Baier B, Chatterjee M, et al. Pasotuxizumab, a BiTE® immune therapy for castration-resistant prostate cancer:
Phase I, dose-escalation study findings. Immunotherapy. 2021; 13(2):125-141.
Hupe M C, Philippi C, Roth D, Kumpers C, Ribbat-Idel J, Becker F, et al. Expression of prostate-specific membrane antigen (PSMA) on biopsies is an independent risk stratifier of prostate cancer patients at time of initial diagnosis. Front Oncol. 2018; 8:623.
Kinoshita Y, Kuratsukuri K, Landas S, Imaida K, Rovito P M Jr, Wang C Y, et al. Expression of prostate-specific membrane antigen in normal and malignant human tissues. World J Surg. 2006; 30(4):628-636.
Lee D W, Santomasso B D, Locke F L, Ghobadi A, Turtle C J, Brudno J N, et al. ASTCT consensus grading for cytokine release syndrome and neurologic toxicity associated with immune effector cells. Biol Blood Marrow Transplant. 2019; 25(4):625-638.
Li J, Piskol R, Ybarra R, Chen Y J, Li J, Slaga D, et al. CD3 bispecific antibody-induced cytokine release is dispensable for cytotoxic T cell activity. Sci Transl Med. 2019 Sep. 4; 11(508):eaax8861.
Ma Y, Wang Q, Dong Q, Zhan L, Zhang J. How to differentiate pseudoprogression from true progression in cancer patients treated with immunotherapy. Am J Cancer Res. 2019 Aug. 1; 9(8):1546-1553.
Morris M J, Rowe S P, Gorin M A, Saperstein L, Pouliot F, Josephson D, et al. Diagnostic performance of 18F-DCFPyL-PET/CT in men with biochemically recurrent prostate cancer: results from the CONDOR phase III, multicenter study. Clin Cancer Res. 2021; 27(13):3674-3682.
Neelapu S S, Tummala S, Kebriaei P, Wierda W, Gutierrez C, et al. Chimeric antigen receptor T-cell therapy—assessment and management of toxicities. Nat Rev Clin Oncol. 2018 January; 15(1):47-62.
Philipp N, Kazerani M, Nicholls A, et al. T-cell exhaustion induced by continuous bispecific molecule exposure is ameliorated by treatment-free intervals. Blood. 2022 Sep. 8; 140(10):1104-1118.
Rogatko A, Schoeneck D, Jonas W, Tighiouart M, Khuri F R, Porter A. Translation of innovative designs into phase I trials. J Clin Oncol. 2007 Nov. 1; 25(31):4982-6.
Ryan C J, Smith M R, Fizazi K, Saad F, Mulders P F, Sternberg C N, et al. Abiraterone acetate plus prednisone versus placebo plus prednisone inlotherapy-naive men with metastatic castration-resistant prostate cancer (COU-AA-302): final overall surv121 andomizedsis of a randomised, double-blind, placebo-controlled phase 3 study. Lancet Oncol. 2015; 16(2):152-160.
Saber H, Del Valle P, Ricks T K, Leighton J K. An FDA oncology analysis of CD3 bispecific constructs and first-in-human dose selection. Regul Toxicol Pharmacol. 207; 90:144-152.
Sartor O, de Bono J, Chi K N, Fizazi K, Herrmann K, Rahbar K, et al. Lutetium-177-PSMA-617 for metastatic castration-resistant prostate cancer. N Engl J Med. 2021; 385(12):1091-1103.
Scher H I, Morris M J, Stadler W M, Higano C, Basch E, Fizazi K, et al. Trial design and objectives for castration-resistant prostate cancer: updated recommendations from the Prostate Cancer Clinical Trials Working Group 3. J Clin Oncol. 2016; 34(12):1402-1418.
Sheehan B, Guo C, Neeb A, Paschalis A, Sandhu S, de Bono J S. Prostate-specific membrane antigen biology in lethal prostate cancer and its therapeutic implications. Eur Urol Focus. 2021; 52405-4569(21)00168-1.
Siegel R L, Miller K D, Fuchs H E, Jemal A. Cancer Statistics, 2021. CA Cancer J Clin. 2021; 71(1):7-33.
Silver D A, Pellicer I, Fair W R, Heston W D, and Cordon-Cardo. Prostate-specific Membrane Antigen Expression in Normal and Malignant Tissues. Clin Cancer Res. 1997; 3:81-85.
The Protein Atlas, 2022.
Tran B, Horvath L, Dorff T, Rettig M, Lolkema P M, Machiels J-P, et al. Results from a phase I study of AMG 160, a half-life extended (HLE), PSMA-targeted, bispecific T-cell engager (BiTE@) immune therapy for metastatic castration-resistant prostate cancer (mCRP C). Ann Onco 1. 2020; 31(suppl_4):5507.
Zaorsky N G, Calais J, Fanti S, Tilki D, Dorff T, Spratt D E, et al. Salvage therapy for prostate cancer after radical prostatectomy. Nat Rev Urol. 2021; 18(11):643-668.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for treating cancer comprising administering to a subject in need thereof a first dose and a target dose of an isolated recombinant polypeptide complex, wherein the first dose is at least about 100 μg and the target dose is higher than the first dose, and wherein the isolated recombinant polypeptide complex comprises a first chain with an amino acid sequence having at least 80% sequence identity to SEQ ID NO: 1 and a second chain with an amino acid sequence having at least 80% sequence identity to SEQ ID NO: 2.

2. The method of claim 1, wherein the first dose is about 100 μg to about 1 mg.

3. The method of claim 1, wherein the first dose is at least about 150 μg.

4. The method of claim 1, wherein the first dose is at least about 200 μg.

5. The method of claim 1, wherein the first dose is at least about 250 μg.

6. The method of claim 1, wherein the first dose is at least about 300 μg.

7. The method of claim 1, wherein the first dose is at least about 400 μg.

8. The method of claim 1, wherein the first dose is at least about 500 μg.

9. The method of claim 1, wherein the first dose is at least about 600 μg.

10. The method of claim 1, wherein the first dose is at least about 700 μg.

11. The method of claim 1, wherein the first dose is at least about 800 μg.

12. The method of claim 1, wherein the first dose is at least about 900 μg.

13. The method of claim 1, wherein the first dose is about 100 μg.

14. The method of claim 1, wherein the first dose is about 150 μg.

15. The method of claim 1, wherein the first dose is about 200 μg.

16. The method of claim 1, wherein the first dose is about 250 μg.

17. The method of claim 1, wherein the first dose is about 300 μg.

18. The method of claim 1, wherein the first dose is about 400 μg.

19. The method of claim 1, wherein the first dose is about 500 μg.

20. The method of claim 1, wherein the first dose is about 600 μg.

21. The method of claim 1, wherein the first dose is about 700 μg.

22. The method of claim 1, wherein the first dose is about 800 μg.

23. The method of claim 1, wherein the first dose is about 900 μg.

24. The method of claim 1, wherein the first dose is about 1 mg.

25. The method of claim 1, wherein the target dose is about 450 μg to about 10 mg.

26. The method of claim 1, wherein the target dose is at least about 450 μg.

27. The method of claim 1, wherein the target dose is at least about 750 μg.

28. The method of claim 1, wherein the target dose is at least about 1 mg.

29. The method of claim 1, wherein the target dose is at least about 1.5 mg.

30. The method of claim 1, wherein the target dose is at least about 2 mg.

31. The method of claim 1, wherein the target dose is at least about 2.5 mg.

32. The method of claim 1, wherein the target dose is at least about 3 mg.

33. The method of claim 1, wherein the target dose is at least about 4 mg.

34. The method of claim 1, wherein the target dose is at least about 5 mg.

35. The method of claim 1, wherein the target dose is at least about 6 mg.

36. The method of claim 1, wherein the target dose is at least about 7 mg.

37. The method of claim 1, wherein the target dose is at least about 8 mg.

38. The method of claim 1, wherein the target dose is at least about 9 mg.

39. The method of claim 1, wherein the target dose is about 450 μg.

40. The method of claim 1, wherein the target dose is about 750 μg.

41. The method of claim 1, wherein the target dose is about 1 mg.

42. The method of claim 1, wherein the target dose is about 1.5 mg.

43. The method of claim 1, wherein the target dose is about 2 mg.

44. The method of claim 1, wherein the target dose is about 2.5 mg.

45. The method of claim 1, wherein the target dose is about 3 mg.

46. The method of claim 1, wherein the target dose is about 4 mg.

47. The method of claim 1, wherein the target dose is about 5 mg.

48. The method of claim 1, wherein the target dose is about 6 mg.

49. The method of claim 1, wherein the target dose is about 7 mg.

50. The method of claim 1, wherein the target dose is about 8 mg.

51. The method of claim 1, wherein the target dose is about 9 mg.

52. The method of claim 1, wherein the target dose is about 10 mg.

53. The method of claim 1, further comprising administering to the subject a second dose of the isolated recombinant polypeptide complex, wherein the second dose is higher than the first dose and lower than the target dose, and wherein the second dose is administered between the first dose and the target dose.

54. The method of claim 53, wherein the second dose is about 300 μg to about 5 mg.

55. The method of claim 53, wherein the second dose is at least about 300 μg.

56. The method of claim 53, wherein the second dose is at least about 400 μg.

57. The method of claim 53, wherein the second dose is at least about 500 μg.

58. The method of claim 53, wherein the second dose is at least about 600 μg.

59. The method of claim 53, wherein the second dose is at least about 700 μg.

60. The method of claim 53, wherein the second dose is at least about 800 μg.

61. The method of claim 53, wherein the second dose is at least about 900 μg.

62. The method of claim 53, wherein the second dose is at least about 1 mg.

63. The method of claim 53, wherein the second dose is at least about 2 mg.

64. The method of claim 53, wherein the second dose is at least about 3 mg.

65. The method of claim 53, wherein the second dose is at least about 4 mg.

66. The method of claim 53, wherein the second dose is about 300 μg.

67. The method of claim 53, wherein the second dose is about 400 μg.

68. The method of claim 53, wherein the second dose is about 500 μg.

69. The method of claim 53, wherein the second dose is about 600 μg.

70. The method of claim 53, wherein the second dose is about 700 μg.

71. The method of claim 53, wherein the second dose is about 800 μg.

72. The method of claim 53, wherein the second dose is about 900 μg.

73. The method of claim 53, wherein the second dose is about 1 mg.

74. The method of claim 53, wherein the second dose is about 1.2 mg.

75. The method of claim 53, wherein the second dose is about 1.5 mg.

76. The method of claim 53, wherein the second dose is about 2 mg.

77. The method of claim 53, wherein the second dose is about 2.5 mg.

78. The method of claim 53, wherein the second dose is about 3 mg.

79. The method of claim 53, wherein the second dose is about 3.5 mg.

80. The method of claim 53, wherein, the second dose is about 4 mg.

81. The method of claim 53, wherein the second dose is about 4.5 mg.

82. The method of claim 53, wherein the second dose is about 5 mg.

83. The method of claim 53, wherein the first dose, the second dose, and the target dose are about 100 μg, 300 μg, and 450 μg, respectively.

84. The method of claim 53, wherein the first dose, the second dose, and the target dose are about 100 μg, 300 μg, and 1 mg, respectively.

85. The method of claim 53, wherein the first dose, the second dose, and the target dose are about 200 μg, 600 μg, and 2 mg, respectively.

86. The method of claim 53, wherein the first dose, the second dose, and the target dose are about 300 μg, 1 mg, and 3 mg, respectively.

87. The method of claim 53, wherein the first dose, the second dose, and the target dose are about 400 μg, 1.2 mg, and 4 mg, respectively.

88. The method of claim 53, wherein the first dose, the second dose, and the target dose are about 500 μg, 1.5 mg, and 5 mg, respectively.

89. The method of claim 53, wherein the first dose, the second dose, and the target dose are about 600 μg, 2 mg, and 6 mg, respectively.

90. The method of claim 53, wherein the first dose, the second dose, and the target dose are about 700 μg, 2.5 mg, and 7 mg, respectively.

91. The method of claim 53, wherein the first dose, the second dose, and the target dose are about 800 μg, 3 mg, and 8 mg, respectively.

92. The method of claim 53, wherein the first dose, the second dose, and the target dose are about 900 μg, 4 mg, and 9 mg, respectively.

93. The method of claim 53, wherein the first dose, the second dose, and the target dose are about 1 mg, 5 mg, and 10 mg, respectively.

94. The method of claim 53, wherein the first dose, the second dose, or the target dose is administered weekly.

95. The method of claim 53, wherein the first dose, the second dose, or the target dose is administered once every two weeks.

96. The method of claim 53, wherein the first dose, the second dose, or the target dose is administered once every three weeks.

97. The method of claim 53, wherein the first dose, the second dose, and the target dose are administered weekly.

98. The method of claim 53, wherein the first dose and the second dose are administered weekly, and wherein the target dose is administered once every two weeks.

99. The method of claim 53, wherein the first dose and the second dose are administered weekly, and wherein the target dose is administered once every three weeks.

100. The method of claim 53, wherein the method comprises a treatment cycle that starts on day 1.

101. The method of claim 100, wherein the treatment cycle is 21 days or 28 days.

102. The method of claim 100, wherein the first dose, the second dose, and the target dose are administered on day 1, one of day 7, 8 or 9, and one of day 14, 15, or 16, respectively, of the treatment cycle.

103. The method of claim 100, wherein the first dose, the second dose, and the target dose are administered on day 1, day 4, and day 8, respectively, of the treatment cycle.

104. The method of claim 102, wherein the target dose is administered weekly after the one of day 14, 15, or 16, of the treatment cycle.

105. The method of claim 102, wherein the target dose is administered once every two weeks after the one of day 14, 15, or 16, of the treatment cycle.

106. The method of claim 102, wherein the target dose is administered once every three weeks after the one of day 14, 15, or 16, of the treatment cycle.

107. The method of claim 103, wherein the target dose is administered weekly after day 8 of the treatment cycle.

108. The method of claim 103, wherein the target dose is administered once every two weeks after day 8 of the treatment cycle.

109. The method of claim 103, wherein the target dose is administered once every three weeks after day 8 of the treatment cycle.

110. The method of claim 1, wherein the target dose is administered at least once, at least twice, at least 3 times, at least 4 times, at least 5 times, at least 6 times, at least 7 times, at least 8 times, at least 9 times, at least 10 times, at least 15 times, at least 20 times, at least 25 times, at least 30 times, at least 35 times, at least 40 times, at least 45 times, at least 50 times, or more than 50 times.

111. The method of claim 100, further comprising at least 2 treatment cycles, at least 3 treatment cycles, at least 4 treatment cycles, at least 5 treatment cycles, at least 6 treatment cycles, at least 7 treatment cycles, at least 8 treatment cycles, at least 9 treatment cycles, at least 10 treatment cycles, or more than 10 treatment cycles.

112. The method of claim 100, wherein the first dose, the second dose, and the target dose are administered on day 1, the one of day 7, 8 or 9, and the one of day 14, 15, or 16, respectively, of the treatment cycle, and wherein the target dose is administered weekly after day 14, 15, or 16 of the treatment cycle.

113. The method of claim 100, wherein the first dose, the second dose, and the target dose are administered on day 1, the one of day 7, 8 or 9, and the one of day 14, 15, or 16, respectively, of the treatment cycle, and wherein the target dose is administered once every two weeks after day 14, 15, or 16 of the treatment cycle.

114. The method of claim 100, wherein the first dose, the second dose, and the target dose are administered on day 1, the one of day 7, 8 or 9, and the one of day 14, 15, or 16, respectively, of the treatment cycle, and wherein the target dose is administered once every three weeks after day 14, 15, or 16 of the treatment cycle.

115. The method of claim 100, wherein the first dose, the second dose, and the target dose are administered on day 1, day 4, and day 8, respectively, of the treatment cycle, and wherein the target dose is administered weekly after day 8 of the treatment cycle.

116. The method of claim 100, wherein the first dose, the second dose, and the target dose are administered on day 1, day 4, and day 8, respectively, of the treatment cycle, and wherein the target dose is administered once every two weeks after day 8 of the treatment cycle.

117. The method of claim 100, wherein the first dose, the second dose, and the target dose are administered on day 1, day 4, and day 8, respectively, of the treatment cycle, and wherein the target dose is administered once every three weeks after day 8 of the treatment cycle.

118. The method of claim 1, wherein the cancer comprises a cell that expresses or overexpresses prostate specific membrane antigen (PSMA).

119. The method of claim 1, wherein the cancer comprises cells that are dependent on endothelial cells of neovasculature that express or overexpress PSMA.

120. The method of claim 1, wherein the cancer comprises prostate cancer, lung cancer, breast cancer, colorectal cancer, or renal cell carcinoma.

121. The method of claim 1, wherein the cancer comprises prostate cancer.

122. The method of claim 1, wherein the cancer comprises metastatic castration resistant prostate cancer (mCRPC).

123. The method of claim 122, wherein the progression of mCRPC is documented according to guidelines of the Prostate Cancer Clinical Trials Working Group 3 (PCWG3), Response Evaluation Criteria in Solid Tumors 1.1 (RECIST 1.1), or both.

124. The method of claim 1, wherein the cancer comprises non-small cell lung cancer (NSCLC), or small cell lung cancer (SCLC).

125. The method of claim 1, wherein the subject is a male of at least 18 years old.

126. The method of claim 1, wherein the subject is a male of about 46 to about 85 years old.

127. The method of claim 1, wherein the subject is a male of about 69 years old.

128. The method of claim 1, wherein the subject is treated with a prior cancer therapy before the administering.

129. The method of claim 128, wherein the prior cancer therapy comprises 2 to 6 cancer therapies.

130. The method of claim 128, wherein the prior cancer therapy comprises about 4 cancer therapies.

131. The method of claim 128, wherein the prior cancer therapy comprises a taxane, a radioligand therapy, an anti-androgen therapy, an androgen receptor pathway inhibitor therapy, a poly-adenosine diphosphate ribose polymerase (PARP) inhibitor, or a combination thereof.

132. The method of claim 131, wherein the anti-androgen therapy comprises a radioligand therapy that targets PSMA.

133. The method of claim 128, wherein the prior cancer therapy comprises a taxane and an anti-androgen therapy.

134. The method of claim 133, wherein the cancer is mCRPC, and wherein the mCRPC progresses after the prior cancer therapy.

135. The method of claim 128, wherein the prior cancer therapy comprises no taxane.

136. The method of claim 1, wherein the subject has adequate organ function.

137. The method of claim 1, wherein the subject has resolving acute effects of any prior therapy to baseline severity of Common Terminology Criteria for Adverse Events (CTCAE) grade of no more than 1.

138. The method of claim 1, wherein the subject has adenocarcinoma of the prostate that is confirmed histologically or cytologically.

139. The method of claim 1, wherein the subject is positive for a baseline prostate-specific membrane antigen positron emission tomography (PSMA-PET).

140. The method of claim 1, wherein the subject has a baseline PSA level of about 1 ng/ml to about 10000 ng/ml.

141. The method of claim 1 wherein the subject has a baseline PSA level of about 158 ng/ml.

142. The method of claim 1, wherein the subject is or is not evaluable according to guidelines of response evaluation criteria in solid tumors 1.1 (RECIST 1.1) or PCWG3.

143. The method of claim 1, wherein the subject has or does not have a bone metastasis, a lymph node metastasis, or a visceral metastasis.

144. The method of claim 143, wherein the visceral metastasis comprises a live metastasis, a lung metastasis, an adrenal metastasis, an abdominal metastasis, or a retroperitoneal metastasis, or a combination thereof.

145. The method of claim 1, wherein the cancer does not respond to a treatment with a radioligand therapy that targets PSMA before the administering.

146. The method of claim 1, wherein the cancer responds to a treatment with a radioligand therapy that targets PSMA before the administering.

147. The method of claim 146, wherein the treatment comprises lutetium Lu 177 vipivotide tetraxetan.

148. The method of claim 1, wherein the subject does not have a prior solid organ transplant procedure before the administering.

149. The method of claim 1, wherein the subject is not treated with a chimeric antigen receptor T (CAR-T) cell therapy that targets PSMA or a T cell engager therapy that targets PSMA, before the administering.

150. The method of claim 1, wherein the subject does not have clinically significant cardiovascular disease.

151. The method of claim 1, wherein the subject does not have an active and clinically significant infection.

152. The method of claim 151, wherein the infection comprises a bacterial infection, a viral infection, a fungal infection, a mycobacterial infection, or a combination thereof.

153. The method of claim 1, wherein the administering comprises administering intravenously.

154. The method of claim 1, wherein the subject exhibits a cytokine release syndrome (CRS) no more than grade 2.

155. The method of claim 1, wherein the subject exhibits no treatment related adverse event (TRAE) that is related to a CRS.

156. The method of claim 1, wherein the administering comprises administering to the subject a second treatment.

157. The method of claim 156, wherein the second treatment comprises lutetium Lu 177 vipivotide tetraxetan, enzalutamide, abiraterone acetate (ABI), chemotherapy, radium Ra 223 dichloride, a poly-adenosine diphosphate ribose polymerase inhibitor (PARPI), pembrolizumab, or a combination thereof.

158. The method of claim 157, wherein the second treatment comprises enzalutamide.

159. The method of claim 158, wherein the administering results in a synergistic effect on the cancer compared with administering the isolated recombinant polypeptide complex alone or administering enzalutamide alone.

160. The method of claim 159, wherein the synergistic effect comprises a therapeutic effect that is better than a therapeutic effect resulting from administering the isolated recombinant polypeptide complex alone or a therapeutic effect resulting from administering enzalutamide alone.

161. The method of claim 159, wherein the cancer is refractory, non-responsive, or resistant, to treatment with enzalutamide alone before the administering.

162. The method of claim 157, wherein the PARPI comprises olaparib, rucaparib, niraparib, talazoparib, or a combination thereof.

163. The method of claim 1, wherein the administering results in a therapeutic effect in prostate specific antigen (PSA) response, best RECIST response, best response by PCWG3 criteria, best PET response, radiographic progression free survival (rPFS), overall response rate, overall survival, bone pain, tumor size, or a combination thereof, after the administering.

164. The method of claim 163, wherein the subject exhibits a reduction of at least about 30% in PSA level after the administering.

165. The method of claim 163, wherein the subject exhibits a reduction of at least about 50% in PSA level after the administering.

166. The method of claim 163, wherein the subject exhibits a reduction of at least about 70% in PSA level after the administering.

167. The method of claim 163, wherein the subject exhibits a reduction of at least about 90% in PSA level after the administering.

168. The method of claim 163, wherein the subject exhibits a reduction of about 30% to about 100% in PSA level after the administering.

169. The method of claim 163, wherein the subject exhibits a higher reduction in PSA level when the first dose increases.

170. The method of claim 53, wherein the subject exhibits a higher reduction in PSA level when the second dose increases.

171. The method of claim 163, wherein the subject exhibits a higher reduction in PSA level when the target dose increases.

172. The method of claim 163, wherein the subject exhibits a higher reduction in PSA level when the first dose is at least about 200 μg compared with when the first dose is at least about 100 μg.

173. The method of claim 163, wherein the subject exhibits a reduction of about 30% to about 100% in PSA level at about 2 weeks after the administering of at least 200 μg of the first dose.

174. The method of claim 173, wherein the reduction in PSA level continues throughout the treatment with the method.

175. The method of claim 163 wherein the subject exhibits a better RECIST response when the first dose increases.

176. The method of claim 53, wherein the subject exhibits a better RECIST response when the second dose increases.

177. The method of claim 163, wherein the subject exhibits a better RECIST response when the target dose increases.

178. The method of claim 163, wherein the subject exhibits a better RECIST response when the first dose is at least about 200 μg compared with when the first dose is at least about 100 μg.

179. The method of claim 163, wherein a reduction in PSA level correlates with an occurrence of a CRS.

180. The method of claim 163, wherein the subject exhibits a reduction of about 0% to about 100% in tumor size after the administering.

181. The method of claim 163, wherein the subject exhibits a reduction of about 5% to about 75% in tumor size after the administering.

182. The method of claim 163, wherein the subject exhibits a reduction in bone skeletal lesion measured by PCWG3 criteria after the administering.

183. The method of claim 163, wherein the change in tumor size is measured by RECIST 1.1.

184. The method of claim 163, wherein the change in PSMA level is measured by standardized uptake value (SUV) by PSMA-PET.

185. The method of claim 163, wherein the subject exhibits a RECIST partial response when the first dose is at least about 100 μg, at least about 200 μg, at least about 300 μg, at least about 400 μg, or at least about 500 μg.

186. The method of claim 163, wherein the subject exhibits a RECIST partial response when the target dose is at least about 100 μg to at least about 10 mg.

187. The method of claim 163, wherein the subject exhibits a reduction of 0%-100% in SUV measured by PSMA-PET.

188. The method of claim 163, wherein the subject exhibits a reduction of about 72% in SUV measured by PSMA-PET.

189. The method of claim 163, wherein the subject exhibits about 80% in best PSA reduction.

190. The method of claim 163, wherein the subject exhibits a reduction in pain related to cancer.

191. The method of claim 190, wherein the subject exhibits a reduction in bone pain.

192. The method of claim 163, wherein the subject exhibits a best PET response.

193. The method of claim 163, wherein the subject exhibits a best RECIST response or a best response measured by PCWG3.

194. The method of claim 1, wherein the method further comprises treating the subject with a therapy for CRS, diarrhea, chills, alanine transaminase (ALT) increase, anaemia, aspartate aminotransferase (AS) increase, fatigue, decreased appetite, nausea, headache, blood bilirubin increase, hypoalbuminaemia, hypocalcaemia, hypophosphataemia, leukopenia, white blood cell count decrease, myalgia, platelet count decrease, thrombocytopenia, pyrexia, vomiting, blood alkaline phosphatase increase, dysgeusia, hypomagnesaemia, lipase increase, stomatitis, or a combination thereof.

195. The method of claim 53, wherein the isolated recombinant polypeptide complex is cleaved by a cancer specific protease to generate an active T cell engager and a cleavage fragment after the administering.

196. The method of claim 1, wherein the administering provides a plasma concentration of the isolated recombinant polypeptide complex that is below preclinical activity threshold.

197. The method of claim 195, wherein the administering provides a plasma concentration of the active T cell engager that is below preclinical activity threshold.

198. The method of claim 1, wherein the administering of at least one dose of about 100 μg of the isolated recombinant polypeptide complex provides a maximum plasma concentration (Cmax) of the isolated recombinant polypeptide complex in an amount up to about 34 ng/ml.

199. The method of claim 1, wherein the administering of at least one dose of about 300 μg of the isolated recombinant polypeptide complex provides a Cmax of the isolated recombinant polypeptide complex in an amount up to about 175 ng/ml.

200. The method of claim 1, wherein the administering of at least one dose of about 450 μg of the isolated recombinant polypeptide complex provides a Cmax of the isolated recombinant polypeptide complex in an amount of up to about 131 ng/ml.

201. The method of claim 1, wherein the administering of at least one dose of about 1 mg of the isolated recombinant polypeptide complex provides a Cmax of the isolated recombinant polypeptide complex in an amount up to about 283 ng/ml.

202. The method of claim 1, wherein the administering provides a Cmax of the isolated recombinant polypeptide complex in an amount that correlates with a dose being administered.

203. The method of claim 201, wherein the Cmax of the isolated recombinant polypeptide complex increases when the dose being administered increases.

204. The method of claim 1, wherein the administering provides a Cmax of the isolated recombinant polypeptide complex in an amount that correlates with the number of doses that the subject has received.

205. The method of claim 204, wherein the Cmax of the isolated recombinant polypeptide complex increases when the number of doses that the subject has received increases.

206. The method of claim 195, wherein the administering provides a Cmax of the cleavage fragment in an amount up to about 4.5 ng/ml, and wherein the first dose, the second dose and the target dose being administered are about 100 μg, about 300 μg, and about 450 μg, respectively.

207. The method of claim 195, wherein the administering provides a Cmax of the cleavage fragment in an amount up to about 14 ng/ml, and wherein the first dose, the second dose and the target dose being administered are about 100 μg, about 300 μg, and about 1 mg, respectively.

208. The method of claim 196, wherein the administering provides a Cmax of the cleavage fragment in an amount that correlates with the target dose being administered.

209. The method of claim 208, wherein the Cmax of the cleavage fragment increases when the target dose being administered increases.

210. The method of claim 195, wherein the administering provides a Cmax of the cleavage fragment in an amount that correlates with the number of doses that the subject has received.

211. The method of claim 210, wherein the Cmax of the cleavage fragment increases when the number of doses that the subject has received increase.

212. The method of claim 131, wherein the prior cancer therapy comprises lutetium Lu 177 vipivotide tetraxetan.

213. The method of claim 131, wherein the prior cancer therapy comprises olaparib, rucaparib, niraparib, talazoparib, or a combination thereof.

214. The method of claim 1, wherein the subject has a Homologous Recombination Repair (HRR) gene mutation, a BReast Cancer (BRCA) gene mutation, a BRCA1 gene mutation, a BRCA2 gene mutation, a homeobox B13 (HOXB13) gene mutation, an ataxia telangiectasia mutated (ATM) gene mutation, or a combination thereof.
